(19) United States
(12) Patent Application Publication
(10) Pub. No.: US 2026/0275334 A1
(43) Pub. Date: Sep. 17, 2026

(54) COMPOSITIONS AND METHODS FOR TREATMENT OF MONOGENIC NEURODEVELOPMENTAL DISORDER

(71) Applicant: PYC Therapeutics Limited, Nedlands, Western Australia (AU)

(72) Inventors: Ianthe Pitout, Nedlands (AU); Rebecca Simmons, Nedlands (AU); Susan Fletcher, Nedlands (AU)

(21) Appl. No.: 18/868,554

(22) PCT Filed: Jun. 5, 2023
(86) PCT No.: PCT/AU2023/050486

§ 371 Date: Nov. 22, 2024

(30) Foreign Application Priority Data
Jun. 7, 2022 (AU) 2022901557
Sep. 26, 2022 (AU) 2022902778
Oct. 17, 2022 (AU) 2022903040

(51) Int. Cl. C12N 15/113; A61K 31/712; A61K 31/7125; A61K 47/62; A61P 25/28

(52) U.S. Cl. CPC C12N 15/113; A61K 31/712; A61K 31/7125; A61K 47/62; A61P 25/28; C12N 2310/11; C12N 2310/315; C12N 2310/321; C12N 2310/3513

(57) ABSTRACT

Described herein are antisense oligonucleotides, vectors, and related compositions and methods for increasing endoge-nous expression of SHANK3 protein and uses thereof for conditions associated with SHANK3 haploinsufficiency such as Phelan-McDermid syndrome.

The present application claims priority from AU 2022901557 filed on 7 Jun. 2022, AU 2022902778 filed on 26 Sep. 2022, and AU 2022903040 filed on 17 Oct. 2022, the entire contents of each of which are incorporated herein by reference.

### TECHNICAL FIELD

The present disclosure generally is directed to oligonucleotides and related compositions and methods for treating conditions associated with mutations in the SHANK3 gene.

### BACKGROUND

SHANK3 is a widely expressed scaffolding protein enriched in the post-synaptic density of excitatory synapses in the brain. SHANK3 recruits and stabilizes ionotropic and metabotropic glutamate receptors (AMPA, NMDA, mGluR) to the post-synaptic density. SHANK3 gene mutations/deletions and SHANK3 haploinsufficiency underlie the rare genetic neurodevelopmental disorder, Phelan-McDermid syndrome, and have been attributed to 0.5%-1% of autism spectrum disorders (ASD), 2% of intellectual disability diagnoses, and 0.6%-2.16% of atypical schizophrenia diagnoses.

Phelan-McDermid syndrome is characterized by intellectual disability of varying degrees, neonatal hypotonia, absent to severely delayed speech development, moderate to profound developmental delay, motor regression and minor dysmorphic features. Approximately 14-70% of affected individuals develop seizures ranging from mild to severe. Other complications include kidney abnormalities, gastrointestinal issues, reduced perspiration and risk of overheating, lack of perception of pain, arachnoid cysts, or other comorbid neuropsychiatric illnesses. Children are typically diagnosed in early childhood, often due to significant delays in reaching early developmental milestones. There is currently no effective treatment for conditions caused by SHANK3 haploinsufficiency such as Phelan-McDermid syndrome. Thus, there is an ongoing need to provide effective compositions and methods for treating such conditions.

### SUMMARY

The SHANK3 gene includes 22 exons spanning 58 kb of genomic DNA on the terminal end of chromosome 22 (22q13 region) and its major protein product is a 1,607 amino acid polypeptide. There are at least six known isoforms that are temporally and spatially specific and have distinct functions at the synapse. SHANK3 contains five protein-protein interaction domains, and each isoform contains distinct combinations of these five domains.

While not wishing to be bound by theory, SHANK3 haploinsufficiency due to loss of function gene mutation(s), including nonsense, missense and frameshift mutations, as well as partial or whole gene deletions, results in insufficient protein production.

The present disclosure provides antisense oligonucleotides (ASO), antisense RNA (AR) expression vectors, and related compositions and methods to increase SHANK3 protein levels by modulating the translational efficiency or the stability of SHANK3 mRNA to increase the level of SHANK3 mRNAs encoding functional SHANK3 isoforms. Also disclosed are methods for treating conditions associated with SHANK3 haploinsufficiency.

Accordingly, in one aspect provided herein is an antisense oligonucleotide that binds within a targeted portion of the:
(i) 5′ untranslated region (UTR) of a SHANK3 mRNA;
(ii) 5′-proximal non-coding region (PNCR) of a SHANK3 pre-mRNA; or
(iii) 3′ UTR of a SHANK3 mRNA;
whereby binding of the antisense oligonucleotide within the targeted portion of the mRNA or pre-mRNA in a mammalian cell results in an increased level of SHANK3 protein in the mammalian cell.

In a related aspect provided herein is vector for expression, in a mammalian neuron, of an antisense RNA (AR) that binds within a targeted portion of the:
(i) 5′ UTR of a SHANK3 mRNA;
(ii) 5′ PNCR of a SHANK3 pre-mRNA; or
(iii) 3′ UTR of a SHANK3 mRNA;
whereby binding of the AR within the targeted portion of the RNA in a mammalian cell results in an increased level of SHANK3 protein in the mammalian cell. In some examples the vector includes a neuron-selective promoter for driving expression of the AR in the mammalian neuron. In some examples the neuron-selective promoter is selective for expression in a neuron type selected from the list consisting of: cortical glutamatergic neurons, cortical GABAergic neurons, hippocampal glutamatergic neurons, and striatal inhibitory neurons. In some examples the vector includes an inducible promoter. In some examples the vector is a non-viral vector. In some examples a non-viral vector is provided as a composition comprising a transfection agent. In other examples the vector is a viral vector. In some examples, where the vector is a viral vector, the viral vector is a recombinant virus selected from the group consisting of: adeno-associated virus (AAV), adenovirus, lentivirus, and anellovirus.

In some examples the nucleotide sequence of the ASO or AR corresponds to any one of SEQ ID NOs: 293, 299, 301, 302, 304-309, 311, 313, 315, 318, 606, 797, 1193, 1195, 1847, 1934-1937, 2858, 2874, 3510, 12644, 12666, 12669, 12671, 12688, or 12690. In some examples of any of the ASOs, vectors, or compositions, the binding of the ASO or AR is within a targeted portion of the 5′ UTR corresponding to SEQ ID NO: 1. In other examples of any of the foregoing methods, ASOs, vectors, or compositions, the binding of the ASO or AR is within a targeted portion of the 5′ PNCR corresponding to SEQ ID NO:3. In other examples of any of the foregoing methods, ASOs, vectors, or compositions, the binding of the ASO or AR is within a targeted portion of the 3′ UTR corresponding to SEQ ID NO:2.

In some examples the nucleotide sequence of the ASO or AR is at least 60%, 65%, 70%, 75%, 80%, 85%, 90%, 95%, or 100% complementary to the nucleotide sequence of the targeted portion over the length of the ASO or the AR. In some examples the nucleotide sequence of the ASO or AR corresponds to any one of SEQ ID NOs: 5-622, 4175-4181, or 4184-4186. In some examples the nucleotide sequence of the ASO or AR corresponds to any one of SEQ ID NOs: 559, 606, or 4178-4181. In other examples the nucleotide sequence of the antisense oligonucleotide or the AR corresponds to any one of SEQ ID NOs: 1935-4168, 4182, 4183, 12646-12654, or 12664-12671. In some examples the nucleotide sequence of the ASO or AR corresponds to any one of SEQ ID NOs: 1935-1937, or 2849. In other examples the nucleotide sequence of the ASO or AR corresponds to any one of SEQ ID NOs: 623-1934, 4169-4174, 12645, 12655-12663, or 12688. In some examples the nucleotide sequence of the ASO or AR corresponds to any one of SEQ ID NOs: 1847, 1852, 1934, 12661-12663, or 12688.

In some examples any of the foregoing ASOs include a backbone modification. In some examples the backbone modification includes a phosphorothioate linkage or a phosphorodiamidate linkage. In other examples the ASO includes a phosphorodiamidate morpholino, a locked nucleic acid, a peptide nucleic acid, or a 2′-O-modification such as a 2′-O-methyl, a 2′-Fluoro, or a 2′-O-methoxyethyl moiety. In some examples the ASO includes at least one modified sugar moiety. In other examples each sugar moiety in the ASO is a modified sugar moiety. In some examples the ASO includes a 2′-O-methoxyethyl moiety. In other examples each nucleotide of the ASO includes a 2′-O-methoxyethyl moiety.

In some examples of any of the foregoing ASOs or vectors, the nucleotide sequence of the ASO or AR is 10 to 50 nucleotides, 15 to 40 nucleotides, 17 to 30 nucleotides, 18 to 40 nucleotides, 17 to 25 nucleotides, 20 to 35 nucleotides, 20 to 30 nucleotides, 22 to 30 nucleotides, 24 to 30 nucleotides, 25 to 30 nucleotides, or 26 to 30 nucleotides in length. In some examples where the ASO is 17 to 30 nucleotides in length, the ASO includes one or more phosphorodiamidate morpholino moieties.

In some examples any of the foregoing ASOs are linked to a functional moiety. In some examples the functional moiety includes a delivery moiety. In some examples the delivery moiety is selected from the group consisting of: lipids, polyethers, peptides, carbohydrates, receptor binding domains (RBDs), and antibodies. In some examples, wherein the ASO includes a delivery moiety, the delivery moiety includes a cell-penetrating peptide (CPP). In some examples the delivery moiety includes a N-acetylgalactosamine (GalNAc) or glycan moiety. In some examples the delivery moiety includes a fatty acid or lipid moiety. In some embodiments the fatty acid chain length is about C8 to C20. In other examples the functional moiety includes a stabilising moiety. In some examples the functional moiety is covalently linked to the ASO. In other examples the functional moiety is non-covalently linked to the ASO. In some examples the functional moiety is linked to the 5′ end of the ASO. In other examples the functional moiety is linked to the 3′ end of the ASO. In some examples any of the foregoing ASOs also include a delivery nanocarrier, wherein the nanocarrier is complexed with the ASO. In some examples the delivery nanocarrier is selected from the group consisting of: lipoplexes, liposomes, exosomes, inorganic nanoparticles, and DNA nanostructures. In some examples the delivery nanocarrier includes a lipid nanoparticle (LNP) encapsulating the ASO.

In a related aspect provided herein is a pharmaceutical composition that includes any of the foregoing ASOs, vectors, or compositions and a pharmaceutically acceptable excipient.

In a further related aspect provided herein is a method for preventing or treating a condition associated with SHANK3 haploinsufficiency, the method comprising administering to a subject in need thereof a therapeutically effective amount of the foregoing pharmaceutical composition. In some examples the condition to be treated is Phelan-McDermid syndrome, an autism spectrum disorder, schizophrenia, or an intellectual disability. In some examples the condition to be treated is Phelan-McDermid syndrome. In some examples the subject to be treated is a human subject.

In a further aspect provided herein is the use of any of the foregoing antisense oligonucleotides, vectors, or compositions in the manufacture of a medicament for prevention or treatment of a condition associated with SHANK3 haploinsufficiency.

In some examples of the foregoing methods of treatment or uses, the level of SHANK3 protein in at least a plurality of cells in the subject is increased about 1.1 to about 5 fold in cells (e.g., neurons) in vitro or ex vivo, e.g., 1.2 fold, 1.3 fold, 1.5 fold, 1.7 fold, 2 fold, 2.2 fold, 2.5 fold, 2.7 fold, 3 fold, 3.3 fold, 3.5 fold, 4 fold, 4.3 fold, 4.5 fold, 4.7 fold, or another increase in SHANK3 protein levels from about 1.1 fold to about 5 fold in cells in a subject compared to the level in the absence of the pharmaceutical 25 composition.

In yet another aspect provided herein is a genetically modified cell comprising any of the foregoing ASOs or vectors. In some examples the genetically modified cell is a mammalian cell. In some examples the genetically modified mammalian cell is a human cell. In some examples the genetically modified mammalian cell is a neuron or a neural progenitor. In some examples, the genetically modified mammalian cell is a neuron selected from the group consisting of: cortical glutamatergic neurons, cortical GABAergic neurons, hippocampal glutamatergic neurons, and striatal inhibitory neurons. In some examples the genetically modified mammalian cell is from a cell line. In some examples the cell line is a human induced pluripotent stem cell (hiPSC) line or a cell line derived from neurons.

It is also known that SHANK3 mRNA is subject to alternative splicing, which can have an impact on the overall level of canonical SHANK3 mRNA and protein levels. The term “alternative splicing” refers to a process whereby exons, or part of an exon of a gene, or introns or part of an intron, may be included within or excluded from the final mRNA transcript. Mature noncanonical mRNA transcripts can be non-productive due to a frame shift that may induce degradation of the transcript via the nonsense mediated decay pathway. In other cases, translation of the noncanonical mRNA can produce a truncated but non-functional protein. Alternative splicing of the SHANK3 pre-RNA transcripts can downregulate overall SHANK3 mRNA and protein expression.

Introns are removed from maturing transcripts by a large RNA-protein complex termed the spliceosome, which orchestrates complex interactions between primary transcripts, small nuclear RNAs (snRNAs) and a large number of proteins. Spliceosomes assemble on each intron in an ordered manner, starting with recognition of the 5′ splice site (5 ‘ss) by U1 snRNA or the 3’ splice site (3′ ss) by the U2 pathway, which involves binding of the U2 auxiliary factor (U2AF) to the 3′ ss region to facilitate U2 binding to the branch point sequence (BPS). U2AF is a stable heterodimer composed of a U2AF2-encoded 65-kD subunit (U2AF65), which binds the polypyrimidine tract (PPT), and a U2AF1-encoded 35-kD subunit (U2AF35), which interacts with highly conserved AG dinucleotides at 3′ ss and stabilizes U2AF65 binding. In addition to the BPS/PPT unit and 3′ ss/5′ ss, accurate splicing requires auxiliary sequences or structures that activate or repress splice site recognition, known as intronic or exonic splicing enhancers or silencers. These elements allow genuine splice sites to be distinguished from cryptic or pseudo-splice sites, which have the same sequence motifs as authentic sites but outnumber them by an order of magnitude in the genomes of higher eukaryotes.

Accordingly, in a different aspect provided herein is a method for increasing the amount of functional SHANK3 protein in a mammalian cell expressing a SHANK3 pre-mRNA, the method comprising contacting the cell with an antisense oligonucleotide or antisense RNA that binds to a targeted portion of the SHANK3 pre-mRNA to modulate splicing of a SHANK3 mRNA derived therefrom, whereby the level of SHANK3 mRNA encoding full length, functional SHANK3 protein is increased in the mammalian cell.

In a further aspect provided herein is antisense oligonucleotide that binds to a targeted portion of a SHANK3 pre-mRNA to modulate splicing of the SHANK3 mRNA, whereby the level of SHANK3 mRNA encoding full length, functional SHANK3 is increased.

In a related aspect provided herein is a vector for expression, in a mammalian neuron, of an antisense RNA (AR) that binds within a targeted portion of a SHANK3 pre-mRNA to modulate splicing of the SHANK3 mRNA, whereby the level of SHANK3 mRNA encoding full length, functional SHANK3 is increased. In some examples the nucleotide sequence of the ASO or AR used to modulate splicing corresponds to any one of SEQ ID NOs: 4187-12693 or 12695-3953.

### BRIEF DESCRIPTION OF THE ACCOMPANYING DRAWINGS

FIG. 1—A set of exemplary SHANK3 PMOs induce variable SHANK3 protein upregulation

Normalized fold-change in expression of SHANK3 protein assessed by western blotting. SHANK3 protein expression is shown relative to the level in transfection control cells (no ASO transfection control). SH-SY5Y cells transfected with PMOs 4-10 (SEQ ID NOs: 4169, 4170, 1934 and 4171-4174 respectively) and 19-37 (SEQ ID NOs: 4184-4186, 4175, 559, 4176, 4177, 598, 4178-4183, 606, 1935, 1936, 2849 and 1937 respectively) at 25 μM and 50 μM. SHANK3 protein (190 kDa) is normalized to the total amount of protein loaded. Data are presented as mean (n=3-6 biological replicates, with 1-2 technical replicates per experiment).

FIG. 2—A subset of exemplary SHANK3 PMOs induce robust upregulation of SHANK3 protein

Normalized fold-change in expression of SHANK3 protein assessed by western blotting. SHANK3 protein expression is shown relative to the level in transfection control cells (no ASO transfection control). SH-SY5Y cells transfected with PMOs 6 (SEQ ID NO: 1934), 33-35 (SEQ ID NOs: 606, 1935 and 1936, respectively), 37 (SEQ ID NOs: 1937), at 25 μM and 50 μM demonstrate upregulation of SHANK3 protein, relative to transfection control SH-SY5Y cells (no ASO transfection control). Data are presented as mean±SEM (n=3-6 biological replicates, with 1-2 technical replicates per experiment). Statistical significance calculated as two-way unpaired t-test between treatment and transfection control cells (no ASO transfection control); *: p≤0.05, **: p≤0.01.

FIG. 3—Screening of PMOs targeting the 5′ UTR, 3′ UTR, or retained introns of SHANK3 in human neuroblastoma cell line

SH-SY5Y cells were transfected with PMOs targeting the 5′ UTR, 3′ UTR, or retained introns of SHANK3 at 25 μM and 50 μM via electroporation (NEON, ThermoFisher Scientific), as per the manufacturer's instructions. Total protein was harvested (96 hours) from transfected cells using 15% SDS lysis buffer. SHANK3 protein (190 kDa) expression was analysed by western blotting using rabbit-SHANK3 polyclonal antibody (Bethyl Labs, BLA304178A-T) at 1:10 000 in 5% BSA in TBST. Experimental controls are identified as TFC (transfection control, no ASO), UTC (untreated control), +control (rat cerebellum lysate positive control and NTC (non-targeted negative controls (Smnl and scrambled PMO). Housekeepers used for normalization include Vinculin (116 kDa; mouse-monoclonal antibody at 1:200 in 5% BSA in TBST, Sigma Aldrich, V9131) and Beclin1 (60 kDa; rabbit-polyclonal antibody at 1:2 000 in 5% BSA in TBST, Cell Signalling Technology, S3495). Blots are representative of n=2-5 biological replicates. Corresponding SEQ ID NOs through (A) and (B) are indicated above each lane.

FIG. 4—A set of exemplary SHANK3 PMOs induce variable SHANK3 protein upregulation by targeting the 5′ UTR and putative extended 5′ UTR of SHANK3 transcripts

SH-SY5Y cells were transfected with PMOs targeting the 5′ UTR and putative extended 5′ UTR of SHANK3 at 25 μM and 50 μM via electroporation (NEON, ThermoFisher Scientific). Normalized fold-change in expression of SHANK3 protein was assessed by western blotting. SHANK3 protein expression is shown relative to the level in transfection control cells (no PMO, zap). Data are presented as mean±SEM (n=2-5 biological replicates). ASOs (other than positive controls) are identified by their SEQ ID NO and are listed in 5′-3′ target site order, as schematically illustrated in FIG. 14. Control treatments are identified as NTC (Non-targeted control PMO), TFC (transfection control) and UTC (untreated control). SHANK3 protein (190 kDa) is normalized to the total amount of protein loaded.

FIG. 5—A set of exemplary SHANK3 PMOs induce variable SHANK3 protein upregulation by targeting the 3′ UTR of SHANK3 transcripts

SH-SY5Y cells were transfected with PMOs targeting the 3′ UTR of SHANK3 at 25 μM and 50 μM via electroporation (NEON, ThermoFisher Scientific). Normalized fold-change in expression of SHANK3 protein was assessed by western blotting. SHANK3 protein expression is shown relative to the level in transfection control cells (no PMO, zap). Data are presented as mean±SEM (n=2-5 biological replicates). ASOs (other than positive controls) are identified by their SEQ ID NO and are listed in 5′-3′ target site order, as schematically illustrated in FIG. 17. Control treatments are identified as NTC (Non-targeted control PMO), TFC (transfection control) and UTC (untreated control). SHANK3 protein (190 kDa) is normalized to the total amount of protein loaded.

FIG. 6—A subset of exemplary SHANK3 PMOs induce robust upregulation of SHANK3 protein by targeting the 5′ and 3′ UTRs of SHANK3 transcripts

SH-SY5Y cells were transfected with PMOs targeting the 5′ and 3′ UTR of SHANK3 at 25 μM and 50 μM via electroporation (NEON, ThermoFisher Scientific). Normalized fold-change in expression of SHANK3 protein was assessed by western blotting. SHANK3 protein expression is shown relative to the level in transfection control cells (no PMO, zap). Data are presented as mean±SEM (n=2-5 biological replicates). ASOs (other than positive controls) are identified by their SEQ ID NO and are listed in 5′-3′ target site order, as diagrammed in FIGS. 14 and 17, and control treatments are identified as NTC (Non-targeted control PMO), TFC (transfection control) and UTC (untreated control). SHANK3 protein (190 kDa) is normalized to the total amount of protein loaded.

FIG. 7—A set of exemplary SHANK3 PMOs induce variable SHANK3 protein upregulation by modulating splicing of SHANK3 transcripts

SH-SY5Y cells were transfected with PMOs that modulate alternative splicing of SHANK3 at 25 μM and 50 μM via electroporation (NEON, ThermoFisher Scientific). Normalized fold-change in expression of SHANK3 protein was assessed by western blotting. SHANK3 protein expression is shown relative to the level in transfection control cells (no PMO, zap). Data are presented as mean±SEM (n=2-5 biological replicates). ASOs (other than positive controls) are identified by their SEQ ID NO and are listed in 5′-3′ target site order, as diagrammed in FIGS. 15 and 16. Control treatments are identified as NTC (Non-targeted control PMO), TFC (transfection control) and UTC (untreated control). SHANK3 protein (190 kDa) is normalized to the total amount of protein loaded.

FIG. 8—A subset of exemplary SHANK3 PMOs induce robust upregulation of SHANK3 protein by modulating splicing of SHANK3 transcripts

SH-SY5Y cells were transfected with PMOs that modulate alternative splicing of SHANK3 at 25 μM and 50 μM via electroporation (NEON, ThermoFisher Scientific). Normalized fold-change in expression of SHANK3 protein was assessed by western blotting. SHANK3 protein expression is shown relative to the level in transfection control cells (no PMO, zap). Data are presented as mean±SEM (n=2-5 biological replicates). ASOs (other than positive controls) are identified by their SEQ ID NO and are listed in 5′-3′ target site order, as illustrated in FIGS. 15 and 16. Control treatments are identified as NTC (Non-targeted control PMO), TFC (transfection control) and UTC (untreated control). SHANK3 protein (190 kDa) is normalized to the total amount of protein loaded.

FIG. 9—Screening of MOEs targeting the 5′ UTR or 3′ UTR of SHANK3 in human neuroblastoma cell line

A panel of MOEs targeting the 5′ UTR, 3′ UTR, or retained introns of SHANK3 were screened in SH-SY5Y cells via electroporation (NEON, ThermoFisher Scientific), as per the manufacturer's instructions. Protein was harvested (96 hours), using 15% SDS protein lysis buffer. SHANK3 (190 kDa) expression was assessed by western blot using rabbit-SHANK3 polyclonal antibody (Bethyl Labs, BLA304-178A-T) at 1:10,000 in 5% BSA in TBST buffer. Beclin1 (60 kDa) and Vinculin (116 kDa) were used as housekeeper proteins and were assessed using rabbit-Beclin1 monoclonal antibody at 1:2,000 in 5% BSA in TBST (Cell Signalling Technology, S3495) and mouse-Vinculin monoclonal antibody at 1:200 in 5% BSA in TBST (Sigma-Aldrich, V9131). Control treatments are identified as NTC (Non-targeted control PMO), TFC (transfection control), UTC (untreated control) and rat cerebellum lysate positive control (+control). SEQ ID NOs for each MOE are indicated above each lane. Blots are representative of n=1-3 biological replicates.

FIG. 10—A set of exemplary SHANK3 MOEs induce variable SHANK3 protein upregulation by targeting the 5′ UTR and putative extended 5′ UTR of SHANK3 transcripts

SH-SY5Y cells were transfected with 2.5 μM, 5.0 μM and/or 25 μM of MOE designed to target the 5′ UTR or putative extended 5′ UTR of SHANK3 via electroporation (NEON, ThermoFisher Scientific). SHANK3 protein levels were assessed by western blot and expression was normalized to a housekeeper (HK) protein, Beclin 1. Fold-change in SHANK3 expression was measured relative to the transfection control (TFC; no ASO). A subset of MOEs at 5 μM (SEQ ID NO: 3510, 299, 301, 302, 305, 306, 309 and 318) and 25 μM (SEQ ID NO: 293, 305, 306, 307, 308, 311, 313 and 315) induced protein upregulation ≥1.5-fold relative to the TFC. Data are presented as mean±SEM (n=1-3 biological replicates, with 1-2 technical replicates per experiment).

FIG. 11—A subset of exemplary SHANK3 MOEs induce variable upregulation of SHANK3 protein by targeting the 3′ UTR of SHANK3 transcripts.

SH-SY5Y cells were transfected with a panel of MOEs targeting the 3′ UTR of SHANK3 via electroporation (NEON, ThermoFisher Scientific). Normalized fold-change in expression of SHANK3 protein (at 96 hours) was assessed by western blotting. SHANK3 protein expression in transfected and un-transfected (UTC) cells is shown relative to the transfection control (TFC; no ASO). MOEs were tested at concentrations of 2.5 μM, 5.0 μM, or 25 μM, although not all concentrations were tested for each MOE. Among the sequences tested, one MOE (SEQ ID NO: 1193) induced upregulation of SHANK3 protein at both the lower (5.0 μM) and higher (25 μM) concentrations, relative to the TFC. A subset of MOEs (SEQ ID NOs: 797, 1194 and 1195) induced SHANK3 upregulation only when used at 25 μM. Data are presented as mean±SEM (n=1-3 biological replicates, with 1-2 technical replicates per experiment).

FIG. 12—Screening of an expanded panel of SHANK3 MOEs targeting the 5′ UTR and 3′ UTR by Lipofectamine™

A panel of MOEs targeting the 5′ UTR or 3′ UTR were screened in SH-SY5Y cells using Lipofectamine™ 3000 (Life Technologies) to facilitate transfection, as per the manufacturer's instructions. Total protein was harvested (96 hours) from the transfected cells and an untreated control (UTC) using 15% SDS protein lysis buffer, and expression of SHANK3 was assessed by western blot. (A) SHANK3 expression was normalized to housekeeper (HK) proteins and the fold-change in its expression was measured against the transfection control (TFC; no ASO). On average, none of the screened MOEs induced ≥1.5-fold upregulation of SHANK3, relative to the TFC. Data are presented as mean±SEM (n=2-4 biological replicates, 1-2 technical replicates per experiment). Corresponding SEQ ID NOs for each MOE tested are indicated under each graph set. (B) Representative western blot using rabbit anti-SHANK3 polyclonal antibody (Bethyl Labs, A304-178A-T) at1:10 000 in 5% BSA in TBST to detect SHANK3. HK proteins were measured using rabbit anti-Beclin1 monoclonal antibody at 1:2 000 in 5% BSA in TBST (Cell Signalling Technology, S3495) and mouse anti-Vinculin monoclonal antibody at 1:200 in 5% BASA in TBST (Sigma-Aldrich, V9131). Corresponding SEQ ID NOs for each MOE tested are indicated over each lane.

FIG. 13—A subset of exemplary SHANK3 MOEs induce robust upregulation of SHANK3 protein by targeting the 5′ and 3′ UTRs of SHANK3 transcripts

A subset of MOEs targeting the 5′ and 3′ UTRs of SHANK3 were tested for their ability to upregulate SHANK3 protein expression in SH-SY5Y cells. Total protein was harvested (96 hours) from the transfected cells and an untreated control (UTC) using 15% SDS protein lysis buffer. Expression of SHANK3 was assessed by western blot and normalised to total protein levels (Revert™ 700 Total Protein Stain, LI-COR). A transfection control (TFC; no ASO) was used to calculate the fold-change in SHANK3 expression. A subset of MOEs (SEQ ID NOs: 3510, 299, 301, 302, 304, 305, 309, 318 and 1193) caused upregulation at 5.0 μM, relative to the TFC. Select MOEs were also tested at 25 μM and were shown to further increase SHANK3 expression ≥1.5-fold relative to the TFC (SEQ ID NOs: 293, 305-308, 311, 313, 315, 316, 797 and 1195). Data are presented as mean±SEM (n=1-2 biological replicates, with 1-2 technical replicates per experiment).

FIG. 14—PMO 5′ UTR diagram

Binding sites of PMOs targeting the putative extended 5′ UTR and canonical 5′ UTR of SHANK3 transcripts. Grey rectangles above upper-case sequence (ACGT) represent exons, or exon segments; dashed lines above lower-case sequence (agct) represent introns, or intron segments. Ellipses ( . . . ) indicate where part of an exon or intron sequence is omitted from this diagram. =Where necessary, the span of the absent sequence between two shown sequences is written (e.g., in intron 1-1152 bp-). Black bars indicate the target sites of the PMOs, which are labelled with the corresponding SEQ IDs; grey bars indicate the target site of a single PMO (SEQ ID 598) that bridges exon 3 and exon 4, with asterisks showing where the two target site segments are contiguous in the mature spliced mRNA. SHANK3 exon/intron boundaries and sequence are derived from ENSEMBL transcript reference sequence ENST00000262795.6.

FIG. 15—PMO retained intron diagram (1 of 2)

Binding sites of PMOs targeting the exons of mature SHANK3 transcripts. Grey rectangles above upper-case sequence (ACGT) represent exons, or exon segments; dashed lines above lower-case sequence (agct) represent introns, or intron segments. Ellipses ( . . . ) indicate where part of an exon or intron sequence is omitted from this diagram. Black bars indicate the target sites of PMOs with sequences corresponding to the labelled SEQ IDs. SHANK3 exon/intron boundaries and sequence are derived from ENSEMBL transcript reference sequence ENST00000262795.6

FIG. 16—PMO retained intron diagram (2 of 2)

Binding sites of PMOs targeting the exons of mature SHANK3 transcripts. Grey rectangles above upper-case sequence (ACGT) represent exons, or exon segments dashed lines above lower-case sequence (agct) represent introns, or intron segments. Ellipses ( . . . ) indicate where part of an exon or intron sequence is omitted from this diagram. Black bars indicate the target sites of PMOs with sequences corresponding to the labelled SEQ IDs. SHANK3 exon/intron boundaries and sequence are derived from ENSEMBL transcript reference sequence ENST00000262795.6

FIG. 17—PMO 3′ UTR diagram

Binding sites of PMOs targeting the 3′ UTR of SHANK3 transcripts. Grey rectangles above upper-case sequence (ACGT) represent segments of exon 23. Ellipses ( . . . ) denote where part of a sequence has been omitted from the diagram, and the span of the absent sequence is indicated (i.e., 1128 bp). Black bars represent the target sites of a given PMOs, which are labelled with the correspondingSEQ IDs. SHANK3 exon boundaries and sequence are derived from ENSEMBL transcript reference sequence ENST00000262795.6

FIG. 18—MOE 5′ UTR and 3′ UTR diagram

Binding sites of MOEs targeting the 5′ UTR (top) and 3′ UTR (middle, bottom) of mature SHANK3 transcripts. Grey rectangles above upper-case sequence (ACGT) represent exons or exon segments, with vertical black lines indicating exon-exon junctions. Ellipses ( . . . ) denote where part of an exon or intron sequence is omitted from this diagram. Where necessary, the span of the absent sequence is also indicated. Black bars represent the target sites of each MOE,, which are labelled with the corresponding SEQ IDs. SHANK3 exon/intron boundaries and numbering are derived from GenBank transcript reference sequence NM_001372044.2.

FIG. 19—A subset of exemplary SHANK3 PMOs induce robust transcript modulation of SHANK3

SH-SY5Y cells were transfected with a subset of PMOs designed to target the 5′ UTR or putative extended 5′ UTR of SHANK3 that demonstrated ≥1.5-fold-change upregulation of SHANK3 protein from western blot assessment. SH-SY5Y cells were transfected with PMOs at 25 μM and 50 μM via electroporation (NEON, ThermoFisher Scientific), as per the manufacturer's instructions. Cells were harvested (96 hours) and RNA was extracted using MagMAX™-96 Total RNA Isolation Kit (AM1830, ThermoFisher Scientific). Target engagement was determined by the percentage modulation of altered SHANK3 transcript relative to TFC (transfection control) and UTC (untreated control), indicated by RT-PCR gel bands (LabChip GXII, PerkinElmer). Data are presented at mean±SEM (n=3 biological replicates).

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Each example of the present disclosure described herein is to be applied mutatis mutandis to each and every other example unless specifically stated otherwise.

Those skilled in the art will appreciate that the disclosure herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure, as described herein.

The present disclosure is performed without undue experimentation using, unless otherwise indicated, conventional techniques of molecular biology, microbiology, virology, recombinant DNA technology, peptide synthesis in solution, solid phase peptide synthesis, and immunology. Such techniques are described and explained throughout the literature in sources such as Perbal 1984, Sambrook et al., 2001, Brown (editor) 1991, Glover and Hames (editors) 1995 and 1996, Ausubel et al. including all updates until present, Coligan et al. (editors) (including all updates until present), Maniatis et al. 1982, Gait (editor) 1984, Hames and Higgins (editors) 1984, Freshney (editor) 1986.

The term “and/or”, e.g, “X and/or Y” shall be understood to mean either “X and Y” or “X or Y” and shall be taken to provide explicit support for both meanings or for either meaning.

The term “about”, unless stated to the contrary, refers to +/−20%, more preferably +/−10%, of the designated value. For the avoidance of doubt, the term “about” followed by a designated value is to be interpreted as also encompassing the exact designated value itself (for example, “about 10” also encompasses 10 exactly).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “antisense oligonucleotide” “antisense oligomer” or “ASO,” as used herein, encompasses oligonucleotides and any other oligomeric molecule that comprises nucleobases capable of hybridizing to a complementary sequence on a target RNA transcript, including, but not limited to, those that do not comprise a sugar moiety, such as in the case of a peptide nucleic acid (PNA). Preferably, the ASO is an ASO that is resistant to nuclease cleavage or degradation.

The phrase “binds to a targeted portion” or “binds within a targeted portion,” in reference to an ASO or AR, as used herein, refers to specific hybridization between the ASO or AR nucleotide sequence and a target nucleotide sequence that is complementary within the ranges set forth herein. In some examples, specific hybridization occurs where, under ex vivo conditions, the hybridization occurs under high stringency conditions. By “high stringency conditions” is meant that the ASO or AR, under such ex vivo conditions, hybridize to a target sequence in an amount that is detectably stronger than non-specific hybridization. High stringency conditions, then, are conditions that distinguish a polynucleotide with an exact complementary sequence, or one containing only a few scattered mismatches from a random sequence that happened to have a few small regions (e.g., 1-5 bases) that matched the probe. Such small regions of complementarity are more easily melted than a full-length complement of 12-17 or more bases, and moderate stringency hybridization makes them easily distinguishable. In one example, high stringency conditions include, for example, low salt and/or high temperature conditions, such as provided by about 0.02-0.1 M NaCl or the equivalent, at temperatures of about 50-−70° C. The skilled person will appreciate that under in vivo conditions, the specificity of hybridization between an ASO or an AR and its target sequence is defined in terms of the level of complementarity between the ASO or an AR and the target sequence to which it hybridizes within a cell.

The term “peptide” is intended to include compounds composed of amino acid residues linked by amide bonds. A peptide may be natural or unnatural, ribosome translated or synthetically derived. Typically, a peptide will consist of between 2 and 200 amino acids. For example, the peptide may have a length in the range of 10 to 20 amino acids or 10 to 30 amino acids or 10 to 40 amino acids or 10 to 50 amino acids or 10 to 60 amino acids or 10 to 70 amino acids or 10 to 80 amino acids or 10 to 90 amino acids or 10 to 100 amino acids, including any length within said range(s). The peptide may comprise or consist of fewer than about 150 amino acids or fewer than about 125 amino acids or fewer than about 100 amino acids or fewer than about 90 amino acids or fewer than about 80 amino acids or fewer than about 70 amino acids or fewer than about 60 amino acids or fewer than about 50 amino acids.

Peptides, as referred to herein, include “inverso” peptides in which all L-amino acids are substituted with the corresponding D-amino acids, “retro-inverso” peptides in which the sequence of amino acids is reversed and all L-amino acids are replaced with D-amino acids.

Peptides may comprise amino acids in both L- and/or D-form. For example, both L- and D-forms may be used for different amino acids within the same peptide sequence. In some examples the amino acids within the peptide sequence are in L-form, such as natural amino acids. In some examples the amino acids within the peptide sequence are a combination of L- and D-form. Further, peptides may comprise unusual, but naturally occurring, amino acids including, but not limited to, hydroxyproline (Hyp), beta-alanine, citrulline (Cit), ornithine (Orn), norleucine (Nle), 3-nitrotyrosine, nitroarginine, pyroglutamic acid (Pyr). Peptides may also incorporate unnatural amino acids including, but not limited to, homo amino acids, N-methyl amino acids, alpha-methyl amino acids, beta (homo) amino acids, gamma amino acids, and N-substituted glycines. Peptides may be linear peptides or cyclic peptides.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical bond or a disulfide bond. Examples of non-covalent bonds include hydrogen bonds, ionic bonds, Van der Waals forces, and hydrophobic interactions.

Percentage amino acid sequence identity with respect to a given amino acid sequence is defined as the percentage of amino acid residues in a candidate sequence that are identical to the amino acid residues in the reference sequence, after aligning the sequences and introducing gaps, if necessary, to achieve the maximum percent sequence identity, and not considering any conservative substitutions as part of the sequence identity. Amino acid sequence identity may be determined using the EMBOSS Pairwise Alignment Algorithms tool available from The European Bioinformatics Institute (EMBL-EBI), which is part of the European Molecular Biology Laboratory. This tool is accessible at the website located at www.ebi.ac.uk/Tools/emboss/align/. This tool utilizes the Needleman-Wunsch global alignment algorithm (Needleman and Wunsch, 1970). Default settings are utilized, which include Gap Open: 10.0 and Gap Extend 0.5. The default matrix “Blosum62” is utilized for amino acid sequences and the default matrix. Percent (%) or percentage “nucleic acid sequence identity” with respect to the nucleotide sequences disclosed herein is defined as the percentage of nucleotides in a candidate sequence that are identical with the nucleotides in the reference sequence, after aligning the sequences and introducing gaps, if necessary, to achieve the maximum percent sequence identity. Alignment for purposes of determining percent nucleic acid sequence identity can be achieved in various ways that are known in the art, for instance, using publicly available computer software such as BLAST or ALIGN. The skilled person can readily determine appropriate parameters for measuring alignment, including any algorithms needed to achieve maximal alignment over the full length of the sequences being compared.

The term “cell penetrating peptide” (CPP) refers to a peptide that is capable of crossing a cellular membrane. In one example, a CPP is capable of translocating across a mammalian cell membrane and entering into a cell. In another example, a CPP may direct a conjugate to a desired subcellular compartment. Thus, a CPP may direct or facilitate penetration of a molecule of interest across a phospholipid, mitochondrial, endosomal, lysosomal, vesicular, or nuclear membrane. A CPP may be translocated across the membrane with its amino acid sequence complete and intact, or alternatively partially degraded.

A CPP may direct a molecule of interest, such as an ASO disclosed herein, from outside a cell through the plasma membrane, and into the cytoplasm or a desired subcellular compartment. Alternatively, or in addition, a CPP may direct a molecule of interest across the blood-brain, trans-mucosal, hematoretinal, skin, gastrointestinal and/or pulmonary barriers.

The term “peptide ligand” or “receptor binding domain” refers to a peptide that is capable of binding to a membrane surface receptor to enable translocation of the peptide across a cellular membrane. In one example a peptide ligand may enable translocation across the cellular membrane via the natural endocytosis of the targeted receptor. In another example the peptide ligand may utilise a complementary mechanism of translocation across the cellular membrane including utilising a conjugated CPP. In one example, a peptide ligand is capable of translocating across a mammalian cell membrane and to enter a cell. In another example, a peptide ligand may direct a conjugate to a desired subcellular compartment. Thus, a peptide ligand may direct or facilitate cellular uptake of a molecule of interest across a phospholipid, mitochondrial, endosomal, lysosomal, vesicular, or nuclear membrane. A peptide ligand may be translocated across the membrane with its amino acid sequence complete and intact, or alternatively partially degraded.

A peptide ligand via its binding to a target receptor may direct a molecule of interest, such as an ASO disclosed herein, from outside a cell through the plasma membrane, and into the cytoplasm or a desired subcellular compartment. Alternatively, or in addition, a peptide ligand via its binding to a target receptor may direct a molecule of interest across a relevant biological barrier, e.g., the blood-brain, trans-mucosal, hematoretinal, skin, gastrointestinal, and/or pulmonary barriers.

### Compositions for Increasing SHANK3 Protein Levels

Secondary structures and upstream open reading frames in the 5′ UTR of SHANK3 mRNA can reduce translational efficiency and transcript stability thereby limiting protein production. In addition, the SHANK3 5′ UTR includes 3 exons, exon 2 contains sequences that can affect translational efficiency, which ultimately affects the level of SHANK3 protein. Exclusion of SHANK3 exon 2 during splicing of the the 5′ PNCR shortens the resulting 5′ UTR, removing elements that reduce translational efficiency. Further, microRNAs (miRNAs) typically bind to complimentary RNA sequences in the 3′ untranslated region (3′ UTR) and regulate gene expression by stimulating either mRNA degradation or translational repression. Both classes of mechanisms lead to diminished expression of a gene. Despite such “inefficiencies,” in a genetic background of two functional (wild type) alleles, the level of productive RNA transcripts and translation yield a sufficient level of functional protein for a given gene. However, in the case of rare, monogenic diseases, the loss of one functional allele, e.g., SHANK3 allele, can result in haploinsufficiency and the associated disease.

While not wishing to be bound by theory, it is believed that ASOs targeting sequences within the 5′ UTR of SHANK3 mRNA can enhance translation, e.g., by reducing/disrupting the formation of secondary structures that would otherwise reduce translational efficiency. ASOs targeting the 5′ PNCR can enhance SHANK3 transcript translational efficiency. Further, it is believed that an antisense sequence at least partly complementary to the binding sites for miRNAs located within the SHANK3 mRNA 3′ UTR will hybridize to the 3′ UTR and sterically hinder (“mask”) access of these miRNAs to their binding sites, thereby resulting in an increased level of SHANK3 mRNA and ultimately allowing increased translation of SHANK3 protein.

Accordingly, disclosed herein is an ASO that binds within a targeted portion of the: (i) 5′ UTR of a SHANK3 mRNA; (ii) 5′ PNCR of a SHANK3 pre-mRNA; or (iii) 3′ UTR of a SHANK3 mRNA;
whereby binding of the antisense oligonucleotide within the targeted portion in a mammalian cell results in an increased level of SHANK3 protein in the mammalian cell.

For reference, the nucleotide sequence of the canonical human SHANK3 pre-mRNA transcript (“SHANK3-201”) is provided herein as SEQ ID NO:4. The nucleotide sequence of the 5′ UTR of the canonical human SHANK3 mRNA transcript is provided herein as SEQ ID NO:1. The nucleotide sequence of the 5′ PNCR of the canonical SHANK3 pre-mRNA is provided herein as SEQ ID NO:3. The nucleotide sequence of the 3′ UTR of the canonical human SHANK3 mRNA is provided herein as SEQ ID NO:2 (Appendix).

### Antisense Oligonucleotides (ASOs) and Antisense RNAs (ARs)

In some examples of the compositions and methods described herein, ASOs and ARs have a sequence that is completely or nearly completely complementary across its length to the target sequence. ASOs and ARs are designed so that they bind (hybridize) to a target RNA sequence (e.g., a targeted portion of a mRNA transcript) and remain hybridized under physiological conditions. Selection of suitable sequences for ASOs and ARs generally avoids, where possible, similar nucleic acid sequences in other (i.e., off-target) locations in the genome or in cellular mRNAs or miRNAs, such that the likelihood the ASO or AR will hybridize at such sites is limited. In some examples, ASOs disclosed herein are useful for attenuating the formation of SHANK3 mRNA secondary structures, particularly in the 5′ UTR region that interfere with translation. In other examples, ASOs disclosed herein bind to a targeted region within the 5′ PNCR of a SHANK3 pre-mRNA, e.g., within an intronic sequence or partly within an intronic sequence and partly within flanking exonic sequence. In other examples, ASOs disclosed herein mask access of miRNAs to their target binding sites in the SHANK3 3′ UTR thereby reducing the level of miRNA-dependent SHANK3 mRNA destabilization. Thus, the ASOs disclosed herein result in a net increase in the level of canonical SHANK3 mRNA and consequently the level of functional SHANK3 protein.

In some examples, ASOs or ARs “specifically hybridize” to or are “specific” to a target nucleic acid or a targeted portion of a SHANK3 mRNA 5′ UTR or 3′ UTR, or a SHANK3 pre-mRNA 5′ PNCR. At a given ionic strength and pH, the Tₘ is the temperature at which 50% of a target sequence hybridizes to a complementary oligonucleotide.

ASO and AR sequences are “complementary” to their target sequences when hybridization occurs in an antiparallel configuration between two single-stranded polynucleotides. Complementarity is quantifiable in terms of the proportion (e.g., the percentage) of bases in opposing strands that are expected to form hydrogen bonds with each other, according to generally accepted base-pairing rules. The nucleotide sequence of an ASO or AR need not be 100% complementary to that of its target nucleic acid to hybridize. In certain examples, the nucleotide sequences of ASOs or ARs in the compositions disclosed herein can be at least 60%, at least 65%, at least 70%, at least 75%, at least 80%, at least 85%, at least 90%, at least 95%, at least 96%, at least 97%, at least 98%, or at least 99% sequence complementary to the nucleotide sequence of the targeted portion of an RNA transcript over the length of the ASO or AR nucleotide sequence. For example, an ASO or AR in which 18 of 20 nucleotides of ASO or AR sequence are complementary to a target region, and would therefore specifically hybridize, would represent 90 percent complementarity. In such an example, the remaining non-complementary nucleotides of the ASO or AR could be clustered together or interspersed with complementary nucleotides and need not be contiguous. Complementarity of an ASO or AR sequence to a target nucleotide sequence (expressed as “percent complementarity” to its target sequence; or “percent identity” to its reverse complement sequence) can be determined routinely using algorithms known in the art, as exemplified in the BLAST programs (basic local alignment search tools) and PowerBLAST programs (Altschul, et al., 1990, J. Mol. Biol., 215:403-410; Zhang et al., 1997, Genome Res., 7:649-656).

In some examples, an ASO or AR does not hybridize to all nucleotides in a target sequence and the nucleotide positions at which it does hybridize may be contiguous or noncontiguous. ASOs or ARs may hybridize over one or more segments of a SHANK3 mRNA 5′ UTR or 3′ UTR; one or more segments of a SHANK3 mRNA 5′ PNCR, such that intervening or adjacent segments are not involved in the hybridization event (e.g., a loop structure or hairpin structure may be formed).

In some examples, the ASOs or ARs disclosed herein are complementary to a targeted portion of the canonical SHANK3 mRNA 5′ UTR sequence corresponding to SEQ ID NO:1. In other examples, the ASOs or ARs disclosed herein are complementary to a targeted portion of the canonical SHANK3 pre-mRNA 5′ PNCR sequence corresponding to SEQ ID NO:3. In other examples, the ASOs or ARs disclosed herein are complementary to a targeted portion of the canonical SHANK3 mRNA 3′ UTR sequence corresponding to SEQ ID NO:2.

In some examples the nucleotide sequence of the ASO or the AR is at least 60%, 65%, 70%, 75%, 80%, 85%, 90%, 95%, or 100% complementary to the nucleotide sequence of the targeted portion of the SHANK3 mRNA over the length of the ASO or the AR. In some examples the nucleotide sequence of the ASO or the AR comprises a sequence that: (a) has at least about 40% identity to about 60% identity of the nucleotide sequence of an ASO or AR sequence disclosed herein, e.g., 45% identity, 48% identity, 50% identity, 52% identity, 55% identity, 58% identity or another sequence identity from about 40% identity to about 60% identity to the entire length of the sequence of any ASO or AR disclosed herein; and (b) comprises a contiguous sequence of at least 8 bases to 16 bases that is 100% identical to a contiguous sequence of at least 8 to 16 bases in any one of the ASO or AR sequences disclosed herein, e.g., 100% sequence-identical to a contiguous 9 bases, 10 bases, 11 bases, 12 bases, 13 bases, 14 bases, 15 bases, or 16 bases of an ASO or AR sequence disclosed herein. The ASOs or ARs for use in the compositions described herein may be of any length suitable for specific hybridization to a target sequence. In some examples, the nucleotide sequence of the ASOs or ARs consist of 8 to 50 nucleotides. For example, the ASO or AR sequence can be 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleotides in length. In some examples, the nucleotide sequence of the ASOs or ARs consist of 8 to 50 nucleotides. For example, the ASO or AR sequence can be 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleotides in length. In some examples, the ASOs consist of more than 50 nucleotides, but no more than 100 nucleotides in length. In some examples, the ASO or AR nucleotide sequence is from 8 to 50 nucleotides, 8 to 40 nucleotides, 8 to 35 nucleotides, 8 to 30 nucleotides, 8 to 25 nucleotides, 8 to 20 nucleotides, 8 to 15 nucleotides, 9 to 50 nucleotides, 9 to 40 nucleotides, 9 to 35 nucleotides, 9 to 30 nucleotides, 9 to 25 nucleotides, 9 to 20 nucleotides, 9 to 15 nucleotides, 10 to 50 nucleotides, 10 to 40 nucleotides, 10 to 35 nucleotides, 10 to 30 nucleotides, 10 to 25 nucleotides, 10 to 20 nucleotides, 10 to 15 nucleotides, 11 to 50 nucleotides, 11 to 40 nucleotides, 11 to 35 nucleotides, 11 to 30 nucleotides, 11 to 25 nucleotides, 11 to 20 nucleotides, 11 to 15 nucleotides, 12 to 50 nucleotides, 12 to 40 nucleotides, 12 to 35 nucleotides, 12 to 30 nucleotides, 12 to 25 nucleotides, 12 to 20 nucleotides, 12 to 15 nucleotides, 13 to 50 nucleotides, 13 to 40 nucleotides, 13 to 35 nucleotides, 13 to 30 nucleotides, 13 to 25 nucleotides, 13 to 20 nucleotides, 14 to 50 nucleotides, 14 to 40 nucleotides, 14 to 35 nucleotides, 14 to 30 nucleotides, 14 to 25 nucleotides, 14 to 20 nucleotides, 15 to 50 nucleotides, 15 to 40 nucleotides, 15 to 35 nucleotides, 15 to 30 nucleotides, 15 to 25 nucleotides, 15 to 20 nucleotides, 17 to 30 nucleotides, 17 to 25 nucleotides, 17 to 20 nucleotides, 20 to 50 nucleotides, 20 to 40 nucleotides, 20 to 35 nucleotides, 20 to 30 nucleotides, 20 to 25 nucleotides, 25 to 50 nucleotides, 25 to 40 nucleotides, 25 to 35 nucleotides, or 25 to 30 nucleotides in length. In some examples, the ASOs or ARs are 17 nucleotides in length. In other examples, the ASOs or ARs are 20 nucleotides in length. In some examples, the nucleotide sequence of the ASO or AR nucleotide is 25 nucleotides in length.

In other examples the ASOs or ARs comprise at least 10 contiguous nucleotides of an ASO or AR sequence described herein. In some examples ASOs or ARs comprise at least 10 contiguous nucleotides (subsequence) from each of two or more ASO or AR sequences described herein, where the two or more subsequences are not contiguous in a SHANK3 mRNA sequence.

In some examples for each occurrence of “G” in an ASO or AR sequence disclosed herein, the “G” is guanosine or inosine. In some examples for each occurrence of “T” in an ASO or AR sequence disclosed herein, the “T” is any one of: thymidine, inosine, uracil, or an isomeric or modified form of uracil (e.g., pseudouridine or N1-methyl-pseudouridine). In some examples for each occurrence of “C” in an ASO or AR sequence disclosed herein, the C is cytosine or a modified form of cytosine (e.g., 5′-methyl cytosine).

In some examples the nucleotide sequence of the ASO or AR comprises the sequence of any one of SEQ ID NOs: 293, 299, 301, 302, 304-309, 311, 313, 315, 318, 606, 797, 1193, 1195, 1847, 1934-1937, 2858, 2874, 3510, 12644, 12666, 12669, 12671, 12688, or 12690. In some examples the nucleotide sequence of the ASO or AR comprises the sequence of any one of SEQ ID NOs: 5-622, 4175-4181, or 4184-4186. In some examples the nucleotide sequence of the ASO or AR comprises the sequence of any one of SEQ ID NOs: 5-188, 191-622, 4175-4181, or 4184-4186. In some examples the nucleotide sequence of the ASO or AR comprises the sequence of any one of SEQ ID NOs: 559, 606, or 4178-4181. In other examples the nucleotide sequence of the ASO or AR comprises the sequence of any one of SEQ ID NOs: 1935-4168, 4182, 4183, 12646-12654, or 12664-12671. In some examples the nucleotide sequence of the ASO or AR comprises the sequence of any one of SEQ ID NOs: 1935-1937, 2849, 2858, 2864, 2874, 3510, 12647, 12648, or 12664-12671. In other examples the nucleotide sequence of the ASO or AR comprises the sequence of any one of SEQ ID NOs: 623-1934,4169-4174, 12645, 12655-12663, or 12688. In some examples the nucleotide sequence of the ASO or AR comprises the sequence of SEQ ID NO:1934.

In some examples the nucleotide sequence of the ASO or AR consists of the nucleotide sequence of any one of SEQ ID NOs: 5-4186, 12646-12654, or 12664-12671. Sequences for the foregoing SEQ ID NOs are provided in Tables 4 and 5 in the Appendix.

### ASO Chemistry and Modifications

The ASOs used in the compositions described herein may comprise naturally-occurring nucleotides, nucleotide analogues, modified nucleotides, or any combination thereof. The term “naturally occurring nucleotides” includes deoxyribonucleotides and ribonucleotides. The term “modified nucleotides” includes nucleotides with modified or substituted sugar groups and/or having a modified backbone. In some examples, all the nucleotides of an ASO are modified nucleotides. Chemical modifications of ASOs or components of ASOs that are compatible with the compositions and methods described herein are known in the art as disclosed in, e.g., in U.S. Pat. Nos. 8,258,109, 5,656,612, U.S. Patent Publication No. 2012/0190728, and Roberts et al., 2020, Nature Rev. Drug Disc., 19:673-694.

One or more nucleotides of an ASO may be any naturally occurring, unmodified nucleobase such as adenine, guanine, cytosine, thymine, uracil and inosine, or any synthetic or modified nucleobase that is sufficiently similar to an unmodified nucleobase such that it is capable of hydrogen bonding with a nucleobase present on a target RNA transcript. Examples of suitable modified nucleobases include, but are not limited to, hypoxanthine, xanthine, 7-methylguanine, 5, 6-dihydrouracil, 5-methylcytosine, and 5-hydroxymethoylcytosine.

ASOs include a “backbone” structure, that refers to the connection between nucleotides/monomers of the ASO. In naturally occurring oligonucleotides, the backbone comprises a 3′-5′ phosphodiester linkage connecting sugar moieties of adjacent nucleotides. Suitable types of backbone linkages for the ASOs described herein include, but are not limited to, phosphodiester, phosphorothioate, phosphorodithioate, phosphorodiamidate, phosphoroselenoate, phosphorodiselenoate, phosphoroanilothioate, phosphoraniladate, phosphoramidate, and the like. In some examples, the backbone modification is a phosphorothioate linkage. In other examples, the backbone modification is a phosphorodiamidate linkage. See, e.g., Roberts et al. supra; and Agrawal (2021), Biomedicines, 9:503. In some examples, the backbone structure of the ASO does not contain phosphorous-based linkages, but rather contains peptide bonds, for example in a peptide nucleic acid (PNA), or linking groups including carbamate, amides, and linear and cyclic hydrocarbon groups.

In some examples, the stereochemistry at each of the phosphorus internucleotide linkages of the ASO backbone is random. In other examples, the stereochemistry at each of the phosphorus internucleotide linkages of the ASO backbone is controlled and is not random. For example, U.S. Pat. No. 9,605,019 describes methods for independently selecting the handedness of chirality at each phosphorous atom in an oligonucleotide. In some examples, an ASO used in the compositions and methods provided herein, including, but not limited to, the ASOs the sequences of which are disclosed herein as SEQ ID NOs: 5-39535 is an ASO having phosphodiester internucleotide linkages that are not random. In some examples, a composition or composition used in the methods disclosed herein comprises a pure diastereomeric ASO. In other examples, the composition comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some examples, the ASO has a non-random mixture of Rp and Sp configurations at its phosphorus internucleotide linkages. In some examples, an ASO used in the compositions and methods disclosed herein, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp.

In some examples, the ASOs described herein contain a sugar moiety that comprises ribose or deoxyribose, or a modified sugar moiety or sugar analog, including a morpholine ring. Suitable examples of modified sugar moieties include, but are not limited to, 2′ substitutions such as 2′-O-modifications, 2′-O-methyl(2′-O-Me), 2′-O-methoxyethyl(2′MOE), 2′-O-aminoethyl, 2′F, N3′→P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. In some examples, the sugar moiety modification is selected from among 2′-O-Me, 2′F, and 2′MOE. In other examples, the sugar moiety modification is an extra bridge bond, such as in a locked nucleic acid (LNA). In some examples the sugar analog contains a morpholine ring, such as phosphorodiamidate morpholino (PMO). In some examples, the sugar moiety comprises a ribofuransyl or 2′deoxyribofuransyl modification. In some examples, the sugar moiety comprises 2′4′-constrained 2′-O-methyloxyethyl (cMOE) modifications. In some examples, the sugar moiety comprises cEt 2′, 4′ constrained 2′-O ethyl BNA modifications. In other examples, the sugar moiety comprises tricycloDNA (tcDNA) modifications. In some examples, the sugar moiety comprises ethylene nucleic acid (ENA) modifications. In some examples, the sugar moiety comprises 2′-O-(2-N-methylcarbamoylethyl) (MCE). Modifications are known in the art as exemplified in Jarver, et al., 2014, Nucleic Acid Therapeutics, 24 (1): 37-47.

In some examples, each constituent nucleotide of the ASO is modified in the same way, e.g., every linkage of the backbone of the ASO comprises a phosphorothioate linkage, or each ribose sugar moiety comprises a 2′-O-methyl modification. In other examples, a combination of different modifications is used, e.g., an ASO comprising a combination of phosphorodiamidate linkages and sugar moieties comprising morpholine rings (morpholinos).

In some examples, the ASO comprises one or more backbone modifications. In some examples, the ASO comprises one or more sugar moiety modification. In some examples, the ASO comprises one or more backbone modifications and one or more sugar moiety modifications. In some examples, the ASO comprises a 2′MOE modification and a phosphorothioate backbone. In some examples, the ASO comprises a peptide nucleic acid (PNA).

In some preferred examples, the ASO comprises a phosphorodiamidate morpholino (PMO).

The skilled person in the art will appreciate that ASOs may be modified in order to achieve desired properties or activities of the ASO or reduce undesired properties or activities of the ASO. In some examples, an ASO is modified to alter one or more properties. For example, such modifications can: enhance binding affinity to a target sequence on a pre-mRNA transcript; reduce binding to any non-target sequence; reduce degradation by cellular nucleases (e.g., RNase H); improve uptake of an ASO into a cell and/or particular subcellular compartments; alter the pharmacokinetics or pharmacodynamics of the ASO; and/or modulate the half-life of the ASO in vivo.

In some examples, the ASOs comprise one or more 2′-O-(2-methoxyethyl) (MOE) phosphorothioate-modified nucleotides, which have been shown to confer significantly enhanced resistance of ASOs to nuclease degradation and increased bioavailability.

Methods for synthesis and chemical modification of ASOs, as well as synthesis of ASO conjugates is well known in the art, and such ASOs are available commercially.

In some examples, a composition (e.g., a pharmaceutical composition) provided here includes two or more ASOs with different chemistries but complementary to the same targeted portion of the SHANK3 mRNA 5′ UTR or 3′ UTR, or the SHANK3 pre-mRNA 5′ PNCR. In other examples, a composition comprises two or more ASOs that are complementary to different targeted portions of the 5′ UTR, 3′ UTR, or 5′ PNCR.

In some examples, the compositions disclosed herein include ASOs that are linked to a functional moiety. In some examples, the functional moiety is a delivery moiety, a targeting moiety, a detection moiety, a stabilizing moiety, or a therapeutic moiety. In some examples the functional moiety includes a delivery moiety or a targeting moiety. In some examples the functional moiety includes a stabilizing moiety. In some preferred examples the functional moiety is a delivery moiety.

Suitable delivery moieties include, but are not limited to, lipids, polyethers, peptides, carbohydrates, glycans, receptor binding domains (RBDs), and antibodies.

In some examples, the delivery moiety includes a cell-penetrating peptide (CPP). Suitable examples of CPPs are described in, e.g., PCT/AU2020/051397. In some examples the amino acid sequence of the CPP comprises or consists of: RRSRTARAGRPGRNSSRPSAPR (SEQ ID NO:12694). In other examples, the delivery moiety includes a RBD.

In other examples, the delivery moiety includes a carbohydrate. In some examples, a carbohydrate delivery moiety is selected from among N-acetylgalactosamine (GalNAc), N-Ac-Glucosamine (GluNAc), glycan, and a mannose. In one example, the carbohydrate delivery moiety comprises GalNac or a glycan moiety.

In other examples, the delivery moiety includes a lipid. Examples of suitable lipids as delivery moieties include, but are not limited to, cholesterol moiety, a cholesteryl moiety, and aliphatic lipids. In some examples the delivery moiety includes a fatty acid or lipid moiety. In some embodiments the fatty acid chain length is about C8 to C20. Examples of suitable fatty acid moieties and their conjugation to oligonucleotides are found in, e.g., International Patent Publication WO 2019232255 and in Prakash et al., (2019).

In further examples, the delivery moiety includes an antibody, as described in, e.g., Dugal-Tessier et al., (2021).

Suitable examples of stabilizing moieties include, but are not limited to, polyethylene glycol (PEG), poly (oligo (ethylene glycol) methyl ether methacrylate) (POEGMA), and Poly(2-oxazoline)s (POx).

In some examples, where an ASO is linked to a functional moiety, the functional moiety is covalently linked to the ASO. In other examples, the functional moiety is non-covalently linked to the ASO.

Functional moieties can be linked to one or more of any nucleotides in an ASO at any of several positions on the sugar, base or phosphate group, as understood in the art and described in the literature, e.g., using a linker. Linkers can include a bivalent or trivalent branched linker. In some examples, the functional moiety is linked to the 5′ end of the ASO. In other examples, the functional moiety is linked to the 3′ end of the ASO.

In some examples compositions comprising any of the ASOs disclosed herein also include a delivery nanocarrier complexed with ASO. In some examples, a delivery nanocarrier is selected from among lipoplexes, liposomes, exosomes, inorganic nanoparticles, and DNA nanostructures. In other examples the delivery nanocarrier includes a lipid nanoparticle encapsulating the ASO. Various delivery ASO-nanocarrier complex formats are known in the art, as reviewed in, e.g., Roberts et al., supra.

### Vectors for Expression of SHANK3 Antisense RNA (AR)

In some examples provided herein is a vector for expression, in a mammalian neuron or other cell type, of an antisense RNA (AR) that binds within a targeted portion of the: (i) 5′ UTR of a SHANK3 mRNA; (ii) 5′ PNCR of a SHANK3 pre-mRNA; or (iii) 3′ UTR of a SHANK3 mRNA; whereby binding of the AR within the targeted portion in a mammalian cell results in an increased level of SHANK3 protein in the mammalian cell.

In some examples, the promoter used in the expression vector is a neuron type-selective promoter for driving expression of the AR in the mammalian cell. In some examples, the neuronal cell type-selective promoter is selective for expression in neurons selected from the list consisting of: cortical glutamatergic neurons, cortical GABAergic neurons, hippocampal glutamatergic neurons, and striatal inhibitory neurons.

In some the promoter is an inducible promoter, e.g., inducible by a ligand-regulated transactivator such as the tet-inducible rtTA, which allows titration of AR transcription in a target mammalian cell. In some examples, the promoter driving AR expression is a U6 or other Pol III promoter, which is particularly suitable for transcription of short RNA sequences such AR sequences disclosed herein. In some examples, an expression vector utilizes hybrid promoter systems, e.g., a Tet-O-regulated U6 promoter system as described in Lin et al. (2004), FEBS Letters, 577 (2004) 376-380. In some examples, where both cell type-specificity and inducibility of an AR expression vector are desired, a two-part expression system is used in which expression of a ligand-regulated transactivator is driven by a cell type-selective promoter and expression of an AR disclosed herein is driven by a promoter regulated by the ligand-regulated transactivator.

In some examples, the expression vectors used in the compositions disclosed herein are non-viral expression vectors, e.g., plasmid vectors, minicircle DNA vectors, linear amplicon expression cassettes, and the like.

In some examples, composition containing a non-viral expression virus further comprises a transfection agent. Exemplary transfection agents for transfection include, but are not limited to, jet-PEI® (available from Polyplus-Transfection® SA, Strasbourg, France); TurboFect in vivo Transfection Reagent (ThermoFisher), and cationic derivatives of polyisoprenoid alcohols (PTAI) as described in, e.g., Rak et al., (2016).

In other examples, the expression vectors to be used are viral vectors, i.e., non-replicative recombinant viruses suitable for expression of an AR disclosed herein.

Preferably, the recombinant virus for expression of the SHANK3 AR is a DNA virus. Suitable types of DNA viruses include adeno-associated virus (AAV), adenovirus, lentivirus, herpes simplex virus (HSV), and anelloviruses. Methods for design, production, and use of such types of recombinant DNA viruses are established in the art, as exemplified in Fukazawa et al., (2010), International J of Mol. Med, 25 (1), 3-10, and in “Gene Therapy Protocols” for adenovirus; “Adeno-Associated Virus: Methods and Protocols” for AAV; Cody et al (2013), Journal of Genetic Syndromes & Gene Therapy, 4 (1), 126, and “Herpes Simplex Virus: Methods and Protocols” for HSV; “Gene Therapy Protocols Vol. 1: Production and In Vivo Applications of Gene Transfer Vectors”; and Merten et al. (2016), Molecular Therapy—Methods & Clinical Development, 3, 16017, and Emeagi et al. (2013), Current Molecular Medicine 13 (4), 602-625 for lentivirus. In some preferred examples, the viral vector is a recombinant AAV.

### Genetically Modified Cells

Also provided herein are genetically modified cells. In some examples the genetically modified cells are genetically modified bacterial cells (e.g., recombinant E. coli, for amplifying an AR expression vector disclosed herein). In other examples the genetically modified cells are genetically modified mammalian cells that have been transfected with any of the ASOs or non-viral AR expression vectors; or transduced with any of the viral AR expression vectors disclosed herein. In some examples, the genetically modified mammalian cells are ex vivo, e.g., as a cultured cell population. In other examples, the genetically modified mammalian cells are in vivo, e.g., in a mouse. In some examples, the genetically modified mammalian cells are human cells.

In some examples the genetically modified mammalian cells are neurons or neural progenitors. Suitable examples of neurons include, but are not limited to, cortical glutamatergic neurons, cortical GABAergic neurons, hippocampal glutamatergic neurons, and striatal inhibitory neurons. In some examples such primary cell types can be obtained by differentiation of a human pluripotent stem cell line, e.g., an hiPSC line or a human embryonic stem cell (hESC) line. Methods for obtaining a variety of different neuronal cell types is known in the art, as reviewed in, e.g., Alia et al., (2019), Fitzgerald et al., (2020) and Kim et al. (2014). In other examples, the genetically modified mammalian cells are derived from a cell line. In some examples the cell line is pluripotent stem cell line (e.g., hiPSCs or hESCs) or a neuronal cell line. Suitable neuronal or neuronal stem cell lines include, but are not limited to, SH-SY5Y, NTera, CTX0E16, ReNcell VM, ReNcell Cx. In some preferred examples, the genetically modified mammalian cells express SHANK3 endogenously.

The genetically modified cells disclosed herein can be genetically modified by any of a number of methods and strategies known in the art, e.g., transient transfection, stable transfection, and viral transduction. In some examples transfection with ASOs or non-viral vectors is carried out by nucleofection. In other examples transfection of cells is by lipofection.

### Pharmaceutical Compositions

Also provided herein are pharmaceutical compositions comprising any of the foregoing ASOs, non-viral expression vectors, and viral expression vectors disclosed herein, and formulated with at least a pharmaceutically acceptable excipient, including a carrier, filler, preservative, adjuvant, solubilizer and/or diluent.

Pharmaceutical compositions containing any of the ASOs or expression vector compositions described herein, for use in the methods disclosed herein, can be prepared according to conventional techniques well known in the pharmaceutical industry and described in the published literature. In some examples, a pharmaceutical composition for treating a subject comprises a therapeutically effective amount of any ASO or expression vector disclosed herein.

Pharmaceutically acceptable salts are suitable for use in contact with the tissues of humans and lower animals without undue toxicity, irritation, allergic response, etc., and are commensurate with a reasonable benefit/risk ratio. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

In some examples, pharmaceutical compositions are formulated into any of a number of possible dosage routes or forms including, but not limited to, intravenous administration, intrathecal administration magna administration, tablets, capsules, gel capsules, liquid syrups, and soft gels. In some examples, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some examples, a pharmaceutical formulation disclosed herein is provided in a form including, but not limited to, a solution, emulsion, microemulsion, foam or liposome-containing formulation (e.g., cationic or noncationic liposomes).

In some examples, pharmaceutical formulations comprising any of the ASOs or expression vectors described herein may comprise one or more penetration enhancers, carriers, excipients or other active or inactive ingredients as appropriate and known to the skilled person. In some examples, where a pharmaceutical composition includes liposomes, such liposomes can also include sterically stabilized liposomes, e.g., liposomes comprising one or more specialized lipids. These specialized lipids result in liposomes with enhanced circulation lifetimes. In some examples, a sterically stabilized liposome comprises one or more glycolipids or is derivatized with one or more hydrophilic polymers, such as PEG moiety. In some examples, a surfactant is included in the pharmaceutical formulation.

In some examples, a pharmaceutical composition also includes a penetration enhancer to enhance the delivery of ASOs or non-viral expression vectors, e.g., to aid diffusion across cell membranes and/or enhance the permeability of a lipophilic drug. In some examples, the penetration enhancers include a surfactant, a fatty acid, a bile salt, or a chelating agent.

In some examples, where administration is via a systemic route, e.g., intravenous, the method also includes a step to facilitate transfer of any of the ASOs or vectors described herein across the blood brain barrier (BBB) into the CNS, and especially into the brain. In some examples the BBB is transiently disrupted, e.g., by administration of one or more antibodies that disrupt Netrin-1 binding to Unc5B as described in Boyé et al., (2022).

In some examples, a pharmaceutical composition comprises a dose of ASOs or non-viral vectors ranging from about 0.01 mg/kg to 20 mg/kg, e.g., 0.05 mg/kg, 0.1 mg/kg, 0.2 mg/kg, 0.5 mg/kg, 1 mg/kg, 3 mg/kg, 5 mg/kg, 8 mg/kg, 10 mg/kg, 15 mg/kg, or another dose ranging from about 0.01 mg/kg to 20 mg/kg. In some examples, where an ASO disclosed herein is to be administered directly into the CNS or brain, e.g., by intracerebroventricular administration, the total dose ranges from about 50 mg to about 500 mg, e.g., 60 mg, 70 mg, 80 mg, 100 mg, 120 mg, 150 mg, 180 mg, 200 mg, 220 mg, 250 mg, 270 mg, 290 mg, 300 mg, 350 mg, 400 mg, 450 mg, or another dose from about 50 mg to about 500 mg. This dose range corresponds to approximately 0.050 mg/cm³ of brain volume to about 0.42 mg/cm³ of brain volume assuming an average human brain volume of about 1200 cm³.

In some examples, a pharmaceutical composition comprises multiple ASOs or AR expression vectors. In some examples, a pharmaceutical composition comprises, in addition to ASOs or AR expression vectors, another drug or therapeutic agent suitable for treatment of a subject suffering from SHANK3 haploinsufficiency.

### Methods

As described herein, a number of conditions (e.g., Phelan-McDermid syndrome) are associated with insufficient levels of functional SHANK3. Accordingly, the methods described herein include a method for preventing or treating a condition associated with SHANK3 haploinsufficiency by administering to the subject a therapeutically effective amount of a pharmaceutical composition comprising any of the ASOs or expression vectors disclosed herein. Likewise, in some examples, any of the ASOs or AR expression vectors disclosed herein are used in the manufacture of a medicament for treating a condition associated with SHANK3 haploinsufficiency. In some examples the condition associated with SHANK3 haploinsufficiency to be prevented or treated by the methods or with the compositions disclosed herein is Phelan-McDermid syndrome, an autism spectrum disorder, schizophrenia, or an intellectual disability. In some preferred examples, the condition is Phelan-McDermid syndrome.

Also provided herein is a method for increasing the amount of functional SHANK3 protein in a mammalian cell expressing SHANK3 mRNA, the method comprising contacting the cell with any of the ASOs or expression vectors disclosed herein.

In some examples, administration to a subject or contact with cells in vitro or ex vivo with any of the ASOs, AR expression vectors, or pharmaceutical compositions disclosed herein increases the level of SHANK3 protein about 1.1 to about 5 fold in cells (e.g., neurons) in vitro or ex vivo, e.g., 1.2 fold, 1.3 fold, 1.5 fold, 1.7 fold, 2 fold, 2.2 fold, 2.5 fold, 2.7 fold, 3 fold, 3.3 fold, 3.5 fold, 4 fold, 4.3 fold, 4.5 fold, 4.7 fold, or another increase in SHANK3 protein levels from about 1.1 fold to about 5 fold in cells in a subject or in vitro or in vivo.

Suitable routes of administration for treatment with the compositions, pharmaceutical compositions, or medicaments disclosed herein include, but are not limited to, intravenous, intra-arterial, intraparenchymal, intracerebroventricular, intra-cisterna magna, intrathecal, intravenous, intra-arterial, subcutaneous, and topical.

As the skilled person will understand, the treatment methods disclosed herein include administration of the compositions and pharmaceutical compositions disclosed herein in a therapeutically effective amount to a subject (e.g., a human subject). The terms “effective amount” or “therapeutically effective amount,” as used herein, refer to a sufficient amount of a disclosed ASO, non-viral or viral expression vector being administered to relieve to some extent one or more of the symptoms and/or clinical indicia associated with SHANK3 haploinsufficiency in a particular disease or health condition. In some examples, an “effective amount” for therapeutic uses is the amount of one of the foregoing agents required to provide a clinically significant decrease in disease symptoms to prevent disease symptoms without undue adverse side effects. Examples of suitable symptoms to be reduced by the treatment methods provided herein included, but are not limited to, seizures, anxiety, repetitive behaviors, learning and memory deficits, and impaired sociability. An appropriate “effective amount” in any individual case may be determined using techniques, such as a dose escalation study. The term “therapeutically effective amount” includes, for example, a prophylactically effective amount. It is understood that “an effective amount” or “a therapeutically effective amount” can vary from subject to subject, due to variation in metabolism of the compound of any age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial. Where more than one therapeutic agent is used in combination, a “therapeutically effective amount” of each therapeutic agent can refer to an amount of the therapeutic agent that would be therapeutically effective when used on its own, or may refer to a reduced amount that is therapeutically effective by virtue of its combination with one or more additional therapeutic agents.

### Combination Treatments

The pharmaceutical compositions comprising any of the ASOs or AR expression vectors, disclosed herein, can also be used in combination with other agents of therapeutic value in the treatment of a condition associated with SHANK3 haploinsufficiency. In general, other agents do not necessarily have to be administered in the same pharmaceutical composition, and may, because of different physical and chemical characteristics, preferably be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

Compositions and pharmaceutical compositions comprising ASOs and/or expression vectors, and an additional therapeutic agent may be administered concurrently (e.g., simultaneously, essentially simultaneously or within the same treatment protocol) or sequentially, depending upon the stage and progression of the SHANK3 haploinsufficiency-associated condition to be treated, the condition of the patient, and the choice of specific therapeutic agents used. The determination of the order of administration, and the number of repetitions of administration of each therapeutic agent during a treatment protocol, is well within the knowledge of the skilled physician after evaluation of the disease being treated and the condition of the patient.

It is known to those of skill in the art that therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

For combination therapies, dosages of co-administered therapeutic agents will of course vary depending on the type of co-agents employed, ASO or expression vector, and the disease stage of the patient to be treated.

Pharmaceutical compositions comprising ASOs, ARs, or expression vectors, and an additional therapeutic agent that make up a combination therapy disclosed herein may be a combined dosage form or in separate dosage forms intended for substantially simultaneous administration. The pharmaceutical compositions that make up the combination therapy may also be administered sequentially, with either therapeutic agent being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of various physiological parameters may also be evaluated to determine the optimal dose interval.

Examples of suitable therapeutic agents for co-administration with a composition or a pharmaceutical composition disclosed herein include, but are not limited to, Growth Hormone, Insulin-Like Growth Factor-1, Risperidone, Lumateperone, sodium valproate, lithium, and D-serine.

### EXAMPLES

### Example 1: Identification of SHANK3 Target Sequences

Identification of annotated and expressed transcripts that could generate the canonical transcript of the SHANK3 gene was performed by the sequence alignment of all SHANK3 protein-coding and NMD transcripts described in Gencode v38. An ASO sequence “micro-walk” of 25-mers and 17mers in 3-base increments was performed across the 5′ PNCR of the SHANK3 canonical pre-RNA transcript and across the 5′ UTR and 3′ UTR of the SHANK3 canonical mRNA transcript (ENST00000262795.6). The resulting ASO sequences correspond to SEQ ID NOs: 5-4186, 12646-12671, and 12688, which are provided in Table 4 (Appendix).

### Example 2: Measurement of SHANK3 Protein Upregulation by PMOs Targeting of SHANK3 mRNA 5′ UTR or 3′ UTR in a Neuronal Cell Line

A subset of the above-identified ASO sequences were synthesized as phosphorodiamidate morpholino oligonucleotides (PMOs 4-37) targeting the canonical SHANK3 mRNA 5′ UTR, 5′ PNCR, and 3′ UTR regions. The PMOs, corresponding SEQ ID NOs, and target regions are shown in Table 1 below. The antisense PMOs were electroporated into SH-SY5Y neuronal cell line cultures using the NEON® electroporation system (ThermoFisher) at concentrations of 25 μM and 50 μM and incubated for 96 hours. At this time, total protein was extracted using 15% SDS complete lysis reagent and the levels of SHANK3 protein assessed by western blot using a mouse anti-SHANK3 monoclonal antibody (Merck, Cat. No. SAB520004) at a dilution of 1:1000 in 5% BSA in TBST buffer followed by goat anti-mouse IgG H&L antibody (Abcam, Cat. number ab216776). Expression levels of the SHANK3 protein were compared to no PMO-transfected cells (untreated), control treated with transfection conditions and scrambled/non-targeting control sequences. The average signal from SHANK3 following imaging analysis was normalized to the average signal of total protein and ‘housekeeper gene’ proteins (e.g., vinculin, beclin, SRSF4).

TABLE 1
Exemplary PMOs Tested for Ability to
Increase SHANK3 Protein Levels

| | SEQ | SHANK3 |
|---|---|---|
| | ID | Region |
| PMO | NO | (mRNA or pre-mRNA) |
| PMO4 | 4169 | 3′UTR |
| PMO5 | 4170 | 3′UTR |
| PMO6 | 1934 | 3′UTR |
| PMO7 | 4171 | 3′UTR |
| PMO8 | 4172 | 3′UTR |
| PMO9 | 4173 | 3′UTR |
| PMO10 | 4174 | 3′UTR |
| PMO19 | 4184 | 5′UTR |
| PMO20 | 4185 | 5′UTR |
| PMO21 | 4186 | 5′UTR |
| PMO22 | 4175 | 5′UTR |
| PMO23 | 559 | 5′UTR |
| PMO24 | 4176 | 5′UTR |
| PMO25 | 4177 | 5′UTR |
| PMO26 | 598 | 5′UTR |
| PMO27 | 4178 | 5′UTR |
| PMO28 | 4179 | 5′UTR |
| PMO29 | 4180 | 5′UTR |
| PMO30 | 4181 | 5′UTR |
| PMO31 | 4182 | PNCR |
| PMO32 | 4183 | PNCR |
| PMO33 | 606 | 5′UTR |
| PMO34 | 1935 | PNCR |
| PMO35 | 1936 | PNCR |
| PMO36 | 2849 | PNCR |
| PMO37 | 1937 | PNCR |
| PMO40 | 1847 | 3′UTR |
| PMO41 | 12662 | 3′UTR |
| PMO42 | 12661 | 3′UTR |
| PMO43 | 12688 | 3′UTR |
| PMO44 | 1852 | 3′UTR |
| PMO45 | 12663 | 3′UTR |
| PMO63 | 12664 | PNCR |
| PMO64 | 12665 | PNCR |
| PMO65 | 2858 | PNCR |
| PMO66 | 12666 | PNCR |
| PMO67 | 12667 | PNCR |
| PMO68 | 2864 | PNCR |
| PMO69 | 12668 | PNCR |
| PMO70 | 2874 | PNCR |
| PMO71 | 12669 | PNCR |
| PMO72 | 12670 | PNCR |
| PMO73 | 12671 | PNCR |

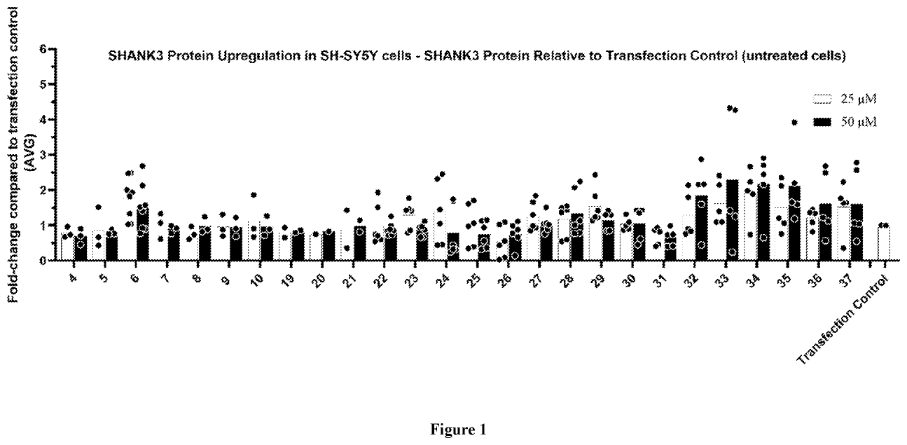

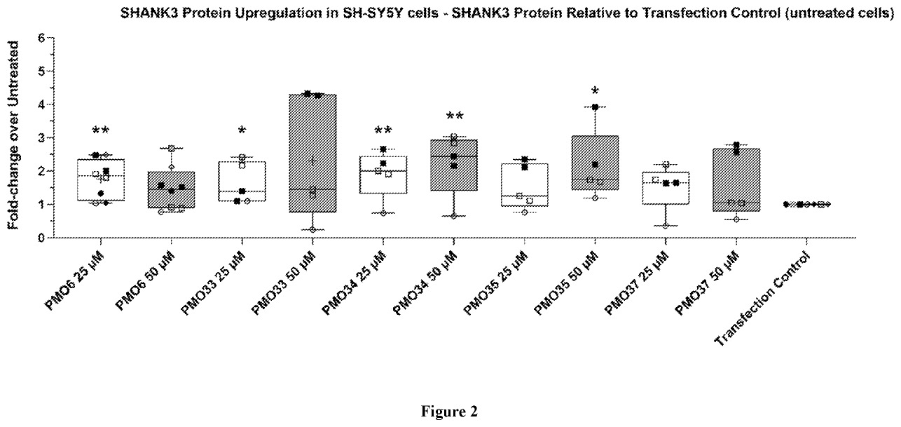

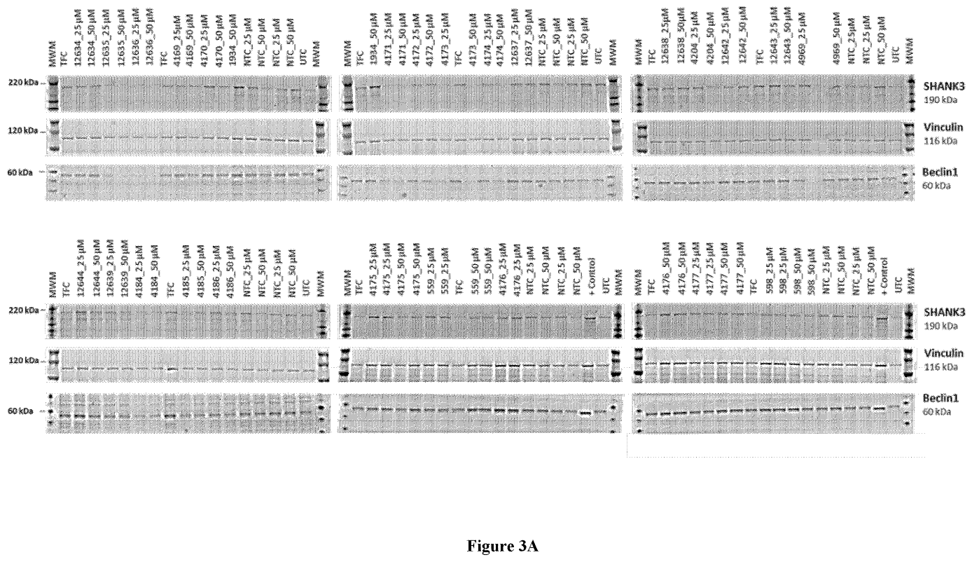

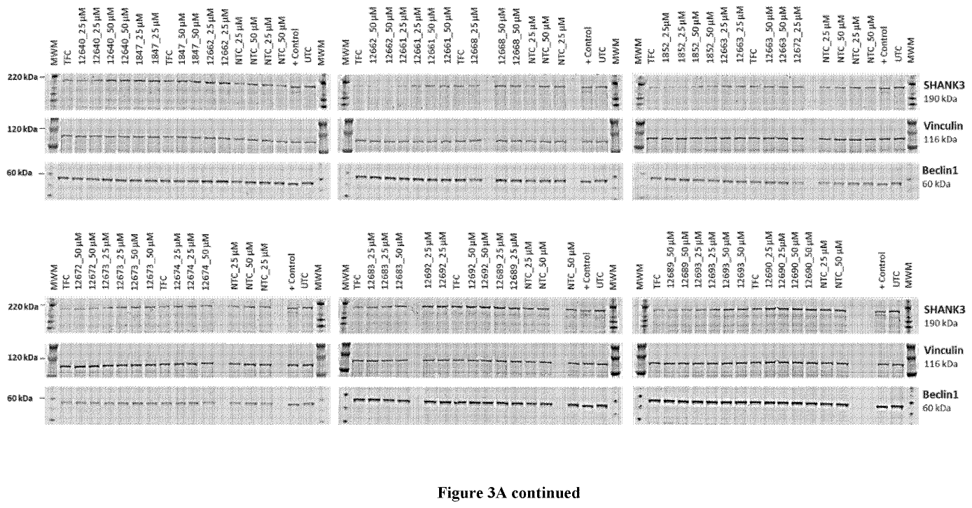

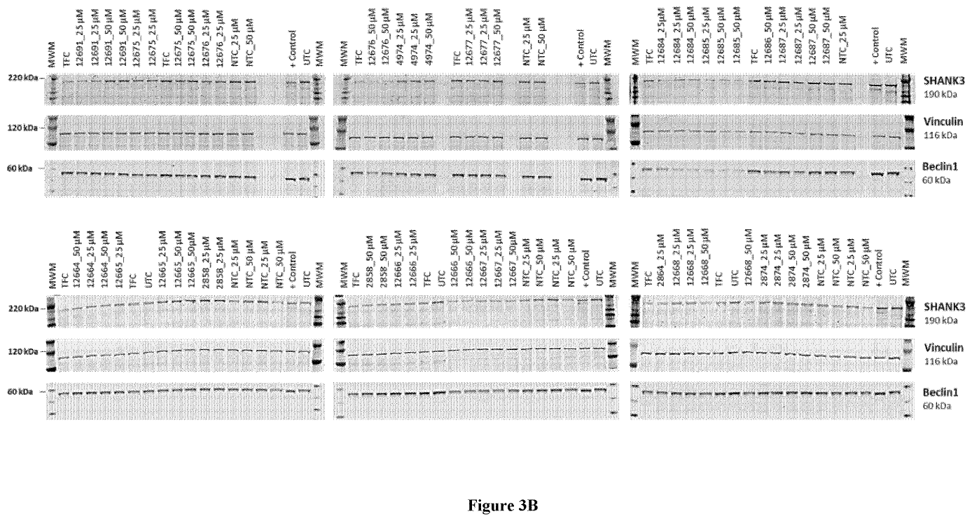

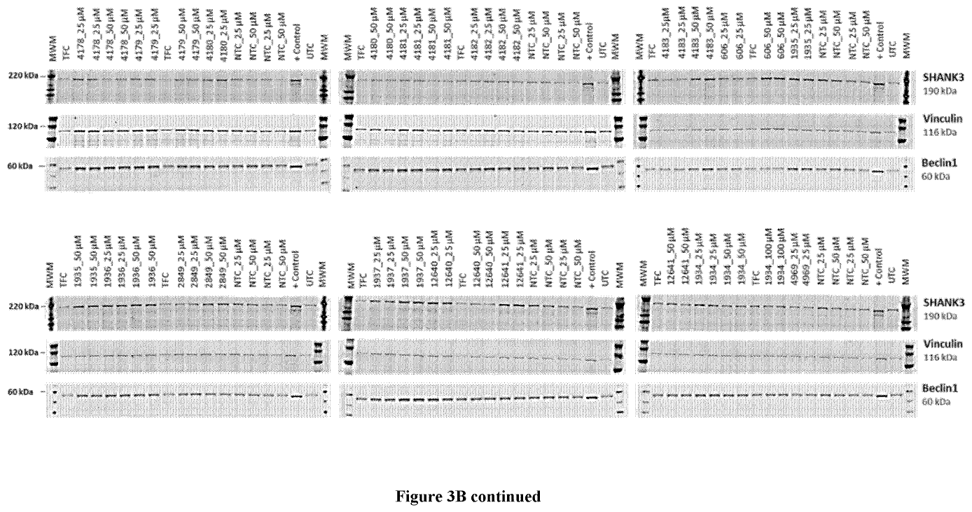

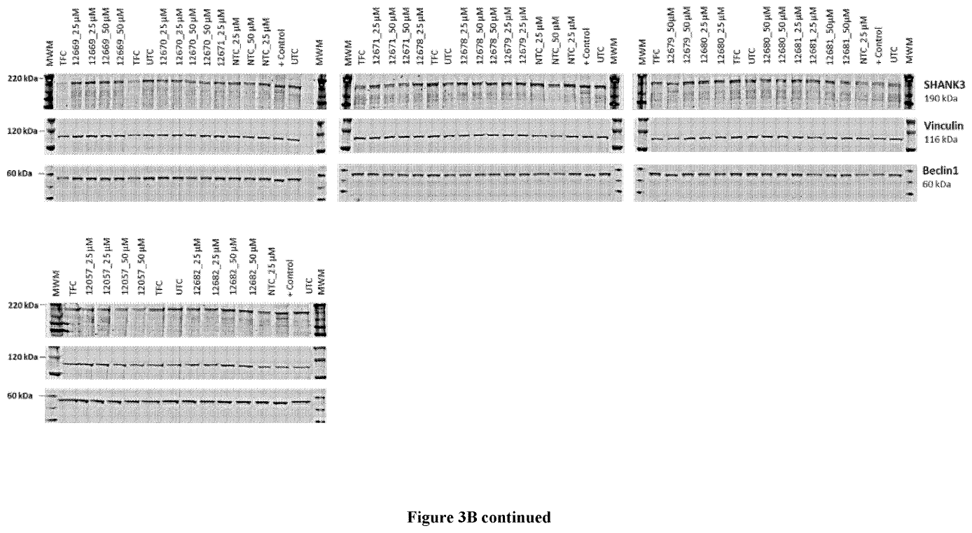

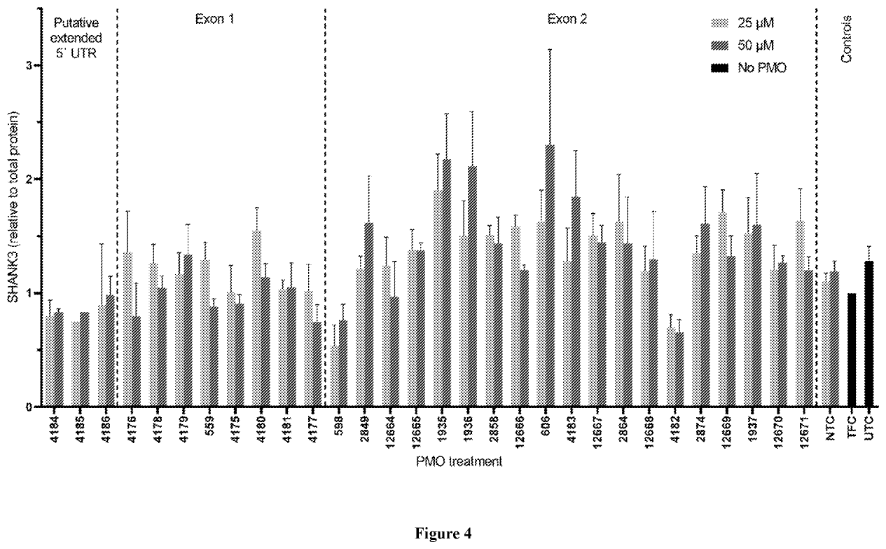

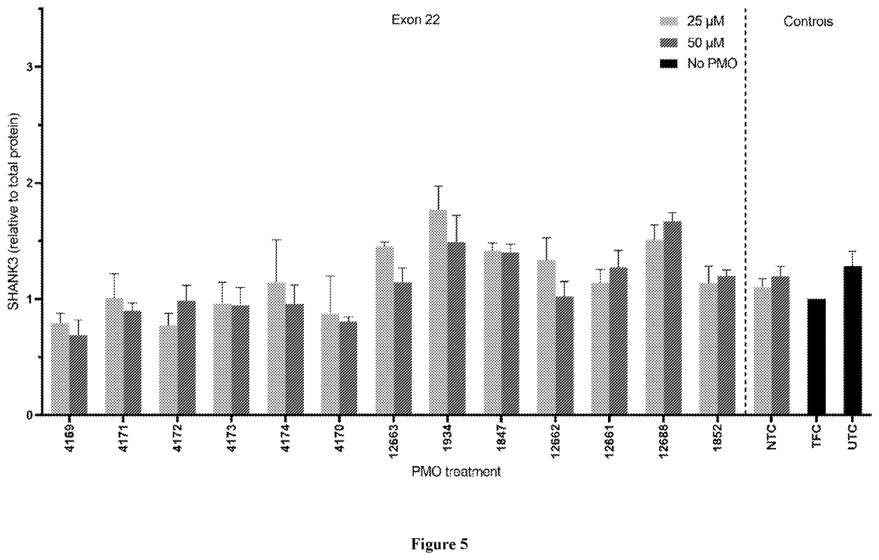

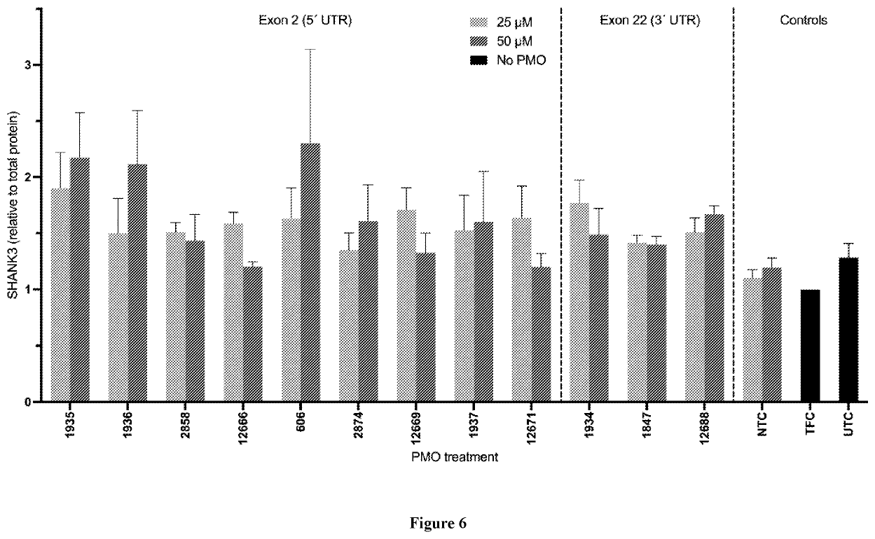

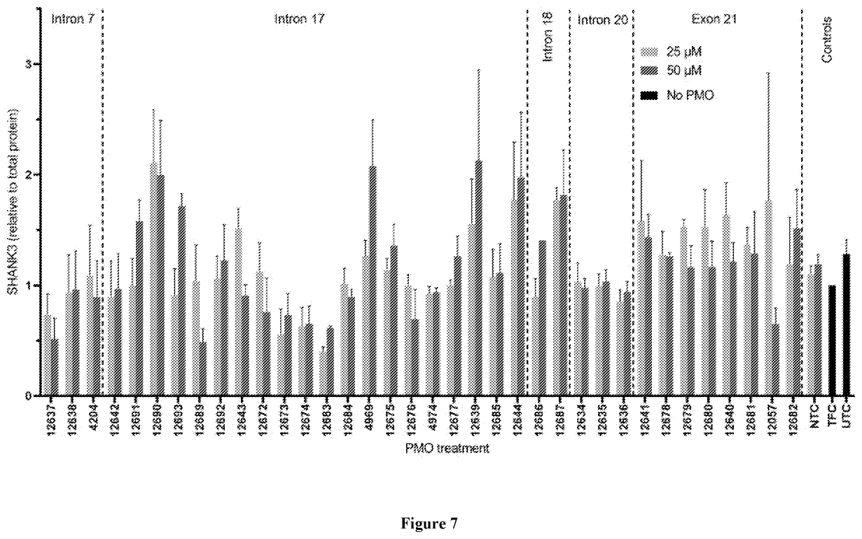

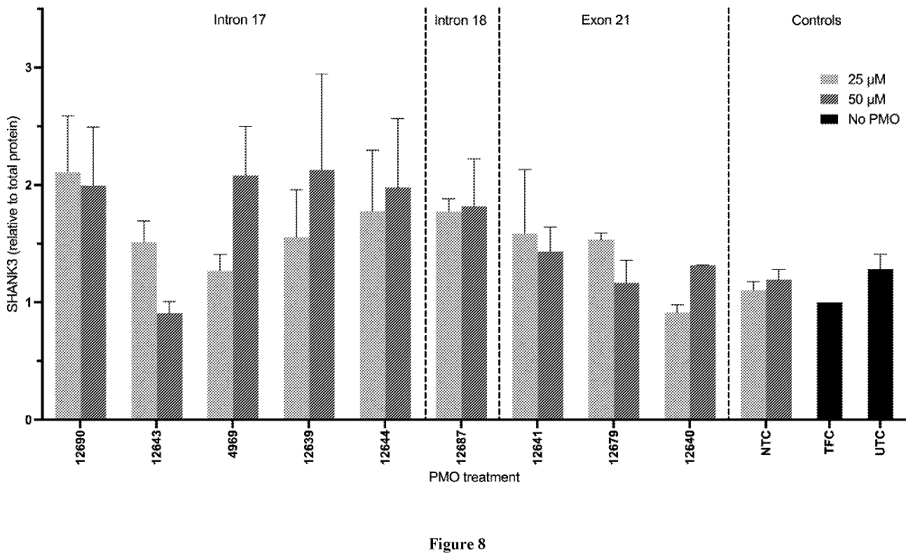

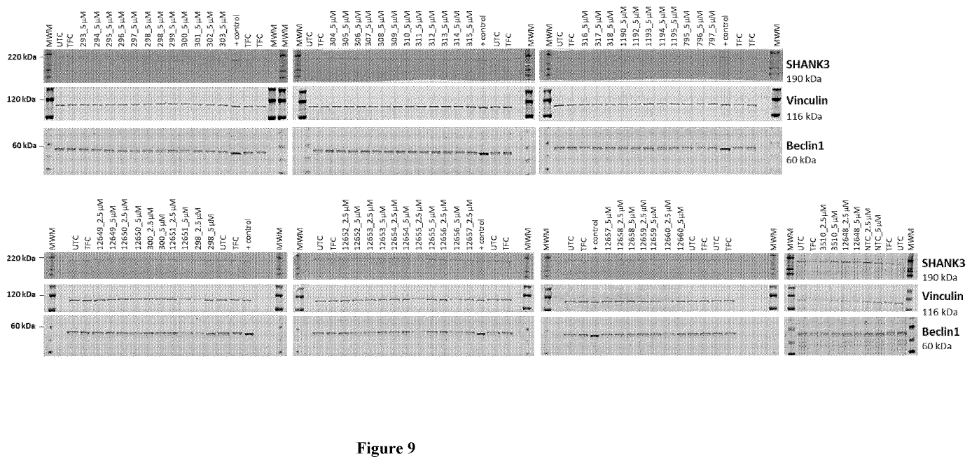

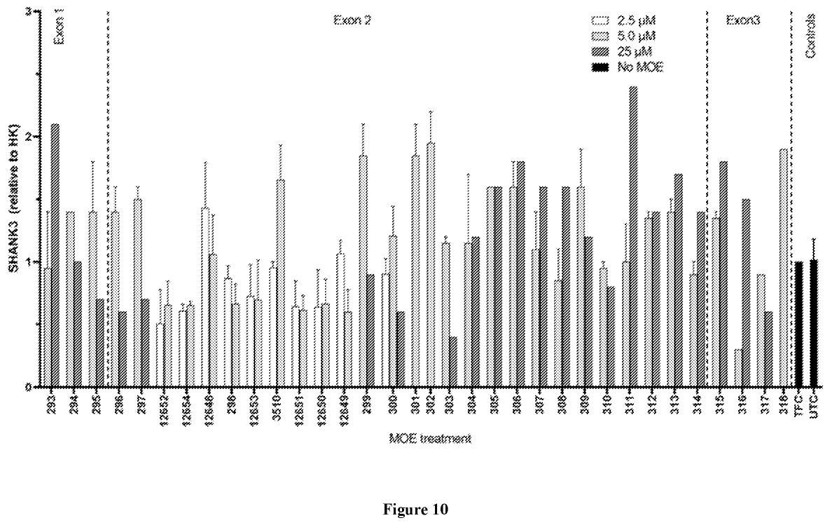

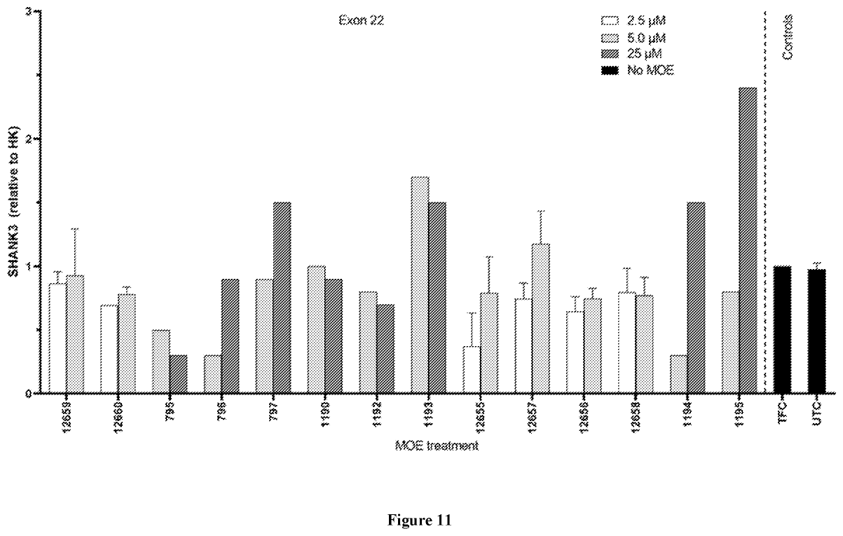

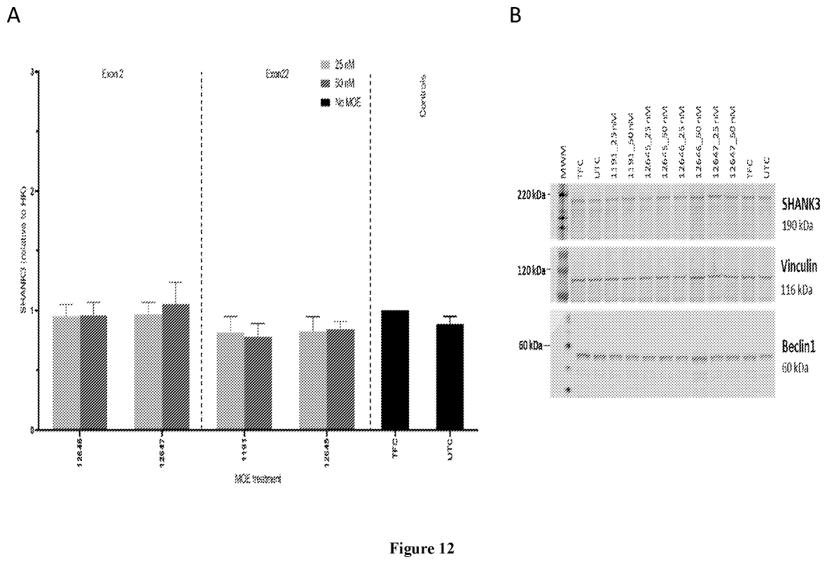

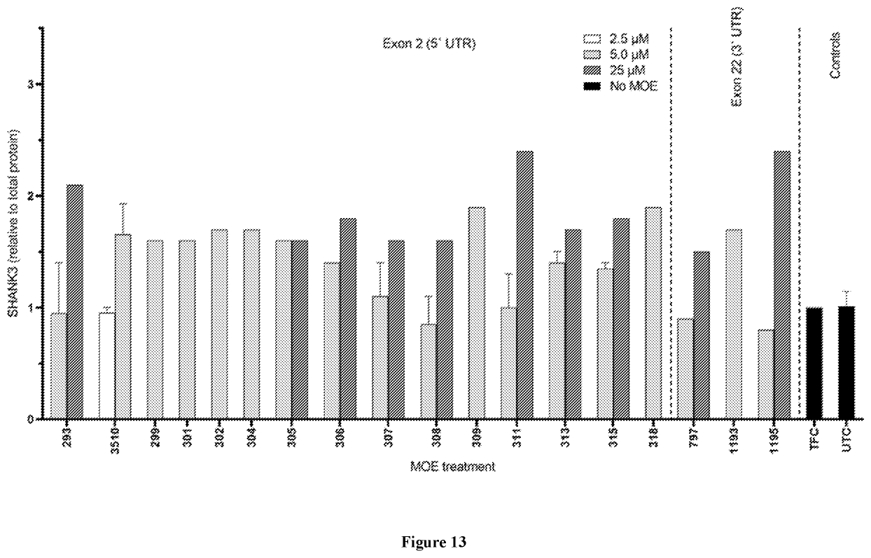

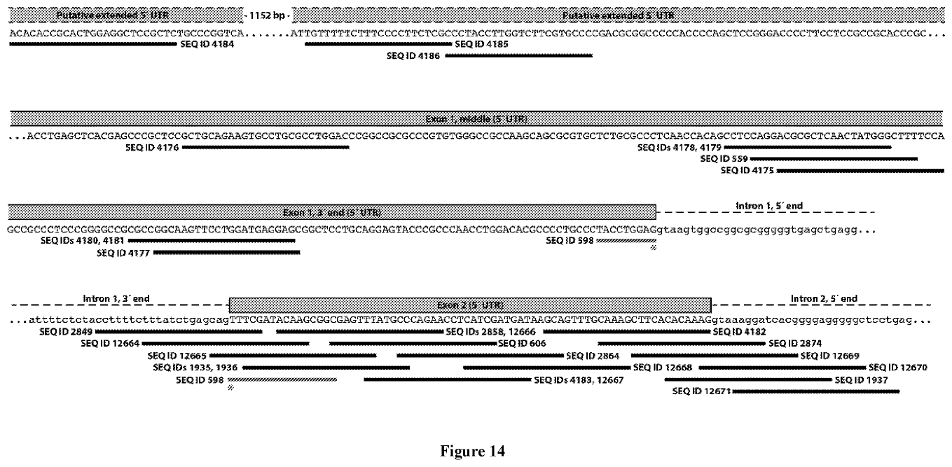

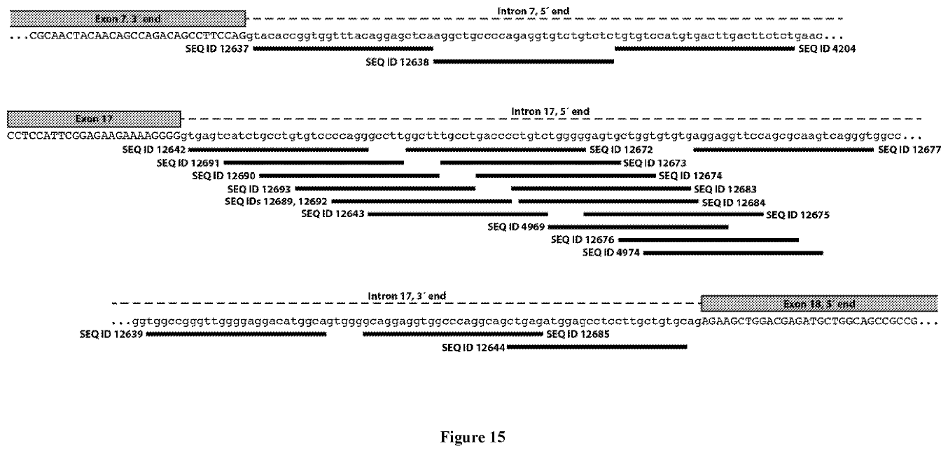

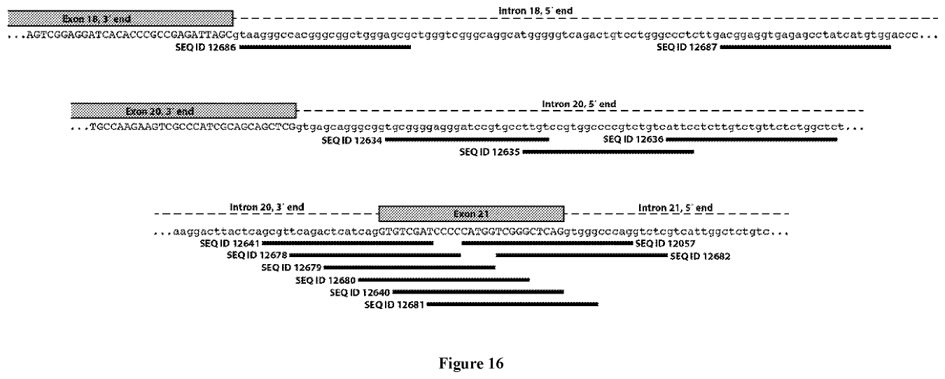

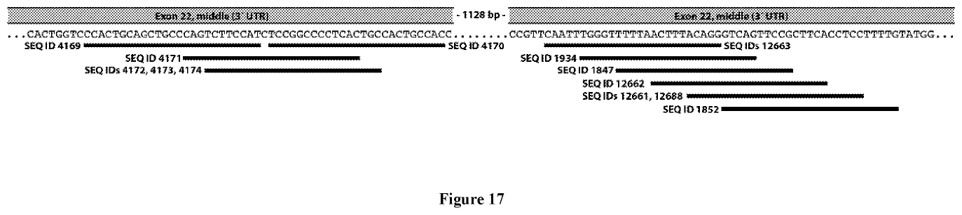

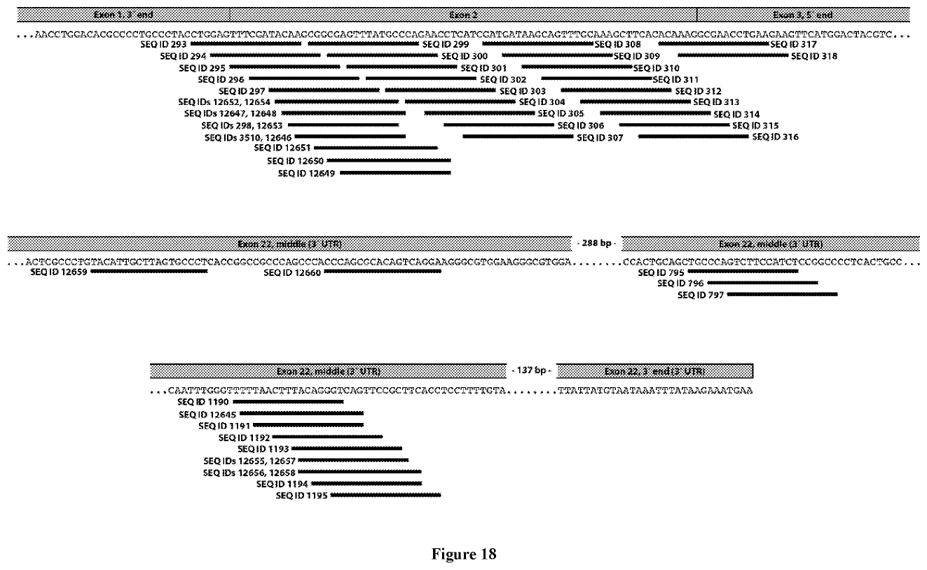

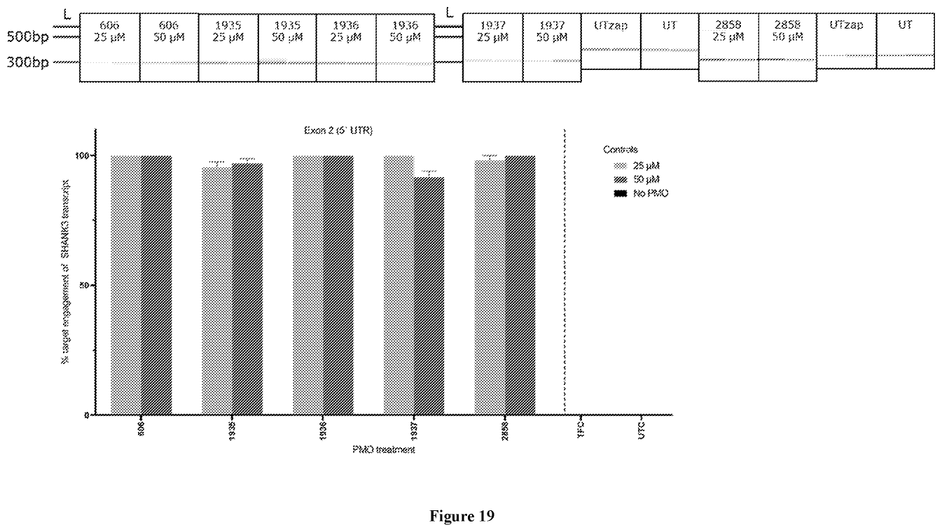

As shown in FIGS. 1, 4, and 5 some of the tested PMOs induced increased SHANK3 protein levels relative to controls and other PMOs failed to induce any change in SHANK3 protein levels. As shown in FIGS. 2 and 6, a subset of the PMOs increased SHANK3 protein levels from about 50% higher than controls to greater than four fold.

### Example 3: Measurement of SHANK3 Protein Upregulation by MOE Oligonucleotides Targeting SHANK3 mRNA 5′ UTR or 3′ UTR in a Neuronal Cell Line

A subset of the above-identified ASO sequences were synthesized as 2′-O-(2-methoxyethyl) (MOE)-modified oligonucleotides, which also contained a fully phosphorothioated backbone (“MOEs” 1-6, 13-60) targeting the canonical SHANK3 mRNA 5′ UTR, 5′ PNCR, and 3′ UTR regions. The numbered MOEs, corresponding SEQ ID NOs, and target regions are shown in Table 2 below. The antisense MOEs were electroporated into SH-SY5Y neuronal cell line cultures using the NEON® electroporation system (ThermoFisher) at concentrations of 2.5 μM and 5.0 μM and incubated for 96 hours. At this time, total protein was extracted using 15% SDS complete lysis reagent and the levels of SHANK3 protein assessed by western blot using a mouse anti-SHANK3 monoclonal antibody (Merck, Cat. No. SAB520004) at a dilution of 1:1000 in 5% BSA in TBST buffer followed by goat anti-mouse IgG H&L antibody (Abcam, Cat. number ab216776). Expression levels of the SHANK3 protein were compared to no MOE-transfected cells (untreated), control treated with transfection conditions and scrambled/non-targeting control sequences. The average signal from SHANK3 following imaging analysis was normalized to the average signal of total protein and ‘housekeeper gene’ proteins (e.g., vinculin, beclin, SRSF4)

TABLE 2
Exemplary MOEs Tested for Ability to
Increase SHANK3 Protein Levels

| | SEQ | SHANK3 |
|---|---|---|
| | ID | Region |
| MOE | NO | (mRNA or pre-mRNA) |
| MOE1 | 1191 | 3′UTR |
| MOE2 | 12645 | 3′UTR |
| MOE3 | 12646 | PNCR |
| MOE4 | 12647 | PNCR |
| MOE5 | 3510 | PNCR |
| MOE6 | 12648 | PNCR |
| MOE13 | 12649 | PNCR |
| MOE14 | 12650 | PNCR |
| MOE15/34 | 300 | 5′UTR |
| MOE16 | 12651 | PNCR |
| MOE17/32 | 298 | PNCR |
| MOE18 | 12652 | PNCR |
| MOE19 | 12653 | PNCR |
| MOE20 | 12654 | PNCR |
| MOE21 | 12655 | 3′UTR |
| MOE22 | 12656 | 3′UTR |
| MOE23 | 12657 | 3′UTR |
| MOE24 | 12658 | 3′UTR |
| MOE25 | 12659 | 3′UTR |
| MOE26 | 12660 | 3′UTR |
| MOE27 | 293 | 5′UTR |
| MOE28 | 294 | 5′UTR |
| MOE29 | 295 | 5′UTR |
| MOE30 | 296 | 5′UTR |
| MOE31 | 297 | 5′UTR |
| MOE33 | 299 | 5′UTR |
| MOE35 | 301 | 5′UTR |
| MOE36 | 302 | 5′UTR |
| MOE37 | 303 | 5′UTR |
| MOE38 | 304 | 5′UTR |
| MOE39 | 305 | 5′UTR |
| MOE40 | 306 | 5′UTR |
| MOE41 | 307 | 5′UTR |
| MOE42 | 308 | 5′UTR |
| MOE43 | 309 | 5′UTR |
| MOE44 | 310 | 5′UTR |
| MOE45 | 311 | 5′UTR |
| MOE46 | 312 | 5′UTR |
| MOE47 | 313 | 5′UTR |
| MOE48 | 314 | 5′UTR |
| MOE49 | 315 | 5′UTR |
| MOE50 | 316 | 5′UTR |
| MOE51 | 317 | 5′UTR |
| MOE52 | 318 | 5′UTR |
| MOE53 | 1190 | 3′UTR |
| MOE54 | 1192 | 3′UTR |
| MOE55 | 1193 | 3′UTR |
| MOE56 | 1194 | 3′UTR |
| MOE57 | 1195 | 3′UTR |
| MOE58 | 795 | 3′UTR |
| MOE59 | 796 | 3′UTR |
| MOE60 | 797 | 3′UTR |

As shown in FIGS. 10, 11, and 12 some of the tested MOEs induced increased SHANK3 protein levels relative to controls and other MOEs failed to induce any change in SHANK3 protein levels. As shown in FIG. 13, a subset of the MOEs increased SHANK3 protein levels from about 50% higher than controls to greater than four fold.

### Example 4: Identification of SHANK3 Target Intron/Exon Sequences

Identification of annotated and expressed transcripts that could generate the canonical transcript of the SHANK3 gene was performed by the sequence alignment of all SHANK3 protein-coding and NMD transcripts described in Gencode v38. An ASO sequence “micro-walk” of 25-mers in 5 bp increments of distance was performed over the sequences of intron 7, 17, 21, and exon 21 of the ENST00000262795.6 pre-mRNA transcript and designed to target the intronic splice enhancer motif to mediate exclusion of the retained intron or part thereof and generate productive SHANK3 mRNA transcript. The resulting ASO sequences correspond to SEQ ID NOs: 4187-12644.

### Example 5: Measurement of SHANK3 Protein Upregulation by PMOs Targeting of SHANK3 Pre-mRNA in a Neuronal Cell Line

The above-identified ASO sequences 25 nucleotides in length are synthesized as phosphorodiamidate morpholino oligonucleotides (PMOs). Antisense PMOs targeting intron 7, 18, or 21 (as described in Example 1) are electroporated into the naive SH-SY5Y neuronal cell line cultures using the NEON® electroporation system (ThermoFisher) at concentrations of 25 μM and 50 μM and incubated for 96 hours. At this time, total protein is extracted using 15% SDS complete lysis reagent and the levels of SHANK3 protein assessed by western blot using a mouse anti-SHANK3 monoclonal antibody (Merck, Cat. No. SAB520004) at a dilution of 1:1000 in 5% BSA in TBST buffer followed by goat anti-mouse IgG H&L antibody (Abcam, Cat. number ab216776). Expression levels of the SHANK3 protein are compared to no-PMO transfected cells (UT).

TABLE 3
Exemplary PMOs Inducing
Increased SHANK3 Protein Levels

| | | SHANK3 |
|---|---|---|
| | SEQ | Region |
| | ID | (mRNA or pre- |
| PMO | NO | mRNA) |
| PMO1 | 12634 | RI21 |
| PMO2 | 12635 | RI21 |
| PMO3 | 12636 | RI21 |
| PMO11 | 12637 | RI7 |
| PMO12 | 12638 | RI7 |
| PMO13 | 4204 | RI7 |
| PMO14 | 12642 | RI17 |
| PMO15 | 12643 | RI17 |
| PMO16 | 4969 | RI17 |
| PMO17 | 12644 | RI17 |
| PMO18 | 12639 | RI21 |
| PMO38 | 12640 | RI21 |
| PMO39 | 12641 | RI21 |
| PMO46 | 12672 | RI17 |
| PMO47 | 12673 | RI17 |
| PMO48 | 12674 | RI17 |
| PMO49 | 12683 | RI17 |
| PMO50 | 12692 | RI17 |
| PMO51 | 12689 | RI17 |
| PMO52 | 12693 | RI17 |
| PMO53 | 12690 | RI17 |
| PMO54 | 12691 | RI17 |
| PMO55 | 12675 | RI17 |
| PMO56 | 12676 | RI17 |
| PMO57 | 4974 | RI17 |
| PMO58 | 12677 | RI17 |
| PMO59 | 12684 | RI17 |
| PMO60 | 12685 | RI17 |
| PMO61 | 12686 | RI17 |
| PMO62 | 12687 | RI17 |
| PMO74 | 12678 | RI21 |
| PMO75 | 12679 | RI21 |
| PMO76 | 12680 | RI21 |
| PMO77 | 12681 | RI21 |
| PMO78 | 12057 | RI21 |
| PMO79 | 12682 | RI21 |

As shown in FIGS. 7 and 8 some of the tested PMOs induced increased SHANK3 protein levels relative to controls and other PMOs failed to induce any change in SHANK3 protein levels.

It will be appreciated by persons skilled in the art that numerous variations and/or modifications may be made to the invention as shown in the specific examples without departing from the spirit or scope of the invention as broadly described. The present examples are, therefore, to be considered in all respects as illustrative and not restrictive.

All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

Any discussion of documents, acts, materials, devices, articles or the like that have been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Alia et al., (2019), Frontiers in Neuroscience, 13:684.
Boyé et al., (2022), Nature Communications, 13:1169.doi.org/10.1038/s41467-022-28785-9.
Dugal-Tessier et al., (2021), J Clin Med., 10 (4): 838.
Fitzgerald et al., (2020), Stem Cells, 38 (11): 1375-1386.
Kim et al., (2014), Frontiers in Neuroscience, 8:109.
Prakash et al., (2019), Nucleic Acids Research, 47 (12): 6029-6044.
Rak et al., (2016), J Gene Med, 18 (11-12): 331-342.

APPENDIX
Sequences and SEQ ID NOS

SHANK3 mRNA Canonical Transcript 5′ UTR Sequence (SHANK3-201
ENST00000262795.7)
SEQ ID NO: 1
CACCACTCAGGCCAGGCCAGTGGCCTTGGGAGGGGCCTGTGATGCTGGGACCACAGTTCCTGG
GCAGGGAGCAACCGTCTAGGCGTGGGGAGAACGCAGGACGTGACCCACACACCGCACTGGAGG
CTCCGCTCTGCCCGGCTCCGGGACCCCTTCCTCCGCCGCACCCGCCCCGGTGGTCCGCGGATG
CCCGCCCTTGCCGCTCAGCCACTCCCCCCGCACCGAGGCCTAGGACTCCCCaggcgccgagct
gagccggggccgATGCAGCTGAGCCGCGCCGCCGCCGCCGCCGCCGCCGCCCCTGCGGAGCCC
CCGGAGCCGCTGTCCCCCGCGCCGGCCCCGGCCCCGGCCCCCCCCGGCCCCCTCCCGCGCAGC
GCGGCCGACGGGGCTCCGGCGGGGGGGAAGGGGGGGCCGGGGCGCCGCGCGCGGAGTCCCCGG
GCGCTCCGTTCCCCGGCGCGAGCGGCCCCGGCCCGGGCCCCGGCGCGGGGATGGACGGCCCCG
GGGCCAGCGCCGTGGTCGTGCGCGTCGGCATCCCGGACCTGCAGCAGACGGTGAGCCCCGCCG
CCCTGGGCCCGGCCGTGCCCCTGCGCTCCCCGCCCGGGATTCCCCCACCCCCGCCGGGCGCGC
CCGGCGCCCGGGACCCCCGGCCCACGGCTACTCACCCCTCCCCCGCCGCCTCCGCCGGGACCC
TCCCCATCGCCAGGGCGGGGCCCCGGGAAGCCCGGCCCCTGGGGCGGGGCTTCGGCCGCGGTT
CGCGGAGGCGCGGGGTCCCGGGCGCCGGCACCCGAGCCCCGGACTCCTTCGGCGGGGGCCCGG
GGCTCGGCACCCCGCATGGGGCCGGCGGGGCGGGTCCGCGCTCCCGGGACCTGAGCTCACGAG
CCCGCTCCGCTGCAGAAGTGCCTGCGCCTGGACCCGGCCGCGCCCGTGTGGGCCGCCAAGCAG
CGCGTGCTCTGCGCCCTCAACCACAGCCTCCAGGACGCGCTCAACTATGGGCTTTTCCAGCCG
CCCTCCCGGGGCCGCGCCGGCAAGTTCCTGGATGAGGAGCGGCTCCTGCAGGAGTACCCGCCC
AACCTGGACACGCCCCTGCCCTACCTGGAGTTTCGATACAAGCGGCGAGTTTATGCCCAGAAC
CTCATCGATGATAAGCAGTTTGCAAAGCTTCACACAAAGGCGAACCTGAAGAAGTTC
SHANK3 mRNA Canonical Transcript 3′ UTR Sequence (SHANK3-201
ENST00000262795.7)
SEQ ID NO: 2
CGCCCCACCCCCACTCCCGCCCCGGCCGTGCCCTGCCGGCAGGGCCCCCCACCCCCACCCCGGGCCGCGG
GCTCGGCCTGCCCCTTACGACGGCGCCCGGGCCAGGAATGTTGCATGAATCGTCCTGTTTGCTGTTGCTC
GGAGACTCGCCCTGTACATTGCTTAGTGCCCTCACCGGCCGCCCAGCCCACCCAGCGCACAGTCAGGAAG
GGCGTGGACCAGGGAGGCTGGGGCGGGAGGTGCCGGGGGTGGGGTGCCCTAGCGTGACCACCTCCTTCGC
AGCTCCTGGTGGCCATTCTCCCAGAGGGGGAACCTAGTCCAGCATGCGAGGTCAGGACCCGCCTTGGTGA
CTCGGGGGGAGGGGGGAGACATTGGGATTCTCGATGGGGGCCAAGGAGCCCCCCTGTTTTGCATATTTTA
ATCCACTCTATATTTGGAACGAGAAAAGGAACAAATATCTCTGTCCGTAATAGTTTCCTCTCCCCTCCCT
TCTACTTCCACTGGTCCCACTGCAGCTGCCCAGTCTTCCATCTCCGGCCCCTCACTGCCACTGCCACCCC
ACAACGGGGCAGGGGACGCTCCAGCTGGTCTGGGGTTGGCCAGGGCCCTAGTGGCCCGCCCTGGGGCCCC
AGCTCGGCCCCTCGCCTCGCTGAGCTCTAGTGTGCCCCACCGACCCTTCAGGTGCTGCTCGTGGTGGGAG
GGGCGGCAGGCCGCGGGTCCTGCTGTGCACCCGCGGGACCAGCCGGCCTGGGAGACCATCGGCCGGGGGG
GATGAGGGCAGGGCCCTGCCGCTCCACCGCAGCCATCTTCCTCACAGGGTCTCTCCCCAAGGAGGGGGCT
AGCTTGGTCCCCATGCTCTTGGGCAACTACAGCAGAGAAGCCTCCCTGCCTTGGACCCCAAAGTCTCCTG
TCCTGCCCTTTATGTGTGTGGGTGAAACTGGGTGCGTCTGAGCACGTGGGAGCCGTGTGTGTGCCTGATT
ACTGAGTGGCCACCAGGGGCCGCTCTGGACTAGCGCGGGGCCGTGGAGGCGTGCACCGTGTGCATGCGTG
GGGTGTACCTGTGAGAGCACCCTGTCTCCTCTTCCAAAGAAAGTCAGAGGCCATCCTGCACCCTGGGTCC
AGCTGTTTGCCCAGCCTGTCCTTCCAGAGCCTCACCCAGCCTGAGCGGGGTTCCCTGGTGAATCCCTGCT
GCTTGGGGAGGCCCCAAGGGCCCCTTGGAGGCAGCGCCCCCACCTTGGGCTTCTGAGGGCATCATAGGGG
GACCCCTAGAGTCAGTTCACCACAGGCCCTGGGGAGAGTCAAAGACCCCCGAGGGTGCCCAGCCCCCCAC
ACTGTGACTCCTCACACTCAGCGATGACCTGTGGGGTGGGGGGCCCTGGGACGTTTTTAAACCTAGGGTT
TGGAGTCTGGACTAAGCTCCATCCACGTCACTCACAAGTTTCTGTTTATATTTCTAGCTTTTTTTAATAA
AATAAAAAAAAAAAGAAAACAGAAGTTTTCACAACCCAGGGGCCTGGCACGCCGGTCTGTGCCTGCCCGC
CCCGCCCTGGCCCACCGGCCCCACTCCCTGGGCACAGAGTCACACCCACTCATCCTTCCGCCAACAGTCC
AGGTCACACAGCAGCAGTCACTGTAACAGACTGCCACATACACACTCGGTCTCACACTCACCTGTGGGTT
TTGGTTCCGTTCAATTTGGGTTTTTAACTTTACAGGGTCAGTTCCGCTTCACCTCCTTTTGTATGGAGTT
CCATCCGGGGGGTTTCACCCCCTGCTCCAGTCCTGAGGCCTCCTGACCCTGACGTTGTGATACGCCCCAC
AGAGATCTATGTTTCTTATATTATTATTATTGATAATAATTATTATAATATTATTATGTAATAAATTTAT
AAGAAATG
SHANK3 5′ PNCR of SHANK3 pre-RNA (SHANK3-201 ENST00000262795.7)
SEQ ID NO: 3
GCTTCGGCCGCGGTTCGCGGAGGCGCGGGGTCCCGGGCGCCGGCACCCGAGCCCCGGACTCCTTCGGCGG
GGGCCCGGGGCTCGGCACCCCGCATGGGGCCGGCGGGGCGGGTCCGCGCTCCCGGGACCTGAGCTCACGA
GCCCGCTCCGCTGCAGAAGTGCCTGCGCCTGGACCCGGCCGCGCCCGTGTGGGCCGCCAAGCAGCGCGTG
CTCTGCGCCCTCAACCACAGCCTCCAGGACGCGCTCAACTATGGGCTTTTCCAGCCGCCCTCCCGGGGCC
GCGCCGGCAAGTTCCTGGATGAGGAGCGGCTCCTGCAGGAGTACCCGCCCAACCTGGACACGCCCCTGCC
CTACCTGGAGgtaagtggccggcgcgggggtgagctgaggagcgcgcagggtggatcaccaagccccgtg
gcgggaccagtgaagggcacggcagtggggaaacacaagtgggaggggtgaggggtggaggctgtgtgtg
tgtgtgtgtgtgtgtgtgtgtggtgctgtgtgcagagtgcagtgagcgtgtacagggtgcagcgagcgga
cacagtgtatgcgatgagtaggcgcggtgtgtgcagtgagcgggcagggcgagcagtaaggatgtacagt
gtgggcagtgtgcgagcatgtgtagtgagcaggcagtgtgtgcagttagcaggcacagtgtgtgcagtga
gtgggcagtgtgcacagcatgtacagtgtgagtggtgtgtgctgtgtgccgtgagcatgtgtagtgagtg
ggcacagtgcagtcagtgtgcacaaagtatgcagtgggcacgtaccgtgtgtgcagtgagtggtgtgcag
tgtgtagtgtgcagtgagcagtgtgtacagcattgcagtgtggggcagtgagtacagtgtgagtgttgtg
attgtggtaagcaggatgcgcagtatacagtgaacagtgtgcacagtgtgtgcagtgtgggctgtgtgcc
acagagtcagtgtggtgtgtgtagtttgaacagtgtgtgcattgagcagcatggatggtgtggacgctga
gcattgttctccagggaggagtgtgagcacgagagagtgccagaggggtgtgtggtgtgagcagggctat
ctgtgtgcacgtttgttcctttctccagctgtgaagtcttgtgaaggccaaccaagtcccctcctttact
cacccatgcatggtgtgaagatgtattgagtgccttgctaggcatggggacgtagacggggtcagtcctg
tggaaggtcttggagtttggcagggtggaggggggtgcccaactgcagtgggcattgaataaagattttg
tgagctgagctcaaagttgggcgggcctccgtggtggcacaggaaagtggggtcagttctacctggagag
tggagggggatgtctaggatggtaagcctggaatcaggccttcagagaggagtgggattttgccgagaat
cctggggatgggaaggcgacgggacagtgcaggctgcgggcagctaggcacgtgccttccatcgggctgc
atcatgcctgagtgtggtgggtgcatggcagctgttagctctgtccactgtggtagtatgactgatggtg
tgtacaggagggcagtgaggggtgcggtgtggccagcatgagcgggacggggtttgtgcatggactcact
tgctcagccggggtgggggcattttctctaccttttctttatctgagcagTTTCGATACAAGCGGCGAGT
TTATGCCCAGAACCTCATCGATGATAAGCAGTTTGCAAAGCTTCACACAAAGgtaaaggatcacggggag
ggggctcctgaggttccctcctgcctccctgtggctgctgtcccccaccccagcttggggctgaccacag
tcccccagctttagctcagtccatttccccatcatcaggggcccagagcctgtactgggtgtggctgaag
ggctgggcacagattcctggccccatggttggggtcaggtggtacagtgatgtgttccagttagatcaga
ctcttgccgacccccctggcttaggggctggagtgtcctgtgagaagctgggtggagagggagtggacaa
gcatctgatgtgatggctgtcgggacaaggcacccagcatcgagtggtcgaccagtgctaggcatttgtg
attagacctctcattaatcctcttccagagggactattataaccctgtttcacagatggggacacagagt
acccgactgtgtagacagtgaagctggggctgaacccagatctgtctgattccaggccctgtgcccaggc
atgcctttgaggtgtctactgctgggtgccacccccaactgggcctgaccccaaatgctcttgagggggg
caccttactatttcccgctgaatgtcagaggggcagggtgggtgccacagcccctccccaggggcttcca
cccgcagctcacagtccagcaggcacttgtttgctggatactttatgggcggtctcgagctcaggagagg
ggtcagaatggagggtctctggagcccaggagaggaatcagaactgtcagccctgtgtcctaatggtccc
atcagtgactttggagcctcaggcttctgtgccaggcttttctgctgccccagggcaggcggtggtcaac
ggccttggcactatggctgggcagagtccactgtggaaaggtccccctctcctgccactggccctcactt
gaccttgtcagcctgggttcctaccccatggccacctctccctccggatctctcttcagtgaccaacaag
acatgagtgactcactctgaagtaggtctcgtttgtttttaggatgagcctgaacttcttacatagcccg
tgtttctgcttgcctgggctccagctggcctttccctctcccctggtagtgagcacttcctcctctttca
ctgcctggcccgcctgaggaggttgcctcccaaggcaggggtccctatggcaccctcccctatttctgag
gtacactgggtgtttatggaagggccccggcctttggccagggcaccttgccttgtgtgtgtggtgtccg
tagtgccggctggggaagtgaggccttgtggggtgagtgaataaactgggtgaatgagtacaaggccatc
aaagtcatgtcacaggatgggtccctggggtgggggtctgcggtggggtgagctggagaggaagatgggc
cgaagaaggagcaggttgtctcagggtgctgaggattcagggctgctgtggggctggttcctgcctgggt
ggacctcgggctcttctaggccaggcaggtcggggcagcagggccggagagacggagccagggcagtgac
tgggcctggagtgggggacttgcttggggccccaccagggtgacctggccttggtgaggggctacttggg
tacaagctgacagtcgtgactggtttggctatcagggcgcaaggaagggctttgagccgtgcatgggccc
acccgagtgggaattggggccgtggtgggagtgcaggaccgtggttgacaattgtgatgtcaggtgacag
gtcagactggtagggatgtggcgggggttgcctgaaggtggcctgaggcttgccggaggaaggcgggtga
tgttcagatgatggaggccttggtgccaggctgactgacggccggtgttccagGCGAACCTGAAGAAGTT
C
SHANK3 pre-mRNA Canonical sequence (SHANK3-201 ENST00000262795.7)
SEQ ID NO: 4
GCTTCGGCCGCGGTTCGCGGAGGCGCGGGGTCCCGGGCGCCGGCACCCGAGCCCCGGACTCCTTCGGCGG
GGGCCCGGGGCTCGGCACCCCGCATGGGGCCGGCGGGGCGGGTCCGCGCTCCCGGGACCTGAGCTCACGA
GCCCGCTCCGCTGCAGAAGTGCCTGCGCCTGGACCCGGCCGCGCCCGTGTGGGCCGCCAAGCAGCGCGTG
CTCTGCGCCCTCAACCACAGCCTCCAGGACGCGCTCAACTATGGGCTTTTCCAGCCGCCCTCCCGGGGCC
GCGCCGGCAAGTTCCTGGATGAGGAGCGGCTCCTGCAGGAGTACCCGCCCAACCTGGACACGCCCCTGCC
CTACCTGGAGgtaagtggccggcgcgggggtgagctgaggagcgcgcagggtggatcaccaagccccgtg
gcgggaccagtgaagggcacggcagtggggaaacacaagtgggaggggtgaggggtggaggctgtgtgtg
tgtgtgtgtgtgtgtgtgtgtggtgctgtgtgcagagtgcagtgagcgtgtacagggtgcagcgagcgga
cacagtgtatgcgatgagtaggcgcggtgtgtgcagtgagcgggcagggcgagcagtaaggatgtacagt
gtgggcagtgtgcgagcatgtgtagtgagcaggcagtgtgtgcagttagcaggcacagtgtgtgcagtga
gtgggcagtgtgcacagcatgtacagtgtgagtggtgtgtgctgtgtgccgtgagcatgtgtagtgagtg
ggcacagtgcagtcagtgtgcacaaagtatgcagtgggcacgtaccgtgtgtgcagtgagtggtgtgcag
tgtgtagtgtgcagtgagcagtgtgtacagcattgcagtgtggggcagtgagtacagtgtgagtgttgtg
attgtggtaagcaggatgcgcagtatacagtgaacagtgtgcacagtgtgtgcagtgtgggctgtgtgcc
acagagtcagtgtggtgtgtgtagtttgaacagtgtgtgcattgagcagcatggatggtgtggacgctga
gcattgttctccagggaggagtgtgagcacgagagagtgccagaggggtgtgtggtgtgagcagggctat
ctgtgtgcacgtttgttcctttctccagctgtgaagtcttgtgaaggccaaccaagtcccctcctttact
cacccatgcatggtgtgaagatgtattgagtgccttgctaggcatggggacgtagacggggtcagtcctg
tggaaggtcttggagtttggcagggtggaggggggtgcccaactgcagtgggcattgaataaagattttg
tgagctgagctcaaagttgggcgggcctccgtggtggcacaggaaagtggggtcagttctacctggagag
tggagggggatgtctaggatggtaagcctggaatcaggccttcagagaggagtgggattttgccgagaat
cctggggatgggaaggcgacgggacagtgcaggctgcgggcagctaggcacgtgccttccatcgggctgc
atcatgcctgagtgtggtgggtgcatggcagctgttagctctgtccactgtggtagtatgactgatggtg
tgtacaggagggcagtgaggggtgcggtgtggccagcatgagcgggacggggtttgtgcatggactcact
tgctcagccggggtgggggcattttctctaccttttctttatctgagcagTTTCGATACAAGCGGCGAGT
TTATGCCCAGAACCTCATCGATGATAAGCAGTTTGCAAAGCTTCACACAAAGgtaaaggatcacggggag
ggggctcctgaggttccctcctgcctccctgtggctgctgtcccccaccccagcttggggctgaccacag
tcccccagctttagctcagtccatttccccatcatcaggggcccagagcctgtactgggtgtggctgaag
ggctgggcacagattcctggccccatggttggggtcaggtggtacagtgatgtgttccagttagatcaga
ctcttgccgacccccctggcttaggggctggagtgtcctgtgagaagctgggtggagagggagtggacaa
gcatctgatgtgatggctgtcgggacaaggcacccagcatcgagtggtcgaccagtgctaggcatttgtg
attagacctctcattaatcctcttccagagggactattataaccctgtttcacagatggggacacagagt
acccgactgtgtagacagtgaagctggggctgaacccagatctgtctgattccaggccctgtgcccaggc
atgcctttgaggtgtctactgctgggtgccacccccaactgggcctgaccccaaatgctcttgagggggg
caccttactatttcccgctgaatgtcagaggggcagggtgggtgccacagcccctccccaggggcttcca
cccgcagctcacagtccagcaggcacttgtttgctggatactttatgggcggtctcgagctcaggagagg
ggtcagaatggagggtctctggagcccaggagaggaatcagaactgtcagccctgtgtcctaatggtccc
atcagtgactttggagcctcaggcttctgtgccaggcttttctgctgccccagggcaggcggtggtcaac
ggccttggcactatggctgggcagagtccactgtggaaaggtccccctctcctgccactggccctcactt
gaccttgtcagcctgggttcctaccccatggccacctctccctccggatctctcttcagtgaccaacaag
acatgagtgactcactctgaagtaggtctcgtttgtttttaggatgagcctgaacttcttacatagcccg
tgtttctgcttgcctgggctccagctggcctttccctctcccctggtagtgagcacttcctcctctttca
ctgcctggcccgcctgaggaggttgcctcccaaggcaggggtccctatggcaccctcccctatttctgag
gtacactgggtgtttatggaagggccccggcctttggccagggcaccttgccttgtgtgtgtggtgtccg
tagtgccggctggggaagtgaggccttgtggggtgagtgaataaactgggtgaatgagtacaaggccatc
aaagtcatgtcacaggatgggtccctggggtgggggtctgcggtggggtgagctggagaggaagatgggc
cgaagaaggagcaggttgtctcagggtgctgaggattcagggctgctgtggggctggttcctgcctgggt
ggacctcgggctcttctaggccaggcaggtcggggcagcagggccggagagacggagccagggcagtgac
tgggcctggagtgggggacttgcttggggccccaccagggtgacctggccttggtgaggggctacttggg
tacaagctgacagtcgtgactggtttggctatcagggcgcaaggaagggctttgagccgtgcatgggccc
acccgagtgggaattggggccgtggtgggagtgcaggaccgtggttgacaattgtgatgtcaggtgacag
gtcagactggtagggatgtggcgggggttgcctgaaggtggcctgaggcttgccggaggaaggcgggtga
tgttcagatgatggaggccttggtgccaggctgactgacggccggtgttccagGCGAACCTGAAGAAGTT
CATGGACTACGTCCAGCTGCATAGCACGGACAAGGTGGCACGCCTGTTGGACAAGGGGCTGGACCCCAAC
TTCCATGACCCTGACTCAGGAGgtgaggagtggagtcggggaggggcatggcctttgcgcggctgggagc
ctgacccttatctgtctgtgaacccagAGTGCCCCCTGAGCCTCGCAGCCCAGCTGGACAACGCCACGGA
CCTGCTAAAGGTGCTGAAGAATGGTGGTGCCCACCTGGACTTCCGCACTCGCGATGGGCTCACTGCCGTG
CACTGTGCCACACGCCAGCGGAATGCGGCAGCACTGACGgtcagtgagggcggggcctggcctggagggg
ctcttgcctggtgatggggctgggggcagctgggcctggtgtggatactgaggctgctcaccctcagACC
CTGCTGGACCTGGGGGCTTCACCTGACTACAAGGACAGCCGCGGCTTGACACCCCTCTACCACAGCGCCC
TGGGGGGTGGGGATGCCCTCTGCTGTGAGCTGCTTCTCCACGACCACGCTCAGCTGGGGATCACCGACGA
GAATGGCTGGCAGGAGATCCACCAGgtgtgcagggagccgaggtggggtcccggcctctgtgtgctgggt
tgggggtcctggctctgtctgtaggggtgggggccctagcctctgcccagggaccctacagcaccttgct
cttcccccagGCCTGCCGCTTTGGGCACGTGCAGCATCTGGAGCACCTGCTGTTCTATGGGGCAGACATG
GGGGCCCAGAACGCCTCGGGGAACACAGCCCTGCACATCTGTGCCCTCTACAACCAGgtgcgactgtgtg
tcctgcacatgcctgcaccagcgagtgtgcatatacttgcctcttctgggggtgtatgtgtgtgtgggca
cacaggtgaccctgtacggtgattgcatgtgtgcaccgagtgtggatatacttgcctgttctgggggtgt
acgtgtgtttgtgtgcacacaggtgaccctgtacagtgattgcatgcgtgcaccagggagtgtggatata
cttgcctgttctggggttgtacatgtgtgtgcacacagatgaccctgtacagtgattgtatgcgtgcacc
agtgagtgtggatatacttgcctgttctgggggtgtacgtgtgtttgtgtgcacacaggtgaccctgtac
agtgattgcatgtgtgcaccagggagtgtggatatacttgcctgttctgggggtgtacacgtgtgtttgc
acacagatgaccctgtacagtgattgcatgcgtgcaccagggagtgtggatatacttgcctgttctgggg
gtgtacacgtgtgtgtgcacacagatgaccctgtacagtgattgcatgcgtgcaccaggtagtgtggata
tacttgcctgttctgggggtgtacacgtgtgtttgcacacagatgaccttgtacagtgattgcatgcgtg
caccagggagtgtggatatacttgtctgttctgggggtgtacacgtgtgtttgcacacagatgaccctgt
acagtgattgcatgcgtgcaccagggagtgtggatatacttgcctgttctgggggtgtacacgtgtgttt
gcacacaggtgaccctgtacagtgatcgtacacgtgtaccagggagtgtggatatacttgcctgttctgg
ggttgtacgtgtgtgtgtgcacacagatgactctgtacagtgattgcatgcgtgcaccaggtagtgtgga
tatacttgtctgttctgggggtgtacacgtgtgtttgcacacagatgaccttgtacagtgattgcatgcg
tgcaccagggagtgtggatatacttgcctgttctgggggtgtacatgtgtgtgcacacagatgaccctgt
acagtgattgcatgcgtgcaccagggagtgtggatatacttgcctgttctgggggtgtacacgtgtgttt
gcacacaggtgaccctgtacagtgattgtacacgtgtaccagggagtgtggatatacttgcctgttctgg
ggttgtacgtgtgtgtgtgcacacagatgactctgtacagtgattgcatgcgtgcaccaggtagtgtgga
tatacttgtctgttctgggggtgtacacgtgtgtttgcacacagatgaccttgtacagtgattgcatgcg
tgcaccagggagtgtggatatacttgcctgttctgggggtgtacatgtgtgtgcacacagatgaccctgt
acactgattgcatgcgtgcaccagggagtgtggatatacttgcctgttctgggggtgtacatgtgtgtgc
acacagatgaccctgtacagtgattgcatgcgtgcaccagggagtgtggatatacttgcctgttctgggg
gtgtacatgtgtgtgcacacagatgaccctgtacactgattgcatgcgtgcaccagggagtgtggatata
cttgcctgttctggtgttgtacatgtgtgtgtgtgcacacagatgactctacagtgattgtatgcgtgca
gcaggtagtgtggatatacttgcctgttctgggggtgtacacgtgtgtttgcacacagatgaccctgtac
agtgattgtatgcgtgtaccagggagtgtggatatacttgcctgttctgggggtgtacatgtgtgtgcac
acagatgaccctgtacagtgattgtatgcgtgcaccaggtagtgtggatatacttgcctgttctgggggt
gtacacgtgtgtttgcacacagatgaccctgtacagtgattgcatgcgtgcaccagggagtgtggatata
cttgcctgttctggggttgtacatgtgtctgtgtgcacacagatgactctgtacagtgattgtatgcatg
taccagtgagtgtggatatacttgtctgttctgggggtgtacgtgtgtttgtgtgcacacaggtgaccct
gtacagtgattgtacacgtgtaccagggagtgtggatatacttgcctgttctgggggtgtacgtgtgttt
gtgtgcacacaggtgaccctgtacagtgattgcatgcgtgcaccagggagtgtggatatacttgcctgtt
ctgggggtgtacacgtgtgtgcacacagatgaccctgtacagtgattgtacacgtgtaccagggagtgtg
gatatacttgcctgttctgggggtgtacacgtgtgtttgcacacagatgaccctgtacagtgattgtaca
cgtgtaccagggagtgtggatatacttgcctgttctgggggtgtacacgtgtgtgcacacagatgaccct
gtacagtgattgtacacgtgtaccagggagtgtggatatacttgcctgttctgggggtgtacacgtgtgt
ttgcacacagatgaccctgtacagtgattgtacacgtgtaccagggagtgtggatatacttgcctgttct
gggggtgtacatgtgtgtgcacacagatgaccctgtacagtgattgcatgcgtgcactagggagtgtgga
tatacttgcctgttctgggggtgtacacatgtgtttgcacacagatgaccctgtacagtgattgtacacg
tgtatcagggagtgtggatatacttgcctgttctgggggtgtacacgtgtgtttgcacacagatgaccct
gtacagtgattgtacacgtgtaccagggagtgtggatatacttgcctgttctgggggtgtacatgtgtgt
gcacacagatgaccctgtacagtgattgcatgcgtgcaccagggagtgtggatatacttgcctgttctgg
gggtgtacacgtgtgtgtgcacacaggtgaccctgtacagtgattgcatgcgtgcaccagggagtgtgga
tatacttgcctgttctgggggtgtacacgtgtgtgtgcacacaggtgatcctgtacagtgattgcatgcg
tgcaccagggagtgtggatatacttgcctgttctgggggtgtacacatgtgtttgcacacagatgaccct
gtacagtgattgcatgcgtgcaccagggagtgtggatatacttgcctgttctgggggtgtacacgtgtgt
gtgcacacaggtgaccctgtacagtgattgcatgcgtgcaccagggagtgtggatatacttgcctgttct
gggggtgtacacgtgtgtgtgcacacaggtgaccctgtacagtgattgcatgcgtgcaccagggagtgtg
gatatacttgcctgttctgggggtgtacatgtgtgtgcacacaggtgaccctgtacagtgattgcatgcg
tgcaccagggagtgtggaaatacttgcctgttctgggggtgtacacgtgtgtgtgcacacagatgaccct
gtacagtgattgtgtatgtgcatccctgcctctgtgccatggtatatatatgtgctctgtgtcctgcagt
gagttgtggctgcagcacagcctcataggcatatgtgtgcacatttgttctctgaacacacaggggcttc
acatgtgtgcacgtgtgttctgaataaccaggtatgaattgggtacatctaggccctctgggccaggtga
gacctgagcgtgtatacctactggcttgtctctgcaactcaggtgtacatggaacaaataggtgtgagtc
cgtgtgtgtgagcctgtgccctgcgcacgccatgtgtgcattcctgtgtgcgcatgtgctgttgtgctcg
gatggtctctccagccacccagctgtgattccctcttccccgcaacagGAGAGCTGTGCTCGTGTCCTGC
TCTTCCGTGGAGCTAACAGGGATGTCCGCAACTACAACAGCCAGACAGCCTTCCAGgtacaccggtggtt
tacaggagctcaaggctgccccagaggtgtctgtctctgtgtccatgtgacttgacttctctgaaccttg
gttcttccctggaaggccctaagggagcacctcccccaggactgcccacaggaggtgttgggggacgagc
ccagcacgcgaggggtatttggtgttgatgttcccttcgtcccctcgccagggagagaggagggtcagca
gggctctggggcaggggtatggggaaaatgagaagactggggtgacaggtgtgggtctgaccccccaacc
ccgagagaccagcaggggtgcagaagccaaactgcagagggggtggagaggggggtggtggaggggggtg
gtggagaggggggtggtggagggggatggtggaggggggatggtggaggggggtggtggagagggggtgg
tggagaggggggtggtggagaggggtgtgatcctagccgctgatgtaattcaggggaggtttccgggccc
tttctctggccaggttggttggcactgatgaccaggtggacgtggtctctgacttggagtttgttgggga
gtcgggaatattgtgggggtttgcggaccaggaacagagccagaactcctctattttcagtaggagtgag
atgtgggaagagtggtcaggctgctgaggttgtgtggtcatagaaggtggaggcagcagtgggctgccag
atgggtttgggcacactgggaagcagatacgcccgggcctgttggatgactgggtgcagggtcacaggaa
ggggaggatgagcttggcagcttgggcagctgcggggccttggctgagatgggaaacatggctgcatgtt
gggacagaccagtgtgcccgtttgcttgggactgctggttttagctctgaacttctgtccaggcagcccc
tcagtcccaggcagacagagggtgggtcactccagttatagggtgtgcctgtggcatcccagttacagac
aagagtgtgtgtcagtagagaagaggaatgggctggggtctccggggcccccaaccaaggaagactccac
acacgaactggagaaggagcagctggggctggctggcgaggcaaagctggtgctgctcagcccctccctg
ctcctcaaggccttgacctcccctttccctcagGTGGCCATCATCGCAGGGAACTTTGAGCTTGCAGAGG
TTATCAAGACCCACAAAGACTCGGATGTTGgtgagttctgcccacctgggcgaccctgctgaatgtagat
tcgtgtggttttctggggcccagcagatgtgggggtcagtggtgacataaaggggccccacaccccacat
ctacactgtgtggccagtggtctctgagtggccactgcgggcatgaggaacgggccacagggcctgtgtt
gagtgaacattgtttttggtctgggagaagagcatggtgaggtttgggctgtgggccggggctagcattc
agcttagggggcagcataggttgagagtggggttgaagttagggtcagagtccctgtttcccgttaaatc
ctaggaagagccagcaggagacatgggggtggtggacctcacttcaccctggagcaagcatgagtcttgg
tggtccgtagcaagctctcagcatggaaagatcatgtaaacgacacataaggaaaacaacagtgatggga
aagcagaatggggctgggaatagtgccatagccatcctcagggcaaggagctctgagccccctgctgcct
gcgagtagaggaaagcctggtgtgtgcgtgactcacggtcctggaaggaaaaccaggagacacacacgtg
gcctcagccaggatacccgagcctcttggaggtctctggtgggtgtggggccaggctgggaccgtcctgt
tgtagctgccggagtcccctctgggagagttgtagctgccggagtcccctctgggagagttgctgctggc
caggctgggaccgtcctgttgtagctgccggagtcccctctgggagagttgtagctgccggagtcccctc
tgggagagttgctgctggccaggctgggacggtcctgttgtagctgccggagtcccctctgggagagttg
ctgctggccaggctgggaccgtcctgttgtagctgccggagtcccctctgggagagttgtagctgccgga
gtcccctctgggagagttgctgctggccaggctgggacggtcctgttgtagctgccggagtcccctctgg
gagagttgtagctggccaggctgggaccgtcctgttgtagctgccggagtcccctctgggagagttgtag
ctgccggagtcccctctgggagagttgctgctggccaggctgggatggtcctgttgtagctgccggagtc
ccctctgggagagttgctgctggccaggctgggaccgtcctgttgtagctgctggagtcccctctgggag
agttgtagctgccggagtcccctctgggagagttgctgctggccaggctgggaccgtcctgttgtagctg
ccggagtcccctctgggagagttgctgctggccaggctgggacggtcctgttgtagctgccggagtcccc
tctgggagagttgctgttggccaggctgggaccgtcctgttgtagctgccggagtcccctctgggagagt
tgtagctgccggagtcccctctgggagagttgctgctggccaggctgggaccgtcctgttgtagctgccg
gagtcccctctgggagagttgctgctggccaggctgggaccatcctgttgtagctgccggagtcccctct
gggagagttgtagctggccaggctgggaccgtcctgttgtagctgccggagtcccctctgggagagttgc
tgctggccaggctgggactgtcctgttgtagctgccggagtcccctctgggagagttgctgctggccagg
ctgggaccgtcctgttgtagctgccggagtcccctctgggagagttgctgctggccaggctgggactgtc
ctgttgtagctgccggagtcccctctgggagagttgtagctgccggagtcccctctgggagagttgctgc
tggccaggctgggacggtcctgttgtagctgccggagtcccctctgggagagttgctgctggccaggctg
ggacggtcctgttgtagctgccggagtcccctctgggagagttgtagctggccaggctgggaccgtcctg
ttgtagctgccggagtcccctctgggagagttgctgctggccaggctgggaccgtcctgttgtagctgcc
ggagtcccctctgggagagttgtagctgccggagtcccctctgggagagttgctgctggccaggctggga
ccgtcctgttgtagctgccggagtcccctctgggcgagttgctgctggccaggctgggacggtcctgttg
tagctgccggagtcccctctgggagagttgctgctggccaggctgggaccatcctgttgtagctgccgga
gtcccctctggtgcctgtgcctctctctggtatatggactcccaggacccccttcctgccatgagtgttt
actcgtgggtgtccccattttttgtttttgttttaatttgaagtgacagcgggcagcaaacgtctggagc
agatgttagggttggactctcacttggttctaatgtgaggtcatggtcagggctggagttgaattttgga
ctggggttaagattaaggtcggtttcatgtttaagggcccttcttctgtagagttccccctgaacatccc
cctgtaaggcttggttatgagcccctccaccccaggctgtgtcagttgaaccatctgaggctgtggcaga
ccctcggtgaatgccacagctgctctcaaggccactgcagggaagtgtgtgttgaggaagtaactgggcc
agtgggatgggtaggatcggagtccagggtcaggtcaggggagaactgtggtaaggtttgggtttgcata
aggggcagcagatagttacttctggtttgggctggtgttttggaggtggcccttagtgttggaaggccct
ggtttgtgcatgtgtggggcctcttcctctttttttttttttttttgagatggagtctgcctatgtagcc
caggcaggagtgcagtggcgcgatcttggctcactgcaagctccgcctcctgggttctcaccattctcct
gcctcagcctcccgagtagctgggattacaggtgcccaccatcatgcctggctaattttctgtattttta
gtagagacggggtttcaccgtgttagccaggatggtcttgatctcctgacctcgtgatccacccgcctcg
gcttcccaaagtgctgggattacaggcatgagccactgtgcccggccagggcctcctcttctgcctataa
ttgtctaaccgtgggaaagctgtctgggacctctgcgcctcagtttctcttaagtgaaacatggtctctt
atggtcatttgcatccccttccccctgtggtgtcctgtggcgtttgatcctgggtggggctggactcttg
ccggtggccttggtctagttgcttggagggagggcgggaaggccccatctgcccctcatctgtgcgttgt
ggacagataccccagggctcttggagtcaccatggttgcgacttactggctatccaacctaggtgtgctt
gcttggttggagttagacacacgtccctcagtggtaaggagcaggcacctgtgtcagttcatcctgatgt
ggtcgtcggctccatgggccaacaacacccttacctgatcaggtgccacacacagacactagggtgacac
cgccagatttccctgagtacaggcagcatatttgtcaggatttaatacaagccccttatctcaatttgcc
ctctcaacaaccccccaaggagtgattagactgttttacggagaaggaaactgaggcacagagggggtta
attcagacctgattgattcagggtctgcctttgtcactcccatgccctccgtacacatttgctccaagga
caggacagtcatgacttgctacatcctatgcccactggggtcccagagtggggcctgtacacagaagagg
aactggaaggaggtgccgccttctctggtgggcaggtgccacccggtgggatctggtagactgggacctg
tgtaacccttcaggcaacaggatagcttgctgcctctcgacagccttgcttgccccctctagactgacag
gccttgggtggggtccctggcctgggcgtggaatgggcatcccacagtgcttttgatgagtaccttgggg
aaatgcttctccagggtgctgtcatgcccatgagaacacacctgtgcctgggagtgtgtgcctgagagag
ggtaagagacaatgtggacccaaagagtgacccgacgtgacagaggcaaggaagcagaggtgaccacttc
taggccagaatgattcagtagcgttctgtatattcacggtgttgtgcagccgattctattgaattcaaaa
cattttcatcaccacaaaagaagaccctggacacattaagcaataattctgcatcaattaccctctccct
ccaactctgtcagccattaacacactttcgtctctgtggactatagatttacctattcttgatatttcat
gtcaatggaatcatataataagtgacacttagtgtctggctgcttttactcaacatgtttttgagcttca
ccttgtaggatatatcactgcttcattccttcttatggctgaataatatttcactgttggtataaatgac
attttgtttatgcattcatctgtcaatggatatgtgggtttctaccttttgactgttttgaatattgctg
ctgtcaacatgtgtgtacatgtacttgtttgaatatctattttcaatctcttaggtatatacctaagaat
ggaattttggaatcatatgataattctatgtttaacttcttgaggaactgccaaactgttttccaccatt
ttgcattcccaccagtgatgtatgaggcttccggttttttcacatcttcaccaacagttgttactatctt
ttttttttgaccgtagccatcctagtaagtgtgaaatggtatttcattgtgattttgacttgtatttccc
tagtgactaatgatgttttgcatcttttcatgtgcttattgggcatttgtatatcttctttggagaaaag
tcaagtcctttgtccatttttaaattgggtcatttatttttgttgttgagttgtaagggttctttatata
ttctgaatactaaacttgtgtggaatatgtgatttgtaaatattttctctgtagaataggagatccttgt
cacttttttgataatgtcctttgatgtacaagaatttttaattttgataaagttcaaggtatctattttt
ttcttttgttgttcatgcttttggtgtcataggtcacgagggtttatcctctaggttttcttctaagaat
tttacggttttcactcttaggtcactgatccattttaagttttaacttctgtttatggtgtgaggtaggg
agccatcttcattattttgcatatgaatatccaattgtccagcaccatttgttgaagagactattctttt
cccattgaatggtcttggcacccttgttgaaaatgaattgatcctagatatgtgggtttatttggactct
caattctatcccagtggtctacatgtatattcttatgccaggaccacatggtttgattactgcagctttg
tagtaactttttatttttatttttttggagatggagttttgctcttgtcacccaggctggagtgcaatgg
cgcagtctcagctcactgcaacctccgcctcccaggttcaagcaattctcctgcctcagcctccaaagta
gctgggattacaggtgtgtgccaccatgcccagctaagttttgtattattagtagagatggggttttacc
atgttgggcaggctggtctcgaactcttgacctcaggtgatccacccgcctctgtctcccaaagtgttgg
gattacaggcatgagccactgcacccggagtgtagtaacttttgaaatcaggaggtttgagtgcttcaac
ttgttcttatttgttatcgttttggctaatcagggcccctggcaattccatatagacttgaacattgctt
ttctatttgtgcaaaaagggacattggaattttgcattgaatctgtcatcactctgggtgacattaacat
tttaacaccattgtcttccaattaatgaacatgggatgtccttttatttatttaggtcatctctaagcag
tgttttgtcattttcagtatatagatttttttacttccttagttaaatttattcttgattattttcttct
tctggatgctattataaatggaattattttcttaatttcctttttggattgttcattgctggtgtgtaaa
aacaagtgatttttgtgtgctgatcttgtgccctacaactttgctgaatttgtttattagctctagtagc
tcttttggtgtattctttgggattttctacatataggatcatgccatctgcaaatagagatagttttact
tcttccttcccaatttgggtgccttttttttaatttttatttttgcctgtctagaactttcagtacggtg
ttgaataccagtagtaaaagcaggcatctttgccttgttcctgatcttagggggaaagcattcaatcttt
caccattgtgtatggtgctatctatgtgtgttttttttgtttgtttgttttttgttttttaaatatggaa
tgcttcattaatttgcatgtcatccttgtgcaggggacatgctaatctctgaatcattctaattttagta
tatgtgctgcagaagcaagcacttcggatttttaataaatttcctttattatgttgaggaagttactagc
ttctattactagtttcctgagtgtttttataatgaaagagtgttggattatggcagatgctttttatgca
tcaattgaaatgatcatttttccccctttgttctattaatgtgatgtattatattgattacattagtctg
tagatttcttttcctgcaatgcccttatctggctttggtagcagggtaatactggcctcgtgaaatgagg
gagtgttccctcctcttctgttttttggaagagtttgagaatttgtgtcaattctttaaatttctagtag
agtgcaccagtgatgccatctggtggtggacttttcttcgttgggaggcttgtggttaaggattcaatct
ctttgcttgttataggtctgtttaaattttctgtttcttcttgagtcagttttggtaatttgtgtttttc
tagaaatttgtctacctcatctagcttttctatttgttggcatgtaattgttcatagtgttatattcctt
tttaacttctgtaacactgatagtaagtccccaaatttcatttgtgattttagttatttctgtccatctt
ctctccttactagtcaatctagccaaaggtttgtcaattatgttgatattgtcaaagaaacaacctttta
tttccttgattctccgctgtttttctcttttgtttatctctgctctaatcttactgtttccttccttctg
ctagcttttctttcttttgctagtttggtttgctcttctttctcttgtttttttgtttgtttctttttgg
tctttctctagtctgtccgtccctccgtccctccctctcttgcttccttcctctttattttgtttgctct
tctttctctagtttcttcttttcttctctctctttttttttttggagatggagtttcgctcttgttgccc
aggctgtagtgcaatggtgtgatctcagctcaccacaacctccgccaactgggttcaagcgattctcctg
cctcagcctcccgagtagctgggattacagggacccaccaccatgcctggctaattttgtattttttaga
ggcagagtttcttcatgttggtcaggctggtctcgaactcccaacctcaggtgatctgcctgcctcggcc
tcccaagtgctgggattacaggcatgagccactgtgcccggcctcttttcttttcttttattggttcatt
catacagacaaggtctcactatgttgctaggctggtcttgagctcctgggctgaagtgatgctcccgctt
tggcctcccaaagtgctgggattacaggcatgagccactgagcctcgccttctcgtttcttttttgtttc
ttttttttcgtgagatggagtttcactcttggtgcccaggcggtagtgcaatggcgcaatcttggctcac
caccacctccgcctcctgggttcaagcaattctcatgcctcagcctcctgagtagctgggattacaggca
tgcactgccacacccggccaatttttgtattattagtagagacggggtttctccatgttggtcaggctgg
tttcgaactcccgacctcaagtgatctacctgccttggcctcccaaagtgctaggattacaggcgtgagc
cactgaacccggccttctcttgtttcttaagatgtataattaggttattgatttgagattgttttccttt
ttaaaagtaagtgtttacagcgttgtttccctttaagtactgcttttgctccatatgttgtatttttgtt
cttgtttgtcttaaagtgttttctaattttccttatgattgatttcttttttctctttctttcttttttt
tttttaagagatgggatctcactctatggcccaggctggagtgccctggcttgattttggctcactgcag
cctcaacctcctagtctcaagccatcctcccacctcagcttcctgagtagctgtaactacagggtgcagc
accacacctggctttttctattttttatagagatgggggtgtcactatgttgtccaggctggtctcaaac
tcctgacttcaagtgatcctcctgccttgacctcccaaagtgctgggattataggcttgagccaccattt
cttttttgagcctggtttcttttttgacacattggttgcttaggagtatggtgtttaatttccacatatt
tgtgaattttccagttttctttcacttctcgatttctagctttgtttcgttgtggttgaaaaagatactt
tgtatgatttcagtcttttaaaactttatcaagacttgttttgcggccaaacatacagtctgtctgggag
aatgttccatgtgtactcgagaagaattcttgaatatattctgctcttgttgggtggcatgttctgtacg
tatcgttgagtctaattggtttaagtcttctatgtccttattgatctctccagatgttctacccattatt
gaaagtaaggtattaaagtttcttactattattgtggaactgtctatttctctcttaaattgtgttgttt
gtatcatattttgatgttctgttgttcggtgtgtatatgttagtaattgttaatcaacatataatgtcct
tctctgtctcttgttacagtttttatcttaaagtttattttgtatgatgttaatataaccacccagctct
cttttggttactatttacattagattgctttttttatcctttcactttgaacctatttgtgtctttggat
ttaaagtgattattttgtaagtagcatatagttggataatactttttaatcactatctgctttttaagta
aaattctattggaacacaaacacacctatctgcttgtatgttgtttgtggtggttttcatgctgcggtgg
cagagttgagtcattgcaacacagactatatagcttgcaaagccttaagtagttactgtccaattctttc
cagaaaaggtttgctgatccctggtctagaatgtcttttatagtttccctggttatagttaactgcaaat
ggtcctgttcagcagtagtctggtgagattcgtcacatgtagcctgttttgtctctcctgaagcttgtga
aatgcagttcttagagcatatctgagcaccctcaaggcccctttcggctgagaggagaagcaggcttcta
cctctgaagctagggtgagaggcagtttgactgggatgtggccccttaatggtagcttcatttgtagggt
ggccaactcttttggtttgcctggaaccaaggcagttcctggaacataggatttagttttaacatctgga
aagcctgggcaaactggacaagttggtcacccagctggctgttcagagtcttacctatgcccccttaccc
cagTACCATTCAGGGAAACCCCCAGCTATGCGAAGCGGCGGCGACTGGCTGGCCCCAGTGGCTTGGCATC
CCCTCGGCCTCTGCAGCGCTCAGCCAGCGATATCAACCTGAAGGGGGAGGCACAGCCAGCAGCTTCTCCT
GGACCCTCGCTGAGAAGCCTCCCCCACCAGCTGCTGCTCCAGCGGCTGCAAGAGGAGAAAGATCGTGACC
GGGATGCCGACCAGGAGAGCAACATCAGTGGCCCTTTAGCAGGCAGGGCCGGCCAAAGCAAGATCAGgta
ggagggggctggcaggccctggaggggttgggagggtggggtgccgggacctgagccaggaggagccagc
accgggaggcagagagaggcctggtggtgcctagcacctgtgtagtgatgggctagggcttcctggttgg
ggaagggaagaaggcatctctggaggtggggacaggcacatgcacgctgatttccggttggagctgggct
tgccatgaggacagtgggcagttgagggagcgtgttaggtggagagggtatgctcctacctgtgatttag
aaaacacactgcattgtcaggcatggctgggaagagtcagggctggtggcctgtggtaagggggacaggg
atggagttgccactccagtagggcccaaaacctacaggtaagccccagccctgtccccagggtatactca
tggtttcccccagccctggcacacctacaggactctgtggcttctattctttaccctcaccctgaggcct
gggtccctgaggcagggatgcctggaccggagcccactggcctctctgggctgtgcattgggtgctgagg
tgggcgcactggctctcagccactttgtgcctgggcttctcatcagtaagtgggaatagtcctacctaag
tcattgtctttgggaagccttccagaatgccctgggcaggtgagggatcacctggcagagtctggcaact
gccggtaaaagcacctgccctccctgtgccttgaaggcaggcaccgtctttgtcgtttcctagtttcatc
agagccaggcactgaggagggtgtttggcagacattgttgagtggaagagttacataaacagtatgttgt
tgtcaggatggccgccggcctttacagttctgtcagtccaggcccgctgttgggctgaatggcatgtgta
tactagctcctttaaccttgacgaacgtacatgttcatgctctattaataccatgttacagatgagcgtc
ctggtacgcaggtggggcctgtgacgggtccagagtcacagagctacggatcagcttcactgggccccag
gtagacccaatccccaactttctaagcctgagtgttcagcagagggctcagggtgcagcagggcctatgg
aaggcgccccttcctgtgctgccgcccctgctctgcagcctggtctcagcgatgtctccgtgtctgtctt
tgcctgggcttgttggactcgcaaggctggctgtactgccccaaatctcaattctcccctgaagctttct
tatagacccgcggattttctaagggctgggcggcccagtggctgaaagaacacattctgcattccggatg
tttccatcccgcagaaaggctgctcctgagttggaggcacgcgttgccacaggctcctccctgcattcat
tttttgtacgtgttcattcatcctttcagcagacacaaaactaagacctgctgtgtgaggggctccagct
cacctgctctgtggggatggcagaattctgttttttccttcctcgcaaacatttattgggtgcctggtgc
gtcagaggtggtgggtctcaggaagttgacagccgagggcccgggctaagcagcagttaggtgggcagtg
atccccttatggggctgccctggtgagtggacaagggtctgcagctctcgggaggggctgaggaagttcg
gtgccccgaggagagaagtcaccccctgggggaggcgaggctgggccctgcaccgcgcgggtcgctgcgc
cctctgtcggttgagccggaccgggacccgacccccatcagccccccagtatcacacggggcacggcggg
ggagtttgggctgagagcccgtcacttaacagctggcccaggggtcaggatgttagagtagttcaggtgt
tggcaactgcgtgacggacagcccacaggaggggaaggagaggtgccccgtgtgactagagcgtagcaaa
ctgccaagcccgagggagcctggctcttggaaaagcgggtggctctggcctgcctgggtactagggagcc
actgcaggcttctgagcagagccccagtggaggagtgagagctcaggctctaccccagtctgggagtggt
ctggggaaggtgggcatcacgcagtgggcgtgggcaggggccggtgtccaggacgaggggacccaggcct
agagggggactgggcacccagcgatccgggccctggacctggaggggtggggggggcgcccctccctccc
gttcaccggctccaggcggctttgctggtgcccgaagcccccgccccatcccccgctcccactaggctgc
cctgaccaccgtcccgcgtccgcgtccgaactccccctcccgggggtcggcggcgaggggagggcgggag
ggagggcgcgagggccgccaccaccgcccgcagagggaggagcccggccgtggaggaggcggggcgcggg
gcggccgcggcatggagcgagcctggcgcgcccaggagctgtattcgaattcgagctcggttCCCCGCGC
CCCCTGCGCCCCCCGCACCGCCGCCCCGGGGCCCGAAGCGGAAACTTTACAGCGCCGTCCCCGGCCGCAA
GTTCATCGCCGTGAAGGCGCACAGCCCGCAGGGTGAAGGCGAGATCCCGCTGCACCGCGGCGAGGCCGTG
AAGGgtgaggggcgcgggggggcgcgggggggcgggcccggcgcggggagggggcggcgccgcgcgcggt
gctggccgggccggggcagtggctctggggtctcctctgccggggcggccctgggcccttgtgggatccc
tggtgtcacggtgaagggctctgcctggggaaggttcctgccgtgcgggtccctccggtgctctgtcgtt
ccgggctccctgtgtcaccacggaggctcctctctcgccacgggcgtttctgtgtccccgggggtctctg
cctgaaggaccctgtcccattacagagcttccttgcattgcggggttcccgtggcacttctgcagcttct
ccattggaggcccctgcgatgtgggggacccttgccgtcgtggggtgtctgtgaccgtcatgtgggtgtt
tgtgttatgcaggcttctgtcaccggggcttcctgtgttctggggggatcgcgtgccatgacaaacccct
ctcattctgggggtctcggggccatcactgggctcctagcctcagggccggctgaggtggaaacagccca
gctggtgcatcacgtggcctcacccactggccacagcacgatgaccccgagctctcggcagtgacccctg
ggtgggtgacagagccaggatgggggtcggttgaaggggctgggggagcatggtcagctgggggtggggg
cagcagcaggagtgtggcccctgcccctgcctgcgcccctccccgagtgtgtccatctgtgtgtctctct
gtcccccacatgcccaccctgtgccgagcccatctgttcctttctctcttctgcgtggatcccaaaatct
tcccagggaaaaagctgggagaaagtgggaagggaaggagggaaagggcagggggtgggtgggcagaacc
tgctcctgaggtggggtaggcgcccagctctgctccccactgacggcctgtctggcttcttcctccagTG
CTCAGCATTGGGGAGGGCGGTTTCTGGGAGGGAACCGTGAAAGGCCGCACGGGCTGGTTCCCGGCCGACT
GCGTGGAGGAAGTGCAGATGAGGCAGCATGACACACGGCCTGgtgagtgaccccacggctccccgggcag
ctcccaaggggaccaccccttccagtttccctttgttctctcttggtgctaaatccacatggatattcat
agagaaaagactagaggtaaactcaaacaaacactcaaagtagatgcaaacttgtgtacatgacacagac
acgtgtgcacacactctgcatatacttgggaacatgcgtgtatgtaacctgacgcttcacgtgcagtgga
gacacataggacgtgtgtgacggggcctctgtctgtgtgatcccacattgactcccagtgacttgcaccc
caccagcacagtccttcagaaacaccaggtgtgtggaacgcatgattcctgcgtagctggcagacatgta
aggaggtcagtgtgagaaaagaggcgtttttccaaagtggacagattgtccaagtgggcagagcaggcag
gcccggagcagccaagaggaaatgaggccagttgggcctggaggtgccctggtgagtgccctggggcagg
gatggtctgagggcagcaggtgaggctgggctggtcctcagcctgcagggaggatgtgaaggtgaaggct
gcagctcctgggttggtgagtggctgctgtccagccctgctgaccatctggtcctttgggggcccccggg
ctggagctgggtgcgtgtcttgggggtgcccttgcaaggaaccctcaggggtcccgggaggcccccagat
ccatgcatgtgcttctgtcctggagagctggtgggccaagcaaacctctcctgagtgatggtcactgggg
gccatcggtggtgtgtctggatcaagggtgcatgcaccctccctctgcatgtgaagggctcaggcctggg
gttactgtgtccccatctctgtgtccccacctctgagagtttcccagcgactccacccctgtacggcctg
gacccctgccctgtgctgagctcagcagaggcccagggaggcaggagcttcgccactgaccttttcctgg
gccggtgccctttcctccttccttggccttgttctgccttgctctgactggtggcttagagtgtggaagg
gacttggccccctgttctcagcccgcggagggtggatagggcagggtccagatgggaaatggtttcagag
atttgggccctggttcagacagtatgggagaggggaggggaaggagaggtagggggatccacacctgtgg
cgggagcgggtcttgagctcccccacgtggtggtgaggaaggttctggtttgaggactgtgtggagtgag
gggcatttggacctacgtctgaagctgacggggagggaggggggtttaggctggagtcagaattggagag
cagcaaatgttttttaggtgtttgtcatgtgctgggccctgtgctgggagccgggatcacggaggctcag
ctggaagccttgtgggagcagtggctgtgacgggggccgtgtgagcgtgggagctccctgggctggcctg
aaggagtcagagagggcctcaaaacagaggcattgtctgagctgagtcctctaggtggtcagcggctggg
gctgaggggacagccgtgtgtgcagggtccgtgtggcgctctacagctgtgtgtcagcacggcctgctct
gagtgcctggcacagttttagcacttctacacgtataagtcgttgactcatcccagcagtcccaagaggt
ggggccacttgttgttattccatttctagggggtcacatggccctgaggggcaaagtcttgtttgcaaca
caggcagcaagttctatgtgagtgaaacgcctgcatggggtgaggggcgaggctggtgtggggaccacgg
ggtggtcaggtggcatggagtcaagtgacagtgtggcatcatggcggagggaacccagccgcagtgtctg
attgtgcaggttgtgtaggatcaaagcccgggctgggcagagcagagcaatggagaggagggttcaggtc
tgggctccagccagcagccatggtgtgtgtgacctggggagtgagtgtcagaggtcccggggtagacggg
agtatttcatgagtgggcagcgggagagggacctgcggaggctgcctgcgaagggcagggagtgggagca
gtgacagtgccctctgaccttggctgcaggccctgctggccacactgcgcagagcctctgctctgggatc
ccggggccgagttctgccagaggccagagtgcagcccaggaacaggaacctgcttccaacaggcccagat
ctgctccccccaccccccaaaaaaatccatttccgggactggagtggggacagaaaaggacttaaaggtt
agggaggggacatagtccctgtgcgtgcaggggctggtgcccagagccggtgcctgctgtgggatcggga
ggcgagtcctggtcacttacccagcagagagcagtctcccacggcacctcctggcaggcagagggtggag
ctgtgagggagggaatggagaagcgaggtcaggtgtgttctggaggcccagcaggagcaggccctgtgcc
ttccctcccctccctccttccctttctttgcagtaaggtcagcacttgggtgggtagtaggagatggctg
tgttgggcattttctcttcagctggcttttatgtgtcttattaacaaaactggaaaaatgagtttgtgtt
atgtaatccatacaatttgtttggttgacaacatctgaaaacaggaggctggtcaagtgcaagtgtggcg
ggttcttcagcaggcagcaccatgacacttccgtgcctctgtcgacgtggagaagggtgtgatggaggaa
aggaggggtctgatgccccacctgagtggtgatgaggcttggcctgagctggccttaggttcagcgggat
gaggggtgcaggtgtggttggggaatgccaggataagatataggtcatcagaaccaaggggatcctccaa
gttggatttctctattctatatgggcgggcttagagggggctgttgacttactgagagtcacacaacgta
gtagctgggacctggaatccagcccgatacttttttgatatctgtgccttctgatagaaataggcactct
ttcctctttccgtccggccactgctccacctatgtttacagccatctatcctttgactcacccacccatc
ccaccatcatcttcttaaccgttgactcaccagccaacccaatccaccacccctcccttcctccatccat
ccacctctccacctacctgtctacacacccagtccatccatccatctacccatgtatctgtctgctcttt
gacctatctacccatggccctctccttccctccctccatccactggtccagtgcctttcaatccaccctt
ctgtccgtctatctcagatgcacctgtccaccatttactctcctagtcagcatcccttctgtccgtctat
ctcagatgcacctgtccaccatttactctcctagtcagcatgcagaatgcctgtgacgtgccaggtgctg
tgctgggtgctaggatcacagagataaagaacatgtggcccttaccctcaaggagctcacaattagtggg
ggaaacaggtcatcgaaagctgcaggttatctttctgatctcatttcctgccactctctctagctccctc
tgctccagctgcacaggctctgaactctctcaaatgctgcaggctgatgcctgccgtgggcttcccctga
ccacccccgctcctgggaagccctttgcccacatatccacaggctgctccccatcctcagcctctgctca
gccctcccgtgggggtgaggcctttgcgtgcctttctctgccctccctgatctgctcgctggtctcctga
gcacttgccagcattcgcatgctatgtctttatcctctgtttatcctgtctcccagaacagattataaac
cccacgagtgcagagactttgttctcttctctgctgtatttgcagagctggtacacagtaggagctcaat
aaatacttgctgaaggaaaacatggatcttgagaaatgctaccaagagaggaaatgaggaaggtgctgag
ggatgaaagcgaggcagtggtggtgctgcctgtggggcctttgcggtcctggagagtggagctgcggggc
tgggaaggctggtgggtggggctcgcaggcagctggaggagctcgtgggagcagagaggtcatggctgga
ggtgtggctcgaaccttcaggtcagtgagggtggagtggtgggcagacagctggctagcttggtgctggc
catgtgggacttgagatcaagaggacagttggggaccaagctctgtgggatgcatcgaggtcagggcagg
ggacccagcccacacacctagatcccctgccccactcacagttagtctgaagcaagggaccccatgaggt
tgagcatcaaggagctattgtgaagaagttggtgaagacagaacgctgcttccatgccagcgcctatagg
cacatagcacggcatttgtacatgagtgtgggtaatgcagattcagatgtgcacacacacatgcatgaac
acacctcacacgcacatgacggcaggtcacacagacacatgagcacggcacttgcacacagtgggctcat
atagagacacgtgcatgagcacagcatctacacatgaccacggatctctcacacacacacgtatgtgcac
acccccctctttgctcgcagccttggggctcacttacgctctgtggacggctctggccttgtttggactc
ctggttcagcagctacttaggaaagagatgagctgctgccccctcttgggccctgacccacggaggtcag
aattccctacatcatcccctcagccttcttacctctggacccaggactccaggatttggggccccctact
tccatctggacacaaccaggccaagggggtgtgtgtgggcagagactggtgaccagcatgggtgagggca
gttgaggcaggccagctggaggccacgactgtccatcagctcccgatactcccttcagAAACGCGGGAGG
ACCGGACGAAGCGGCTCTTTCGGCACTACACAGTGGGCTCCTACGACAGCCTCACCTCACACAGgtacgt
gcagggaccctggctggcgggagcatgcatgtgggacgtggggcaggacctgcagtgtagagggaggcca
ggtatgaagaggtcagaggagggcaagggaatgtgcctggccgacctctcttctgctgcccggcctgtca
gtcccggggtgactgcagggtggactgtggccggccaacctcgaggcagggcttacctcactcctccctg
ctttccttcatcagCGATTATGTCATTGATGACAAAGTGGCTGTCCTGCAGAAACGGGACCACGAGGGCT
TTGGTTTTGTGCTCCGGGGAGCCAAAGgtaatggggagtgggtgcccgggggtcaggcaggcaggggctg
cacaccgctcagggatgtcttcagcttcttgcttctgcccccaactctgctctgccccacactccctccc
tgccttgcccctgcggctcccgcttttggccccggctttcctggttcctctgctcggtcgcgaccgtcgt
ggtttgcaacgtcccggcccaggcggcaattccttcccttcgttgttttccactgtctttgctctagagt
ctaaccgagtgggggcttttctggcatgggcaaagggagttttgcagagggtcatgtctaggatocttgg
gccggctcgtccctgcgctgtggctccccgtggccctcccccagccccgggtcactgccccccgcagtca
tctctttcctgtgctgagccactcggaggttgctgtgtggcagccgcccccacccgaacctagctggtga
agcgccttcctaattgccccccgcagCAGAGACCCCCATCGAGGAGTTCACGCCCACGCCAGCCTTCCCG
GCGCTGCAGTATCTCGAGTCGGTGGACGTGGAGGGTGTGGCCTGGAGGGCCGGGCTGCGCACGGGAGACT
TCCTCATCGAGgtgaggtcgttctggccggtgctgcccagtgggatgctgagccctgagccctgtggtgg
acgtgcctggggcgtccccacccagctgcctgtctatcccagGTGAACGGGGTGAACGTGGTGAAGGTCG
GACACAAGCAGGTGGTGGCTCTGATTCGCCAGGGTGGCAACCGCCTCGTCATGAAGGTTGTGTCTGTGAC
AAGGAAGCCAGAAGAGGACGGGGCTCGGCGCAGAGgtgaggggtcacgcttcaggcctctgtgcccaaac
tttcccctgaccttagacctttgacttcaggcccacattcctcttcccatccctgacccccaactgctga
catcagattcctgaacctcaatgaaccacccacacgtgtgggctgggacccctcccgatccctggtgcta
gactgccagtcctgtcccctggcctgaggcctttgacccctgacccctgacctcaggctgcccctcctca
gatccctgccccctccttcccggcgttcccccagcccctggcgtggcagagccctccttccctacaccgc
cagcagctgcctggaggccggggttgccatagcaactgtgaggttgacgctgccgcgctgcttattgtcg
gagggaagggggaggcggcacctgagggaagaggaaaagcgtcttctccccgcgacgacgcggcacagcc
tgggcccaggaggctttgccggccaggcccagcccaggcccgtggcttggctgaccacaccctggcccca
caaggggcagtggccacccgggcctggggggagccctggagcagcaggagccccgcccgcaaggagaccc
ctcccagcccccaccccatggctctggtgcccccttgcttagccctcgaagcctgcagggtgggtgctca
ggccagagccagccggcaccgtgacccccgccagcacccttgatcgtctcctgaggccctgattctgggg
gttcccagggcccctgaggccctgaccgcccgcccccccccccccgcactccctgagctccttgagccat
gaagaagtttgcgtccagccgtaacctgaacaagatcctggcacagtgcgactcatcctcgcgggagtac
gaggagatccaggcggtggagcgcaagtggcacctgcacctggccacgccgcgccgcctgctgctggaca
ggaggtccaaggcctccctcttctttgcagCCCCACCGCCCCCCAAGAGGGCCCCCAGCACCACACTGAC
CCTGCGCTCCAAGTCCATGACAGCTGAGCTCGAGGAACTTGgtgagtggcgggggtggcggtggaggtgg
acgcaggtggacggtccatgatgggcagacagggccgggagacacaggtggtacgggagggcaggcggtc
cagccagagggtaggaaggacaggtgggtacaagagcaggtcagaggtgaatgggtgggtgctggcgcga
tggggaaatttggtgcttgtggggtgaggagagggtggcatgtggaaggggtgagatgtgagaggctggg
gatggacagtggggtgtccaggacagatgacggggagcagggcctggcccaccagtatgtttgcatgtgt
gtgcatgtgtgtgcgtgtgctgcctctggttttccacgtctccactttgactttactcctgcatttcatc
caagtgaggagagagcggtagtgaggtgaccagggtctgctgctggctctgccaggcactggctgtgtca
gctctgtgtttattttaccctcttggtgactgtgcactcattgacaggtggccctgctgaccccacaggc
cacaggagtactgtcggtgggatgatggggcagggcctcgagcgcggcaggaggggagcaggagtgtgtt
ggggtttgatggggcatgttctgggcacaagccctggttgaccatcaacagcctcttcatgggcaggtct
tatatatgtgtccattaacaaccttgtcatgtgcacgtccagatatacttgatcatgtcctgacgtgaca
gtcagtagcctcttcatgggactgtcccattacgtgtgaccgttgtcctcactgtgggtatgtgctatac
atgtggccatcaacagcctcgtcatgggcatgtcccactgtacgtgacagttgacaggctctttctttct
ttttttttttctttttttttttttttgagacagtttcactcttgttgcccaggctggagtgcaatgacac
gttctcggctcactgcaacctccacttcttgggttcaagtgattctcctgcctcagcctcccaagtagct
gggattacaggcacccgccaccatgcacggctaatttttttttttttttttttgtatttttagtagaggc
ggggtgtcaccatgttggccgggctggtctcgaactcctgacctcaggtaatctgcctgcctcggcctcc
caaagtgctgggattacaggcgtgaggctctttctttctgtgatctgagcccagttctgttcccagactt
aactctgcctctggtcatagaaggaaccccaactttggtcctaaactgaaccctgaagtcctggccacag
gaagggaccccattctggttctaggctgagccctatccctggcttcagtcttggttctaccccagccata
gaaggaacccttttcctggtccccagaattatccctgacctcactcatgaaccaagccctgaccctgtcc
ttgccacaggctgagctgccccatgccaagcaggtggtcttaactgcagtttagttcagggtggagaaca
gctaccaacgtccattgggctgacgtggtcagaattttcaccatagctattccaaataaaataaaaccca
aagaaaaaaacaaaaaaccccatgtagataattcagaatgaaaaggagagaaaccaaagccagaggcttc
catagctcgttatctagtacgcaggggcaaggggttggggtcctgaggcccccacaaggctgggagacgt
ggctggacctgtgagaagcgggaaggttaaaaaccaagcccaaccactggaattagcttcgaaagaactg
gaaatcagagaagaatatgaaaaacattaaaataaagtaaattgtcacaaagattaaaagaactaacaga
actaatgagagaataggacagaacaaacaagcaaaaaaagaataagcacattttagaatgattctttttt
ttttttttttttttttagactgagtctcgttctattaccagctggagtgcagtggcgcgatctcggctca
ctgcatcctccgcctcccgggttcaagtgattctcttggctcagcctcctgagtagctgggattacaggc
gcacaacaccacacccagctaattttttgtatttttagtagagatggggtttcaccatgttggccaggct
ggtctcgaactgctgaccttgtgattcacctgcctcggcctcccaaagtgctgggattacaggcgtgagc
cactgcgcccagccagaaggattcttaaagaactgttattgaaacaaaaagtgtagtcactgagataaaa
aagaaagccatgcctgggataaacagtacactagacccaggtgaagaaagaaacagcaagttagaagata
aatctgaggttacccagaatatagcacagaaatataaggagatagaaaatatcaaagagcagttaggaca
cacacacacaaaaatcacatagataattcaatgggactattgggaaaatcccagcatctacctaatgcaa
ggcgtggaagcaaaaataaaatgttgcctggaggcaatatttggagatgagatggcttaagaatttccga
aagttgaacaaagacatgagtcttcagtttaaagaaatataagtctcagcaaggaaataaaaacaaatct
gtatccacatctttcatagtaaaactgtagaatgctgtgagcaagcagaaatctttaaaacaatgagaga
ggctgggtgcggtggctcatgcctgtactctcagcactctgggaggccaagacaggtggatcacttgagt
ctaggagtttgagaccagcctggggaacatggcgaaaccctatctctactaaaaatacaaaaattatcct
ggcatggtggtgtgtgcctgtggtcccagctactcaggaggctgaggtgggaggattgcttaagcctggg
acgtggaggttgcagtgagccaagatcatgccagtgcactccagcctgggcaacagagcgagaccctgtc
tcaaaacaaaacaaagcaaaaaatgatgagagagagagagagatgggctgctggatgtcttagacgggag
cagtggtccaactgacaacagacttctcatcagcagtggcagatgacagtagatgatggaagaagagctt
caaagtgtggagtcaaaattcttttccacctagaattctataccccgctaatcctgctcattcaggagtg
aggcagagaggctaatcctagacctctggcactcgggtcatgagggtccaaggtcagtcacggggagctg
acctgtcaattgaagatccaaactgatcaataagccagcaccagtaggcagctgggtcccaggattctcc
tgccagaggctgtcacatagccacagtcagctgcatgttcagaagggactcttctattcagcaccacggg
gcttttgggcagcctgagatccgtaatcctgggacccagggactctgccctatggaaacggaaggactgc
gctttgctctctgggcttttctgcctgaagaaactggtgaatgaagacgctcctttccctcccctctggg
ctgagtgttccctcttctgcaacaagcctcaggacaggttgctggagtcactttgcagagcccggtcttc
cagaggagttgctcacagcttcaggccaccataattttccaggcttttttctgagcagagtctgggggag
gatgaagggacagcttagcgcagagcctctacccacacttctggagctcaggaagccaccctgtccccag
aagacagctgtcctgggaagccagcagggcttctggaagtcccctctggaaatgcctgatttgaggacgc
catgccagcatcagggtttccccaggtacctaagggaccatcccacaacccagtgtttttgctttcagaa
gatagaaatgaggcctgttggtcactgctggcatgaacacatcagaactgcctcacactgtcaagagcta
ttagagccctttaggatccatagtctatcgagaaggtgcctggacacctaaaaagaaaagaaagtgaggt
gtcagagaaagcaggtgcaagtcggaggctcctgtccaggtggctgtttgctgctcggaggatgtgggag
aggcatggccctgttggaggaaggggagggaaggggtatcctgtgggggaatggggtgagcaaaggctgg
tgcctggaaaatggagggttgcagagctgggagtgagaggaaggggctgggtggggaccctcctgagata
ggtggtgagagagagaagagggcagaacacccctcctcccctcagcactgggggctcctcatggtcgcca
catgcatgtctgctcggtttgtaccaggaatgtgctggggccacaggtcagtggagacccaggccaggga
aggccctgtggcgggtccaagcccttcctgtgtattcacgttcccgggagaactctggacttccctgtaa
tttctgggcccctttttcttttgtgatgagaacatcagggtaggtgccacacccagcatctatgtgagtg
gccttgtcctggaaaaggtttctgtgccaaacacacttgccctgccttttaagaccgactttgaacagaa
cacagaggtgaccaccttaattttcagcgacacctgaacttgttttctatggggttttgccttttttctg
aacaaacgtttgactgagccaagcatgatggagttgtgcatatctgtgtgtgtgtgcgtgtctgtgcgtt
ggtgagcggacacgcgctgtctgcacctgcgtgcatgcggcacacatgtctgtgcgtgggtgagtggaca
cgtgctgtgtgcacctgcatgcgtgtggcacacgtgtgtgcatgggtgagcggatacgcactgtgtgcgc
ctgcatgcgtgtggcacacatgtctgtgcgtgggtgagcagacgcgctgtgtgcacctgcatgcgtgtgg
cacacgtgtgtgcgtgggtgagcggatacgcgctgtatgcacctgcgtgcatgcagcacacgtctgtgcg
tgggtgagcggatacgcgctgtgtgcgcctgcatgcgtgtggcacacgtctgtgcgtgggtgagcggata
tgcagtgtgcgcctgcatgcgtgtggcacacgtgtgtgcgtgggtgagcggatacgcgctgtgtgcccct
gcgtgcatgcagcacacatgtctgtgcgtgggtgagtggacatgcgctgtgtgcacctgcatgcatgtgg
cacacacacacaagtccctgcttgtggttgtttcctccttggctttcaaggcatgtaccaactgactcca
gagttgggcagatttgctattcacggtctgataaagaggtgtaggctgggggcagatttgggaggtcatg
gtttttgggggaggtggtggtgagagggatggggtggggaaatgagtgtggataatggggcccgggtgcc
gagcctgcctcttactccctttactctgtttcttgattccaagCCTCCATTCGGAGAAGAAAAGGGGgtg
agtcatctgcctgtgtccccagggccttggctttgcctgacccctgtctgggggagtgctggtgtgtgag
gaggttccagcgcaagtcagggtggcctggaagcctgggctgcaccctcctgttggccttctccctgggc
atctggtaggggcatgagagaagggcctgcaaggatggatggggagttcccaggggtgtgaccctctgag
actgtgatggccttgggggctcaggtgagcctggggcctgtgagctgtgcagatagcctgatggaggcag
cccgtggggaggagctgggagagccccgtggaggccactgcatgcaccatggcccggacagcaggaggga
aagggctccgagggtcccagagggggagctctagggagcagggagactgggccgtgttggcgtgcacgtt
gcttctttctgctggtcctctcggggcctgtgcttgggttgtgggacctactcccgtgctggttggttgt
agggccaagcccagagctgatggtcccttcagagcaggggtctgagtcccacagcactggggaaggagct
tggagctcccctggccaaagaccccaggctgttgactaccctcctgaacccccgttcaggcctccctcta
ccctttggggctgctccctgtatgtcctttatgttcagcctggttgcaggggagggcgcattcaggtggg
gacagtcacctgggcctgggtactgaggtccactgtctggtgaagtcctgggacccggcttccggcagcc
tcctctgcctctcgggtggatgggaatcagggcccagacagaggcttgaggtggctttctccacccaggc
ctgtatatttgctccacgtccgtgtacctgatgcccatccctccctctctctcgacccactgggctttcc
ctgggtgaggacacatctgccaggcccttggcccagccctcttgaggtccccttgctgcctcttgcccca
gcccaggtgaagggcttccccagtgtcttctcaggtattaaaaacaggtgacaaggtctcctccagtgac
cttttctgcatgcctttggggcagacatacaagctatttctggaatcttctggctctgccctctagctcg
ttagtggggctcccagggtgctgtgttgcagcccaggtgctgctccagttgtggaagaagtgatggctgg
ttgatcatcccgcaggctggggccctgttgatcaaacagctgcaaacccagctgtgtcctgctgtgcacc
cagagggccctgggagtgagcgggtccacttggttcctgatgggctttgtttcccttaattatctaaaat
tatttatttaagaatatatcctctaatattggggggagatttatgtcaacctcatcagtttgtaatttgc
agaattcttgatgaatcctttcccttttgaaaacacttctcacccggctccagcaccatcgccggatgac
cagaaacagctgctcactaaacgttatgtgcgtcccgctgtgggtctctgggatgggtcttccagaccta
gagagacagcctctcctagcccgtggctcaggcggcaacgtgtctcccatgatgctgagccgtgtgtgcc
tcacggagttttctctccattcatatctttacttctgcagtttccggggagtatggtgcttaagtctcat
ctcttacttcctagtcagagatttatggcaatttcgcttcagtttgctcctgggttggataagggtgatg
ataaaatcctcgtgtagttgtgggatgagaggagtggtttgcataaagtcccgcatactctagaaatcac
caagaacttaacagatggtggcaatggtgacttcaactcccaaaaagaaatcattcacttcatgacaaaa
gcaaatgagaagtcccctcaaaccagcagcccctggtctgtacctgggcccctgtcacctggtggcactg
ggagctgtgcacaggcccagatatctgcagttggagcctgttttgctggtaaactcttgtatcgttcagg
acacaggctgggcgctgatggaacacagagaacagtatttgctctgttctgcagacccggaagctgtctg
gggagatggggcacacagacatgaattgtactgaattgtgtccagtgccagcacagttgtaagcctttct
tctgtgctaattctcactagagacacgtgagcgagcactgtttttatgcctgttttatcaatgaggacac
agaggtcacaaagaggttatgtcaggacagagtcagtgtacagggtgggtctggtctgggttgggtacag
gacagtcttggggagttccttccttccagcaatgctaagcttggggagttccttccttccttccagcaat
gctaagcagattgcagaggtagaatcagcaggtgctgtgaacagctgcagattggagttggaaggagtag
gaggtgtccagtctgaccgttgaaattctccaacaaggggccaagacaagagtgaaaggtgccaagacaa
gtgtggccagacagtggtccctctagggttggcatagccccaaagagtggagaagggcttggggttgggt
gcagcctgagtgatgggtggggtgcagtggggctgctgaatgagagggccggtgcagcagccaggtggac
atgagtgctgccccagcccttgctgcatgctgcacgcactggtcctcagtggacccacagggggcgagag
ctgcttatgtcctgggggagccatccaaccagggctctgatggtcctgagatacacgtattagtttaaca
gtttccaagtcactttgatcttctgtggaaacctgcttgtacatcagggtctgtcctcgggctgtgaccc
gctgtgaacagcttcccttccagtgcggtacagatagccaagagtggcctgtatgtgctgagaacagtga
caggacgaagggaaatggagggaaatccagggagactgtggtctctgaatcgtttgtctgtcctgtgccc
tggtcttctccctcatggtcagagtggcctttccttaagtgaggtccacagggtcacctggagtctcctt
gaggctagagaggctgcctggggtggggtctgagttctggggtgagggccttgtcttgggagcctggggt
gggagctcaaggagggaggggagcggtggcctctgtcagcatcacgggtggccgggttggggaggacatg
gcagtggggcaggaggtggcccaggcagctgagatggagcctccttgctgtgcagAGAAGCTGGACGAGA
TGCTGGCAGCCGCCGCAGAGCCAACGCTGCGGCCAGACATCGCAGACGCAGACTCCAGAGCCGCCACCGT
CAAACAGAGGCCCACCAGTCGGAGGATCACACCCGCCGAGATTAGCgtaagggccacgggcggctgggag
cgctgggtcgggcaggcatgggggtcagactgtcctgggccctcttgacggaggtgagagcctatcatgt
ggacccctctccagaggggtgtgtgcaccccatggcctctctcagagactcttggggctgtctctgcccc
ctaaatggccacagctcagcacctgctgggtttgctcttgtagcttttggtgcctctgccttcaggcagt
ccctgtctcacgcctgagaccaccaaggtaccccagctccgctgagaacacgacaaagcatgtctctgtt
ccctgcccacttgccaacccggcgccttctaggctcctcccccaccctcacccagggtcggccagggtct
gcacccaccctgcccaacaccgtctacactgtaccaccatctgcactgcccacacagcccaggctccaat
gggctgcatcctctgctccctgagggcagagtccagacgtgattcctgggtccaggcacccacaggcact
agtgccattggagtgagagcgtggggtgtctcacctctggcttaggaggaggactgggggcccccagtgc
cctggaacctccatattcccctccctgacccccacagTCATTGTTTGAACGCCAGGGCCTCCCAGGCCCA
GAGAAGCTGCCGGGCTCCTTGCGGAAGGGGATTCCACGGACCAAGTCTGTAGgtatggctgcgctgtggg
gctgcatggggtggggaggaacggggctggggccggcagggtggacttgggtttgaaggacactgcctct
ctctgcccataactggggtttccagtttccctctcagtcctgtctcttcctggccccagctaaaggatct
catacgttgatggacatgtggggattaggccttccccaacccagagctttcccccggcagccgacactcc
ctgtccagtgggcaccgccccccatcgcctcatccctcccatgggcagtctcatccctgtccccagctgc
cactccctgtccactgggcacccccacctccccatcacctctcatccctcccatgggcagcctcatccct
gtccccagctgccactccctgtccactgggcacccccacctccccatcacctctcatccttcccatgggc
agcctcatccctgtccccagctcagctgcctccatcgcggttgctcccttgcagcccaagtgcatgtgaa
gtttctgaccctcaaaccccctgaacttgcctctcccctacttctccatgtattgctgtctccccccttc
acagccagttttccccaaaaagtcacctctactgtccatcttgtctcccgtcccccaccggctcctcagc
ctgcggcagacttcctctctccatacccaaattgaaactgctcccaccagggtcaccggcggcctccagg
gccttcctccccatggatgcatggccagcactcctgctgacctcagctggcatttggtttctttgctcct
tcttccgtgaaacgctcctccccaaagctttaggacaacacttactggttttcccctcctttctcagatg
gcacctatttcaaccctgccgccccagcctcccaaatatgggaccttttcaaggctgtgtcttcagcctc
cttcccctctgtgcctcaccactccccagccaccatcacactccacactcaccgtctcctttcctgaccc
agatggggacatgtaacaagccctgcccactgtgcatccctccagacatccactctcccgtcccctgccc
acctgtgcatctgtccacccacacacctgcccacttgaccgtccgttgtccatacaaaccccagcacacc
tcccatctgttacacataggagtaaacctctcagcacttactacgggctggacactgagcatttaatact
tagaataaccatattgaggttgagaccactagtacctccattttacagatgagaaaactgaagcaaggag
ggtaaatgacctgcccaagatcacacagctgcccaaactcggattcctcacctgcctcaggaccctgcca
atttgtgttcacacaaggccgatcctgggccaagtctggggcttggttagggaatgtgacgtgtgcagag
agctcagcgggcacattgtgggtgccagggctcacggaccaatggagcccactggggggctccaagcaca
cctggtgtcacagaaggctgtgtgtgtcggggtgggggtacttttgagctaagatgtgcaagataaatgg
gagtttctgggcgataatggggtgaactgacagagcagtgtggaaaaggcatatgggtgtgtgacctggt
ccctcagagactgccaagagttatttctgtaggtgggattgtggggaagaaggctggtgaggctgcagct
actttggagctgagaagcgctctcccctgctctccctggcctaaacactgagtgtcttccatggtttctt
tcttatcctctgcctcacctgatgtgtcggggtgggtggcctcacagccctgccctgcacaggtctcatc
accttgcagctggaccatggctttgttctcatgactgcctgccctggctctgccctctggtccatccctc
tggccaaatcaggctttccgcagagctgctatcaccccatatccgtccagcttgaccctctgtgcactcc
tcattgcctcagggccctggagatcggccccagtgctctcctgcttcatctggaaacatttgtctctgcc
tcccttgacctgggtcttctgactcttgctgttcctgggcccgaccctgcaggtctaggtccatgcacct
gccctgcaggccttgagccactctcctaggactgccagttctgcactggtccctcaggcaccctcttgta
ctcggttgatggatgggtggacaggtgctcctttttggctcctcttggcctgctgggcctcagctgccag
catgcacattggttgggttgtcagactccccaggcccctgccccatgccctggctctgttgctctgggca
cacgatgcagcccttgaaacatcaggattttgatccataaacatggagaccacctattacccacctccaa
ggaccactctgagtctcccccggtagatggcactcacagaccgcgtggcatgctgggagtgctcagcagc
tgccagtgttagtagacgtagatgctgcctcttgaggttggcacacagtgggtgctcgagaaatgctctc
tgctatgacctaggcagacaaagagggtctgcctctgcccccaaggggtcccacccccgccccagatggt
atcagaacagattccccattccatgctgaggcaagtgacttttctttaccctaagtcagaacttggcatg
ctattagcattaagtttccccttatttcatctttgcacgtcactcccgccttctgcatcctcaaggtgcg
ctcattctgcgtcgctccatctaacacataggctgtgggtgttcactgcgtgttcatacacagtgcaggg
aatgcagcaggagacccaggacgaactgggcacaatttctctgtccagcggttggggatggggggaaggc
agacctaaaaatcacgatgatgatgacctcggcccccttcccccattcccaggatactgctgcagttcag
ccctgcactgtaaaggtgaaatcgccccacaatagttgcaggctgtcctcttgacatgaggaaactgaag
ttcctagaggcttctgaagttcctggcagatctaaaattcaagcctaggtctgtacacaaaagcccaccc
ttggtaccttggctacaaagagatattatgcccagcaagaagcagtgtagtactggaggcctcattagca
ctgcggtggctggaggaggatgcgatcaggaacaggcagtctgtattttagcgagttcataaaaatgttg
accaatctcagagtgaggctgggaacaggagcaccacgagacctgggtgtgtgggtgtggggccgtccgc
cccctgcagcctcactgaacacacctgcagcacgtgtgtccgcatcatgatgtccacaggggtgttcagg
aggttgtgtcaggttctgctggacagcaggagcatgagggccctgagcactccagcccctgcacaggacc
ctaacaatgatgtctgagcaaggcatgcagaggggggttactgaagctcaaaggggctcagggcctggcc
tggggtcacgctgccagtaggggttaaaatgaaacatgctgggtgctcagcctgcagttccattttgatt
gatccgtccgttcttcccttcacccactcacccatgcgggtctctgggaggagcctctcaaggcttcctc
cacagggcacctccttacctgggtgggcattaggtctttctctgctggaggcctggctccagtggccaca
gccctgctgcccctcaccctgccagtgtgtcactcctgctcatttccgactatcttcctctcacctctgg
ttgcctccagggctggtacttctgcgtcggccttgttgattctggggtggggagccctgtactggcccct
ccaagcccctcagcagttctgtccccatgtcttgcggggctgtccctgcctttctgggatatcttctcag
ggcctgcttgatgaccctgggtttgggcaggtcttggccccaacccaggccgtcagagtttgtgtccttt
ctcaggggtccccggtggggccctcctccatcctgtaactgaacgcacacctctctcctgtcctcttcac
aagagccctccccgtgcagccctgggcctggggcacagagcttgggcatccagggaccagcccagaccag
ggtcttgctcggagcccgggctctgggctccctgtttctcccctgccctccattccccgcccaccacggg
tcccaaccccatcttcccgagcattctagctcctcgcgccgggttctgccgcgggcgtccattgtgtccg
gacggtggcttccccggggtggagtcgggtcaaggctggcctctgtgggagggggttgccggggtcccca
ggaacctctccgaaggcagcaccaccccccgcccagcgccctggctggtctcaccggcccttccgtccgc
agGGGAGGACGAGAAGCTGGCGTCCCTGCTGGAAGGGCGCTTCCCGCGGAGCACCTCGATGCAAGACCCG
GTGCGCGAGGGTCGCGGCATCCCGCCCCCGCCGCAGACCGCGCCGCCTCCCCCGCCCGCGCCCTACTACT
TCGACTCGGGGCCGCCCCCGGCCTTCTCGCCGCCGCCCCCGCCGGGCCGCGCCTACGACACGGTGCGCTC
CAGCTTCAAGCCCGGCCTGGAGGCGCGCCTGGGCGCGGGCGCTGCCGGCCTGTACGAGCCGGGCGCGGCC
CTCGGCCCGCTGCCGTATCCCGAGCGGCAGAAGCGCGCGCGCTCCATGATCATCCTGCAGGACTCGGCGC
CCGAGTCGGGCGACGCCCCTCGACCCCCGCCCGCGGCCACCCCGCCCGAGCGACCCAAGCGCCGGCCGCG
GCCGCCCGGCCCCGACAGCCCCTACGCCAACCTGGGCGCCTTCAGCGCCAGCCTCTTCGCTCCGTCCAAG
CCGCAGCGCCGCAAGAGCCCCCTGGTGAAGCAGCTGCAGGTGGAGGACGCGCAGGAGCGCGCGGCCCTGG
CCGTGGGCAGCCCCGGTCCCGGCGGCGGCAGCTTCGCCCGCGAGCCCTCCCCGACCCACCGCGGTCCGCG
CCCGGGTGGCCTCGACTACGGCGCGGGCGATGGCCCGGGGCTCGCGTTCGGCGGCCCGGGCCCGGCCAAG
GACCGGCGGCTGGAGGAGCGGCGCCGCTCCACTGTGTTCCTGTCCGTGGGGGCCATCGAGGGCAGCGCCC
CCGGCGCGGATCTGCCATCCCTACAGCCCTCCCGCTCCATCGACGAGCGCCTCCTGGGGACCGGCCCCAC
CGCCGGCCGCGACCTGCTGCTGCCCTCCCCGGTGTCTGCCCTGAAGCCGTTGGTCAGCGGCCCGAGCCTG
GGGCCCTCGGGTTCCACCTTCATCCACCCACTCACCGGCAAACCCCTGGACCCCAGCTCACCCCTGGCCC
TTGCCCTGGCTGCCCGAGAGCGAGCTCTGGCCTCCCAGGCGCCCTCCCGGTCCCCCACACCCGTGCACAG
TCCCGACGCCGACCGCCCCGGACCCCTGTTTGTGGATGTACAGGCCCGGGACCCAGAGCGAGGGTCCCTG
GCTTCCCCGGCTTTCTCCCCACGGAGCCCAGCCTGGATTCCTGTGCCTGCTCGCAGGGAGGCAGAGAAGG
TCCCCCGGGAGGAGCGGAAGTCACCCGAGGACAAGAAGTCCATGATCCTCAGCGTCCTGGACACATCCCT
GCAGCGGCCAGCTGGCCTCATCGTTGTGCACGCCACCAGCAACGGGCAGGAGCCCAGCAGGCTGGGGGGG
GCCGAAGAGGAGCGCCCGGGCACCCCGGAGTTGGCCCCGGCCCCCATGCAGTCAGCGGCTGTGGCAGAGC
CCCTGCCCAGCCCCCGGGCCCAGCCCCCTGGTGGCACCCCGGCAGACGCCGGGCCAGGCCAGGGCAGCTC
AGAGGAAGAGCCAGAGCTGGTGTTTGCTGTGAACCTGCCACCTGCCCAGCTGTCGTCCAGCGATGAGGAG
ACCAGGGAGGAGCTGGCCCGAATTGGGTTGGTGCCACCCCCTGAAGAGTTTGCCAACGGGGTCCTGCTGG
CCACCCCACTCGCTGGCCCGGGCCCCTCGCCCACCACGGTGCCCAGCCCGGCCTCAGGGAAGCCCAGCAG
TGAGCCACCCCCTGCCCCTGAGTCTGCAGCCGACTCTGGGGTGGAGGAGGCTGACACACGCAGCTCCAGC
GACCCCCACCTGGAGACCACAAGCACCATCTCCACGGTGTCCAGCATGTCCACCTTGAGCTCGGAGAGCG
GGGAACTCACTGACACCCACACCTCCTTCGCTGACGGACACACTTTTCTACTCGAGAAGCCACCAGTGCC
TCCCAAGCCCAAGCTCAAGTCCCCGCTGGGGAAGGGGCCGGTGACCTTCAGGGACCCGCTGCTGAAGCAG
TCCTCGGACAGCGAGCTCATGGCCCAGCAGCACCACGCCGCCTCTGCCGGGCTGGCCTCTGCCGCCGGGC
CTGCCCGCCCTCGCTACCTCTTCCAGAGAAGGTCCAAGCTATGGGGGGACCCCGTGGAGAGCCGGGGGCT
CCCTGGGCCTGAAGACGACAAACCAACTGTGATCAGTGAGCTCAGCTCCCGCCTGCAGCAGCTGAACAAG
GACACGCGTTCCCTGGGGGAGGAACCAGTTGGTGGCCTGGGCAGCCTGCTGGACCCTGCCAAGAAGTCGC
CCATCGCAGCAGCTCGgtgagcagggcggtgcggggagggatccgtgccttgtccgtggccccgtctgtc
attcctcttgtctgttctctggctctcctgttcctccttgttctgttccattccttctgtggcaaccccc
aacccgcccccgccatccacctctgaatccggtcttgcttggcctgcccgagagaggaggttcttctggg
cgtctgacacgtcagggttgctgctcactgtgtccctgttggtgccagaagtgggagctgggctcccctc
agagactcaggttgggcatgaggcgcctccatggccctcctggaggctcttggccccagggattcctgtg
agttctctctctccctcccgacacaggctgtctttaccattaagggttgttttgtgttttactttggagg
tacgcctccctcctcccatcctctctgtggccatgtggctgcccagtgtggctgataccctctgccttac
agctgcctcctgccttgcttcctctggcggcttggagcacactgactccttttcttttgggggatctgcc
actaactccctatttcccatcccagaaacatttgtctcttggcaccaccttagaatcccttaaacatgga
ttcccgtgtgtcatttctaaagtgcagtaagaagatagatggaagtcacgccatctccctgcactccatc
tgccctcttgccttctccccaccacttccccgtctgtcctgcccctgcccagaaggatctgagcaggggc
ctggcttgctgctgaggctgtcacagctgccagagccctgggctgatcccataggtctttccttgaggac
ccgactcccaaggctccttccaaagaggagcccttcgggcccgtgggctgcatggatgctggcggcagag
ctggtcatcccccacccgcccccttgtctgcctttttaaagctgcttttgccttctgtgcccctaggtct
cccctctcctctttgggtctgggggggtggtatgtggatgccacctcttgactcctgcttcttgctgcct
ggaagaccaacctagtgggccccgtactgtcagccttggaggacagagttcacagcgtagcaacgtgttc
agaacttaaggactttgcaggtcttacaaaggcctggccattctaccttctttagttcaggattcaaaag
acaggtaggagcttgggaagctcatgaggcctctcctaaggtcccgggatgctgcctccagctcctgtca
tcctggggaattgctctggggtcctctccccttttagccttttccaactctcagccaaactggaaagccc
tcttcccagcagtgcagtgttgaaggtgcccgtagaatgggtgttataatcagagtgagcagcctggtcc
taggcctctgtacaggaccagacccctgaggctggggtctcctgacccacacctgaccagcccccatctt
ccctctctgcttctccctccgctcttctctgcctcttggtcttgatgaaaatcaaagccattttaaaaag
tgcatagcacagtgcctggcctggttcgggccctcaataaacatttcttaaatggatgaaagaacaaagc
aaaatgcaaatgctgtgttttgtgatttgagatctagggaggtggcttaggacaaaaacccacagaagga
cttactcagcgttcagactcatcagGTGTCGATCCCCCATGGTCGGGCTCAGgtgggcccaggtctcgtc
attggctctgtcctctcctgtgaggcagctgcagcagttgcagggcagggtcaggggattcctcagccgg
aacctctggcacttccccttctctgagttcatcctcccacggtgctctttagcattctgtcttgatcagg
gtacggttctcttagcccttggctctggctttcaccaggaccctgtttgtttttctttctcagatgtggg
atggggaaggacaagaccagcagccctgacccttacgtgacgtctgttcttttactcagtagttcctgca
aaattctgatctctgattgggttcatgtgggtcctctgcttttccctgactgatcactggggtctgggga
catagggctgtcatcggccaagcttgagtcctctgttctcccaggagccagggggatgggtccgcccacc
tagcaagcaggtgctgagagtgaggggagggggctctccagatggaagtccagtgctgtcccttgagtaa
gtagatgctggacagcctgtagcaaccaatgttctgtgacacggcccccactggcatcagcaactcactt
ccttgccggtcattggcttggagccatcagagggccctgatgtcggtgctcaggaggtcacagcttagtg
ctgtatccctcccctgtgaagtgtatttacagcgagccaactgcacaggctatcgcgaggctgcacacgg
catcacctgtgaagcacctggtaggcccagggctggggcctgtggacccctcactggtgtgagctgccat
tgtctttgctgtcactgacgacacctaggtgaggtcttagagtcctttgtgggaaaacaagcctgttgga
tgcagcactggagacggtcctgccgtatcagtttgtcttcacaccgttctgttagacggacactaacaga
atcctcatgttccagacaatgaaactgaggtaccagagagcttccttgcagacagaaaacggtggtctgc
ctgaccatgagccctgctctctggctgccgtaaccagggccgctgttgttaagccccttctgatgacaca
caatgggcttttgcccagtaacgcccctccacagaagtggcaggtgtgtgcatggcacagactcaccccc
gctgtttactggtcatgtttgtcataagtagcacctggtgccacctgagtggcctacccgggcctgcttt
tccttctcagtttgcctctgtgttaccttctgttccagctcactcttcctttcaccctcttgcgctcctt
ctcgccctcagacctgggctgaactacagccatctggaggggcccctgcccaccctgggcccttaacttc
cggtgcttctggctgcaggcagcggagcggagcacaggacacttggtgatgtctgagtccagtcctgttt
cagatctggtccttgtgtgcccaccgtggctgttgtcagctcttgtcctgcggcccctaatggctggagt
ccggtactcactgctcacttagggataaaactgctggtggaactgcctaaagtgtgggaggttctgacat
ccaggtatggctgcttccttcccagggccagtcgtggcagaggcgctaggtcaccctcagggccataccc
tgagctgtgtctccagccccttgtgttccacagtgtttgtctcccaggcccatccttctcctccctgacc
gacaccatgaggaagggaatgcccacccaccttttccagagatggctcattcctgatgtgctttagccac
aaggtgttctgtggcctgagagctttctggacaagttaagctgtcctgtatgtcgtggaaccaaagctcg
gtaacctgtggaaaaatgatgagctctggggccacctttgattttcaggggaacctggaggatgtggcac
tctcatgctgcctctggaaggtccaggaccttcggccttttgaatcctcgatcccagattgtccacctgc
tctgtgctcccagctccaagtctagctctgctggacagaccattcatctgcccagagcccctcctcggtc
tctgggccccacacctgactctgaggctacatttgggcatgattcagacctgactccgagccccacaact
ggccacaggcaccccatctgtctgaggctctcacttgtctctgagaccctggcttcacacctggctctaa
actccatttgtcttccaggccccgtgctcggatctgaatcccatgtggggtctgaggtccatagtaaggc
tcacgtctgccttggcacttagctttacatgggactctcacatctggttttgtaggtaaagttcatggca
aagtagctttggaaacacaaaaagaggttctttgctgcagtacttctggtgcctttgacaagcttgtgtg
gaggagggcactgtgtgccgtggtctccagacctgctagagtcagttcctaggtctcccagaagacccat
gttcctcaaaatggtcaagaagccctgccctggtggcctgctgcagaccctgcctgctaatatatttgtc
agggctctggacaaagcactgtgtgcttggtggatcacactggtaagcagggccaaggccagcagggcct
gctcttttgaaacccagactgaatgtgtcacatacctgccagtgggggcccctgcaggccacatgcagct
ggggcaggtgtgttcccaggatccctgaaccctgttcggggagtaggaaggaggtacagaggaggggtca
taggacactggggcccatcagagctggagctgggaacaggcggggtgccgaggagcaagacgtgttaccg
cagaggatgcactggaagcttccgagcccatgtctggttttgtgctcctctgagaattagttcccttaat
gaggcccctagtttgcaaaaggccacaggcctagaccggccccacgtgagtgcagtggcagaaggcccga
ggcagtatcccgggcccgagtgtggacattttcctgcctgttggccctagtgggctgactgagttgggca
cgcctggcagggacatgtcatacatactatttttgcattttgttagtgcatctcgtattcctggctttgt
gctaggtaccaggaggcaaggagaactggtaagaagtggggtgaagggcctgagaccatgaacggtctgg
atccagaaatggcaaggggtaaagagaaggtctgggttgatattaggagttagccattggctgtagggta
aaaagaggagctgatggtggctcccaggctctgtgtgtggcaatgacagggtgccgttcactgagttggg
gatggagggacatgtgtggataccccagtagaggtgttggactcatcagaggctgtctgggtctcacatg
tagcattcctgggcacactggccatagcaaagggtgtgagtggataatgttgcccaggagaaactgcaaa
ctaagaggggagctctccgaaaatccatctaggaaggaagaggcctgggaaagaatgtctcgaagggaaa
catgaacaggtcagcagcggggtcttgctgaccgcctggccaactgtggggcagagggcggagtggggag
ggtgtggcaaggccagacctctctcactggctgagagtcactgggaaacagtgagacggtgcaggattca
aatgtaggtggaaagacgtggggccgtgggggcctcagtgtgatctggactcagcctcttcagcgtggct
gctggaggtgttcgtgggtgacggtgcctggtgaagtatcatgtgttagagggtgggtggcccagaagct
ctctgggaggctggcaggtgctgtgatgtggctgtttggcggccggagcctggccagggaggtggccaga
cggctgagagtgacagtgggtgtgcagagaggccatggaaacatggaagtcagacatggacactgctcgg
gtttgtgccctggggatggtgtgggcagtaccctcttccccaagagaaagaggagaagcagacacctggg
ccatgggtgaccccaggcccctccctcctgtgccctggggcctttggcaggcacagccttccttgggcgt
tttctcttggcctcctgacatctccagcatgctctgggaggcgggagcaatgtgaatggctctcctcaca
tctcttgccctcgggcccctggctccgtgggtgcctctggagccagcctgccctctgcccaggtctccag
ctcagcccctgccagtactacccagacaggcacaactctgctctgcccagctcggacctcctggagccca
gaccctgctctgggctccggccgagcctcctggctgcgctgcccgcgcctgccctgtgcctctgccttca
ctgctgtggtcctgcaactcatcagtgtgcttcagtcacacctgagactccaggtacacctgccatcaca
actccactgaaagctaagccttggggcaggggcagggccagggtaggtgtccatcctgtgatacaggtcc
ccaggccagggccactttgttggggctggtgggtggaggccaggtgcatattcctggtcctgcccctctc
tgggtgtccacagtcaggtccctctgagtgattctctcggagtctcctctggcaagcccctccagcagtc
accaccacctgggcacccacccccaggggaccccccgacccaacaccacctggcacctccccttgggaat
ccccccaaccttggctgcctccccactttggcccactgtgcaggtggatccatggaggccaggtccaatg
ggagcaaagacagcttattctggtggttggtgcctgtgctgaggcccacctcaggtgttcttgaagcccc
tttctgtttgggggcacagtgtggcctcttcaggaagttctgctctggatctgagcagctgggaggaggc
agtagggacagaggctgaggagaggacagaaatgaggtcccatgggagagacagagctcagatctccaca
gagggctgacctctcacaggtgggagacaaaagacgaaacctgctttaaggggagccccatgcgtgtgtg
gggcaggcactggtgatgctgtgtggtgaggacagcctttaaggggagccccatgcctgtatggggcagg
gactggtgatgctgtgtggtgaggacagcacagaggttggggtggggggactcagcctgggcaggggcag
ggagagggggtgggagggccactggagaggggcccggccaaagaggatgtcaggggctcagcacccccag
gcggccaggcacctgccgagcactgcagccacgcttgtggttcacctgcacttggagactctcgcacagg
cgcctctcctccaccagcagctccagctttgtccctcccacagcagctgggccctctgcacctgagttcc
tccctccacacctggctgcccattgtgtggttgcagctgtgggtcgcgtgggcctggccggctaactctc
tctctcattgctctctcctgcccatctgcatgtggctgctctgtcgagctgcgccgtggtcccgagcgcc
ggctgtgtaagtgagcatgcccatcccatgcctcttgccgtccacacgctgtgcctgtctgcctgggtct
ctgctctgcgtggctgtgagaggctctggtgccactgacagccccttgaggcttcccctgacacagtggg
gatagggatgggaatgggaggacatgggagtgggttttctctggagctatcaccccaggtaggcctccca
ccacggcagagccaaggaggggctagagctctagggtcctgtcaggtgaggctgggaagtgagctgccat
cggttcttgtgtgtgtgcgtgcgtgcatgtgtgcgtgttgcgtgtctgcgtgtgtgtgctgctgccaccg
cagcatgtctgtaacgcgtgtgggcaccgttgctgctgttgtgtgccgtctgtgcaggaggctgcctttg
tgttgagggtgtgcacggcctcacacctgccctgcatgtgctgctgctccatacgggtacgagccctgcc
tagtgtctgtctcctgtgtcacggacctgttcaacctcgtgctgctgccagcctttatcgcgactcagct
gtccctggaacctgcccaggatcccctgggtcttctcatgagagcagagctgtgtggggggtgggcggtg
aagggtactgcccaagtctcagcgtcccgggtatctgtggatcccgccatgcccagagccggtgtcggag
gctggcaggagggagaagcccgccctttgccatgagaggctgtcttttctttggttgggctgcacttgga
gcctggatggagtggagggggccaccagtcattcctcatattccagccagtcgctggctctggtcccagg
ggccaaagaaaagggccagggtaaccgtaggatcccaccctttatttcttcctctggccgggctactccc
gccagccgcagccccagcccgtttcctcctggaccctgcccgctcccctccgcccgtccccccttggctg
tgcgcccctcacctggcgctgacccctctccctccgcagGCTCTTCAGCAGCCTCGGTGAGCTGAGCTCC
ATTTCAGCGCAGCGCAGCCCCGGGGGCCCGGGCGGCGGGGCCTCGTACTCGGTGAGGCCCAGTGGCCGCT
ACCCCGTGGCGAGACGCGCCCCGAGCCCGGTGAAGCCCGCGTCGCTGGAGCGGGTGGAGGGGCTGGGGGC
GGGCGCGGGGGGCGCAGGGCGGCCCTTCGGCCTCACGCCCCCCACCATCCTCAAGTCGTCCAGCCTCTCC
ATCCCGCACGAGCCCAAGGAGGTGCGCTTCGTGGTGCGCAGCGTGAGCGCGCGCAGTCGCTCCCCCTCGC
CGTCGCCGCTGCCCTCGCCCGCGTCCGGCCCCGGCCCCGGCGCCCCCGGCCCACGCCGACCCTTCCAGCA
GAAGCCGCTGCAGCTCTGGAGCAAGTTCGACGTGGGCGACTGGCTGGAGAGCATCCACCTAGGCGAGCAC
CGCGACCGCTTCGAGGACCATGAGATAGAAGGCGCGCACCTACCCGCGCTTACCAAGGACGACTTCGTGG
AGCTGGGCGTCACGCGCGTGGGCCACCGCATGAACATCGAGCGCGCGCTCAGGCAGCTGGACGGCAGCTG
ACGCCCCACCCCCACTCCCGCCCCGGCCGTGCCCTGCCGGCAGGGCCCCCCACCCCCACCCCGGGCCGCG
GGCTCGGCCTGCCCCTTACGACGGCGCCCGGGCCAGGAATGTTGCATGAATCGTCCTGTTTGCTGTTGCT
CGGAGACTCGCCCTGTACATTGCTTAGTGCCCTCACCGGCCGCCCAGCCCACCCAGCGCACAGTCAGGAA
GGGCGTGGACCAGGGAGGCTGGGGCGGGAGGTGCCGGGGGTGGGGTGCCCTAGCGTGACCACCTCCTTCG
CAGCTCCTGGTGGCCATTCTCCCAGAGGGGGAACCTAGTCCAGCATGCGAGGTCAGGACCCGCCTTGGTG
ACTCGGGGGGAGGGGGGAGACATTGGGATTCTCGATGGGGGCCAAGGAGCCCCCCTGTTTTGCATATTTT
AATCCACTCTATATTTGGAACGAGAAAAGGAACAAATATCTCTGTCCGTAATAGTTTCCTCTCCCCTCCC
TTCTACTTCCACTGGTCCCACTGCAGCTGCCCAGTCTTCCATCTCCGGCCCCTCACTGCCACTGCCACCC
CACAACGGGGCAGGGGACGCTCCAGCTGGTCTGGGGTTGGCCAGGGCCCTAGTGGCCCGCCCTGGGGCCC
CAGCTCGGCCCCTCGCCTCGCTGAGCTCTAGTGTGCCCCACCGACCCTTCAGGTGCTGCTCGTGGTGGGA
GGGGCGGCAGGCCGCGGGTCCTGCTGTGCACCCGCGGGACCAGCCGGCCTGGGAGACCATCGGCCGGGGG
GGATGAGGGCAGGGCCCTGCCGCTCCACCGCAGCCATCTTCCTCACAGGGTCTCTCCCCAAGGAGGGGGC
TAGCTTGGTCCCCATGCTCTTGGGCAACTACAGCAGAGAAGCCTCCCTGCCTTGGACCCCAAAGTCTCCT
GTCCTGCCCTTTATGTGTGTGGGTGAAACTGGGTGCGTCTGAGCACGTGGGAGCCGTGTGTGTGCCTGAT
TACTGAGTGGCCACCAGGGGCCGCTCTGGACTAGCGCGGGGCCGTGGAGGCGTGCACCGTGTGCATGCGT
GGGGTGTACCTGTGAGAGCACCCTGTCTCCTCTTCCAAAGAAAGTCAGAGGCCATCCTGCACCCTGGGTC
CAGCTGTTTGCCCAGCCTGTCCTTCCAGAGCCTCACCCAGCCTGAGCGGGGTTCCCTGGTGAATCCCTGC
TGCTTGGGGAGGCCCCAAGGGCCCCTTGGAGGCAGCGCCCCCACCTTGGGCTTCTGAGGGCATCATAGGG
GGACCCCTAGAGTCAGTTCACCACAGGCCCTGGGGAGAGTCAAAGACCCCCGAGGGTGCCCAGCCCCCCA
CACTGTGACTCCTCACACTCAGCGATGACCTGTGGGGTGGGGGGCCCTGGGACGTTTTTAAACCTAGGGT
TTGGAGTCTGGACTAAGCTCCATCCACGTCACTCACAAGTTTCTGTTTATATTTCTAGCTTTTTTTAATA
AAATAAAAAAAAAAAGAAAACAGAAGTTTTCACAACCCAGGGGCCTGGCACGCCGGTCTGTGCCTGCCCG
CCCCGCCCTGGCCCACCGGCCCCACTCCCTGGGCACAGAGTCACACCCACTCATCCTTCCGCCAACAGTC
CAGGTCACACAGCAGCAGTCACTGTAACAGACTGCCACATACACACTCGGTCTCACACTCACCTGTGGGT
TTTGGTTCCGTTCAATTTGGGTTTTTAACTTTACAGGGTCAGTTCCGCTTCACCTCCTTTTGTATGGAGT
TCCATCCGGGGGGTTTCACCCCCTGCTCCAGTCCTGAGGCCTCCTGACCCTGACGTTGTGATACGCCCCA
CAGAGATCTATGTTTCTTATATTATTATTATTGATAATAATTATTATAATATTATTATGTAATAAATTTA
TAAGAAATGAA

TABLE 4
Exemplary ASO or AR Sequences Targeting
SHANK3 mRNA 5′ UTR or 3′ UTR or SHANK3 5′ PNCR

| SEQ ID | | |
|---|---|---|
| NO | ASO Sequence | ASO Name |
| 5 | GCCTGGCCTGAGTGGTG | SHANK3 H1 5UTR(−2178−2162) |
| 6 | CTGGCCTGGCCTGAGTG | SHANK3 H1 5UTR(−2175−2159) |
| 7 | CCACTGGCCTGGCCTGA | SHANK3 H1 5UTR(−2172−2156) |
| 8 | AGGCCACTGGCCTGGCC | SHANK3 H1 5UTR(−2169−2153) |
| 9 | CCAAGGCCACTGGCCTG | SHANK3 H1 5UTR(−2166−2150) |
| 10 | CTCCCAAGGCCACTGGC | SHANK3 H1 5UTR(−2163−2147) |
| 11 | CCCCTCCCAAGGCCACT | SHANK3 H1 5UTR(−2160−2144) |
| 12 | AGGCCCCTCCCAAGGCC | SHANK3 H1 5UTR(−2157−2141) |
| 13 | CACAGGCCCCTCCCAAG | SHANK3 H1 5UTR(−2154−2138) |
| 14 | CATCACAGGCCCCTCCC | SHANK3 H1 5UTR(−2151−2135) |
| 15 | CAGCATCACAGGCCCCT | SHANK3 H1 5UTR(−2148−2132) |
| 16 | TCCCAGCATCACAGGCC | SHANK3 H1 5UTR(−2145−2129) |
| 17 | TGGTCCCAGCATCACAG | SHANK3 H1 5UTR(−2142−2126) |
| 18 | CTGTGGTCCCAGCATCA | SHANK3 H1 5UTR(−2139−2123) |
| 19 | GAACTGTGGTCCCAGCA | SHANK3 H1 5UTR(−2136−2120) |
| 20 | CAGGAACTGTGGTCCCA | SHANK3 H1 5UTR(−2133−2117) |
| 21 | GCCCAGGAACTGTGGTC | SHANK3 H1 5UTR(−2130−2114) |
| 22 | CCTGCCCAGGAACTGTG | SHANK3 H1 5UTR(−2127−2111) |
| 23 | CTCCCTGCCCAGGAACT | SHANK3 H1 5UTR(−2124−2108) |
| 24 | TTGCTCCCTGCCCAGGA | SHANK3 H1 5UTR(−2121−2105) |
| 25 | CGGTTGCTCCCTGCCCA | SHANK3 H1 5UTR(−2118−2102) |
| 26 | AGACGGTTGCTCCCTGC | SHANK3 H1 5UTR(−2115−2099) |
| 27 | CCTAGACGGTTGCTCCC | SHANK3 H1 5UTR(−2112−2096) |
| 28 | ACGCCTAGACGGTTGCT | SHANK3 H1 5UTR(−2109−2093) |
| 29 | CCCACGCCTAGACGGTT | SHANK3 H1 5UTR(−2106−2090) |
| 30 | CTCCCCACGCCTAGACG | SHANK3 H1 5UTR(−2103−2087) |
| 31 | GTTCTCCCCACGCCTAG | SHANK3 H1 5UTR(−2100−2084) |
| 32 | TGCGTTCTCCCCACGCC | SHANK3 H1 5UTR(−2097−2081) |
| 33 | TCCTGCGTTCTCCCCAC | SHANK3 H1 5UTR(−2094−2078) |
| 34 | ACGTCCTGCGTTCTCCC | SHANK3 H1 5UTR(−2091−2075) |
| 35 | GTCACGTCCTGCGTTCT | SHANK3 H1 5UTR(−2088−2072) |
| 36 | TGGGTCACGTCCTGCGT | SHANK3 H1 5UTR(−2085−2069) |
| 37 | GTGTGGGTCACGTCCTG | SHANK3 H1 5UTR(−2082−2066) |
| 38 | GGTGTGTGGGTCACGTC | SHANK3 H1 5UTR(−2079−2063) |
| 39 | TGCGGTGTGTGGGTCAC | SHANK3 H1 5UTR(−2076−2060) |
| 40 | CAGTGCGGTGTGTGGGT | SHANK3 H1 5UTR(−2073−2057) |
| 41 | CTCCAGTGCGGTGTGTG | SHANK3 H1 5UTR(−2070−2054) |
| 42 | AGCCTCCAGTGCGGTGT | SHANK3 H1 5UTR(−2067−2051) |
| 43 | CGGAGCCTCCAGTGCGG | SHANK3 H1 5UTR(−2064−2048) |
| 44 | GAGCGGAGCCTCCAGTG | SHANK3 H1 5UTR(−2061−2045) |
| 45 | GCAGAGCGGAGCCTCCA | SHANK3 H1 5UTR(−2058−2042) |
| 46 | CGGGCAGAGCGGAGCCT | SHANK3 H1 5UTR(−2055−2039) |
| 47 | AGGAAGGGGTCCCGGAG | SHANK3 H1 5UTR(−816−800) |
| 48 | CGGAGGAAGGGGTCCCG | SHANK3 H1 5UTR(−813−797) |
| 49 | CGGCGGAGGAAGGGGTC | SHANK3 H1 5UTR(−810−794) |
| 50 | GTGCGGCGGAGGAAGGG | SHANK3 H1 5UTR(−807−791) |
| 51 | CGGGTGCGGCGGAGGAA | SHANK3 H1 5UTR(−804−788) |
| 52 | GGGCGGGTGCGGCGGAG | SHANK3 H1 5UTR(−801−785) |
| 53 | CCGGGGCGGGTGCGGCG | SHANK3 H1 5UTR(−798−782) |
| 54 | CCACCGGGGGGGGTGCG | SHANK3 H1 5UTR(−795−779) |
| 55 | GGACCACCGGGGGGGGT | SHANK3 H1 5UTR(−792−776) |
| 56 | CGCGGACCACCGGGGCG | SHANK3 H1 5UTR(−789−773) |
| 57 | ATCCGCGGACCACCGGG | SHANK3 H1 5UTR(−786−770) |
| 58 | GGCATCCGCGGACCACC | SHANK3 H1 5UTR(−783−767) |
| 59 | GCGGGCATCCGCGGACC | SHANK3 H1 5UTR(−780−764) |
| 60 | AGGGCGGGCATCCGCGG | SHANK3 H1 5UTR(−777−761) |
| 61 | GCAAGGGCGGGCATCCG | SHANK3 H1 5UTR(−774−758) |
| 62 | GCGGCAAGGGCGGGCAT | SHANK3 H1 5UTR(−771−755) |
| 63 | TGAGCGGCAAGGGCGGG | SHANK3 H1 5UTR(−768−752) |
| 64 | GGCTGAGCGGCAAGGGC | SHANK3 H1 5UTR(−765−749) |
| 65 | AGTGGCTGAGCGGCAAG | SHANK3 H1 5UTR(−762−746) |
| 66 | GGGAGTGGCTGAGCGGC | SHANK3 H1 5UTR(−759−743) |
| 67 | GGGGGGAGTGGCTGAGC | SHANK3 H1 5UTR(−756−740) |
| 68 | TGCGGGGGGAGTGGCTG | SHANK3 H1 5UTR(−753−737) |
| 69 | CGGTGCGGGGGGAGTGG | SHANK3 H1 5UTR(−750−734) |
| 70 | CCTCGGTGCGGGGGGAG | SHANK3 H1 5UTR(−747−731) |
| 71 | AGGCCTCGGTGCGGGGG | SHANK3 H1 5UTR(−744−728) |
| 72 | CCTAGGCCTCGGTGCGG | SHANK3 H1 5UTR(−741−725) |
| 73 | AGTCCTAGGCCTCGGTG | SHANK3 H1 5UTR(−738−722) |
| 74 | GGGAGTCCTAGGCCTCG | SHANK3 H1 5UTR(−735−719) |
| 75 | GGCTCAGCTCGGCGCCT | SHANK3 H1 5UTR(−501−485) |
| 76 | CCCGGCTCAGCTCGGCG | SHANK3 H1 5UTR(−498−482) |
| 77 | GGCCCCGGCTCAGCTCG | SHANK3 H1 5UTR(−495−479) |
| 78 | ATCGGCCCCGGCTCAGC | SHANK3 H1 5UTR(−492−476) |
| 79 | TGCATCGGCCCCGGCTC | SHANK3 H1 5UTR(−489−473) |
| 80 | AGCTGCATCGGCCCCGG | SHANK3 H1 5UTR(−486−470) |
| 81 | CTCAGCTGCATCGGCCC | SHANK3 H1 5UTR(−483−467) |
| 82 | CGGCTCAGCTGCATCGG | SHANK3 H1 5UTR(−480−464) |
| 83 | GCGCGGCTCAGCTGCAT | SHANK3 H1 5UTR(−477−461) |
| 84 | GCGGCGCGGCTCAGCTG | SHANK3 H1 5UTR(−474−458) |
| 85 | GCGGCGGCGCGGCTCAG | SHANK3 H1 5UTR(−471−455) |
| 86 | GCGGCGGCGGCGCGGCT | SHANK3 H1 5UTR(−468−452) |
| 87 | GCGGCGGCGGCGGCGCG | SHANK3 H1 5UTR(−465−449) |
| 88 | GCGGCGGCGGCGGCGGC | SHANK3 H1 5UTR(−462−446) |
| 89 | CAGGGGCGGCGGCGGCG | SHANK3 H1 5UTR(−459−443) |
| 90 | CCGCAGGGGCGGCGGCG | SHANK3 H1 5UTR(−456−440) |
| 91 | GGGGCGGCGGCGGCGGC | SHANK3 H1 5UTR(−453−437) |
| 92 | GCAGGGGCGGCGGCGGC | SHANK3 H1 5UTR(−450−434) |
| 93 | TCCGCAGGGGCGGCGGC | SHANK3 H1 5UTR(−447−431) |
| 94 | GGCTCCGCAGGGGCGGC | SHANK3 H1 5UTR(−444−428) |
| 95 | GGGGGCTCCGCAGGGGC | SHANK3 H1 5UTR(−441−425) |
| 96 | TCCGGGGGCTCCGCAGG | SHANK3 H1 5UTR(−438−422) |
| 97 | GGCTCCGGGGGCTCCGC | SHANK3 H1 5UTR(−435−419) |
| 98 | AGCGGCTCCGGGGGCTC | SHANK3 H1 5UTR(−432−416) |
| 99 | GACAGCGGCTCCGGGGG | SHANK3 H1 5UTR(−429−413) |
| 100 | GGGGACAGCGGCTCCGG | SHANK3 H1 5UTR(−426−410) |
| 101 | GCGGGGGACAGCGGCTC | SHANK3 H1 5UTR(−423−407) |
| 102 | GGCGCGGGGGACAGCGG | SHANK3 H1 5UTR(−420−404) |
| 103 | GCCGGCGCGGGGGACAG | SHANK3 H1 5UTR(−417−401) |
| 104 | GGGGCCGGCGCGGGGGA | SHANK3 H1 5UTR(−414−398) |
| 105 | GCCGGGGCCGGCGCGGG | SHANK3 H1 5UTR(−411−395) |
| 106 | GGGGCCGGGGCCGGCGC | SHANK3 H1 5UTR(−408−392) |
| 107 | GCCGGGGCCGGGGCCGG | SHANK3 H1 5UTR(−405−389) |
| 108 | GGGGCCGGGGCCGGGGC | SHANK3 H1 5UTR(−402−386) |
| 109 | GGGGGGGCCGGGGCCGG | SHANK3 H1 5UTR(−399−383) |
| 110 | CCGGGGGGGGCCGGGGC | SHANK3 H1 5UTR(−396−380) |
| 111 | GGGCCGGGGGGGGCCGG | SHANK3 H1 5UTR(−393−377) |
| 112 | AGGGGGCCGGGGGGGGC | SHANK3 H1 5UTR(−390−374) |
| 113 | GGGAGGGGGCCGGGGGG | SHANK3 H1 5UTR(−387−371) |
| 114 | CGCGGGAGGGGGCCGGG | SHANK3 H1 5UTR(−384−368) |
| 115 | CTGCGCGGGAGGGGGCC | SHANK3 H1 5UTR(−381−365) |
| 116 | GCGCTGCGCGGGAGGGG | SHANK3 H1 5UTR(−378−362) |
| 117 | GCCGCGCTGCGCGGGAG | SHANK3 H1 5UTR(−375−359) |
| 118 | TCGGCCGCGCTGCGCGG | SHANK3 H1 5UTR(−372−356) |
| 119 | CCGTCGGCCGCGCTGCG | SHANK3 H1 5UTR(−369−353) |
| 120 | GCCCCGTCGGCCGCGCT | SHANK3 H1 5UTR(−366−350) |
| 121 | GGAGCCCCGTCGGCCGC | SHANK3 H1 5UTR(−363−347) |
| 122 | GCCGGAGCCCCGTCGGC | SHANK3 H1 5UTR(−360−344) |
| 123 | CCCGCCGGAGCCCCGTC | SHANK3 H1 5UTR(−357−341) |
| 124 | CCCCCCGCCGGAGCCCC | SHANK3 H1 5UTR(−354−338) |
| 125 | TTCCCCCCCGCCGGAGC | SHANK3 H1 5UTR(−351−335) |
| 126 | CCCTTCCCCCCCGCCGG | SHANK3 H1 5UTR(−348−332) |
| 127 | CCCCCCTTCCCCCCCGC | SHANK3 H1 5UTR(−345−329) |
| 128 | GGCCCCCCCTTCCCCCC | SHANK3 H1 5UTR(−342−326) |
| 129 | CCCGGCCCCCCCTTCCC | SHANK3 H1 5UTR(−339−323) |
| 130 | CGCCCCGGCCCCCCCTT | SHANK3 H1 5UTR(−336−320) |
| 131 | CGGCGCCCCGGCCCCCC | SHANK3 H1 5UTR(−333−317) |
| 132 | GCGCGGCGCCCCGGCCC | SHANK3 H1 5UTR(−330−314) |
| 133 | CGCGCGCGGCGCCCCGG | SHANK3 H1 5UTR(−327−311) |
| 134 | CTCCGCGCGCGGCGCCC | SHANK3 H1 5UTR(−324−308) |
| 135 | GGACTCCGCGCGCGGCG | SHANK3 H1 5UTR(−321−305) |
| 136 | CGGGGACTCCGCGCGCG | SHANK3 H1 5UTR(−318−302) |
| 137 | GCCCGGGGACTCCGCGC | SHANK3 H1 5UTR(−315−299) |
| 138 | AGCGCCCGGGGACTCCG | SHANK3 H1 5UTR(−312−296) |
| 139 | CGGAGCGCCCGGGGACT | SHANK3 H1 5UTR(−309−293) |
| 140 | GAACGGAGCGCCCGGGG | SHANK3 H1 5UTR(−306−290) |
| 141 | GGGGAACGGAGCGCCCG | SHANK3 H1 5UTR(−303−287) |
| 142 | GCCGGGGAACGGAGCGC | SHANK3 H1 5UTR(−300−284) |
| 143 | CGCGCCGGGGAACGGAG | SHANK3 H1 5UTR(−297−281) |
| 144 | GCTCGCGCCGGGGAACG | SHANK3 H1 5UTR(−294−278) |
| 145 | GCCGCTCGCGCCGGGGA | SHANK3 H1 5UTR(−291−275) |
| 146 | GGGGCCGCTCGCGCCGG | SHANK3 H1 5UTR(−288−272) |
| 147 | GCCGGGGCCGCTCGCGC | SHANK3 H1 5UTR(−285−269) |
| 148 | CGGGCCGGGGCCGCTCG | SHANK3 H1 5UTR(−282−266) |
| 149 | GCCCGGGCCGGGGCCGC | SHANK3 H1 5UTR(−279−263) |
| 150 | GGGGCCCGGGCCGGGGC | SHANK3 H1 5UTR(−276−260) |
| 151 | GCCGGGGCCCGGGCCGG | SHANK3 H1 5UTR(−273−257) |
| 152 | CGCGCCGGGGCCCGGGC | SHANK3 H1 5UTR(−270−254) |
| 153 | CCCCGCGCCGGGGCCCG | SHANK3 H1 5UTR(−267−251) |
| 154 | CATCCCCGCGCCGGGGC | SHANK3 H1 5UTR(−264−248) |
| 155 | GTCCATCCCCGCGCCGG | SHANK3 H1 5UTR(−261−245) |
| 156 | GCCGTCCATCCCCGCGC | SHANK3 H1 5UTR(−258−242) |
| 157 | GGGGCCGTCCATCCCCG | SHANK3 H1 5UTR(−255−239) |
| 158 | CCCGGGGCCGTCCATCC | SHANK3 H1 5UTR(−252−236) |
| 159 | GGCCCCGGGGCCGTCCA | SHANK3 H1 5UTR(−249−233) |
| 160 | GCTGGCCCCGGGGCCGT | SHANK3 H1 5UTR(−246−230) |
| 161 | GGCGCTGGCCCCGGGGC | SHANK3 H1 5UTR(−243−227) |
| 162 | CACGGCGCTGGCCCCGG | SHANK3 H1 5UTR(−240−224) |
| 163 | GACCACGGCGCTGGCCC | SHANK3 H1 5UTR(−237−221) |
| 164 | CACGACCACGGCGCTGG | SHANK3 H1 5UTR(−234−218) |
| 165 | GCGCACGACCACGGCGC | SHANK3 H1 5UTR(−231−215) |
| 166 | GACGCGCACGACCACGG | SHANK3 H1 5UTR(−228−212) |
| 167 | GCCGACGCGCACGACCA | SHANK3 H1 5UTR(−225−209) |
| 168 | GATGCCGACGCGCACGA | SHANK3 H1 5UTR(−222−206) |
| 169 | CGGGATGCCGACGCGCA | SHANK3 H1 5UTR(−219−203) |
| 170 | GTCCGGGATGCCGACGC | SHANK3 H1 5UTR(−216−200) |
| 171 | CAGGTCCGGGATGCCGA | SHANK3 H1 5UTR(−213−197) |
| 172 | CTGCAGGTCCGGGATGC | SHANK3 H1 5UTR(−210−194) |
| 173 | CTGCTGCAGGTCCGGGA | SHANK3 H1 5UTR(−207−191) |
| 174 | CGTCTGCTGCAGGTCCG | SHANK3 H1 5UTR(−204−188) |
| 175 | CGAACCGCGGCCGAAGC | SHANK3 H1 5UTR(+1+17) |
| 176 | CCGCGAACCGCGGCCGA | SHANK3 H1 5UTR(+4+20) |
| 177 | CCTCCGCGAACCGCGGC | SHANK3 H1 5UTR(+7+23) |
| 178 | GCGCCTCCGCGAACCGC | SHANK3 H1 5UTR(+10+26) |
| 179 | CCCGCGCCTCCGCGAAC | SHANK3 H1 5UTR(+13+29) |
| 180 | GACCCCGCGCCTCCGCG | SHANK3 H1 5UTR(+16+32) |
| 181 | CGGGACCCCGCGCCTCC | SHANK3 H1 5UTR(+19+35) |
| 182 | GCCCGGGACCCCGCGCC | SHANK3 H1 5UTR(+22+38) |
| 183 | GGCGCCCGGGACCCCGC | SHANK3 H1 5UTR(+25+41) |
| 184 | GCCGGCGCCCGGGACCC | SHANK3 H1 5UTR(+28+44) |
| 185 | GGTGCCGGCGCCCGGGA | SHANK3 H1 5UTR(+31+47) |
| 186 | TCGGGTGCCGGCGCCCG | SHANK3 H1 5UTR(+34+50) |
| 187 | GGCTCGGGTGCCGGCGC | SHANK3 H1 5UTR(+37+53) |
| 188 | CGGGGCTCGGGTGCCGG | SHANK3 H1 5UTR(+40+56) |
| 189 | GTCCGGGGCTCGGGTGC | SHANK3 H1 5UTR(+43+59) |
| 190 | GGAGTCCGGGGCTCGGG | SHANK3 H1 5UTR(+46+62) |
| 191 | GAAGGAGTCCGGGGCTC | SHANK3 H1 5UTR(+49+65) |
| 192 | GCCGAAGGAGTCCGGGG | SHANK3 H1 5UTR(+52+68) |
| 193 | CCCGCCGAAGGAGTCCG | SHANK3 H1 5UTR(+55+71) |
| 194 | GCCCCCGCCGAAGGAGT | SHANK3 H1 5UTR(+58+74) |
| 195 | CGGGCCCCCGCCGAAGG | SHANK3 H1 5UTR(+61+77) |
| 196 | CCCCGGGCCCCCGCCGA | SHANK3 H1 5UTR(+64+80) |
| 197 | GAGCCCCGGGCCCCCGC | SHANK3 H1 5UTR(+67+83) |
| 198 | GCCGAGCCCCGGGCCCC | SHANK3 H1 5UTR(+70+86) |
| 199 | GGTGCCGAGCCCCGGGC | SHANK3 H1 5UTR(+73+89) |
| 200 | CGGGGTGCCGAGCCCCG | SHANK3 H1 5UTR(+76+92) |
| 201 | ATGCGGGGTGCCGAGCC | SHANK3 H1 5UTR(+79+95) |
| 202 | CCCATGCGGGGTGCCGA | SHANK3 H1 5UTR(+82+98) |
| 203 | GGCCCCATGCGGGGTGC | SHANK3 H1 5UTR(+85+101) |
| 204 | GCCGGCCCCATGCGGGG | SHANK3 H1 5UTR(+88+104) |
| 205 | CCCGCCGGCCCCATGCG | SHANK3 H1 5UTR(+91+107) |
| 206 | CGCCCCGCCGGCCCCAT | SHANK3 H1 5UTR(+94+110) |
| 207 | ACCCGCCCCGCCGGCCC | SHANK3 H1 5UTR(+97+113) |
| 208 | CGGACCCGCCCCGCCGG | SHANK3 H1 5UTR(+100+116) |
| 209 | GCGCGGACCCGCCCCGC | SHANK3 H1 5UTR(+103+119) |
| 210 | GGAGCGCGGACCCGCCC | SHANK3 H1 5UTR(+106+122) |
| 211 | CCGGGAGCGCGGACCCG | SHANK3 H1 5UTR(+109+125) |
| 212 | GTCCCGGGAGCGCGGAC | SHANK3 H1 5UTR(+112+128) |
| 213 | CAGGTCCCGGGAGCGCG | SHANK3 H1 5UTR(+115+131) |
| 214 | GCTCAGGTCCCGGGAGC | SHANK3 H1 5UTR(+118+134) |
| 215 | TGAGCTCAGGTCCCGGG | SHANK3 H1 5UTR(+121+137) |
| 216 | TCGTGAGCTCAGGTCCC | SHANK3 H1 5UTR(+124+140) |
| 217 | GGCTCGTGAGCTCAGGT | SHANK3 H1 5UTR(+127+143) |
| 218 | GCGGGCTCGTGAGCTCA | SHANK3 H1 5UTR(+130+146) |
| 219 | GGAGCGGGCTCGTGAGC | SHANK3 H1 5UTR(+133+149) |
| 220 | AGCGGAGCGGGCTCGTG | SHANK3 H1 5UTR(+136+152) |
| 221 | TGCAGCGGAGCGGGCTC | SHANK3 H1 5UTR(+139+155) |
| 222 | TTCTGCAGCGGAGCGGG | SHANK3 H1 5UTR(+142+158) |
| 223 | CACTTCTGCAGCGGAGC | SHANK3 H1 5UTR(+145+161) |
| 224 | AGGCACTTCTGCAGCGG | SHANK3 H1 5UTR(+148+164) |
| 225 | CGCAGGCACTTCTGCAG | SHANK3 H1 5UTR(+151+167) |
| 226 | AGGCGCAGGCACTTCTG | SHANK3 H1 5UTR(+154+170) |
| 227 | TCCAGGCGCAGGCACTT | SHANK3 H1 5UTR(+157+173) |
| 228 | GGGTCCAGGCGCAGGCA | SHANK3 H1 5UTR(+160+176) |
| 229 | GCCGGGTCCAGGCGCAG | SHANK3 H1 5UTR(+163+179) |
| 230 | GCGGCCGGGTCCAGGCG | SHANK3 H1 5UTR(+166+182) |
| 231 | GGCGCGGCCGGGTCCAG | SHANK3 H1 5UTR(+169+185) |
| 232 | ACGGGCGCGGCCGGGTC | SHANK3 H1 5UTR(+172+188) |
| 233 | CACACGGGCGCGGCCGG | SHANK3 H1 5UTR(+175+191) |
| 234 | GCCCACACGGGCGCGGC | SHANK3 H1 5UTR(+178+194) |
| 235 | GCGGCCCACACGGGCGC | SHANK3 H1 5UTR(+181+197) |
| 236 | TTGGCGGCCCACACGGG | SHANK3 H1 5UTR(+184+200) |
| 237 | TGCTTGGCGGCCCACAC | SHANK3 H1 5UTR(+187+203) |
| 238 | CGCTGCTTGGCGGCCCA | SHANK3 H1 5UTR(+190+206) |
| 239 | ACGCGCTGCTTGGCGGC | SHANK3 H1 5UTR(+193+209) |
| 240 | AGCACGCGCTGCTTGGC | SHANK3 H1 5UTR(+196+212) |
| 241 | CAGAGCACGCGCTGCTT | SHANK3 H1 5UTR(+199+215) |
| 242 | GCGCAGAGCACGCGCTG | SHANK3 H1 5UTR(+202+218) |
| 243 | AGGGCGCAGAGCACGCG | SHANK3 H1 5UTR(+205+221) |
| 244 | TTGAGGGCGCAGAGCAC | SHANK3 H1 5UTR(+208+224) |
| 245 | TGGTTGAGGGCGCAGAG | SHANK3 H1 5UTR(+211+227) |
| 246 | CTGTGGTTGAGGGCGCA | SHANK3 H1 5UTR(+214+230) |
| 247 | AGGCTGTGGTTGAGGGC | SHANK3 H1 5UTR(+217+233) |
| 248 | TGGAGGCTGTGGTTGAG | SHANK3 H1 5UTR(+220+236) |
| 249 | TCCTGGAGGCTGTGGTT | SHANK3 H1 5UTR(+223+239) |
| 250 | GCGTCCTGGAGGCTGTG | SHANK3 H1 5UTR(+226+242) |
| 251 | AGCGCGTCCTGGAGGCT | SHANK3 H1 5UTR(+229+245) |
| 252 | TTGAGCGCGTCCTGGAG | SHANK3 H1 5UTR(+232+248) |
| 253 | TAGTTGAGCGCGTCCTG | SHANK3 H1 5UTR(+235+251) |
| 254 | CCATAGTTGAGCGCGTC | SHANK3 H1 5UTR(+238+254) |
| 255 | AGCCCATAGTTGAGCGC | SHANK3 H1 5UTR(+241+257) |
| 256 | AAAAGCCCATAGTTGAG | SHANK3 H1 5UTR(+244+260) |
| 257 | TGGAAAAGCCCATAGTT | SHANK3 H1 5UTR(+247+263) |
| 258 | GGCTGGAAAAGCCCATA | SHANK3 H1 5UTR(+250+266) |
| 259 | GGCGGCTGGAAAAGCCC | SHANK3 H1 5UTR(+253+269) |
| 260 | GAGGGCGGCTGGAAAAG | SHANK3 H1 5UTR(+256+272) |
| 261 | CGGGAGGGCGGCTGGAA | SHANK3 H1 5UTR(+259+275) |
| 262 | CCCCGGGAGGGCGGCTG | SHANK3 H1 5UTR(+262+278) |
| 263 | CGGCCCCGGGAGGGCGG | SHANK3 H1 5UTR(+265+281) |
| 264 | GCGCGGCCCCGGGAGGG | SHANK3 H1 5UTR(+268+284) |
| 265 | CCGGCGCGGCCCCGGGA | SHANK3 H1 5UTR(+271+287) |
| 266 | TTGCCGGCGCGGCCCCG | SHANK3 H1 5UTR(+274+290) |
| 267 | AACTTGCCGGCGCGGCC | SHANK3 H1 5UTR(+277+293) |
| 268 | AGGAACTTGCCGGCGCG | SHANK3 H1 5UTR(+280+296) |
| 269 | TCCAGGAACTTGCCGGC | SHANK3 H1 5UTR(+283+299) |
| 270 | TCATCCAGGAACTTGCC | SHANK3 H1 5UTR(+286+302) |
| 271 | TCCTCATCCAGGAACTT | SHANK3 H1 5UTR(+289+305) |
| 272 | CGCTCCTCATCCAGGAA | SHANK3 H1 5UTR(+292+308) |
| 273 | AGCCGCTCCTCATCCAG | SHANK3 H1 5UTR(+295+311) |
| 274 | AGGAGCCGCTCCTCATC | SHANK3 H1 5UTR(+298+314) |
| 275 | TGCAGGAGCCGCTCCTC | SHANK3 H1 5UTR(+301+317) |
| 276 | TCCTGCAGGAGCCGCTC | SHANK3 H1 5UTR(+304+320) |
| 277 | TACTCCTGCAGGAGCCG | SHANK3 H1 5UTR(+307+323) |
| 278 | GGGTACTCCTGCAGGAG | SHANK3 H1 5UTR(+310+326) |
| 279 | GGCGGGTACTCCTGCAG | SHANK3 H1 5UTR(+313+329) |
| 280 | TTGGGCGGGTACTCCTG | SHANK3 H1 5UTR(+316+332) |
| 281 | AGGTTGGGCGGGTACTC | SHANK3 H1 5UTR(+319+335) |
| 282 | TCCAGGTTGGGCGGGTA | SHANK3 H1 5UTR(+322+338) |
| 283 | GTGTCCAGGTTGGGCGG | SHANK3 H1 5UTR(+325+341) |
| 284 | GGCGTGTCCAGGTTGGG | SHANK3 H1 5UTR(+328+344) |
| 285 | AGGGGCGTGTCCAGGTT | SHANK3 H1 5UTR(+331+347) |
| 286 | GGCAGGGGCGTGTCCAG | SHANK3 H1 5UTR(+334+350) |
| 287 | TAGGGCAGGGGCGTGTC | SHANK3 H1 5UTR(+337+353) |
| 288 | AGGTAGGGCAGGGGCGT | SHANK3 H1 5UTR(+340+356) |
| 289 | TCCAGGTAGGGCAGGGG | SHANK3 H1 5UTR(+343+359) |
| 290 | AACTCCAGGTAGGGCAG | SHANK3 H1:2 5UTR(+346+362) |
| 291 | CGAAACTCCAGGTAGGG | SHANK3 H1:2 5UTR(+349+365) |
| 292 | TATCGAAACTCCAGGTA | SHANK3 H1:2 5UTR(+352+368) |
| 293 | TTGTATCGAAACTCCAG | SHANK3 H1:2 5UTR(+355+371) |
| 294 | CGCTTGTATCGAAACTC | SHANK3 H1:2 5UTR(+358+374) |
| 295 | CGCCGCTTGTATCGAAA | SHANK3 H2 5UTR(+361+377) |
| 296 | ACTCGCCGCTTGTATCG | SHANK3 H2 5UTR(+364+380) |
| 297 | TAAACTCGCCGCTTGTA | SHANK3 H2 5UTR(+367+383) |
| 298 | GCATAAACTCGCCGCTT | SHANK3 H2 5UTR(+370+386) |
| 299 | TGGGCATAAACTCGCCG | SHANK3 H2 5UTR(+373+389) |
| 300 | TTCTGGGCATAAACTCG | SHANK3 H2 5UTR(+376+392) |
| 301 | AGGTTCTGGGCATAAAC | SHANK3 H2 5UTR(+379+395) |
| 302 | ATGAGGTTCTGGGCATA | SHANK3 H2 5UTR(+382+398) |
| 303 | TCGATGAGGTTCTGGGC | SHANK3 H2 5UTR(+385+401) |
| 304 | TCATCGATGAGGTTCTG | SHANK3 H2 5UTR(+388+404) |
| 305 | TTATCATCGATGAGGTT | SHANK3 H2 5UTR(+391+407) |
| 306 | TGCTTATCATCGATGAG | SHANK3 H2 5UTR(+394+410) |
| 307 | AACTGCTTATCATCGAT | SHANK3 H2 5UTR(+397+413) |
| 308 | GCAAACTGCTTATCATC | SHANK3 H2 5UTR(+400+416) |
| 309 | TTTGCAAACTGCTTATC | SHANK3 H2 5UTR(+403+419) |
| 310 | AGCTTTGCAAACTGCTT | SHANK3 H2 5UTR(+406+422) |
| 311 | TGAAGCTTTGCAAACTG | SHANK3 H2 5UTR(+409+425) |
| 312 | GTGTGAAGCTTTGCAAA | SHANK3 H2 5UTR(+412+428) |
| 313 | TTTGTGTGAAGCTTTGC | SHANK3 H2 5UTR(+415+431) |
| 314 | GCCTTTGTGTGAAGCTT | SHANK3 H2:3 5UTR(+418+434) |
| 315 | TTCGCCTTTGTGTGAAG | SHANK3 H2:3 5UTR(+421+437) |
| 316 | AGGTTCGCCTTTGTGTG | SHANK3 H2:3 5UTR(+424+440) |
| 317 | TTCAGGTTCGCCTTTGT | SHANK3 H2:3 5UTR(+427+443) |
| 318 | TTCTTCAGGTTCGCCTT | SHANK3 H2:3 5UTR(+430+446) |
| 319 | AACTTCTTCAGGTTCGC | SHANK3 H3 5UTR(+433+449) |
| 320 | GGCCACTGGCCTGGCCTGAGTGGTG | SHANK3 H1 5UTR(−2178−2154) |
| 321 | CAAGGCCACTGGCCTGGCCTGAGTG | SHANK3 H1 5UTR(−2175−2151) |
| 322 | TCCCAAGGCCACTGGCCTGGCCTGA | SHANK3 H1 5UTR(−2172−2148) |
| 323 | CCCTCCCAAGGCCACTGGCCTGGCC | SHANK3 H1 5UTR(−2169−2145) |
| 324 | GGCCCCTCCCAAGGCCACTGGCCTG | SHANK3 H1 5UTR(−2166−2142) |
| 325 | ACAGGCCCCTCCCAAGGCCACTGGC | SHANK3 H1 5UTR(−2163−2139) |
| 326 | ATCACAGGCCCCTCCCAAGGCCACT | SHANK3 H1 5UTR(−2160−2136) |
| 327 | AGCATCACAGGCCCCTCCCAAGGCC | SHANK3 H1 5UTR(−2157−2133) |
| 328 | CCCAGCATCACAGGCCCCTCCCAAG | SHANK3 H1 5UTR(−2154−2130) |
| 329 | GGTCCCAGCATCACAGGCCCCTCCC | SHANK3 H1 5UTR(−2151−2127) |
| 330 | TGTGGTCCCAGCATCACAGGCCCCT | SHANK3 H1 5UTR(−2148−2124) |
| 331 | AACTGTGGTCCCAGCATCACAGGCC | SHANK3 H1 5UTR(−2145−2121) |
| 332 | AGGAACTGTGGTCCCAGCATCACAG | SHANK3 H1 5UTR(−2142−2118) |
| 333 | CCCAGGAACTGTGGTCCCAGCATCA | SHANK3 H1 5UTR(−2139−2115) |
| 334 | CTGCCCAGGAACTGTGGTCCCAGCA | SHANK3 H1 5UTR(−2136−2112) |
| 335 | TCCCTGCCCAGGAACTGTGGTCCCA | SHANK3 H1 5UTR(−2133−2109) |
| 336 | TGCTCCCTGCCCAGGAACTGTGGTC | SHANK3 H1 5UTR(−2130−2106) |
| 337 | GGTTGCTCCCTGCCCAGGAACTGTG | SHANK3 H1 5UTR(−2127−2103) |
| 338 | GACGGTTGCTCCCTGCCCAGGAACT | SHANK3 H1 5UTR(−2124−2100) |
| 339 | CTAGACGGTTGCTCCCTGCCCAGGA | SHANK3 H1 5UTR(−2121−2097) |
| 340 | CGCCTAGACGGTTGCTCCCTGCCCA | SHANK3 H1 5UTR(−2118−2094) |
| 341 | CCACGCCTAGACGGTTGCTCCCTGC | SHANK3 H1 5UTR(−2115−2091) |
| 342 | TCCCCACGCCTAGACGGTTGCTCCC | SHANK3 H1 5UTR(−2112−2088) |
| 343 | TTCTCCCCACGCCTAGACGGTTGCT | SHANK3 H1 5UTR(−2109−2085) |
| 344 | GCGTTCTCCCCACGCCTAGACGGTT | SHANK3 H1 5UTR(−2106−2082) |
| 345 | CCTGCGTTCTCCCCACGCCTAGACG | SHANK3 H1 5UTR(−2103−2079) |
| 346 | CGTCCTGCGTTCTCCCCACGCCTAG | SHANK3 H1 5UTR(−2100−2076) |
| 347 | TCACGTCCTGCGTTCTCCCCACGCC | SHANK3 H1 5UTR(−2097−2073) |
| 348 | GGGTCACGTCCTGCGTTCTCCCCAC | SHANK3 H1 5UTR(−2094−2070) |
| 349 | TGTGGGTCACGTCCTGCGTTCTCCC | SHANK3 H1 5UTR(−2091−2067) |
| 350 | GTGTGTGGGTCACGTCCTGCGTTCT | SHANK3 H1 5UTR(−2088−2064) |
| 351 | GCGGTGTGTGGGTCACGTCCTGCGT | SHANK3 H1 5UTR(−2085−2061) |
| 352 | AGTGCGGTGTGTGGGTCACGTCCTG | SHANK3 H1 5UTR(−2082−2058) |
| 353 | TCCAGTGCGGTGTGTGGGTCACGTC | SHANK3 H1 5UTR(−2079−2055) |
| 354 | GCCTCCAGTGCGGTGTGTGGGTCAC | SHANK3 H1 5UTR(−2076−2052) |
| 355 | GGAGCCTCCAGTGCGGTGTGTGGGT | SHANK3 H1 5UTR(−2073−2049) |
| 356 | AGCGGAGCCTCCAGTGCGGTGTGTG | SHANK3 H1 5UTR(−2070−2046) |
| 357 | CAGAGCGGAGCCTCCAGTGCGGTGT | SHANK3 H1 5UTR(−2067−2043) |
| 358 | GGGCAGAGCGGAGCCTCCAGTGCGG | SHANK3 H1 5UTR(−2064−2040) |
| 359 | TGCGGCGGAGGAAGGGGTCCCGGAG | SHANK3 H1 5UTR(−816−792) |
| 360 | GGGTGCGGCGGAGGAAGGGGTCCCG | SHANK3 H1 5UTR(−813−789) |
| 361 | GGCGGGTGCGGCGGAGGAAGGGGTC | SHANK3 H1 5UTR(−810−786) |
| 362 | CGGGGGGGGTGCGGCGGAGGAAGGG | SHANK3 H1 5UTR(−807−783) |
| 363 | CACCGGGGGGGGTGCGGCGGAGGAA | SHANK3 H1 5UTR(−804−780) |
| 364 | GACCACCGGGGGGGGTGCGGCGGAG | SHANK3 H1 5UTR(−801−777) |
| 365 | GCGGACCACCGGGGGGGGTGCGGCG | SHANK3 H1 5UTR(−798−774) |
| 366 | TCCGCGGACCACCGGGGGGGGTGCG | SHANK3 H1 5UTR(−795−771) |
| 367 | GCATCCGCGGACCACCGGGGGGGGT | SHANK3 H1 5UTR(−792−768) |
| 368 | CGGGCATCCGCGGACCACCGGGGCG | SHANK3 H1 5UTR(−789−765) |
| 369 | GGGCGGGCATCCGCGGACCACCGGG | SHANK3 H1 5UTR(−786−762) |
| 370 | CAAGGGCGGGCATCCGCGGACCACC | SHANK3 H1 5UTR(−783−759) |
| 371 | CGGCAAGGGCGGGCATCCGCGGACC | SHANK3 H1 5UTR(−780−756) |
| 372 | GAGCGGCAAGGGCGGGCATCCGCGG | SHANK3 H1 5UTR(−777−753) |
| 373 | GCTGAGCGGCAAGGGCGGGCATCCG | SHANK3 H1 5UTR(−774−750) |
| 374 | GTGGCTGAGCGGCAAGGGCGGGCAT | SHANK3 H1 5UTR(−771−747) |
| 375 | GGAGTGGCTGAGCGGCAAGGGCGGG | SHANK3 H1 5UTR(−768−744) |
| 376 | GGGGGAGTGGCTGAGCGGCAAGGGC | SHANK3 H1 5UTR(−765−741) |
| 377 | GCGGGGGGAGTGGCTGAGCGGCAAG | SHANK3 H1 5UTR(−762−738) |
| 378 | GGTGCGGGGGGAGTGGCTGAGCGGC | SHANK3 H1 5UTR(−759−735) |
| 379 | CTCGGTGCGGGGGGAGTGGCTGAGC | SHANK3 H1 5UTR(−756−732) |
| 380 | GGCCTCGGTGCGGGGGGAGTGGCTG | SHANK3 H1 5UTR(−753−729) |
| 381 | CTAGGCCTCGGTGCGGGGGGAGTGG | SHANK3 H1 5UTR(−750−726) |
| 382 | GTCCTAGGCCTCGGTGCGGGGGGAG | SHANK3 H1 5UTR(−747−723) |
| 383 | GGAGTCCTAGGCCTCGGTGCGGGGG | SHANK3 H1 5UTR(−744−720) |
| 384 | TCGGCCCCGGCTCAGCTCGGCGCCT | SHANK3 H1 5UTR(−501−477) |
| 385 | GCATCGGCCCCGGCTCAGCTCGGCG | SHANK3 H1 5UTR(−498−474) |
| 386 | GCTGCATCGGCCCCGGCTCAGCTCG | SHANK3 H1 5UTR(−495−471) |
| 387 | TCAGCTGCATCGGCCCCGGCTCAGC | SHANK3 H1 5UTR(−492−468) |
| 388 | GGCTCAGCTGCATCGGCCCCGGCTC | SHANK3 H1 5UTR(−489−465) |
| 389 | CGCGGCTCAGCTGCATCGGCCCCGG | SHANK3 H1 5UTR(−486−462) |
| 390 | CGGCGCGGCTCAGCTGCATCGGCCC | SHANK3 H1 5UTR(−483−459) |
| 391 | CGGCGGCGCGGCTCAGCTGCATCGG | SHANK3 H1 5UTR(−480−456) |
| 392 | CGGCGGCGGCGCGGCTCAGCTGCAT | SHANK3 H1 5UTR(−477−453) |
| 393 | CGGCGGCGGCGGCGCGGCTCAGCTG | SHANK3 H1 5UTR(−474−450) |
| 394 | CGGCGGCGGCGGCGGCGCGGCTCAG | SHANK3 H1 5UTR(−471−447) |
| 395 | CGGCGGCGGCGGCGGCGGCGCGGCT | SHANK3 H1 5UTR(−468−444) |
| 396 | CGGCGGCGGCGGCGGCGGCGGCGCG | SHANK3 H1 5UTR(−465−441) |
| 397 | GGGCGGCGGCGGCGGCGGCGGCGGC | SHANK3 H1 5UTR(−462−438) |
| 398 | CAGGGGCGGCGGCGGCGGCGGCGGC | SHANK3 H1 5UTR(−459−435) |
| 399 | CCGCAGGGGCGGCGGCGGCGGCGGC | SHANK3 H1 5UTR(−456−432) |
| 400 | GCTCCGCAGGGGCGGCGGCGGCGGC | SHANK3 H1 5UTR(−453−429) |
| 401 | GGGGCTCCGCAGGGGCGGCGGCGGC | SHANK3 H1 5UTR(−450−426) |
| 402 | CCGGGGGCTCCGCAGGGGCGGCGGC | SHANK3 H1 5UTR(−447−423) |
| 403 | GCTCCGGGGGCTCCGCAGGGGCGGC | SHANK3 H1 5UTR(−444−420) |
| 404 | GCGGCTCCGGGGGCTCCGCAGGGGC | SHANK3 H1 5UTR(−441−417) |
| 405 | ACAGCGGCTCCGGGGGCTCCGCAGG | SHANK3 H1 5UTR(−438−414) |
| 406 | GGGACAGCGGCTCCGGGGGCTCCGC | SHANK3 H1 5UTR(−435−411) |
| 407 | CGGGGGACAGCGGCTCCGGGGGCTC | SHANK3 H1 5UTR(−432−408) |
| 408 | GCGCGGGGGACAGCGGCTCCGGGGG | SHANK3 H1 5UTR(−429−405) |
| 409 | CCGGCGCGGGGGACAGCGGCTCCGG | SHANK3 H1 5UTR(−426−402) |
| 410 | GGGCCGGCGCGGGGGACAGCGGCTC | SHANK3 H1 5UTR(−423−399) |
| 411 | CCGGGGCCGGCGCGGGGGACAGCGG | SHANK3 H1 5UTR(−420−396) |
| 412 | GGGCCGGGGCCGGCGCGGGGGACAG | SHANK3 H1 5UTR(−417−393) |
| 413 | CCGGGGCCGGGGCCGGCGCGGGGGA | SHANK3 H1 5UTR(−414−390) |
| 414 | GGGCCGGGGCCGGGGCCGGCGCGGG | SHANK3 H1 5UTR(−411−387) |
| 415 | GGGGGGCCGGGGCCGGGGCCGGCGC | SHANK3 H1 5UTR(−408−384) |
| 416 | CGGGGGGGGCCGGGGCCGGGGCCGG | SHANK3 H1 5UTR(−405−381) |
| 417 | GGCCGGGGGGGGCCGGGGCCGGGGC | SHANK3 H1 5UTR(−402−378) |
| 418 | GGGGGCCGGGGGGGGCCGGGGCCGG | SHANK3 H1 5UTR(−399−375) |
| 419 | GGAGGGGGCCGGGGGGGGCCGGGGC | SHANK3 H1 5UTR(−396−372) |
| 420 | GCGGGAGGGGGCCGGGGGGGGCCGG | SHANK3 H1 5UTR(−393−369) |
| 421 | TGCGCGGGAGGGGGCCGGGGGGGGC | SHANK3 H1 5UTR(−390−366) |
| 422 | CGCTGCGCGGGAGGGGGCCGGGGGG | SHANK3 H1 5UTR(−387−363) |
| 423 | CCGCGCTGCGCGGGAGGGGGCCGGG | SHANK3 H1 5UTR(−384−360) |
| 424 | CGGCCGCGCTGCGCGGGAGGGGGCC | SHANK3 H1 5UTR(−381−357) |
| 425 | CGTCGGCCGCGCTGCGCGGGAGGGG | SHANK3 H1 5UTR(−378−354) |
| 426 | CCCCGTCGGCCGCGCTGCGCGGGAG | SHANK3 H1 5UTR(−375−351) |
| 427 | GAGCCCCGTCGGCCGCGCTGCGCGG | SHANK3 H1 5UTR(−372−348) |
| 428 | CCGGAGCCCCGTCGGCCGCGCTGCG | SHANK3 H1 5UTR(−369−345) |
| 429 | CCGCCGGAGCCCCGTCGGCCGCGCT | SHANK3 H1 5UTR(−366−342) |
| 430 | CCCCCGCCGGAGCCCCGTCGGCCGC | SHANK3 H1 5UTR(−363−339) |
| 431 | TCCCCCCCGCCGGAGCCCCGTCGGC | SHANK3 H1 5UTR(−360−336) |
| 432 | CCTTCCCCCCCGCCGGAGCCCCGTC | SHANK3 H1 5UTR(−357−333) |
| 433 | CCCCCTTCCCCCCCGCCGGAGCCCC | SHANK3 H1 5UTR(−354−330) |
| 434 | GCCCCCCCTTCCCCCCCGCCGGAGC | SHANK3 H1 5UTR(−351−327) |
| 435 | CCGGCCCCCCCTTCCCCCCCGCCGG | SHANK3 H1 5UTR(−348−324) |
| 436 | GCCCCGGCCCCCCCTTCCCCCCCGC | SHANK3 H1 5UTR(−345−321) |
| 437 | GGCGCCCCGGCCCCCCCTTCCCCCC | SHANK3 H1 5UTR(−342−318) |
| 438 | CGCGGCGCCCCGGCCCCCCCTTCCC | SHANK3 H1 5UTR(−339−315) |
| 439 | GCGCGCGGCGCCCCGGCCCCCCCTT | SHANK3 H1 5UTR(−336−312) |
| 440 | TCCGCGCGCGGCGCCCCGGCCCCCC | SHANK3 H1 5UTR(−333−309) |
| 441 | GACTCCGCGCGCGGCGCCCCGGCCC | SHANK3 H1 5UTR(−330−306) |
| 442 | GGGGACTCCGCGCGCGGCGCCCCGG | SHANK3 H1 5UTR(−327−303) |
| 443 | CCCGGGGACTCCGCGCGCGGCGCCC | SHANK3 H1 5UTR(−324−300) |
| 444 | GCGCCCGGGGACTCCGCGCGCGGCG | SHANK3 H1 5UTR(−321−297) |
| 445 | GGAGCGCCCGGGGACTCCGCGCGCG | SHANK3 H1 5UTR(−318−294) |
| 446 | AACGGAGCGCCCGGGGACTCCGCGC | SHANK3 H1 5UTR(−315−291) |
| 447 | GGGAACGGAGCGCCCGGGGACTCCG | SHANK3 H1 5UTR(−312−288) |
| 448 | CCGGGGAACGGAGCGCCCGGGGACT | SHANK3 H1 5UTR(−309−285) |
| 449 | GCGCCGGGGAACGGAGCGCCCGGGG | SHANK3 H1 5UTR(−306−282) |
| 450 | CTCGCGCCGGGGAACGGAGCGCCCG | SHANK3 H1 5UTR(−303−279) |
| 451 | CCGCTCGCGCCGGGGAACGGAGCGC | SHANK3 H1 5UTR(−300−276) |
| 452 | GGGCCGCTCGCGCCGGGGAACGGAG | SHANK3 H1 5UTR(−297−273) |
| 453 | CCGGGGCCGCTCGCGCCGGGGAACG | SHANK3 H1 5UTR(−294−270) |
| 454 | GGGCCGGGGCCGCTCGCGCCGGGGA | SHANK3 H1 5UTR(−291−267) |
| 455 | CCCGGGCCGGGGCCGCTCGCGCCGG | SHANK3 H1 5UTR(−288−264) |
| 456 | GGGCCCGGGCCGGGGCCGCTCGCGC | SHANK3 H1 5UTR(−285−261) |
| 457 | CCGGGGCCCGGGCCGGGGCCGCTCG | SHANK3 H1 5UTR(−282−258) |
| 458 | GCGCCGGGGCCCGGGCCGGGGCCGC | SHANK3 H1 5UTR(−279−255) |
| 459 | CCCGCGCCGGGGCCCGGGCCGGGGC | SHANK3 H1 5UTR(−276−252) |
| 460 | ATCCCCGCGCCGGGGCCCGGGCCGG | SHANK3 H1 5UTR(−273−249) |
| 461 | TCCATCCCCGCGCCGGGGCCCGGGC | SHANK3 H1 5UTR(−270−246) |
| 462 | CCGTCCATCCCCGCGCCGGGGCCCG | SHANK3 H1 5UTR(−267−243) |
| 463 | GGGCCGTCCATCCCCGCGCCGGGGC | SHANK3 H1 5UTR(−264−240) |
| 464 | CCGGGGCCGTCCATCCCCGCGCCGG | SHANK3 H1 5UTR(−261−237) |
| 465 | GCCCCGGGGCCGTCCATCCCCGCGC | SHANK3 H1 5UTR(−258−234) |
| 466 | CTGGCCCCGGGGCCGTCCATCCCCG | SHANK3 H1 5UTR(−255−231) |
| 467 | GCGCTGGCCCCGGGGCCGTCCATCC | SHANK3 H1 5UTR(−252−228) |
| 468 | ACGGCGCTGGCCCCGGGGCCGTCCA | SHANK3 H1 5UTR(−249−225) |
| 469 | ACCACGGCGCTGGCCCCGGGGCCGT | SHANK3 H1 5UTR(−246−222) |
| 470 | ACGACCACGGCGCTGGCCCCGGGGC | SHANK3 H1 5UTR(−243−219) |
| 471 | CGCACGACCACGGCGCTGGCCCCGG | SHANK3 H1 5UTR(−240−216) |
| 472 | ACGCGCACGACCACGGCGCTGGCCC | SHANK3 H1 5UTR(−237−213) |
| 473 | CCGACGCGCACGACCACGGCGCTGG | SHANK3 H1 5UTR(−234−210) |
| 474 | ATGCCGACGCGCACGACCACGGCGC | SHANK3 H1 5UTR(−231−207) |
| 475 | GGGATGCCGACGCGCACGACCACGG | SHANK3 H1 5UTR(−228−204) |
| 476 | TCCGGGATGCCGACGCGCACGACCA | SHANK3 H1 5UTR(−225−201) |
| 477 | AGGTCCGGGATGCCGACGCGCACGA | SHANK3 H1 5UTR(−222−198) |
| 478 | TGCAGGTCCGGGATGCCGACGCGCA | SHANK3 H1 5UTR(−219−195) |
| 479 | TGCTGCAGGTCCGGGATGCCGACGC | SHANK3 H1 5UTR(−216−192) |
| 480 | GTCTGCTGCAGGTCCGGGATGCCGA | SHANK3 H1 5UTR(−213−189) |
| 481 | CGCCTCCGCGAACCGCGGCCGAAGC | SHANK3 H1 5UTR(+1+25) |
| 482 | CCGCGCCTCCGCGAACCGCGGCCGA | SHANK3 H1 5UTR(+4+28) |
| 483 | ACCCCGCGCCTCCGCGAACCGCGGC | SHANK3 H1 5UTR(+7+31) |
| 484 | GGGACCCCGCGCCTCCGCGAACCGC | SHANK3 H1 5UTR(+10+34) |
| 485 | CCCGGGACCCCGCGCCTCCGCGAAC | SHANK3 H1 5UTR(+13+37) |
| 486 | GCGCCCGGGACCCCGCGCCTCCGCG | SHANK3 H1 5UTR(+16+40) |
| 487 | CCGGCGCCCGGGACCCCGCGCCTCC | SHANK3 H1 5UTR(+19+43) |
| 488 | GTGCCGGCGCCCGGGACCCCGCGCC | SHANK3 H1 5UTR(+22+46) |
| 489 | CGGGTGCCGGCGCCCGGGACCCCGC | SHANK3 H1 5UTR(+25+49) |
| 490 | GCTCGGGTGCCGGCGCCCGGGACCC | SHANK3 H1 5UTR(+28+52) |
| 491 | GGGGCTCGGGTGCCGGCGCCCGGGA | SHANK3 H1 5UTR(+31+55) |
| 492 | TCCGGGGCTCGGGTGCCGGCGCCCG | SHANK3 H1 5UTR(+34+58) |
| 493 | GAGTCCGGGGCTCGGGTGCCGGCGC | SHANK3 H1 5UTR(+37+61) |
| 494 | AAGGAGTCCGGGGCTCGGGTGCCGG | SHANK3 H1 5UTR(+40+64) |
| 495 | CCGAAGGAGTCCGGGGCTCGGGTGC | SHANK3 H1 5UTR(+43+67) |
| 496 | CCGCCGAAGGAGTCCGGGGCTCGGG | SHANK3 H1 5UTR(+46+70) |
| 497 | CCCCCGCCGAAGGAGTCCGGGGCTC | SHANK3 H1 5UTR(+49+73) |
| 498 | GGGCCCCCGCCGAAGGAGTCCGGGG | SHANK3 H1 5UTR(+52+76) |
| 499 | CCCGGGCCCCCGCCGAAGGAGTCCG | SHANK3 H1 5UTR(+55+79) |
| 500 | AGCCCCGGGCCCCCGCCGAAGGAGT | SHANK3 H1 5UTR(+58+82) |
| 501 | CCGAGCCCCGGGCCCCCGCCGAAGG | SHANK3 H1 5UTR(+61+85) |
| 502 | GTGCCGAGCCCCGGGCCCCCGCCGA | SHANK3 H1 5UTR(+64+88) |
| 503 | GGGGTGCCGAGCCCCGGGCCCCCGC | SHANK3 H1 5UTR(+67+91) |
| 504 | TGCGGGGTGCCGAGCCCCGGGCCCC | SHANK3 H1 5UTR(+70+94) |
| 505 | CCATGCGGGGTGCCGAGCCCCGGGC | SHANK3 H1 5UTR(+73+97) |
| 506 | GCCCCATGCGGGGTGCCGAGCCCCG | SHANK3 H1 5UTR(+76+100) |
| 507 | CCGGCCCCATGCGGGGTGCCGAGCC | SHANK3 H1 5UTR(+79+103) |
| 508 | CCGCCGGCCCCATGCGGGGTGCCGA | SHANK3 H1 5UTR(+82+106) |
| 509 | GCCCCGCCGGCCCCATGCGGGGTGC | SHANK3 H1 5UTR(+85+109) |
| 510 | CCCGCCCCGCCGGCCCCATGCGGGG | SHANK3 H1 5UTR(+88+112) |
| 511 | GGACCCGCCCCGCCGGCCCCATGCG | SHANK3 H1 5UTR(+91+115) |
| 512 | CGCGGACCCGCCCCGCCGGCCCCAT | SHANK3 H1 5UTR(+94+118) |
| 513 | GAGCGCGGACCCGCCCCGCCGGCCC | SHANK3 H1 5UTR(+97+121) |
| 514 | CGGGAGCGCGGACCCGCCCCGCCGG | SHANK3 H1 5UTR(+100+124) |
| 515 | TCCCGGGAGCGCGGACCCGCCCCGC | SHANK3 H1 5UTR(+103+127) |
| 516 | AGGTCCCGGGAGCGCGGACCCGCCC | SHANK3 H1 5UTR(+106+130) |
| 517 | CTCAGGTCCCGGGAGCGCGGACCCG | SHANK3 H1 5UTR(+109+133) |
| 518 | GAGCTCAGGTCCCGGGAGCGCGGAC | SHANK3 H1 5UTR(+112+136) |
| 519 | CGTGAGCTCAGGTCCCGGGAGCGCG | SHANK3 H1 5UTR(+115+139) |
| 520 | GCTCGTGAGCTCAGGTCCCGGGAGC | SHANK3 H1 5UTR(+118+142) |
| 521 | CGGGCTCGTGAGCTCAGGTCCCGGG | SHANK3 H1 5UTR(+121+145) |
| 522 | GAGCGGGCTCGTGAGCTCAGGTCCC | SHANK3 H1 5UTR(+124+148) |
| 523 | GCGGAGCGGGCTCGTGAGCTCAGGT | SHANK3 H1 5UTR(+127+151) |
| 524 | GCAGCGGAGCGGGCTCGTGAGCTCA | SHANK3 H1 5UTR(+130+154) |
| 525 | TCTGCAGCGGAGCGGGCTCGTGAGC | SHANK3 H1 5UTR(+133+157) |
| 526 | ACTTCTGCAGCGGAGCGGGCTCGTG | SHANK3 H1 5UTR(+136+160) |
| 527 | GGCACTTCTGCAGCGGAGCGGGCTC | SHANK3 H1 5UTR(+139+163) |
| 528 | GCAGGCACTTCTGCAGCGGAGCGGG | SHANK3 H1 5UTR(+142+166) |
| 529 | GGCGCAGGCACTTCTGCAGCGGAGC | SHANK3 H1 5UTR(+145+169) |
| 530 | CCAGGCGCAGGCACTTCTGCAGCGG | SHANK3 H1 5UTR(+148+172) |
| 531 | GGTCCAGGCGCAGGCACTTCTGCAG | SHANK3 H1 5UTR(+151+175) |
| 532 | CCGGGTCCAGGCGCAGGCACTTCTG | SHANK3 H1 5UTR(+154+178) |
| 533 | CGGCCGGGTCCAGGCGCAGGCACTT | SHANK3 H1 5UTR(+157+181) |
| 534 | GCGCGGCCGGGTCCAGGCGCAGGCA | SHANK3 H1 5UTR(+160+184) |
| 535 | CGGGCGCGGCCGGGTCCAGGCGCAG | SHANK3 H1 5UTR(+163+187) |
| 536 | ACACGGGCGCGGCCGGGTCCAGGCG | SHANK3 H1 5UTR(+166+190) |
| 537 | CCCACACGGGCGCGGCCGGGTCCAG | SHANK3 H1 5UTR(+169+193) |
| 538 | CGGCCCACACGGGCGCGGCCGGGTC | SHANK3 H1 5UTR(+172+196) |
| 539 | TGGCGGCCCACACGGGCGCGGCCGG | SHANK3 H1 5UTR(+175+199) |
| 540 | GCTTGGCGGCCCACACGGGCGCGGC | SHANK3 H1 5UTR(+178+202) |
| 541 | GCTGCTTGGCGGCCCACACGGGCGC | SHANK3 H1 5UTR(+181+205) |
| 542 | CGCGCTGCTTGGCGGCCCACACGGG | SHANK3 H1 5UTR(+184+208) |
| 543 | GCACGCGCTGCTTGGCGGCCCACAC | SHANK3 H1 5UTR(+187+211) |
| 544 | AGAGCACGCGCTGCTTGGCGGCCCA | SHANK3 H1 5UTR(+190+214) |
| 545 | CGCAGAGCACGCGCTGCTTGGCGGC | SHANK3 H1 5UTR(+193+217) |
| 546 | GGGCGCAGAGCACGCGCTGCTTGGC | SHANK3 H1 5UTR(+196+220) |
| 547 | TGAGGGCGCAGAGCACGCGCTGCTT | SHANK3 H1 5UTR(+199+223) |
| 548 | GGTTGAGGGCGCAGAGCACGCGCTG | SHANK3 H1 5UTR(+202+226) |
| 549 | TGTGGTTGAGGGCGCAGAGCACGCG | SHANK3 H1 5UTR(+205+229) |
| 550 | GGCTGTGGTTGAGGGCGCAGAGCAC | SHANK3 H1 5UTR(+208+232) |
| 551 | GGAGGCTGTGGTTGAGGGCGCAGAG | SHANK3 H1 5UTR(+211+235) |
| 552 | CCTGGAGGCTGTGGTTGAGGGCGCA | SHANK3 H1 5UTR(+214+238) |
| 553 | CGTCCTGGAGGCTGTGGTTGAGGGC | SHANK3 H1 5UTR(+217+241) |
| 554 | GCGCGTCCTGGAGGCTGTGGTTGAG | SHANK3 H1 5UTR(+220+244) |
| 555 | TGAGCGCGTCCTGGAGGCTGTGGTT | SHANK3 H1 5UTR(+223+247) |
| 556 | AGTTGAGCGCGTCCTGGAGGCTGTG | SHANK3 H1 5UTR(+226+250) |
| 557 | CATAGTTGAGCGCGTCCTGGAGGCT | SHANK3 H1 5UTR(+229+253) |
| 558 | GCCCATAGTTGAGCGCGTCCTGGAG | SHANK3 H1 5UTR(+232+256) |
| 559 | AAAGCCCATAGTTGAGCGCGTCCTG | SHANK3 H1 5UTR(+235+259) |
| 560 | GGAAAAGCCCATAGTTGAGCGCGTC | SHANK3 H1 5UTR(+238+262) |
| 561 | GCTGGAAAAGCCCATAGTTGAGCGC | SHANK3 H1 5UTR(+241+265) |
| 562 | GCGGCTGGAAAAGCCCATAGTTGAG | SHANK3 H1 5UTR(+244+268) |
| 563 | AGGGCGGCTGGAAAAGCCCATAGTT | SHANK3 H1 5UTR(+247+271) |
| 564 | GGGAGGGCGGCTGGAAAAGCCCATA | SHANK3 H1 5UTR(+250+274) |
| 565 | CCCGGGAGGGCGGCTGGAAAAGCCC | SHANK3 H1 5UTR(+253+277) |
| 566 | GGCCCCGGGAGGGCGGCTGGAAAAG | SHANK3 H1 5UTR(+256+280) |
| 567 | CGCGGCCCCGGGAGGGCGGCTGGAA | SHANK3 H1 5UTR(+259+283) |
| 568 | CGGCGCGGCCCCGGGAGGGCGGCTG | SHANK3 H1 5UTR(+262+286) |
| 569 | TGCCGGCGCGGCCCCGGGAGGGCGG | SHANK3 H1 5UTR(+265+289) |
| 570 | ACTTGCCGGCGCGGCCCCGGGAGGG | SHANK3 H1 5UTR(+268+292) |
| 571 | GGAACTTGCCGGCGCGGCCCCGGGA | SHANK3 H1 5UTR(+271+295) |
| 572 | CCAGGAACTTGCCGGCGCGGCCCCG | SHANK3 H1 5UTR(+274+298) |
| 573 | CATCCAGGAACTTGCCGGCGCGGCC | SHANK3 H1 5UTR(+277+301) |
| 574 | CCTCATCCAGGAACTTGCCGGCGCG | SHANK3 H1 5UTR(+280+304) |
| 575 | GCTCCTCATCCAGGAACTTGCCGGC | SHANK3 H1 5UTR(+283+307) |
| 576 | GCCGCTCCTCATCCAGGAACTTGCC | SHANK3 H1 5UTR(+286+310) |
| 577 | GGAGCCGCTCCTCATCCAGGAACTT | SHANK3 H1 5UTR(+289+313) |
| 578 | GCAGGAGCCGCTCCTCATCCAGGAA | SHANK3 H1 5UTR(+292+316) |
| 579 | CCTGCAGGAGCCGCTCCTCATCCAG | SHANK3 H1 5UTR(+295+319) |
| 580 | ACTCCTGCAGGAGCCGCTCCTCATC | SHANK3 H1 5UTR(+298+322) |
| 581 | GGTACTCCTGCAGGAGCCGCTCCTC | SHANK3 H1 5UTR(+301+325) |
| 582 | GCGGGTACTCCTGCAGGAGCCGCTC | SHANK3 H1 5UTR(+304+328) |
| 583 | TGGGCGGGTACTCCTGCAGGAGCCG | SHANK3 H1 5UTR(+307+331) |
| 584 | GGTTGGGCGGGTACTCCTGCAGGAG | SHANK3 H1 5UTR(+310+334) |
| 585 | CCAGGTTGGGGGGTACTCCTGCAG | SHANK3 H1 5UTR(+313+337) |
| 586 | TGTCCAGGTTGGGCGGGTACTCCTG | SHANK3 H1 5UTR(+316+340) |
| 587 | GCGTGTCCAGGTTGGGCGGGTACTC | SHANK3 H1 5UTR(+319+343) |
| 588 | GGGGCGTGTCCAGGTTGGGCGGGTA | SHANK3 H1 5UTR(+322+346) |
| 589 | GCAGGGGCGTGTCCAGGTTGGGCGG | SHANK3 H1 5UTR(+325+349) |
| 590 | AGGGCAGGGGCGTGTCCAGGTTGGG | SHANK3 H1 5UTR(+328+352) |
| 591 | GGTAGGGCAGGGGCGTGTCCAGGTT | SHANK3 H1 5UTR(+331+355) |
| 592 | CCAGGTAGGGCAGGGGCGTGTCCAG | SHANK3 H1 5UTR(+334+358) |
| 593 | ACTCCAGGTAGGGCAGGGGCGTGTC | SHANK3 H1:2 5UTR(+337+361) |
| 594 | GAAACTCCAGGTAGGGCAGGGGCGT | SHANK3 H1:2 5UTR(+340+364) |
| 595 | ATCGAAACTCCAGGTAGGGCAGGGG | SHANK3 H1:2 5UTR(+343+367) |
| 596 | TGTATCGAAACTCCAGGTAGGGCAG | SHANK3 H1:2 5UTR(+346+370) |
| 597 | GCTTGTATCGAAACTCCAGGTAGGG | SHANK3 H1:2 5UTR(+349+373) |
| 598 | GCCGCTTGTATCGAAACTCCAGGTA | SHANK3 H1:2 5UTR(+352+376) |
| 599 | CTCGCCGCTTGTATCGAAACTCCAG | SHANK3 H1:2 5UTR(+355+379) |
| 600 | AAACTCGCCGCTTGTATCGAAACTC | SHANK3 H1:2 5UTR(+358+382) |
| 601 | CATAAACTCGCCGCTTGTATCGAAA | SHANK3 H2 5UTR(+361+385) |
| 602 | GGGCATAAACTCGCCGCTTGTATCG | SHANK3 H2 5UTR(+364+388) |
| 603 | TCTGGGCATAAACTCGCCGCTTGTA | SHANK3 H2 5UTR(+367+391) |
| 604 | GGTTCTGGGCATAAACTCGCCGCTT | SHANK3 H2 5UTR(+370+394) |
| 605 | TGAGGTTCTGGGCATAAACTCGCCG | SHANK3 H2 5UTR(+373+397) |
| 606 | CGATGAGGTTCTGGGCATAAACTCG | SHANK3 H2 5UTR(+376+400) |
| 607 | CATCGATGAGGTTCTGGGCATAAAC | SHANK3 H2 5UTR(+379+403) |
| 608 | TATCATCGATGAGGTTCTGGGCATA | SHANK3 H2 5UTR(+382+406) |
| 609 | GCTTATCATCGATGAGGTTCTGGGC | SHANK3 H2 5UTR(+385+409) |
| 610 | ACTGCTTATCATCGATGAGGTTCTG | SHANK3 H2 5UTR(+388+412) |
| 611 | CAAACTGCTTATCATCGATGAGGTT | SHANK3 H2 5UTR(+391+415) |
| 612 | TTGCAAACTGCTTATCATCGATGAG | SHANK3 H2 5UTR(+394+418) |
| 613 | GCTTTGCAAACTGCTTATCATCGAT | SHANK3 H2 5UTR(+397+421) |
| 614 | GAAGCTTTGCAAACTGCTTATCATC | SHANK3 H2 5UTR(+400+424) |
| 615 | TGTGAAGCTTTGCAAACTGCTTATC | SHANK3 H2 5UTR(+403+427) |
| 616 | TTGTGTGAAGCTTTGCAAACTGCTT | SHANK3 H2 5UTR(+406+430) |
| 617 | CCTTTGTGTGAAGCTTTGCAAACTG | SHANK3 H2:3 5UTR(+409+433) |
| 618 | TCGCCTTTGTGTGAAGCTTTGCAAA | SHANK3 H2:3 5UTR(+412+436) |
| 619 | GGTTCGCCTTTGTGTGAAGCTTTGC | SHANK3 H2:3 5UTR(+415+439) |
| 620 | TCAGGTTCGCCTTTGTGTGAAGCTT | SHANK3 H2:3 5UTR(+418+442) |
| 621 | TCTTCAGGTTCGCCTTTGTGTGAAG | SHANK3 H2:3 5UTR(+421+445) |
| 622 | ACTTCTTCAGGTTCGCCTTTGTGTG | SHANK3 H2:3 5UTR(+424+448) |
| 623 | GGAGTGGGGGTGGGGCG | SHANK3 H22 3UTR(+593+609) |
| 624 | GCGGGAGTGGGGGTGGG | SHANK3 H22 3UTR(+596+612) |
| 625 | GGGGGGGGAGTGGGGGT | SHANK3 H22 3UTR(+599+615) |
| 626 | GCCGGGGGGGGAGTGGG | SHANK3 H22 3UTR(+602+618) |
| 627 | ACGGCCGGGGGGGGAGT | SHANK3 H22 3UTR(+605+621) |
| 628 | GGCACGGCCGGGGGGGG | SHANK3 H22 3UTR(+608+624) |
| 629 | CAGGGCACGGCCGGGGC | SHANK3 H22 3UTR(+611+627) |
| 630 | CGGCAGGGCACGGCCGG | SHANK3 H22 3UTR(+614+630) |
| 631 | TGCCGGCAGGGCACGGC | SHANK3 H22 3UTR(+617+633) |
| 632 | CCCTGCCGGCAGGGCAC | SHANK3 H22 3UTR(+620+636) |
| 633 | GGGCCCTGCCGGCAGGG | SHANK3 H22 3UTR(+623+639) |
| 634 | GGGGGGCCCTGCCGGCA | SHANK3 H22 3UTR(+626+642) |
| 635 | GGTGGGGGGCCCTGCCG | SHANK3 H22 3UTR(+629+645) |
| 636 | GGGGGTGGGGGGCCCTG | SHANK3 H22 3UTR(+632+648) |
| 637 | GGTGGGGGTGGGGGGCC | SHANK3 H22 3UTR(+635+651) |
| 638 | CGGGGTGGGGGTGGGGG | SHANK3 H22 3UTR(+638+654) |
| 639 | GCCCGGGGTGGGGGTGG | SHANK3 H22 3UTR(+641+657) |
| 640 | GCGGCCCGGGGTGGGGG | SHANK3 H22 3UTR(+644+660) |
| 641 | CCCGCGGCCCGGGGTGG | SHANK3 H22 3UTR(+647+663) |
| 642 | GAGCCCGCGGCCCGGGG | SHANK3 H22 3UTR(+650+666) |
| 643 | GCCGAGCCCGCGGCCCG | SHANK3 H22 3UTR(+653+669) |
| 644 | CAGGCCGAGCCCGCGGC | SHANK3 H22 3UTR(+656+672) |
| 645 | GGGCAGGCCGAGCCCGC | SHANK3 H22 3UTR(+659+675) |
| 646 | AAGGGGCAGGCCGAGCC | SHANK3 H22 3UTR(+662+678) |
| 647 | CGTAAGGGGCAGGCCGA | SHANK3 H22 3UTR(+665+681) |
| 648 | CGTCGTAAGGGGCAGGC | SHANK3 H22 3UTR(+668+684) |
| 649 | CGCCGTCGTAAGGGGCA | SHANK3 H22 3UTR(+671+687) |
| 650 | GGGCGCCGTCGTAAGGG | SHANK3 H22 3UTR(+674+690) |
| 651 | CCCGGGCGCCGTCGTAA | SHANK3 H22 3UTR(+677+693) |
| 652 | TGGCCCGGGCGCCGTCG | SHANK3 H22 3UTR(+680+696) |
| 653 | TCCTGGCCCGGGCGCCG | SHANK3 H22 3UTR(+683+699) |
| 654 | CATTCCTGGCCCGGGCG | SHANK3 H22 3UTR(+686+702) |
| 655 | CAACATTCCTGGCCCGG | SHANK3 H22 3UTR(+689+705) |
| 656 | ATGCAACATTCCTGGCC | SHANK3 H22 3UTR(+692+708) |
| 657 | TTCATGCAACATTCCTG | SHANK3 H22 3UTR(+695+711) |
| 658 | CGATTCATGCAACATTC | SHANK3 H22 3UTR(+698+714) |
| 659 | GGACGATTCATGCAACA | SHANK3 H22 3UTR(+701+717) |
| 660 | ACAGGACGATTCATGCA | SHANK3 H22 3UTR(+704+720) |
| 661 | CAAACAGGACGATTCAT | SHANK3 H22 3UTR(+707+723) |
| 662 | CAGCAAACAGGACGATT | SHANK3 H22 3UTR(+710+726) |
| 663 | CAACAGCAAACAGGACG | SHANK3 H22 3UTR(+713+729) |
| 664 | GAGCAACAGCAAACAGG | SHANK3 H22 3UTR(+716+732) |
| 665 | TCCGAGCAACAGCAAAC | SHANK3 H22 3UTR(+719+735) |
| 666 | GTCTCCGAGCAACAGCA | SHANK3 H22 3UTR(+722+738) |
| 667 | CGAGTCTCCGAGCAACA | SHANK3 H22 3UTR(+725+741) |
| 668 | GGGCGAGTCTCCGAGCA | SHANK3 H22 3UTR(+728+744) |
| 669 | ACAGGGCGAGTCTCCGA | SHANK3 H22 3UTR(+731+747) |
| 670 | TGTACAGGGCGAGTCTC | SHANK3 H22 3UTR(+734+750) |
| 671 | CAATGTACAGGGCGAGT | SHANK3 H22 3UTR(+737+753) |
| 672 | AAGCAATGTACAGGGCG | SHANK3 H22 3UTR(+740+756) |
| 673 | ACTAAGCAATGTACAGG | SHANK3 H22 3UTR(+743+759) |
| 674 | GGCACTAAGCAATGTAC | SHANK3 H22 3UTR(+746+762) |
| 675 | GAGGGCACTAAGCAATG | SHANK3 H22 3UTR(+749+765) |
| 676 | GGTGAGGGCACTAAGCA | SHANK3 H22 3UTR(+752+768) |
| 677 | GCCGGTGAGGGCACTAA | SHANK3 H22 3UTR(+755+771) |
| 678 | GCGGCCGGTGAGGGCAC | SHANK3 H22 3UTR(+758+774) |
| 679 | TGGGCGGCCGGTGAGGG | SHANK3 H22 3UTR(+761+777) |
| 680 | GGCTGGGCGGCCGGTGA | SHANK3 H22 3UTR(+764+780) |
| 681 | GTGGGCTGGGCGGCCGG | SHANK3 H22 3UTR(+767+783) |
| 682 | TGGGTGGGCTGGGCGGC | SHANK3 H22 3UTR(+770+786) |
| 683 | CGCTGGGTGGGCTGGGC | SHANK3 H22 3UTR(+773+789) |
| 684 | GTGCGCTGGGTGGGCTG | SHANK3 H22 3UTR(+776+792) |
| 685 | ACTGTGCGCTGGGTGGG | SHANK3 H22 3UTR(+779+795) |
| 686 | CTGACTGTGCGCTGGGT | SHANK3 H22 3UTR(+782+798) |
| 687 | TTCCTGACTGTGCGCTG | SHANK3 H22 3UTR(+785+801) |
| 688 | CCCTTCCTGACTGTGCG | SHANK3 H22 3UTR(+788+804) |
| 689 | ACGCCCTTCCTGACTGT | SHANK3 H22 3UTR(+791+807) |
| 690 | TCCACGCCCTTCCTGAC | SHANK3 H22 3UTR(+794+810) |
| 691 | TGGTCCACGCCCTTCCT | SHANK3 H22 3UTR(+797+813) |
| 692 | CCCTGGTCCACGCCCTT | SHANK3 H22 3UTR(+800+816) |
| 693 | CCTCCCTGGTCCACGCC | SHANK3 H22 3UTR(+803+819) |
| 694 | CAGCCTCCCTGGTCCAC | SHANK3 H22 3UTR(+806+822) |
| 695 | CCCCAGCCTCCCTGGTC | SHANK3 H22 3UTR(+809+825) |
| 696 | CCGCCCCAGCCTCCCTG | SHANK3 H22 3UTR(+812+828) |
| 697 | CTCCCGCCCCAGCCTCC | SHANK3 H22 3UTR(+815+831) |
| 698 | CACCTCCCGCCCCAGCC | SHANK3 H22 3UTR(+818+834) |
| 699 | CGGCACCTCCCGCCCCA | SHANK3 H22 3UTR(+821+837) |
| 700 | CCCCGGCACCTCCCGCC | SHANK3 H22 3UTR(+824+840) |
| 701 | CACCCCCGGCACCTCCC | SHANK3 H22 3UTR(+827+843) |
| 702 | CCCCACCCCCGGCACCT | SHANK3 H22 3UTR(+830+846) |
| 703 | GCACCCCACCCCCGGCA | SHANK3 H22 3UTR(+833+849) |
| 704 | AGGGCACCCCACCCCCG | SHANK3 H22 3UTR(+836+852) |
| 705 | GCTAGGGCACCCCACCC | SHANK3 H22 3UTR(+839+855) |
| 706 | CACGCTAGGGCACCCCA | SHANK3 H22 3UTR(+842+858) |
| 707 | GGTCACGCTAGGGCACC | SHANK3 H22 3UTR(+845+861) |
| 708 | GGTGGTCACGCTAGGGC | SHANK3 H22 3UTR(+848+864) |
| 709 | GGAGGTGGTCACGCTAG | SHANK3 H22 3UTR(+851+867) |
| 710 | GAAGGAGGTGGTCACGC | SHANK3 H22 3UTR(+854+870) |
| 711 | TGCGAAGGAGGTGGTCA | SHANK3 H22 3UTR(+857+873) |
| 712 | AGCTGCGAAGGAGGTGG | SHANK3 H22 3UTR(+860+876) |
| 713 | AGGAGCTGCGAAGGAGG | SHANK3 H22 3UTR(+863+879) |
| 714 | ACCAGGAGCTGCGAAGG | SHANK3 H22 3UTR(+866+882) |
| 715 | GCCACCAGGAGCTGCGA | SHANK3 H22 3UTR(+869+885) |
| 716 | ATGGCCACCAGGAGCTG | SHANK3 H22 3UTR(+872+888) |
| 717 | AGAATGGCCACCAGGAG | SHANK3 H22 3UTR(+875+891) |
| 718 | GGGAGAATGGCCACCAG | SHANK3 H22 3UTR(+878+894) |
| 719 | TCTGGGAGAATGGCCAC | SHANK3 H22 3UTR(+881+897) |
| 720 | CCCTCTGGGAGAATGGC | SHANK3 H22 3UTR(+884+900) |
| 721 | TCCCCCTCTGGGAGAAT | SHANK3 H22 3UTR(+887+903) |
| 722 | GGTTCCCCCTCTGGGAG | SHANK3 H22 3UTR(+890+906) |
| 723 | CTAGGTTCCCCCTCTGG | SHANK3 H22 3UTR(+893+909) |
| 724 | GGACTAGGTTCCCCCTC | SHANK3 H22 3UTR(+896+912) |
| 725 | GCTGGACTAGGTTCCCC | SHANK3 H22 3UTR(+899+915) |
| 726 | CATGCTGGACTAGGTTC | SHANK3 H22 3UTR(+902+918) |
| 727 | TCGCATGCTGGACTAGG | SHANK3 H22 3UTR(+905+921) |
| 728 | ACCTCGCATGCTGGACT | SHANK3 H22 3UTR(+908+924) |
| 729 | CTGACCTCGCATGCTGG | SHANK3 H22 3UTR(+911+927) |
| 730 | GTCCTGACCTCGCATGC | SHANK3 H22 3UTR(+914+930) |
| 731 | CGGGTCCTGACCTCGCA | SHANK3 H22 3UTR(+917+933) |
| 732 | AGGCGGGTCCTGACCTC | SHANK3 H22 3UTR(+920+936) |
| 733 | CCAAGGCGGGTCCTGAC | SHANK3 H22 3UTR(+923+939) |
| 734 | TCACCAAGGCGGGTCCT | SHANK3 H22 3UTR(+926+942) |
| 735 | GAGTCACCAAGGCGGGT | SHANK3 H22 3UTR(+929+945) |
| 736 | CCCGAGTCACCAAGGCG | SHANK3 H22 3UTR(+932+948) |
| 737 | CCCCCCGAGTCACCAAG | SHANK3 H22 3UTR(+935+951) |
| 738 | CCTCCCCCCGAGTCACC | SHANK3 H22 3UTR(+938+954) |
| 739 | CCCCCTCCCCCCGAGTC | SHANK3 H22 3UTR(+941+957) |
| 740 | CTCCCCCCTCCCCCCGA | SHANK3 H22 3UTR(+944+960) |
| 741 | TGTCTCCCCCCTCCCCC | SHANK3 H22 3UTR(+947+963) |
| 742 | CAATGTCTCCCCCCTCC | SHANK3 H22 3UTR(+950+966) |
| 743 | TCCCAATGTCTCCCCCC | SHANK3 H22 3UTR(+953+969) |
| 744 | GAATCCCAATGTCTCCC | SHANK3 H22 3UTR(+956+972) |
| 745 | CGAGAATCCCAATGTCT | SHANK3 H22 3UTR(+959+975) |
| 746 | CATCGAGAATCCCAATG | SHANK3 H22 3UTR(+962+978) |
| 747 | CCCCATCGAGAATCCCA | SHANK3 H22 3UTR(+965+981) |
| 748 | GGCCCCCATCGAGAATC | SHANK3 H22 3UTR(+968+984) |
| 749 | CTTGGCCCCCATCGAGA | SHANK3 H22 3UTR(+971+987) |
| 750 | CTCCTTGGCCCCCATCG | SHANK3 H22 3UTR(+974+990) |
| 751 | GGGCTCCTTGGCCCCCA | SHANK3 H22 3UTR(+977+993) |
| 752 | GGGGGGCTCCTTGGCCC | SHANK3 H22 3UTR(+980+996) |
| 753 | ACAGGGGGGCTCCTTGG | SHANK3 H22 3UTR(+983+999) |
| 754 | AAAACAGGGGGGCTCCT | SHANK3 H22 3UTR(+986+1002) |
| 755 | TGCAAAACAGGGGGGCT | SHANK3 H22 3UTR(+989+1005) |
| 756 | ATATGCAAAACAGGGGG | SHANK3 H22 3UTR(+992+1008) |
| 757 | AAAATATGCAAAACAGG | SHANK3 H22 3UTR(+995+1011) |
| 758 | ATTAAAATATGCAAAAC | SHANK3 H22 3UTR(+998+1014) |
| 759 | TGGATTAAAATATGCAA | SHANK3 H22 3UTR(+1001+1017) |
| 760 | GAGTGGATTAAAATATG | SHANK3 H22 3UTR(+1004+1020) |
| 761 | ATAGAGTGGATTAAAAT | SHANK3 H22 3UTR(+1007+1023) |
| 762 | AATATAGAGTGGATTAA | SHANK3 H22 3UTR(+1010+1026) |
| 763 | CCAAATATAGAGTGGAT | SHANK3 H22 3UTR(+1013+1029) |
| 764 | GTTCCAAATATAGAGTG | SHANK3 H22 3UTR(+1016+1032) |
| 765 | CTCGTTCCAAATATAGA | SHANK3 H22 3UTR(+1019+1035) |
| 766 | TTTCTCGTTCCAAATAT | SHANK3 H22 3UTR(+1022+1038) |
| 767 | CCTTTTCTCGTTCCAAA | SHANK3 H22 3UTR(+1025+1041) |
| 768 | GTTCCTTTTCTCGTTCC | SHANK3 H22 3UTR(+1028+1044) |
| 769 | TTTGTTCCTTTTCTCGT | SHANK3 H22 3UTR(+1031+1047) |
| 770 | ATATTTGTTCCTTTTCT | SHANK3 H22 3UTR(+1034+1050) |
| 771 | GAGATATTTGTTCCTTT | SHANK3 H22 3UTR(+1037+1053) |
| 772 | ACAGAGATATTTGTTCC | SHANK3 H22 3UTR(+1040+1056) |
| 773 | CGGACAGAGATATTTGT | SHANK3 H22 3UTR(+1043+1059) |
| 774 | TTACGGACAGAGATATT | SHANK3 H22 3UTR(+1046+1062) |
| 775 | CTATTACGGACAGAGAT | SHANK3 H22 3UTR(+1049+1065) |
| 776 | AAACTATTACGGACAGA | SHANK3 H22 3UTR(+1052+1068) |
| 777 | AGGAAACTATTACGGAC | SHANK3 H22 3UTR(+1055+1071) |
| 778 | GAGAGGAAACTATTACG | SHANK3 H22 3UTR(+1058+1074) |
| 779 | GGGGAGAGGAAACTATT | SHANK3 H22 3UTR(+1061+1077) |
| 780 | GGAGGGGAGAGGAAACT | SHANK3 H22 3UTR(+1064+1080) |
| 781 | AAGGGAGGGGAGAGGAA | SHANK3 H22 3UTR(+1067+1083) |
| 782 | TAGAAGGGAGGGGAGAG | SHANK3 H22 3UTR(+1070+1086) |
| 783 | AAGTAGAAGGGAGGGGA | SHANK3 H22 3UTR(+1073+1089) |
| 784 | TGGAAGTAGAAGGGAGG | SHANK3 H22 3UTR(+1076+1092) |
| 785 | CAGTGGAAGTAGAAGGG | SHANK3 H22 3UTR(+1079+1095) |
| 786 | GACCAGTGGAAGTAGAA | SHANK3 H22 3UTR(+1082+1098) |
| 787 | TGGGACCAGTGGAAGTA | SHANK3 H22 3UTR(+1085+1101) |
| 788 | CAGTGGGACCAGTGGAA | SHANK3 H22 3UTR(+1088+1104) |
| 789 | CTGCAGTGGGACCAGTG | SHANK3 H22 3UTR(+1091+1107) |
| 790 | CAGCTGCAGTGGGACCA | SHANK3 H22 3UTR(+1094+1110) |
| 791 | GGGCAGCTGCAGTGGGA | SHANK3 H22 3UTR(+1097+1113) |
| 792 | ACTGGGCAGCTGCAGTG | SHANK3 H22 3UTR(+1100+1116) |
| 793 | AAGACTGGGCAGCTGCA | SHANK3 H22 3UTR(+1103+1119) |
| 794 | TGGAAGACTGGGCAGCT | SHANK3 H22 3UTR(+1106+1122) |
| 795 | AGATGGAAGACTGGGCA | SHANK3 H22 3UTR(+1109+1125) |
| 796 | CGGAGATGGAAGACTGG | SHANK3 H22 3UTR(+1112+1128) |
| 797 | GGCCGGAGATGGAAGAC | SHANK3 H22 3UTR(+1115+1131) |
| 798 | AGGGGCCGGAGATGGAA | SHANK3 H22 3UTR(+1118+1134) |
| 799 | GTGAGGGGCCGGAGATG | SHANK3 H22 3UTR(+1121+1137) |
| 800 | GCAGTGAGGGGCCGGAG | SHANK3 H22 3UTR(+1124+1140) |
| 801 | GTGGCAGTGAGGGGCCG | SHANK3 H22 3UTR(+1127+1143) |
| 802 | GCAGTGGCAGTGAGGGG | SHANK3 H22 3UTR(+1130+1146) |
| 803 | GTGGCAGTGGCAGTGAG | SHANK3 H22 3UTR(+1133+1149) |
| 804 | GGGGTGGCAGTGGCAGT | SHANK3 H22 3UTR(+1136+1152) |
| 805 | TGTGGGGTGGCAGTGGC | SHANK3 H22 3UTR(+1139+1155) |
| 806 | CGTTGTGGGGTGGCAGT | SHANK3 H22 3UTR(+1142+1158) |
| 807 | CCCCGTTGTGGGGTGGC | SHANK3 H22 3UTR(+1145+1161) |
| 808 | CTGCCCCGTTGTGGGGT | SHANK3 H22 3UTR(+1148+1164) |
| 809 | CCCCTGCCCCGTTGTGG | SHANK3 H22 3UTR(+1151+1167) |
| 810 | CGTCCCCTGCCCCGTTG | SHANK3 H22 3UTR(+1154+1170) |
| 811 | GAGCGTCCCCTGCCCCG | SHANK3 H22 3UTR(+1157+1173) |
| 812 | CTGGAGCGTCCCCTGCC | SHANK3 H22 3UTR(+1160+1176) |
| 813 | CAGCTGGAGCGTCCCCT | SHANK3 H22 3UTR(+1163+1179) |
| 814 | GACCAGCTGGAGCGTCC | SHANK3 H22 3UTR(+1166+1182) |
| 815 | CCAGACCAGCTGGAGCG | SHANK3 H22 3UTR(+1169+1185) |
| 816 | ACCCCAGACCAGCTGGA | SHANK3 H22 3UTR(+1172+1188) |
| 817 | CCAACCCCAGACCAGCT | SHANK3 H22 3UTR(+1175+1191) |
| 818 | TGGCCAACCCCAGACCA | SHANK3 H22 3UTR(+1178+1194) |
| 819 | CCCTGGCCAACCCCAGA | SHANK3 H22 3UTR(+1181+1197) |
| 820 | GGGCCCTGGCCAACCCC | SHANK3 H22 3UTR(+1184+1200) |
| 821 | CTAGGGCCCTGGCCAAC | SHANK3 H22 3UTR(+1187+1203) |
| 822 | CCACTAGGGCCCTGGCC | SHANK3 H22 3UTR(+1190+1206) |
| 823 | GGGCCACTAGGGCCCTG | SHANK3 H22 3UTR(+1193+1209) |
| 824 | GGCGGGCCACTAGGGCC | SHANK3 H22 3UTR(+1196+1212) |
| 825 | CAGGGCGGGCCACTAGG | SHANK3 H22 3UTR(+1199+1215) |
| 826 | CCCCAGGGCGGGCCACT | SHANK3 H22 3UTR(+1202+1218) |
| 827 | GGGCCCCAGGGCGGGCC | SHANK3 H22 3UTR(+1205+1221) |
| 828 | CTGGGGCCCCAGGGCGG | SHANK3 H22 3UTR(+1208+1224) |
| 829 | GAGCTGGGGCCCCAGGG | SHANK3 H22 3UTR(+1211+1227) |
| 830 | GCCGAGCTGGGGCCCCA | SHANK3 H22 3UTR(+1214+1230) |
| 831 | GGGGCCGAGCTGGGGCC | SHANK3 H22 3UTR(+1217+1233) |
| 832 | CGAGGGGCCGAGCTGGG | SHANK3 H22 3UTR(+1220+1236) |
| 833 | AGGCGAGGGGCCGAGCT | SHANK3 H22 3UTR(+1223+1239) |
| 834 | GCGAGGCGAGGGGCCGA | SHANK3 H22 3UTR(+1226+1242) |
| 835 | TCAGCGAGGCGAGGGGC | SHANK3 H22 3UTR(+1229+1245) |
| 836 | AGCTCAGCGAGGCGAGG | SHANK3 H22 3UTR(+1232+1248) |
| 837 | TAGAGCTCAGCGAGGCG | SHANK3 H22 3UTR(+1235+1251) |
| 838 | CACTAGAGCTCAGCGAG | SHANK3 H22 3UTR(+1238+1254) |
| 839 | GCACACTAGAGCTCAGC | SHANK3 H22 3UTR(+1241+1257) |
| 840 | GGGGCACACTAGAGCTC | SHANK3 H22 3UTR(+1244+1260) |
| 841 | GGTGGGGCACACTAGAG | SHANK3 H22 3UTR(+1247+1263) |
| 842 | GTCGGTGGGGCACACTA | SHANK3 H22 3UTR(+1250+1266) |
| 843 | AGGGTCGGTGGGGCACA | SHANK3 H22 3UTR(+1253+1269) |
| 844 | TGAAGGGTCGGTGGGGC | SHANK3 H22 3UTR(+1256+1272) |
| 845 | ACCTGAAGGGTCGGTGG | SHANK3 H22 3UTR(+1259+1275) |
| 846 | AGCACCTGAAGGGTCGG | SHANK3 H22 3UTR(+1262+1278) |
| 847 | AGCAGCACCTGAAGGGT | SHANK3 H22 3UTR(+1265+1281) |
| 848 | ACGAGCAGCACCTGAAG | SHANK3 H22 3UTR(+1268+1284) |
| 849 | ACCACGAGCAGCACCTG | SHANK3 H22 3UTR(+1271+1287) |
| 850 | CCCACCACGAGCAGCAC | SHANK3 H22 3UTR(+1274+1290) |
| 851 | CCTCCCACCACGAGCAG | SHANK3 H22 3UTR(+1277+1293) |
| 852 | GCCCCTCCCACCACGAG | SHANK3 H22 3UTR(+1280+1296) |
| 853 | GCCGCCCCTCCCACCAC | SHANK3 H22 3UTR(+1283+1299) |
| 854 | CCTGCCGCCCCTCCCAC | SHANK3 H22 3UTR(+1286+1302) |
| 855 | CGGCCTGCCGCCCCTCC | SHANK3 H22 3UTR(+1289+1305) |
| 856 | CCGCGGCCTGCCGCCCC | SHANK3 H22 3UTR(+1292+1308) |
| 857 | GACCCGCGGCCTGCCGC | SHANK3 H22 3UTR(+1295+1311) |
| 858 | CAGGACCCGCGGCCTGC | SHANK3 H22 3UTR(+1298+1314) |
| 859 | CAGCAGGACCCGCGGCC | SHANK3 H22 3UTR(+1301+1317) |
| 860 | GCACAGCAGGACCCGCG | SHANK3 H22 3UTR(+1304+1320) |
| 861 | GGTGCACAGCAGGACCC | SHANK3 H22 3UTR(+1307+1323) |
| 862 | GCGGGTGCACAGCAGGA | SHANK3 H22 3UTR(+1310+1326) |
| 863 | CCCGCGGGTGCACAGCA | SHANK3 H22 3UTR(+1313+1329) |
| 864 | GGTCCCGCGGGTGCACA | SHANK3 H22 3UTR(+1316+1332) |
| 865 | GCTGGTCCCGCGGGTGC | SHANK3 H22 3UTR(+1319+1335) |
| 866 | CCGGCTGGTCCCGCGGG | SHANK3 H22 3UTR(+1322+1338) |
| 867 | AGGCCGGCTGGTCCCGC | SHANK3 H22 3UTR(+1325+1341) |
| 868 | CCCAGGCCGGCTGGTCC | SHANK3 H22 3UTR(+1328+1344) |
| 869 | TCTCCCAGGCCGGCTGG | SHANK3 H22 3UTR(+1331+1347) |
| 870 | TGGTCTCCCAGGCCGGC | SHANK3 H22 3UTR(+1334+1350) |
| 871 | CGATGGTCTCCCAGGCC | SHANK3 H22 3UTR(+1337+1353) |
| 872 | GGCCGATGGTCTCCCAG | SHANK3 H22 3UTR(+1340+1356) |
| 873 | CCCGGCCGATGGTCTCC | SHANK3 H22 3UTR(+1343+1359) |
| 874 | CCCCCCGGCCGATGGTC | SHANK3 H22 3UTR(+1346+1362) |
| 875 | ATCCCCCCCGGCCGATG | SHANK3 H22 3UTR(+1349+1365) |
| 876 | CTCATCCCCCCCGGCCG | SHANK3 H22 3UTR(+1352+1368) |
| 877 | GCCCTCATCCCCCCCGG | SHANK3 H22 3UTR(+1355+1371) |
| 878 | CCTGCCCTCATCCCCCC | SHANK3 H22 3UTR(+1358+1374) |
| 879 | GGCCCTGCCCTCATCCC | SHANK3 H22 3UTR(+1361+1377) |
| 880 | CAGGGCCCTGCCCTCAT | SHANK3 H22 3UTR(+1364+1380) |
| 881 | CGGCAGGGCCCTGCCCT | SHANK3 H22 3UTR(+1367+1383) |
| 882 | GAGCGGCAGGGCCCTGC | SHANK3 H22 3UTR(+1370+1386) |
| 883 | GTGGAGCGGCAGGGCCC | SHANK3 H22 3UTR(+1373+1389) |
| 884 | GCGGTGGAGCGGCAGGG | SHANK3 H22 3UTR(+1376+1392) |
| 885 | GCTGCGGTGGAGCGGCA | SHANK3 H22 3UTR(+1379+1395) |
| 886 | ATGGCTGCGGTGGAGCG | SHANK3 H22 3UTR(+1382+1398) |
| 887 | AAGATGGCTGCGGTGGA | SHANK3 H22 3UTR(+1385+1401) |
| 888 | AGGAAGATGGCTGCGGT | SHANK3 H22 3UTR(+1388+1404) |
| 889 | GTGAGGAAGATGGCTGC | SHANK3 H22 3UTR(+1391+1407) |
| 890 | CCTGTGAGGAAGATGGC | SHANK3 H22 3UTR(+1394+1410) |
| 891 | GACCCTGTGAGGAAGAT | SHANK3 H22 3UTR(+1397+1413) |
| 892 | AGAGACCCTGTGAGGAA | SHANK3 H22 3UTR(+1400+1416) |
| 893 | GGGAGAGACCCTGTGAG | SHANK3 H22 3UTR(+1403+1419) |
| 894 | TTGGGGAGAGACCCTGT | SHANK3 H22 3UTR(+1406+1422) |
| 895 | TCCTTGGGGAGAGACCC | SHANK3 H22 3UTR(+1409+1425) |
| 896 | CCCTCCTTGGGGAGAGA | SHANK3 H22 3UTR(+1412+1428) |
| 897 | GCCCCCTCCTTGGGGAG | SHANK3 H22 3UTR(+1415+1431) |
| 898 | CTAGCCCCCTCCTTGGG | SHANK3 H22 3UTR(+1418+1434) |
| 899 | AAGCTAGCCCCCTCCTT | SHANK3 H22 3UTR(+1421+1437) |
| 900 | ACCAAGCTAGCCCCCTC | SHANK3 H22 3UTR(+1424+1440) |
| 901 | GGGACCAAGCTAGCCCC | SHANK3 H22 3UTR(+1427+1443) |
| 902 | ATGGGGACCAAGCTAGC | SHANK3 H22 3UTR(+1430+1446) |
| 903 | AGCATGGGGACCAAGCT | SHANK3 H22 3UTR(+1433+1449) |
| 904 | AAGAGCATGGGGACCAA | SHANK3 H22 3UTR(+1436+1452) |
| 905 | CCCAAGAGCATGGGGAC | SHANK3 H22 3UTR(+1439+1455) |
| 906 | TTGCCCAAGAGCATGGG | SHANK3 H22 3UTR(+1442+1458) |
| 907 | TAGTTGCCCAAGAGCAT | SHANK3 H22 3UTR(+1445+1461) |
| 908 | CTGTAGTTGCCCAAGAG | SHANK3 H22 3UTR(+1448+1464) |
| 909 | CTGCTGTAGTTGCCCAA | SHANK3 H22 3UTR(+1451+1467) |
| 910 | TCTCTGCTGTAGTTGCC | SHANK3 H22 3UTR(+1454+1470) |
| 911 | GCTTCTCTGCTGTAGTT | SHANK3 H22 3UTR(+1457+1473) |
| 912 | GAGGCTTCTCTGCTGTA | SHANK3 H22 3UTR(+1460+1476) |
| 913 | AGGGAGGCTTCTCTGCT | SHANK3 H22 3UTR(+1463+1479) |
| 914 | GGCAGGGAGGCTTCTCT | SHANK3 H22 3UTR(+1466+1482) |
| 915 | CAAGGCAGGGAGGCTTC | SHANK3 H22 3UTR(+1469+1485) |
| 916 | GTCCAAGGCAGGGAGGC | SHANK3 H22 3UTR(+1472+1488) |
| 917 | GGGGTCCAAGGCAGGGA | SHANK3 H22 3UTR(+1475+1491) |
| 918 | TTTGGGGTCCAAGGCAG | SHANK3 H22 3UTR(+1478+1494) |
| 919 | GACTTTGGGGTCCAAGG | SHANK3 H22 3UTR(+1481+1497) |
| 920 | GGAGACTTTGGGGTCCA | SHANK3 H22 3UTR(+1484+1500) |
| 921 | ACAGGAGACTTTGGGGT | SHANK3 H22 3UTR(+1487+1503) |
| 922 | AGGACAGGAGACTTTGG | SHANK3 H22 3UTR(+1490+1506) |
| 923 | GGCAGGACAGGAGACTT | SHANK3 H22 3UTR(+1493+1509) |
| 924 | AAGGGCAGGACAGGAGA | SHANK3 H22 3UTR(+1496+1512) |
| 925 | ATAAAGGGCAGGACAGG | SHANK3 H22 3UTR(+1499+1515) |
| 926 | CACATAAAGGGCAGGAC | SHANK3 H22 3UTR(+1502+1518) |
| 927 | ACACACATAAAGGGCAG | SHANK3 H22 3UTR(+1505+1521) |
| 928 | CCCACACACATAAAGGG | SHANK3 H22 3UTR(+1508+1524) |
| 929 | TCACCCACACACATAAA | SHANK3 H22 3UTR(+1511+1527) |
| 930 | GTTTCACCCACACACAT | SHANK3 H22 3UTR(+1514+1530) |
| 931 | CCAGTTTCACCCACACA | SHANK3 H22 3UTR(+1517+1533) |
| 932 | CACCCAGTTTCACCCAC | SHANK3 H22 3UTR(+1520+1536) |
| 933 | ACGCACCCAGTTTCACC | SHANK3 H22 3UTR(+1523+1539) |
| 934 | CAGACGCACCCAGTTTC | SHANK3 H22 3UTR(+1526+1542) |
| 935 | GCTCAGACGCACCCAGT | SHANK3 H22 3UTR(+1529+1545) |
| 936 | CGTGCTCAGACGCACCC | SHANK3 H22 3UTR(+1532+1548) |
| 937 | CCACGTGCTCAGACGCA | SHANK3 H22 3UTR(+1535+1551) |
| 938 | CTCCCACGTGCTCAGAC | SHANK3 H22 3UTR(+1538+1554) |
| 939 | CGGCTCCCACGTGCTCA | SHANK3 H22 3UTR(+1541+1557) |
| 940 | ACACGGCTCCCACGTGC | SHANK3 H22 3UTR(+1544+1560) |
| 941 | CACACACGGCTCCCACG | SHANK3 H22 3UTR(+1547+1563) |
| 942 | GCACACACACGGCTCCC | SHANK3 H22 3UTR(+1550+1566) |
| 943 | CAGGCACACACACGGCT | SHANK3 H22 3UTR(+1553+1569) |
| 944 | AATCAGGCACACACACG | SHANK3 H22 3UTR(+1556+1572) |
| 945 | AGTAATCAGGCACACAC | SHANK3 H22 3UTR(+1559+1575) |
| 946 | CTCAGTAATCAGGCACA | SHANK3 H22 3UTR(+1562+1578) |
| 947 | CCACTCAGTAATCAGGC | SHANK3 H22 3UTR(+1565+1581) |
| 948 | TGGCCACTCAGTAATCA | SHANK3 H22 3UTR(+1568+1584) |
| 949 | TGGTGGCCACTCAGTAA | SHANK3 H22 3UTR(+1571+1587) |
| 950 | CCCTGGTGGCCACTCAG | SHANK3 H22 3UTR(+1574+1590) |
| 951 | GGCCCCTGGTGGCCACT | SHANK3 H22 3UTR(+1577+1593) |
| 952 | AGCGGCCCCTGGTGGCC | SHANK3 H22 3UTR(+1580+1596) |
| 953 | CAGAGCGGCCCCTGGTG | SHANK3 H22 3UTR(+1583+1599) |
| 954 | GTCCAGAGCGGCCCCTG | SHANK3 H22 3UTR(+1586+1602) |
| 955 | CTAGTCCAGAGCGGCCC | SHANK3 H22 3UTR(+1589+1605) |
| 956 | GCGCTAGTCCAGAGCGG | SHANK3 H22 3UTR(+1592+1608) |
| 957 | CCCGCGCTAGTCCAGAG | SHANK3 H22 3UTR(+1595+1611) |
| 958 | GGCCCCGCGCTAGTCCA | SHANK3 H22 3UTR(+1598+1614) |
| 959 | CACGGCCCCGCGCTAGT | SHANK3 H22 3UTR(+1601+1617) |
| 960 | CTCCACGGCCCCGCGCT | SHANK3 H22 3UTR(+1604+1620) |
| 961 | CGCCTCCACGGCCCCGC | SHANK3 H22 3UTR(+1607+1623) |
| 962 | GCACGCCTCCACGGCCC | SHANK3 H22 3UTR(+1610+1626) |
| 963 | GGTGCACGCCTCCACGG | SHANK3 H22 3UTR(+1613+1629) |
| 964 | CACGGTGCACGCCTCCA | SHANK3 H22 3UTR(+1616+1632) |
| 965 | GCACACGGTGCACGCCT | SHANK3 H22 3UTR(+1619+1635) |
| 966 | CATGCACACGGTGCACG | SHANK3 H22 3UTR(+1622+1638) |
| 967 | ACGCATGCACACGGTGC | SHANK3 H22 3UTR(+1625+1641) |
| 968 | CCCACGCATGCACACGG | SHANK3 H22 3UTR(+1628+1644) |
| 969 | CACCCCACGCATGCACA | SHANK3 H22 3UTR(+1631+1647) |
| 970 | GTACACCCCACGCATGC | SHANK3 H22 3UTR(+1634+1650) |
| 971 | CAGGTACACCCCACGCA | SHANK3 H22 3UTR(+1637+1653) |
| 972 | TCACAGGTACACCCCAC | SHANK3 H22 3UTR(+1640+1656) |
| 973 | CTCTCACAGGTACACCC | SHANK3 H22 3UTR(+1643+1659) |
| 974 | GTGCTCTCACAGGTACA | SHANK3 H22 3UTR(+1646+1662) |
| 975 | AGGGTGCTCTCACAGGT | SHANK3 H22 3UTR(+1649+1665) |
| 976 | GACAGGGTGCTCTCACA | SHANK3 H22 3UTR(+1652+1668) |
| 977 | GGAGACAGGGTGCTCTC | SHANK3 H22 3UTR(+1655+1671) |
| 978 | AGAGGAGACAGGGTGCT | SHANK3 H22 3UTR(+1658+1674) |
| 979 | GGAAGAGGAGACAGGGT | SHANK3 H22 3UTR(+1661+1677) |
| 980 | TTTGGAAGAGGAGACAG | SHANK3 H22 3UTR(+1664+1680) |
| 981 | TTCTTTGGAAGAGGAGA | SHANK3 H22 3UTR(+1667+1683) |
| 982 | ACTTTCTTTGGAAGAGG | SHANK3 H22 3UTR(+1670+1686) |
| 983 | CTGACTTTCTTTGGAAG | SHANK3 H22 3UTR(+1673+1689) |
| 984 | CCTCTGACTTTCTTTGG | SHANK3 H22 3UTR(+1676+1692) |
| 985 | TGGCCTCTGACTTTCTT | SHANK3 H22 3UTR(+1679+1695) |
| 986 | GGATGGCCTCTGACTTT | SHANK3 H22 3UTR(+1682+1698) |
| 987 | GCAGGATGGCCTCTGAC | SHANK3 H22 3UTR(+1685+1701) |
| 988 | GGTGCAGGATGGCCTCT | SHANK3 H22 3UTR(+1688+1704) |
| 989 | CAGGGTGCAGGATGGCC | SHANK3 H22 3UTR(+1691+1707) |
| 990 | ACCCAGGGTGCAGGATG | SHANK3 H22 3UTR(+1694+1710) |
| 991 | TGGACCCAGGGTGCAGG | SHANK3 H22 3UTR(+1697+1713) |
| 992 | AGCTGGACCCAGGGTGC | SHANK3 H22 3UTR(+1700+1716) |
| 993 | AACAGCTGGACCCAGGG | SHANK3 H22 3UTR(+1703+1719) |
| 994 | GCAAACAGCTGGACCCA | SHANK3 H22 3UTR(+1706+1722) |
| 995 | TGGGCAAACAGCTGGAC | SHANK3 H22 3UTR(+1709+1725) |
| 996 | GGCTGGGCAAACAGCTG | SHANK3 H22 3UTR(+1712+1728) |
| 997 | ACAGGCTGGGCAAACAG | SHANK3 H22 3UTR(+1715+1731) |
| 998 | AGGACAGGCTGGGCAAA | SHANK3 H22 3UTR(+1718+1734) |
| 999 | GGAAGGACAGGCTGGGC | SHANK3 H22 3UTR(+1721+1737) |
| 1000 | TCTGGAAGGACAGGCTG | SHANK3 H22 3UTR(+1724+1740) |
| 1001 | GGCTCTGGAAGGACAGG | SHANK3 H22 3UTR(+1727+1743) |
| 1002 | TGAGGCTCTGGAAGGAC | SHANK3 H22 3UTR(+1730+1746) |
| 1003 | GGGTGAGGCTCTGGAAG | SHANK3 H22 3UTR(+1733+1749) |
| 1004 | GCTGGGTGAGGCTCTGG | SHANK3 H22 3UTR(+1736+1752) |
| 1005 | CAGGCTGGGTGAGGCTC | SHANK3 H22 3UTR(+1739+1755) |
| 1006 | GCTCAGGCTGGGTGAGG | SHANK3 H22 3UTR(+1742+1758) |
| 1007 | CCCGCTCAGGCTGGGTG | SHANK3 H22 3UTR(+1745+1761) |
| 1008 | AACCCCGCTCAGGCTGG | SHANK3 H22 3UTR(+1748+1764) |
| 1009 | GGGAACCCCGCTCAGGC | SHANK3 H22 3UTR(+1751+1767) |
| 1010 | CCAGGGAACCCCGCTCA | SHANK3 H22 3UTR(+1754+1770) |
| 1011 | TCACCAGGGAACCCCGC | SHANK3 H22 3UTR(+1757+1773) |
| 1012 | GATTCACCAGGGAACCC | SHANK3 H22 3UTR(+1760+1776) |
| 1013 | AGGGATTCACCAGGGAA | SHANK3 H22 3UTR(+1763+1779) |
| 1014 | AGCAGGGATTCACCAGG | SHANK3 H22 3UTR(+1766+1782) |
| 1015 | AGCAGCAGGGATTCACC | SHANK3 H22 3UTR(+1769+1785) |
| 1016 | CCAAGCAGCAGGGATTC | SHANK3 H22 3UTR(+1772+1788) |
| 1017 | TCCCCAAGCAGCAGGGA | SHANK3 H22 3UTR(+1775+1791) |
| 1018 | GCCTCCCCAAGCAGCAG | SHANK3 H22 3UTR(+1778+1794) |
| 1019 | GGGGCCTCCCCAAGCAG | SHANK3 H22 3UTR(+1781+1797) |
| 1020 | CTTGGGGCCTCCCCAAG | SHANK3 H22 3UTR(+1784+1800) |
| 1021 | GCCCTTGGGGCCTCCCC | SHANK3 H22 3UTR(+1787+1803) |
| 1022 | GGGGCCCTTGGGGCCTC | SHANK3 H22 3UTR(+1790+1806) |
| 1023 | CAAGGGGCCCTTGGGGC | SHANK3 H22 3UTR(+1793+1809) |
| 1024 | CTCCAAGGGGCCCTTGG | SHANK3 H22 3UTR(+1796+1812) |
| 1025 | TGCCTCCAAGGGGCCCT | SHANK3 H22 3UTR(+1799+1815) |
| 1026 | CGCTGCCTCCAAGGGGC | SHANK3 H22 3UTR(+1802+1818) |
| 1027 | GGGCGCTGCCTCCAAGG | SHANK3 H22 3UTR(+1805+1821) |
| 1028 | TGGGGGCGCTGCCTCCA | SHANK3 H22 3UTR(+1808+1824) |
| 1029 | AGGTGGGGGCGCTGCCT | SHANK3 H22 3UTR(+1811+1827) |
| 1030 | CCAAGGTGGGGGCGCTG | SHANK3 H22 3UTR(+1814+1830) |
| 1031 | AGCCCAAGGTGGGGGCG | SHANK3 H22 3UTR(+1817+1833) |
| 1032 | AGAAGCCCAAGGTGGGG | SHANK3 H22 3UTR(+1820+1836) |
| 1033 | CTCAGAAGCCCAAGGTG | SHANK3 H22 3UTR(+1823+1839) |
| 1034 | GCCCTCAGAAGCCCAAG | SHANK3 H22 3UTR(+1826+1842) |
| 1035 | GATGCCCTCAGAAGCCC | SHANK3 H22 3UTR(+1829+1845) |
| 1036 | TATGATGCCCTCAGAAG | SHANK3 H22 3UTR(+1832+1848) |
| 1037 | CCCTATGATGCCCTCAG | SHANK3 H22 3UTR(+1835+1851) |
| 1038 | TCCCCCTATGATGCCCT | SHANK3 H22 3UTR(+1838+1854) |
| 1039 | GGGTCCCCCTATGATGC | SHANK3 H22 3UTR(+1841+1857) |
| 1040 | TAGGGGTCCCCCTATGA | SHANK3 H22 3UTR(+1844+1860) |
| 1041 | CTCTAGGGGTCCCCCTA | SHANK3 H22 3UTR(+1847+1863) |
| 1042 | TGACTCTAGGGGTCCCC | SHANK3 H22 3UTR(+1850+1866) |
| 1043 | AACTGACTCTAGGGGTC | SHANK3 H22 3UTR(+1853+1869) |
| 1044 | GTGAACTGACTCTAGGG | SHANK3 H22 3UTR(+1856+1872) |
| 1045 | GTGGTGAACTGACTCTA | SHANK3 H22 3UTR(+1859+1875) |
| 1046 | CCTGTGGTGAACTGACT | SHANK3 H22 3UTR(+1862+1878) |
| 1047 | GGGCCTGTGGTGAACTG | SHANK3 H22 3UTR(+1865+1881) |
| 1048 | CCAGGGCCTGTGGTGAA | SHANK3 H22 3UTR(+1868+1884) |
| 1049 | TCCCCAGGGCCTGTGGT | SHANK3 H22 3UTR(+1871+1887) |
| 1050 | CTCTCCCCAGGGCCTGT | SHANK3 H22 3UTR(+1874+1890) |
| 1051 | TGACTCTCCCCAGGGCC | SHANK3 H22 3UTR(+1877+1893) |
| 1052 | CTTTGACTCTCCCCAGG | SHANK3 H22 3UTR(+1880+1896) |
| 1053 | GGTCTTTGACTCTCCCC | SHANK3 H22 3UTR(+1883+1899) |
| 1054 | GGGGGTCTTTGACTCTC | SHANK3 H22 3UTR(+1886+1902) |
| 1055 | CTCGGGGGTCTTTGACT | SHANK3 H22 3UTR(+1889+1905) |
| 1056 | ACCCTCGGGGGTCTTTG | SHANK3 H22 3UTR(+1892+1908) |
| 1057 | GGCACCCTCGGGGGTCT | SHANK3 H22 3UTR(+1895+1911) |
| 1058 | CTGGGCACCCTCGGGGG | SHANK3 H22 3UTR(+1898+1914) |
| 1059 | GGGCTGGGCACCCTCGG | SHANK3 H22 3UTR(+1901+1917) |
| 1060 | GGGGGGCTGGGCACCCT | SHANK3 H22 3UTR(+1904+1920) |
| 1061 | TGTGGGGGGCTGGGCAC | SHANK3 H22 3UTR(+1907+1923) |
| 1062 | CAGTGTGGGGGGCTGGG | SHANK3 H22 3UTR(+1910+1926) |
| 1063 | TCACAGTGTGGGGGGCT | SHANK3 H22 3UTR(+1913+1929) |
| 1064 | GAGTCACAGTGTGGGGG | SHANK3 H22 3UTR(+1916+1932) |
| 1065 | GAGGAGTCACAGTGTGG | SHANK3 H22 3UTR(+1919+1935) |
| 1066 | TGTGAGGAGTCACAGTG | SHANK3 H22 3UTR(+1922+1938) |
| 1067 | GAGTGTGAGGAGTCACA | SHANK3 H22 3UTR(+1925+1941) |
| 1068 | GCTGAGTGTGAGGAGTC | SHANK3 H22 3UTR(+1928+1944) |
| 1069 | ATCGCTGAGTGTGAGGA | SHANK3 H22 3UTR(+1931+1947) |
| 1070 | GTCATCGCTGAGTGTGA | SHANK3 H22 3UTR(+1934+1950) |
| 1071 | CAGGTCATCGCTGAGTG | SHANK3 H22 3UTR(+1937+1953) |
| 1072 | CCACAGGTCATCGCTGA | SHANK3 H22 3UTR(+1940+1956) |
| 1073 | ACCCCACAGGTCATCGC | SHANK3 H22 3UTR(+1943+1959) |
| 1074 | CCCACCCCACAGGTCAT | SHANK3 H22 3UTR(+1946+1962) |
| 1075 | CCCCCCACCCCACAGGT | SHANK3 H22 3UTR(+1949+1965) |
| 1076 | GGGCCCCCCACCCCACA | SHANK3 H22 3UTR(+1952+1968) |
| 1077 | CCAGGGCCCCCCACCCC | SHANK3 H22 3UTR(+1955+1971) |
| 1078 | GTCCCAGGGCCCCCCAC | SHANK3 H22 3UTR(+1958+1974) |
| 1079 | AACGTCCCAGGGCCCCC | SHANK3 H22 3UTR(+1961+1977) |
| 1080 | AAAAACGTCCCAGGGCC | SHANK3 H22 3UTR(+1964+1980) |
| 1081 | TTTAAAAACGTCCCAGG | SHANK3 H22 3UTR(+1967+1983) |
| 1082 | AGGTTTAAAAACGTCCC | SHANK3 H22 3UTR(+1970+1986) |
| 1083 | CCTAGGTTTAAAAACGT | SHANK3 H22 3UTR(+1973+1989) |
| 1084 | AACCCTAGGTTTAAAAA | SHANK3 H22 3UTR(+1976+1992) |
| 1085 | CCAAACCCTAGGTTTAA | SHANK3 H22 3UTR(+1979+1995) |
| 1086 | ACTCCAAACCCTAGGTT | SHANK3 H22 3UTR(+1982+1998) |
| 1087 | CAGACTCCAAACCCTAG | SHANK3 H22 3UTR(+1985+2001) |
| 1088 | GTCCAGACTCCAAACCC | SHANK3 H22 3UTR(+1988+2004) |
| 1089 | TTAGTCCAGACTCCAAA | SHANK3 H22 3UTR(+1991+2007) |
| 1090 | AGCTTAGTCCAGACTCC | SHANK3 H22 3UTR(+1994+2010) |
| 1091 | TGGAGCTTAGTCCAGAC | SHANK3 H22 3UTR(+1997+2013) |
| 1092 | GGATGGAGCTTAGTCCA | SHANK3 H22 3UTR(+2000+2016) |
| 1093 | CGTGGATGGAGCTTAGT | SHANK3 H22 3UTR(+2003+2019) |
| 1094 | TGACGTGGATGGAGCTT | SHANK3 H22 3UTR(+2006+2022) |
| 1095 | GAGTGACGTGGATGGAG | SHANK3 H22 3UTR(+2009+2025) |
| 1096 | TGTGAGTGACGTGGATG | SHANK3 H22 3UTR(+2012+2028) |
| 1097 | ACTTGTGAGTGACGTGG | SHANK3 H22 3UTR(+2015+2031) |
| 1098 | GAAACTTGTGAGTGACG | SHANK3 H22 3UTR(+2018+2034) |
| 1099 | ACAGAAACTTGTGAGTG | SHANK3 H22 3UTR(+2021+2037) |
| 1100 | TAAACAGAAACTTGTGA | SHANK3 H22 3UTR(+2024+2040) |
| 1101 | ATATAAACAGAAACTTG | SHANK3 H22 3UTR(+2027+2043) |
| 1102 | GAAATATAAACAGAAAC | SHANK3 H22 3UTR(+2030+2046) |
| 1103 | CTAGAAATATAAACAGA | SHANK3 H22 3UTR(+2033+2049) |
| 1104 | AAGCTAGAAATATAAAC | SHANK3 H22 3UTR(+2036+2052) |
| 1105 | AAAAAGCTAGAAATATA | SHANK3 H22 3UTR(+2039+2055) |
| 1106 | TAAAAAAAGCTAGAAAT | SHANK3 H22 3UTR(+2042+2058) |
| 1107 | TATTAAAAAAAGCTAGA | SHANK3 H22 3UTR(+2045+2061) |
| 1108 | TTTTATTAAAAAAAGCT | SHANK3 H22 3UTR(+2048+2064) |
| 1109 | TTATTTTATTAAAAAAA | SHANK3 H22 3UTR(+2051+2067) |
| 1110 | TTTTTATTTTATTAAAA | SHANK3 H22 3UTR(+2054+2070) |
| 1111 | TTTTTTTTATTTTATTA | SHANK3 H22 3UTR(+2057+2073) |
| 1112 | TTTTTTTTTTTATTTTA | SHANK3 H22 3UTR(+2060+2076) |
| 1113 | TTCTTTTTTTTTTTATT | SHANK3 H22 3UTR(+2063+2079) |
| 1114 | GTTTTCTTTTTTTTTTT | SHANK3 H22 3UTR(+2066+2082) |
| 1115 | TCTGTTTTCTTTTTTTT | SHANK3 H22 3UTR(+2069+2085) |
| 1116 | ACTTCTGTTTTCTTTTT | SHANK3 H22 3UTR(+2072+2088) |
| 1117 | AAAACTTCTGTTTTCTT | SHANK3 H22 3UTR(+2075+2091) |
| 1118 | GTGAAAACTTCTGTTTT | SHANK3 H22 3UTR(+2078+2094) |
| 1119 | GTTGTGAAAACTTCTGT | SHANK3 H22 3UTR(+2081+2097) |
| 1120 | TGGGTTGTGAAAACTTC | SHANK3 H22 3UTR(+2084+2100) |
| 1121 | CCCTGGGTTGTGAAAAC | SHANK3 H22 3UTR(+2087+2103) |
| 1122 | GGCCCCTGGGTTGTGAA | SHANK3 H22 3UTR(+2090+2106) |
| 1123 | CCAGGCCCCTGGGTTGT | SHANK3 H22 3UTR(+2093+2109) |
| 1124 | GTGCCAGGCCCCTGGGT | SHANK3 H22 3UTR(+2096+2112) |
| 1125 | GGCGTGCCAGGCCCCTG | SHANK3 H22 3UTR(+2099+2115) |
| 1126 | ACCGGCGTGCCAGGCCC | SHANK3 H22 3UTR(+2102+2118) |
| 1127 | CAGACCGGCGTGCCAGG | SHANK3 H22 3UTR(+2105+2121) |
| 1128 | GCACAGACCGGCGTGCC | SHANK3 H22 3UTR(+2108+2124) |
| 1129 | CAGGCACAGACCGGCGT | SHANK3 H22 3UTR(+2111+2127) |
| 1130 | GGGCAGGCACAGACCGG | SHANK3 H22 3UTR(+2114+2130) |
| 1131 | GGCGGGCAGGCACAGAC | SHANK3 H22 3UTR(+2117+2133) |
| 1132 | CGGGGGGGGCAGGCACA | SHANK3 H22 3UTR(+2120+2136) |
| 1133 | GGGCGGGGGGGGCAGGC | SHANK3 H22 3UTR(+2123+2139) |
| 1134 | CCAGGGCGGGGGGGGCA | SHANK3 H22 3UTR(+2126+2142) |
| 1135 | GGGCCAGGGCGGGGCGG | SHANK3 H22 3UTR(+2129+2145) |
| 1136 | GGTGGGCCAGGGGGGGG | SHANK3 H22 3UTR(+2132+2148) |
| 1137 | GCCGGTGGGCCAGGGCG | SHANK3 H22 3UTR(+2135+2151) |
| 1138 | GGGGCCGGTGGGCCAGG | SHANK3 H22 3UTR(+2138+2154) |
| 1139 | AGTGGGGCCGGTGGGCC | SHANK3 H22 3UTR(+2141+2157) |
| 1140 | GGGAGTGGGGCCGGTGG | SHANK3 H22 3UTR(+2144+2160) |
| 1141 | CCAGGGAGTGGGGCCGG | SHANK3 H22 3UTR(+2147+2163) |
| 1142 | TGCCCAGGGAGTGGGGC | SHANK3 H22 3UTR(+2150+2166) |
| 1143 | CTGTGCCCAGGGAGTGG | SHANK3 H22 3UTR(+2153+2169) |
| 1144 | ACTCTGTGCCCAGGGAG | SHANK3 H22 3UTR(+2156+2172) |
| 1145 | GTGACTCTGTGCCCAGG | SHANK3 H22 3UTR(+2159+2175) |
| 1146 | GGTGTGACTCTGTGCCC | SHANK3 H22 3UTR(+2162+2178) |
| 1147 | GTGGGTGTGACTCTGTG | SHANK3 H22 3UTR(+2165+2181) |
| 1148 | TGAGTGGGTGTGACTCT | SHANK3 H22 3UTR(+2168+2184) |
| 1149 | GGATGAGTGGGTGTGAC | SHANK3 H22 3UTR(+2171+2187) |
| 1150 | GAAGGATGAGTGGGTGT | SHANK3 H22 3UTR(+2174+2190) |
| 1151 | GCGGAAGGATGAGTGGG | SHANK3 H22 3UTR(+2177+2193) |
| 1152 | TTGGCGGAAGGATGAGT | SHANK3 H22 3UTR(+2180+2196) |
| 1153 | CTGTTGGCGGAAGGATG | SHANK3 H22 3UTR(+2183+2199) |
| 1154 | GGACTGTTGGCGGAAGG | SHANK3 H22 3UTR(+2186+2202) |
| 1155 | CCTGGACTGTTGGCGGA | SHANK3 H22 3UTR(+2189+2205) |
| 1156 | TGACCTGGACTGTTGGC | SHANK3 H22 3UTR(+2192+2208) |
| 1157 | GTGTGACCTGGACTGTT | SHANK3 H22 3UTR(+2195+2211) |
| 1158 | GCTGTGTGACCTGGACT | SHANK3 H22 3UTR(+2198+2214) |
| 1159 | GCTGCTGTGTGACCTGG | SHANK3 H22 3UTR(+2201+2217) |
| 1160 | ACTGCTGCTGTGTGACC | SHANK3 H22 3UTR(+2204+2220) |
| 1161 | GTGACTGCTGCTGTGTG | SHANK3 H22 3UTR(+2207+2223) |
| 1162 | ACAGTGACTGCTGCTGT | SHANK3 H22 3UTR(+2210+2226) |
| 1163 | GTTACAGTGACTGCTGC | SHANK3 H22 3UTR(+2213+2229) |
| 1164 | TCTGTTACAGTGACTGC | SHANK3 H22 3UTR(+2216+2232) |
| 1165 | CAGTCTGTTACAGTGAC | SHANK3 H22 3UTR(+2219+2235) |
| 1166 | TGGCAGTCTGTTACAGT | SHANK3 H22 3UTR(+2222+2238) |
| 1167 | ATGTGGCAGTCTGTTAC | SHANK3 H22 3UTR(+2225+2241) |
| 1168 | TGTATGTGGCAGTCTGT | SHANK3 H22 3UTR(+2228+2244) |
| 1169 | GTGTGTATGTGGCAGTC | SHANK3 H22 3UTR(+2231+2247) |
| 1170 | CGAGTGTGTATGTGGCA | SHANK3 H22 3UTR(+2234+2250) |
| 1171 | GACCGAGTGTGTATGTG | SHANK3 H22 3UTR(+2237+2253) |
| 1172 | TGAGACCGAGTGTGTAT | SHANK3 H22 3UTR(+2240+2256) |
| 1173 | GTGTGAGACCGAGTGTG | SHANK3 H22 3UTR(+2243+2259) |
| 1174 | TGAGTGTGAGACCGAGT | SHANK3 H22 3UTR(+2246+2262) |
| 1175 | AGGTGAGTGTGAGACCG | SHANK3 H22 3UTR(+2249+2265) |
| 1176 | CACAGGTGAGTGTGAGA | SHANK3 H22 3UTR(+2252+2268) |
| 1177 | ACCCACAGGTGAGTGTG | SHANK3 H22 3UTR(+2255+2271) |
| 1178 | AAAACCCACAGGTGAGT | SHANK3 H22 3UTR(+2258+2274) |
| 1179 | ACCAAAACCCACAGGTG | SHANK3 H22 3UTR(+2261+2277) |
| 1180 | GGAACCAAAACCCACAG | SHANK3 H22 3UTR(+2264+2280) |
| 1181 | AACGGAACCAAAACCCA | SHANK3 H22 3UTR(+2267+2283) |
| 1182 | TTGAACGGAACCAAAAC | SHANK3 H22 3UTR(+2270+2286) |
| 1183 | AAATTGAACGGAACCAA | SHANK3 H22 3UTR(+2273+2289) |
| 1184 | CCCAAATTGAACGGAAC | SHANK3 H22 3UTR(+2276+2292) |
| 1185 | AAACCCAAATTGAACGG | SHANK3 H22 3UTR(+2279+2295) |
| 1186 | TAAAAACCCAAATTGAA | SHANK3 H22 3UTR(+2282+2298) |
| 1187 | AGTTAAAAACCCAAATT | SHANK3 H22 3UTR(+2285+2301) |
| 1188 | TAAAGTTAAAAACCCAA | SHANK3 H22 3UTR(+2288+2304) |
| 1189 | CTGTAAAGTTAAAAACC | SHANK3 H22 3UTR(+2291+2307) |
| 1190 | ACCCTGTAAAGTTAAAA | SHANK3 H22 3UTR(+2294+2310) |
| 1191 | CTGACCCTGTAAAGTTA | SHANK3 H22 3UTR(+2297+2313) |
| 1192 | GAACTGACCCTGTAAAG | SHANK3 H22 3UTR(+2300+2316) |
| 1193 | GCGGAACTGACCCTGTA | SHANK3 H22 3UTR(+2303+2319) |
| 1194 | GAAGCGGAACTGACCCT | SHANK3 H22 3UTR(+2306+2322) |
| 1195 | GGTGAAGCGGAACTGAC | SHANK3 H22 3UTR(+2309+2325) |
| 1196 | GGAGGTGAAGCGGAACT | SHANK3 H22 3UTR(+2312+2328) |
| 1197 | AAAGGAGGTGAAGCGGA | SHANK3 H22 3UTR(+2315+2331) |
| 1198 | ACAAAAGGAGGTGAAGC | SHANK3 H22 3UTR(+2318+2334) |
| 1199 | CATACAAAAGGAGGTGA | SHANK3 H22 3UTR(+2321+2337) |
| 1200 | CTCCATACAAAAGGAGG | SHANK3 H22 3UTR(+2324+2340) |
| 1201 | GAACTCCATACAAAAGG | SHANK3 H22 3UTR(+2327+2343) |
| 1202 | ATGGAACTCCATACAAA | SHANK3 H22 3UTR(+2330+2346) |
| 1203 | CGGATGGAACTCCATAC | SHANK3 H22 3UTR(+2333+2349) |
| 1204 | CCCCGGATGGAACTCCA | SHANK3 H22 3UTR(+2336+2352) |
| 1205 | ACCCCCCGGATGGAACT | SHANK3 H22 3UTR(+2339+2355) |
| 1206 | GAAACCCCCCGGATGGA | SHANK3 H22 3UTR(+2342+2358) |
| 1207 | GGTGAAACCCCCCGGAT | SHANK3 H22 3UTR(+2345+2361) |
| 1208 | GGGGGTGAAACCCCCCG | SHANK3 H22 3UTR(+2348+2364) |
| 1209 | GCAGGGGGTGAAACCCC | SHANK3 H22 3UTR(+2351+2367) |
| 1210 | GGAGCAGGGGGTGAAAC | SHANK3 H22 3UTR(+2354+2370) |
| 1211 | ACTGGAGCAGGGGGTGA | SHANK3 H22 3UTR(+2357+2373) |
| 1212 | AGGACTGGAGCAGGGGG | SHANK3 H22 3UTR(+2360+2376) |
| 1213 | CTCAGGACTGGAGCAGG | SHANK3 H22 3UTR(+2363+2379) |
| 1214 | GGCCTCAGGACTGGAGC | SHANK3 H22 3UTR(+2366+2382) |
| 1215 | GGAGGCCTCAGGACTGG | SHANK3 H22 3UTR(+2369+2385) |
| 1216 | TCAGGAGGCCTCAGGAC | SHANK3 H22 3UTR(+2372+2388) |
| 1217 | GGGTCAGGAGGCCTCAG | SHANK3 H22 3UTR(+2375+2391) |
| 1218 | TCAGGGTCAGGAGGCCT | SHANK3 H22 3UTR(+2378+2394) |
| 1219 | ACGTCAGGGTCAGGAGG | SHANK3 H22 3UTR(+2381+2397) |
| 1220 | ACAACGTCAGGGTCAGG | SHANK3 H22 3UTR(+2384+2400) |
| 1221 | ATCACAACGTCAGGGTC | SHANK3 H22 3UTR(+2387+2403) |
| 1222 | CGTATCACAACGTCAGG | SHANK3 H22 3UTR(+2390+2406) |
| 1223 | GGGCGTATCACAACGTC | SHANK3 H22 3UTR(+2393+2409) |
| 1224 | GTGGGGCGTATCACAAC | SHANK3 H22 3UTR(+2396+2412) |
| 1225 | TCTGTGGGGCGTATCAC | SHANK3 H22 3UTR(+2399+2415) |
| 1226 | ATCTCTGTGGGGCGTAT | SHANK3 H22 3UTR(+2402+2418) |
| 1227 | TAGATCTCTGTGGGGCG | SHANK3 H22 3UTR(+2405+2421) |
| 1228 | ACATAGATCTCTGTGGG | SHANK3 H22 3UTR(+2408+2424) |
| 1229 | GAAACATAGATCTCTGT | SHANK3 H22 3UTR(+2411+2427) |
| 1230 | TAAGAAACATAGATCTC | SHANK3 H22 3UTR(+2414+2430) |
| 1231 | ATATAAGAAACATAGAT | SHANK3 H22 3UTR(+2417+2433) |
| 1232 | ATAATATAAGAAACATA | SHANK3 H22 3UTR(+2420+2436) |
| 1233 | ATAATAATATAAGAAAC | SHANK3 H22 3UTR(+2423+2439) |
| 1234 | ATAATAATAATATAAGA | SHANK3 H22 3UTR(+2426+2442) |
| 1235 | TCAATAATAATAATATA | SHANK3 H22 3UTR(+2429+2445) |
| 1236 | TTATCAATAATAATAAT | SHANK3 H22 3UTR(+2432+2448) |
| 1237 | TTATTATCAATAATAAT | SHANK3 H22 3UTR(+2435+2451) |
| 1238 | TAATTATTATCAATAAT | SHANK3 H22 3UTR(+2438+2454) |
| 1239 | TAATAATTATTATCAAT | SHANK3 H22 3UTR(+2441+2457) |
| 1240 | TTATAATAATTATTATC | SHANK3 H22 3UTR(+2444+2460) |
| 1241 | ATATTATAATAATTATT | SHANK3 H22 3UTR(+2447+2463) |
| 1242 | ATAATATTATAATAATT | SHANK3 H22 3UTR(+2450+2466) |
| 1243 | ATAATAATATTATAATA | SHANK3 H22 3UTR(+2453+2469) |
| 1244 | TACATAATAATATTATA | SHANK3 H22 3UTR(+2456+2472) |
| 1245 | TATTACATAATAATATT | SHANK3 H22 3UTR(+2459+2475) |
| 1246 | ATTTATTACATAATAAT | SHANK3 H22 3UTR(+2462+2478) |
| 1247 | TAAATTTATTACATAAT | SHANK3 H22 3UTR(+2465+2481) |
| 1248 | TTATAAATTTATTACAT | SHANK3 H22 3UTR(+2468+2484) |
| 1249 | TTCTTATAAATTTATTA | SHANK3 H22 3UTR(+2471+2487) |
| 1250 | CATTTCTTATAAATTTA | SHANK3 H22 3UTR(+2474+2490) |
| 1251 | CTTCATTTCTTATAAAT | SHANK3 H22 3UTR(+2477+2493) |
| 1252 | TGGCTTCATTTCTTATA | SHANK3 H22 3UTR(+2480+2496) |
| 1253 | CCATGGCTTCATTTCTT | SHANK3 H22 3UTR(+2483+2499) |
| 1254 | GAGCCATGGCTTCATTT | SHANK3 H22 3UTR(+2486+2502) |
| 1255 | ACTGAGCCATGGCTTCA | SHANK3 H22 3UTR(+2489+2505) |
| 1256 | GCAACTGAGCCATGGCT | SHANK3 H22 3UTR(+2492+2508) |
| 1257 | CAGGCAACTGAGCCATG | SHANK3 H22 3UTR(+2495+2511) |
| 1258 | AAGCAGGCAACTGAGCC | SHANK3 H22 3UTR(+2498+2514) |
| 1259 | CTCAAGCAGGCAACTGA | SHANK3 H22 3UTR(+2501+2517) |
| 1260 | CCCCTCAAGCAGGCAAC | SHANK3 H22 3UTR(+2504+2520) |
| 1261 | TATCCCCTCAAGCAGGC | SHANK3 H22 3UTR(+2507+2523) |
| 1262 | AAATATCCCCTCAAGCA | SHANK3 H22 3UTR(+2510+2526) |
| 1263 | CACAAATATCCCCTCAA | SHANK3 H22 3UTR(+2513+2529) |
| 1264 | AGACACAAATATCCCCT | SHANK3 H22 3UTR(+2516+2532) |
| 1265 | GACAGACACAAATATCC | SHANK3 H22 3UTR(+2519+2535) |
| 1266 | AGGGACAGACACAAATA | SHANK3 H22 3UTR(+2522+2538) |
| 1267 | ATGAGGGACAGACACAA | SHANK3 H22 3UTR(+2525+2541) |
| 1268 | GGTATGAGGGACAGACA | SHANK3 H22 3UTR(+2528+2544) |
| 1269 | GCCGGTATGAGGGACAG | SHANK3 H22 3UTR(+2531+2547) |
| 1270 | GTGGCCGGTATGAGGGA | SHANK3 H22 3UTR(+2534+2550) |
| 1271 | TGTGTGGCCGGTATGAG | SHANK3 H22 3UTR(+2537+2553) |
| 1272 | GTTTGTGTGGCCGGTAT | SHANK3 H22 3UTR(+2540+2556) |
| 1273 | GAGGTTTGTGTGGCCGG | SHANK3 H22 3UTR(+2543+2559) |
| 1274 | GAGGAGGTTTGTGTGGC | SHANK3 H22 3UTR(+2546+2562) |
| 1275 | GCTGAGGAGGTTTGTGT | SHANK3 H22 3UTR(+2549+2565) |
| 1276 | CAGGCTGAGGAGGTTTG | SHANK3 H22 3UTR(+2552+2568) |
| 1277 | GTCCAGGCTGAGGAGGT | SHANK3 H22 3UTR(+2555+2571) |
| 1278 | GTGGTCCAGGCTGAGGA | SHANK3 H22 3UTR(+2558+2574) |
| 1279 | CATGTGGTCCAGGCTGA | SHANK3 H22 3UTR(+2561+2577) |
| 1280 | CCGGGGGGGGAGTGGGGGTGGGGCG | SHANK3 H22 3UTR(+593+617) |
| 1281 | CGGCCGGGGGGGGAGTGGGGGTGGG | SHANK3 H22 3UTR(+596+620) |
| 1282 | GCACGGCCGGGGGGGGAGTGGGGGT | SHANK3 H22 3UTR(+599+623) |
| 1283 | AGGGCACGGCCGGGGGGGGAGTGGG | SHANK3 H22 3UTR(+602+626) |
| 1284 | GGCAGGGCACGGCCGGGGGGGGAGT | SHANK3 H22 3UTR(+605+629) |
| 1285 | GCCGGCAGGGCACGGCCGGGGCGGG | SHANK3 H22 3UTR(+608+632) |
| 1286 | CCTGCCGGCAGGGCACGGCCGGGGC | SHANK3 H22 3UTR(+611+635) |
| 1287 | GGCCCTGCCGGCAGGGCACGGCCGG | SHANK3 H22 3UTR(+614+638) |
| 1288 | GGGGGCCCTGCCGGCAGGGCACGGC | SHANK3 H22 3UTR(+617+641) |
| 1289 | GTGGGGGGCCCTGCCGGCAGGGCAC | SHANK3 H22 3UTR(+620+644) |
| 1290 | GGGGTGGGGGGCCCTGCCGGCAGGG | SHANK3 H22 3UTR(+623+647) |
| 1291 | GTGGGGGTGGGGGGCCCTGCCGGCA | SHANK3 H22 3UTR(+626+650) |
| 1292 | GGGGTGGGGGTGGGGGGCCCTGCCG | SHANK3 H22 3UTR(+629+653) |
| 1293 | CCCGGGGTGGGGGTGGGGGGCCCTG | SHANK3 H22 3UTR(+632+656) |
| 1294 | CGGCCCGGGGTGGGGGTGGGGGGCC | SHANK3 H22 3UTR(+635+659) |
| 1295 | CCGCGGCCCGGGGTGGGGGTGGGGG | SHANK3 H22 3UTR(+638+662) |
| 1296 | AGCCCGCGGCCCGGGGGGGGGTGG | SHANK3 H22 3UTR(+641+665) |
| 1297 | CCGAGCCCGCGGCCCGGGGTGGGGG | SHANK3 H22 3UTR(+644+668) |
| 1298 | AGGCCGAGCCCGCGGCCCGGGGTGG | SHANK3 H22 3UTR(+647+671) |
| 1299 | GGCAGGCCGAGCCCGCGGCCCGGGG | SHANK3 H22 3UTR(+650+674) |
| 1300 | AGGGGCAGGCCGAGCCCGCGGCCCG | SHANK3 H22 3UTR(+653+677) |
| 1301 | GTAAGGGGCAGGCCGAGCCCGCGGC | SHANK3 H22 3UTR(+656+680) |
| 1302 | GTCGTAAGGGGCAGGCCGAGCCCGC | SHANK3 H22 3UTR(+659+683) |
| 1303 | GCCGTCGTAAGGGGCAGGCCGAGCC | SHANK3 H22 3UTR(+662+686) |
| 1304 | GGCGCCGTCGTAAGGGGCAGGCCGA | SHANK3 H22 3UTR(+665+689) |
| 1305 | CCGGGCGCCGTCGTAAGGGGCAGGC | SHANK3 H22 3UTR(+668+692) |
| 1306 | GGCCCGGGCGCCGTCGTAAGGGGCA | SHANK3 H22 3UTR(+671+695) |
| 1307 | CCTGGCCCGGGCGCCGTCGTAAGGG | SHANK3 H22 3UTR(+674+698) |
| 1308 | ATTCCTGGCCCGGGCGCCGTCGTAA | SHANK3 H22 3UTR(+677+701) |
| 1309 | AACATTCCTGGCCCGGGCGCCGTCG | SHANK3 H22 3UTR(+680+704) |
| 1310 | TGCAACATTCCTGGCCCGGGCGCCG | SHANK3 H22 3UTR(+683+707) |
| 1311 | TCATGCAACATTCCTGGCCCGGGCG | SHANK3 H22 3UTR(+686+710) |
| 1312 | GATTCATGCAACATTCCTGGCCCGG | SHANK3 H22 3UTR(+689+713) |
| 1313 | GACGATTCATGCAACATTCCTGGCC | SHANK3 H22 3UTR(+692+716) |
| 1314 | CAGGACGATTCATGCAACATTCCTG | SHANK3 H22 3UTR(+695+719) |
| 1315 | AAACAGGACGATTCATGCAACATTC | SHANK3 H22 3UTR(+698+722) |
| 1316 | AGCAAACAGGACGATTCATGCAACA | SHANK3 H22 3UTR(+701+725) |
| 1317 | AACAGCAAACAGGACGATTCATGCA | SHANK3 H22 3UTR(+704+728) |
| 1318 | AGCAACAGCAAACAGGACGATTCAT | SHANK3 H22 3UTR(+707+731) |
| 1319 | CCGAGCAACAGCAAACAGGACGATT | SHANK3 H22 3UTR(+710+734) |
| 1320 | TCTCCGAGCAACAGCAAACAGGACG | SHANK3 H22 3UTR(+713+737) |
| 1321 | GAGTCTCCGAGCAACAGCAAACAGG | SHANK3 H22 3UTR(+716+740) |
| 1322 | GGCGAGTCTCCGAGCAACAGCAAAC | SHANK3 H22 3UTR(+719+743) |
| 1323 | CAGGGCGAGTCTCCGAGCAACAGCA | SHANK3 H22 3UTR(+722+746) |
| 1324 | GTACAGGGCGAGTCTCCGAGCAACA | SHANK3 H22 3UTR(+725+749) |
| 1325 | AATGTACAGGGCGAGTCTCCGAGCA | SHANK3 H22 3UTR(+728+752) |
| 1326 | AGCAATGTACAGGGCGAGTCTCCGA | SHANK3 H22 3UTR(+731+755) |
| 1327 | CTAAGCAATGTACAGGGCGAGTCTC | SHANK3 H22 3UTR(+734+758) |
| 1328 | GCACTAAGCAATGTACAGGGCGAGT | SHANK3 H22 3UTR(+737+761) |
| 1329 | AGGGCACTAAGCAATGTACAGGGCG | SHANK3 H22 3UTR(+740+764) |
| 1330 | GTGAGGGCACTAAGCAATGTACAGG | SHANK3 H22 3UTR(+743+767) |
| 1331 | CCGGTGAGGGCACTAAGCAATGTAC | SHANK3 H22 3UTR(+746+770) |
| 1332 | CGGCCGGTGAGGGCACTAAGCAATG | SHANK3 H22 3UTR(+749+773) |
| 1333 | GGGCGGCCGGTGAGGGCACTAAGCA | SHANK3 H22 3UTR(+752+776) |
| 1334 | GCTGGGCGGCCGGTGAGGGCACTAA | SHANK3 H22 3UTR(+755+779) |
| 1335 | TGGGCTGGGCGGCCGGTGAGGGCAC | SHANK3 H22 3UTR(+758+782) |
| 1336 | GGGTGGGCTGGGCGGCCGGTGAGGG | SHANK3 H22 3UTR(+761+785) |
| 1337 | GCTGGGTGGGCTGGGCGGCCGGTGA | SHANK3 H22 3UTR(+764+788) |
| 1338 | TGCGCTGGGTGGGCTGGGCGGCCGG | SHANK3 H22 3UTR(+767+791) |
| 1339 | CTGTGCGCTGGGTGGGCTGGGCGGC | SHANK3 H22 3UTR(+770+794) |
| 1340 | TGACTGTGCGCTGGGTGGGCTGGGC | SHANK3 H22 3UTR(+773+797) |
| 1341 | TCCTGACTGTGCGCTGGGTGGGCTG | SHANK3 H22 3UTR(+776+800) |
| 1342 | CCTTCCTGACTGTGCGCTGGGTGGG | SHANK3 H22 3UTR(+779+803) |
| 1343 | CGCCCTTCCTGACTGTGCGCTGGGT | SHANK3 H22 3UTR(+782+806) |
| 1344 | CCACGCCCTTCCTGACTGTGCGCTG | SHANK3 H22 3UTR(+785+809) |
| 1345 | GGTCCACGCCCTTCCTGACTGTGCG | SHANK3 H22 3UTR(+788+812) |
| 1346 | CCTGGTCCACGCCCTTCCTGACTGT | SHANK3 H22 3UTR(+791+815) |
| 1347 | CTCCCTGGTCCACGCCCTTCCTGAC | SHANK3 H22 3UTR(+794+818) |
| 1348 | AGCCTCCCTGGTCCACGCCCTTCCT | SHANK3 H22 3UTR(+797+821) |
| 1349 | CCCAGCCTCCCTGGTCCACGCCCTT | SHANK3 H22 3UTR(+800+824) |
| 1350 | CGCCCCAGCCTCCCTGGTCCACGCC | SHANK3 H22 3UTR(+803+827) |
| 1351 | TCCCGCCCCAGCCTCCCTGGTCCAC | SHANK3 H22 3UTR(+806+830) |
| 1352 | ACCTCCCGCCCCAGCCTCCCTGGTC | SHANK3 H22 3UTR(+809+833) |
| 1353 | GGCACCTCCCGCCCCAGCCTCCCTG | SHANK3 H22 3UTR(+812+836) |
| 1354 | CCCGGCACCTCCCGCCCCAGCCTCC | SHANK3 H22 3UTR(+815+839) |
| 1355 | ACCCCCGGCACCTCCCGCCCCAGCC | SHANK3 H22 3UTR(+818+842) |
| 1356 | CCCACCCCCGGCACCTCCCGCCCCA | SHANK3 H22 3UTR(+821+845) |
| 1357 | CACCCCACCCCCGGCACCTCCCGCC | SHANK3 H22 3UTR(+824+848) |
| 1358 | GGGCACCCCACCCCCGGCACCTCCC | SHANK3 H22 3UTR(+827+851) |
| 1359 | CTAGGGCACCCCACCCCCGGCACCT | SHANK3 H22 3UTR(+830+854) |
| 1360 | ACGCTAGGGCACCCCACCCCCGGCA | SHANK3 H22 3UTR(+833+857) |
| 1361 | GTCACGCTAGGGCACCCCACCCCCG | SHANK3 H22 3UTR(+836+860) |
| 1362 | GTGGTCACGCTAGGGCACCCCACCC | SHANK3 H22 3UTR(+839+863) |
| 1363 | GAGGTGGTCACGCTAGGGCACCCCA | SHANK3 H22 3UTR(+842+866) |
| 1364 | AAGGAGGTGGTCACGCTAGGGCACC | SHANK3 H22 3UTR(+845+869) |
| 1365 | GCGAAGGAGGTGGTCACGCTAGGGC | SHANK3 H22 3UTR(+848+872) |
| 1366 | GCTGCGAAGGAGGTGGTCACGCTAG | SHANK3 H22 3UTR(+851+875) |
| 1367 | GGAGCTGCGAAGGAGGTGGTCACGC | SHANK3 H22 3UTR(+854+878) |
| 1368 | CCAGGAGCTGCGAAGGAGGTGGTCA | SHANK3 H22 3UTR(+857+881) |
| 1369 | CCACCAGGAGCTGCGAAGGAGGTGG | SHANK3 H22 3UTR(+860+884) |
| 1370 | TGGCCACCAGGAGCTGCGAAGGAGG | SHANK3 H22 3UTR(+863+887) |
| 1371 | GAATGGCCACCAGGAGCTGCGAAGG | SHANK3 H22 3UTR(+866+890) |
| 1372 | GGAGAATGGCCACCAGGAGCTGCGA | SHANK3 H22 3UTR(+869+893) |
| 1373 | CTGGGAGAATGGCCACCAGGAGCTG | SHANK3 H22 3UTR(+872+896) |
| 1374 | CCTCTGGGAGAATGGCCACCAGGAG | SHANK3 H22 3UTR(+875+899) |
| 1375 | CCCCCTCTGGGAGAATGGCCACCAG | SHANK3 H22 3UTR(+878+902) |
| 1376 | GTTCCCCCTCTGGGAGAATGGCCAC | SHANK3 H22 3UTR(+881+905) |
| 1377 | TAGGTTCCCCCTCTGGGAGAATGGC | SHANK3 H22 3UTR(+884+908) |
| 1378 | GACTAGGTTCCCCCTCTGGGAGAAT | SHANK3 H22 3UTR(+887+911) |
| 1379 | CTGGACTAGGTTCCCCCTCTGGGAG | SHANK3 H22 3UTR(+890+914) |
| 1380 | ATGCTGGACTAGGTTCCCCCTCTGG | SHANK3 H22 3UTR(+893+917) |
| 1381 | CGCATGCTGGACTAGGTTCCCCCTC | SHANK3 H22 3UTR(+896+920) |
| 1382 | CCTCGCATGCTGGACTAGGTTCCCC | SHANK3 H22 3UTR(+899+923) |
| 1383 | TGACCTCGCATGCTGGACTAGGTTC | SHANK3 H22 3UTR(+902+926) |
| 1384 | TCCTGACCTCGCATGCTGGACTAGG | SHANK3 H22 3UTR(+905+929) |
| 1385 | GGGTCCTGACCTCGCATGCTGGACT | SHANK3 H22 3UTR(+908+932) |
| 1386 | GGCGGGTCCTGACCTCGCATGCTGG | SHANK3 H22 3UTR(+911+935) |
| 1387 | CAAGGCGGGTCCTGACCTCGCATGC | SHANK3 H22 3UTR(+914+938) |
| 1388 | CACCAAGGCGGGTCCTGACCTCGCA | SHANK3 H22 3UTR(+917+941) |
| 1389 | AGTCACCAAGGCGGGTCCTGACCTC | SHANK3 H22 3UTR(+920+944) |
| 1390 | CCGAGTCACCAAGGCGGGTCCTGAC | SHANK3 H22 3UTR(+923+947) |
| 1391 | CCCCCGAGTCACCAAGGCGGGTCCT | SHANK3 H22 3UTR(+926+950) |
| 1392 | CTCCCCCCGAGTCACCAAGGCGGGT | SHANK3 H22 3UTR(+929+953) |
| 1393 | CCCCTCCCCCCGAGTCACCAAGGCG | SHANK3 H22 3UTR(+932+956) |
| 1394 | TCCCCCCTCCCCCCGAGTCACCAAG | SHANK3 H22 3UTR(+935+959) |
| 1395 | GTCTCCCCCCTCCCCCCGAGTCACC | SHANK3 H22 3UTR(+938+962) |
| 1396 | AATGTCTCCCCCCTCCCCCCGAGTC | SHANK3 H22 3UTR(+941+965) |
| 1397 | CCCAATGTCTCCCCCCTCCCCCCGA | SHANK3 H22 3UTR(+944+968) |
| 1398 | AATCCCAATGTCTCCCCCCTCCCCC | SHANK3 H22 3UTR(+947+971) |
| 1399 | GAGAATCCCAATGTCTCCCCCCTCC | SHANK3 H22 3UTR(+950+974) |
| 1400 | ATCGAGAATCCCAATGTCTCCCCCC | SHANK3 H22 3UTR(+953+977) |
| 1401 | CCCATCGAGAATCCCAATGTCTCCC | SHANK3 H22 3UTR(+956+980) |
| 1402 | GCCCCCATCGAGAATCCCAATGTCT | SHANK3 H22 3UTR(+959+983) |
| 1403 | TTGGCCCCCATCGAGAATCCCAATG | SHANK3 H22 3UTR(+962+986) |
| 1404 | TCCTTGGCCCCCATCGAGAATCCCA | SHANK3 H22 3UTR(+965+989) |
| 1405 | GGCTCCTTGGCCCCCATCGAGAATC | SHANK3 H22 3UTR(+968+992) |
| 1406 | GGGGGCTCCTTGGCCCCCATCGAGA | SHANK3 H22 3UTR(+971+995) |
| 1407 | CAGGGGGGCTCCTTGGCCCCCATCG | SHANK3 H22 3UTR(+974+998) |
| 1408 | AAACAGGGGGGCTCCTTGGCCCCCA | SHANK3 H22 3UTR(+977+1001) |
| 1409 | GCAAAACAGGGGGGCTCCTTGGCCC | SHANK3 H22 3UTR(+980+1004) |
| 1410 | TATGCAAAACAGGGGGGCTCCTTGG | SHANK3 H22 3UTR(+983+1007) |
| 1411 | AAATATGCAAAACAGGGGGGCTCCT | SHANK3 H22 3UTR(+986+1010) |
| 1412 | TTAAAATATGCAAAACAGGGGGGCT | SHANK3 H22 3UTR(+989+1013) |
| 1413 | GGATTAAAATATGCAAAACAGGGGG | SHANK3 H22 3UTR(+992+1016) |
| 1414 | AGTGGATTAAAATATGCAAAACAGG | SHANK3 H22 3UTR(+995+1019) |
| 1415 | TAGAGTGGATTAAAATATGCAAAAC | SHANK3 H22 3UTR(+998+1022) |
| 1416 | ATATAGAGTGGATTAAAATATGCAA | SHANK3 H22 3UTR(+1001+1025) |
| 1417 | CAAATATAGAGTGGATTAAAATATG | SHANK3 H22 3UTR(+1004+1028) |
| 1418 | TTCCAAATATAGAGTGGATTAAAAT | SHANK3 H22 3UTR(+1007+1031) |
| 1419 | TCGTTCCAAATATAGAGTGGATTAA | SHANK3 H22 3UTR(+1010+1034) |
| 1420 | TTCTCGTTCCAAATATAGAGTGGAT | SHANK3 H22 3UTR(+1013+1037) |
| 1421 | CTTTTCTCGTTCCAAATATAGAGTG | SHANK3 H22 3UTR(+1016+1040) |
| 1422 | TTCCTTTTCTCGTTCCAAATATAGA | SHANK3 H22 3UTR(+1019+1043) |
| 1423 | TTGTTCCTTTTCTCGTTCCAAATAT | SHANK3 H22 3UTR(+1022+1046) |
| 1424 | TATTTGTTCCTTTTCTCGTTCCAAA | SHANK3 H22 3UTR(+1025+1049) |
| 1425 | AGATATTTGTTCCTTTTCTCGTTCC | SHANK3 H22 3UTR(+1028+1052) |
| 1426 | CAGAGATATTTGTTCCTTTTCTCGT | SHANK3 H22 3UTR(+1031+1055) |
| 1427 | GGACAGAGATATTTGTTCCTTTTCT | SHANK3 H22 3UTR(+1034+1058) |
| 1428 | TACGGACAGAGATATTTGTTCCTTT | SHANK3 H22 3UTR(+1037+1061) |
| 1429 | TATTACGGACAGAGATATTTGTTCC | SHANK3 H22 3UTR(+1040+1064) |
| 1430 | AACTATTACGGACAGAGATATTTGT | SHANK3 H22 3UTR(+1043+1067) |
| 1431 | GGAAACTATTACGGACAGAGATATT | SHANK3 H22 3UTR(+1046+1070) |
| 1432 | AGAGGAAACTATTACGGACAGAGAT | SHANK3 H22 3UTR(+1049+1073) |
| 1433 | GGGAGAGGAAACTATTACGGACAGA | SHANK3 H22 3UTR(+1052+1076) |
| 1434 | GAGGGGAGAGGAAACTATTACGGAC | SHANK3 H22 3UTR(+1055+1079) |
| 1435 | AGGGAGGGGAGAGGAAACTATTACG | SHANK3 H22 3UTR(+1058+1082) |
| 1436 | AGAAGGGAGGGGAGAGGAAACTATT | SHANK3 H22 3UTR(+1061+1085) |
| 1437 | AGTAGAAGGGAGGGGAGAGGAAACT | SHANK3 H22 3UTR(+1064+1088) |
| 1438 | GGAAGTAGAAGGGAGGGGAGAGGAA | SHANK3 H22 3UTR(+1067+1091) |
| 1439 | AGTGGAAGTAGAAGGGAGGGGAGAG | SHANK3 H22 3UTR(+1070+1094) |
| 1440 | ACCAGTGGAAGTAGAAGGGAGGGGA | SHANK3 H22 3UTR(+1073+1097) |
| 1441 | GGGACCAGTGGAAGTAGAAGGGAGG | SHANK3 H22 3UTR(+1076+1100) |
| 1442 | AGTGGGACCAGTGGAAGTAGAAGGG | SHANK3 H22 3UTR(+1079+1103) |
| 1443 | TGCAGTGGGACCAGTGGAAGTAGAA | SHANK3 H22 3UTR(+1082+1106) |
| 1444 | AGCTGCAGTGGGACCAGTGGAAGTA | SHANK3 H22 3UTR(+1085+1109) |
| 1445 | GGCAGCTGCAGTGGGACCAGTGGAA | SHANK3 H22 3UTR(+1088+1112) |
| 1446 | CTGGGCAGCTGCAGTGGGACCAGTG | SHANK3 H22 3UTR(+1091+1115) |
| 1447 | AGACTGGGCAGCTGCAGTGGGACCA | SHANK3 H22 3UTR(+1094+1118) |
| 1448 | GGAAGACTGGGCAGCTGCAGTGGGA | SHANK3 H22 3UTR(+1097+1121) |
| 1449 | GATGGAAGACTGGGCAGCTGCAGTG | SHANK3 H22 3UTR(+1100+1124) |
| 1450 | GGAGATGGAAGACTGGGCAGCTGCA | SHANK3 H22 3UTR(+1103+1127) |
| 1451 | GCCGGAGATGGAAGACTGGGCAGCT | SHANK3 H22 3UTR(+1106+1130) |
| 1452 | GGGGCCGGAGATGGAAGACTGGGCA | SHANK3 H22 3UTR(+1109+1133) |
| 1453 | TGAGGGGCCGGAGATGGAAGACTGG | SHANK3 H22 3UTR(+1112+1136) |
| 1454 | CAGTGAGGGGCCGGAGATGGAAGAC | SHANK3 H22 3UTR(+1115+1139) |
| 1455 | TGGCAGTGAGGGGCCGGAGATGGAA | SHANK3 H22 3UTR(+1118+1142) |
| 1456 | CAGTGGCAGTGAGGGGCCGGAGATG | SHANK3 H22 3UTR(+1121+1145) |
| 1457 | TGGCAGTGGCAGTGAGGGGCCGGAG | SHANK3 H22 3UTR(+1124+1148) |
| 1458 | GGGTGGCAGTGGCAGTGAGGGGCCG | SHANK3 H22 3UTR(+1127+1151) |
| 1459 | GTGGGGTGGCAGTGGCAGTGAGGGG | SHANK3 H22 3UTR(+1130+1154) |
| 1460 | GTTGTGGGGTGGCAGTGGCAGTGAG | SHANK3 H22 3UTR(+1133+1157) |
| 1461 | CCCGTTGTGGGGGGCAGTGGCAGT | SHANK3 H22 3UTR(+1136+1160) |
| 1462 | TGCCCCGTTGTGGGGTGGCAGTGGC | SHANK3 H22 3UTR(+1139+1163) |
| 1463 | CCCTGCCCCGTTGTGGGGTGGCAGT | SHANK3 H22 3UTR(+1142+1166) |
| 1464 | GTCCCCTGCCCCGTTGTGGGGTGGC | SHANK3 H22 3UTR(+1145+1169) |
| 1465 | AGCGTCCCCTGCCCCGTTGTGGGGT | SHANK3 H22 3UTR(+1148+1172) |
| 1466 | TGGAGCGTCCCCTGCCCCGTTGTGG | SHANK3 H22 3UTR(+1151+1175) |
| 1467 | AGCTGGAGCGTCCCCTGCCCCGTTG | SHANK3 H22 3UTR(+1154+1178) |
| 1468 | ACCAGCTGGAGCGTCCCCTGCCCCG | SHANK3 H22 3UTR(+1157+1181) |
| 1469 | CAGACCAGCTGGAGCGTCCCCTGCC | SHANK3 H22 3UTR(+1160+1184) |
| 1470 | CCCCAGACCAGCTGGAGCGTCCCCT | SHANK3 H22 3UTR(+1163+1187) |
| 1471 | CAACCCCAGACCAGCTGGAGCGTCC | SHANK3 H22 3UTR(+1166+1190) |
| 1472 | GGCCAACCCCAGACCAGCTGGAGCG | SHANK3 H22 3UTR(+1169+1193) |
| 1473 | CCTGGCCAACCCCAGACCAGCTGGA | SHANK3 H22 3UTR(+1172+1196) |
| 1474 | GGCCCTGGCCAACCCCAGACCAGCT | SHANK3 H22 3UTR(+1175+1199) |
| 1475 | TAGGGCCCTGGCCAACCCCAGACCA | SHANK3 H22 3UTR(+1178+1202) |
| 1476 | CACTAGGGCCCTGGCCAACCCCAGA | SHANK3 H22 3UTR(+1181+1205) |
| 1477 | GGCCACTAGGGCCCTGGCCAACCCC | SHANK3 H22 3UTR(+1184+1208) |
| 1478 | GCGGGCCACTAGGGCCCTGGCCAAC | SHANK3 H22 3UTR(+1187+1211) |
| 1479 | AGGGCGGGCCACTAGGGCCCTGGCC | SHANK3 H22 3UTR(+1190+1214) |
| 1480 | CCCAGGGCGGGCCACTAGGGCCCTG | SHANK3 H22 3UTR(+1193+1217) |
| 1481 | GGCCCCAGGGCGGGCCACTAGGGCC | SHANK3 H22 3UTR(+1196+1220) |
| 1482 | TGGGGCCCCAGGGCGGGCCACTAGG | SHANK3 H22 3UTR(+1199+1223) |
| 1483 | AGCTGGGGCCCCAGGGCGGGCCACT | SHANK3 H22 3UTR(+1202+1226) |
| 1484 | CCGAGCTGGGGCCCCAGGGCGGGCC | SHANK3 H22 3UTR(+1205+1229) |
| 1485 | GGGCCGAGCTGGGGCCCCAGGGCGG | SHANK3 H22 3UTR(+1208+1232) |
| 1486 | GAGGGGCCGAGCTGGGGCCCCAGGG | SHANK3 H22 3UTR(+1211+1235) |
| 1487 | GGCGAGGGGCCGAGCTGGGGCCCCA | SHANK3 H22 3UTR(+1214+1238) |
| 1488 | CGAGGCGAGGGGCCGAGCTGGGGCC | SHANK3 H22 3UTR(+1217+1241) |
| 1489 | CAGCGAGGCGAGGGGCCGAGCTGGG | SHANK3 H22 3UTR(+1220+1244) |
| 1490 | GCTCAGCGAGGCGAGGGGCCGAGCT | SHANK3 H22 3UTR(+1223+1247) |
| 1491 | AGAGCTCAGCGAGGCGAGGGGCCGA | SHANK3 H22 3UTR(+1226+1250) |
| 1492 | ACTAGAGCTCAGCGAGGCGAGGGGC | SHANK3 H22 3UTR(+1229+1253) |
| 1493 | CACACTAGAGCTCAGCGAGGCGAGG | SHANK3 H22 3UTR(+1232+1256) |
| 1494 | GGGCACACTAGAGCTCAGCGAGGCG | SHANK3 H22 3UTR(+1235+1259) |
| 1495 | GTGGGGCACACTAGAGCTCAGCGAG | SHANK3 H22 3UTR(+1238+1262) |
| 1496 | TCGGTGGGGCACACTAGAGCTCAGC | SHANK3 H22 3UTR(+1241+1265) |
| 1497 | GGGTCGGTGGGGCACACTAGAGCTC | SHANK3 H22 3UTR(+1244+1268) |
| 1498 | GAAGGGTCGGTGGGGCACACTAGAG | SHANK3 H22 3UTR(+1247+1271) |
| 1499 | CCTGAAGGGTCGGTGGGGCACACTA | SHANK3 H22 3UTR(+1250+1274) |
| 1500 | GCACCTGAAGGGTCGGTGGGGCACA | SHANK3 H22 3UTR(+1253+1277) |
| 1501 | GCAGCACCTGAAGGGTCGGTGGGGC | SHANK3 H22 3UTR(+1256+1280) |
| 1502 | CGAGCAGCACCTGAAGGGTCGGTGG | SHANK3 H22 3UTR(+1259+1283) |
| 1503 | CCACGAGCAGCACCTGAAGGGTCGG | SHANK3 H22 3UTR(+1262+1286) |
| 1504 | CCACCACGAGCAGCACCTGAAGGGT | SHANK3 H22 3UTR(+1265+1289) |
| 1505 | CTCCCACCACGAGCAGCACCTGAAG | SHANK3 H22 3UTR(+1268+1292) |
| 1506 | CCCCTCCCACCACGAGCAGCACCTG | SHANK3 H22 3UTR(+1271+1295) |
| 1507 | CCGCCCCTCCCACCACGAGCAGCAC | SHANK3 H22 3UTR(+1274+1298) |
| 1508 | CTGCCGCCCCTCCCACCACGAGCAG | SHANK3 H22 3UTR(+1277+1301) |
| 1509 | GGCCTGCCGCCCCTCCCACCACGAG | SHANK3 H22 3UTR(+1280+1304) |
| 1510 | CGCGGCCTGCCGCCCCTCCCACCAC | SHANK3 H22 3UTR(+1283+1307) |
| 1511 | ACCCGCGGCCTGCCGCCCCTCCCAC | SHANK3 H22 3UTR(+1286+1310) |
| 1512 | AGGACCCGCGGCCTGCCGCCCCTCC | SHANK3 H22 3UTR(+1289+1313) |
| 1513 | AGCAGGACCCGCGGCCTGCCGCCCC | SHANK3 H22 3UTR(+1292+1316) |
| 1514 | CACAGCAGGACCCGCGGCCTGCCGC | SHANK3 H22 3UTR(+1295+1319) |
| 1515 | GTGCACAGCAGGACCCGCGGCCTGC | SHANK3 H22 3UTR(+1298+1322) |
| 1516 | CGGGTGCACAGCAGGACCCGCGGCC | SHANK3 H22 3UTR(+1301+1325) |
| 1517 | CCGCGGGTGCACAGCAGGACCCGCG | SHANK3 H22 3UTR(+1304+1328) |
| 1518 | GTCCCGCGGGTGCACAGCAGGACCC | SHANK3 H22 3UTR(+1307+1331) |
| 1519 | CTGGTCCCGCGGGTGCACAGCAGGA | SHANK3 H22 3UTR(+1310+1334) |
| 1520 | CGGCTGGTCCCGCGGGTGCACAGCA | SHANK3 H22 3UTR(+1313+1337) |
| 1521 | GGCCGGCTGGTCCCGCGGGTGCACA | SHANK3 H22 3UTR(+1316+1340) |
| 1522 | CCAGGCCGGCTGGTCCCGCGGGTGC | SHANK3 H22 3UTR(+1319+1343) |
| 1523 | CTCCCAGGCCGGCTGGTCCCGCGGG | SHANK3 H22 3UTR(+1322+1346) |
| 1524 | GGTCTCCCAGGCCGGCTGGTCCCGC | SHANK3 H22 3UTR(+1325+1349) |
| 1525 | GATGGTCTCCCAGGCCGGCTGGTCC | SHANK3 H22 3UTR(+1328+1352) |
| 1526 | GCCGATGGTCTCCCAGGCCGGCTGG | SHANK3 H22 3UTR(+1331+1355) |
| 1527 | CCGGCCGATGGTCTCCCAGGCCGGC | SHANK3 H22 3UTR(+1334+1358) |
| 1528 | CCCCCGGCCGATGGTCTCCCAGGCC | SHANK3 H22 3UTR(+1337+1361) |
| 1529 | TCCCCCCCGGCCGATGGTCTCCCAG | SHANK3 H22 3UTR(+1340+1364) |
| 1530 | TCATCCCCCCCGGCCGATGGTCTCC | SHANK3 H22 3UTR(+1343+1367) |
| 1531 | CCCTCATCCCCCCCGGCCGATGGTC | SHANK3 H22 3UTR(+1346+1370) |
| 1532 | CTGCCCTCATCCCCCCCGGCCGATG | SHANK3 H22 3UTR(+1349+1373) |
| 1533 | GCCCTGCCCTCATCCCCCCCGGCCG | SHANK3 H22 3UTR(+1352+1376) |
| 1534 | AGGGCCCTGCCCTCATCCCCCCCGG | SHANK3 H22 3UTR(+1355+1379) |
| 1535 | GGCAGGGCCCTGCCCTCATCCCCCC | SHANK3 H22 3UTR(+1358+1382) |
| 1536 | AGCGGCAGGGCCCTGCCCTCATCCC | SHANK3 H22 3UTR(+1361+1385) |
| 1537 | TGGAGCGGCAGGGCCCTGCCCTCAT | SHANK3 H22 3UTR(+1364+1388) |
| 1538 | CGGTGGAGCGGCAGGGCCCTGCCCT | SHANK3 H22 3UTR(+1367+1391) |
| 1539 | CTGCGGTGGAGCGGCAGGGCCCTGC | SHANK3 H22 3UTR(+1370+1394) |
| 1540 | TGGCTGCGGTGGAGCGGCAGGGCCC | SHANK3 H22 3UTR(+1373+1397) |
| 1541 | AGATGGCTGCGGTGGAGCGGCAGGG | SHANK3 H22 3UTR(+1376+1400) |
| 1542 | GGAAGATGGCTGCGGTGGAGCGGCA | SHANK3 H22 3UTR(+1379+1403) |
| 1543 | TGAGGAAGATGGCTGCGGTGGAGCG | SHANK3 H22 3UTR(+1382+1406) |
| 1544 | CTGTGAGGAAGATGGCTGCGGTGGA | SHANK3 H22 3UTR(+1385+1409) |
| 1545 | ACCCTGTGAGGAAGATGGCTGCGGT | SHANK3 H22 3UTR(+1388+1412) |
| 1546 | GAGACCCTGTGAGGAAGATGGCTGC | SHANK3 H22 3UTR(+1391+1415) |
| 1547 | GGAGAGACCCTGTGAGGAAGATGGC | SHANK3 H22 3UTR(+1394+1418) |
| 1548 | TGGGGAGAGACCCTGTGAGGAAGAT | SHANK3 H22 3UTR(+1397+1421) |
| 1549 | CCTTGGGGAGAGACCCTGTGAGGAA | SHANK3 H22 3UTR(+1400+1424) |
| 1550 | CCTCCTTGGGGAGAGACCCTGTGAG | SHANK3 H22 3UTR(+1403+1427) |
| 1551 | CCCCCTCCTTGGGGAGAGACCCTGT | SHANK3 H22 3UTR(+1406+1430) |
| 1552 | TAGCCCCCTCCTTGGGGAGAGACCC | SHANK3 H22 3UTR(+1409+1433) |
| 1553 | AGCTAGCCCCCTCCTTGGGGAGAGA | SHANK3 H22 3UTR(+1412+1436) |
| 1554 | CCAAGCTAGCCCCCTCCTTGGGGAG | SHANK3 H22 3UTR(+1415+1439) |
| 1555 | GGACCAAGCTAGCCCCCTCCTTGGG | SHANK3 H22 3UTR(+1418+1442) |
| 1556 | TGGGGACCAAGCTAGCCCCCTCCTT | SHANK3 H22 3UTR(+1421+1445) |
| 1557 | GCATGGGGACCAAGCTAGCCCCCTC | SHANK3 H22 3UTR(+1424+1448) |
| 1558 | AGAGCATGGGGACCAAGCTAGCCCC | SHANK3 H22 3UTR(+1427+1451) |
| 1559 | CCAAGAGCATGGGGACCAAGCTAGC | SHANK3 H22 3UTR(+1430+1454) |
| 1560 | TGCCCAAGAGCATGGGGACCAAGCT | SHANK3 H22 3UTR(+1433+1457) |
| 1561 | AGTTGCCCAAGAGCATGGGGACCAA | SHANK3 H22 3UTR(+1436+1460) |
| 1562 | TGTAGTTGCCCAAGAGCATGGGGAC | SHANK3 H22 3UTR(+1439+1463) |
| 1563 | TGCTGTAGTTGCCCAAGAGCATGGG | SHANK3 H22 3UTR(+1442+1466) |
| 1564 | CTCTGCTGTAGTTGCCCAAGAGCAT | SHANK3 H22 3UTR(+1445+1469) |
| 1565 | CTTCTCTGCTGTAGTTGCCCAAGAG | SHANK3 H22 3UTR(+1448+1472) |
| 1566 | AGGCTTCTCTGCTGTAGTTGCCCAA | SHANK3 H22 3UTR(+1451+1475) |
| 1567 | GGGAGGCTTCTCTGCTGTAGTTGCC | SHANK3 H22 3UTR(+1454+1478) |
| 1568 | GCAGGGAGGCTTCTCTGCTGTAGTT | SHANK3 H22 3UTR(+1457+1481) |
| 1569 | AAGGCAGGGAGGCTTCTCTGCTGTA | SHANK3 H22 3UTR(+1460+1484) |
| 1570 | TCCAAGGCAGGGAGGCTTCTCTGCT | SHANK3 H22 3UTR(+1463+1487) |
| 1571 | GGGTCCAAGGCAGGGAGGCTTCTCT | SHANK3 H22 3UTR(+1466+1490) |
| 1572 | TTGGGGTCCAAGGCAGGGAGGCTTC | SHANK3 H22 3UTR(+1469+1493) |
| 1573 | ACTTTGGGGTCCAAGGCAGGGAGGC | SHANK3 H22 3UTR(+1472+1496) |
| 1574 | GAGACTTTGGGGTCCAAGGCAGGGA | SHANK3 H22 3UTR(+1475+1499) |
| 1575 | CAGGAGACTTTGGGGTCCAAGGCAG | SHANK3 H22 3UTR(+1478+1502) |
| 1576 | GGACAGGAGACTTTGGGGTCCAAGG | SHANK3 H22 3UTR(+1481+1505) |
| 1577 | GCAGGACAGGAGACTTTGGGGTCCA | SHANK3 H22 3UTR(+1484+1508) |
| 1578 | AGGGCAGGACAGGAGACTTTGGGGT | SHANK3 H22 3UTR(+1487+1511) |
| 1579 | TAAAGGGCAGGACAGGAGACTTTGG | SHANK3 H22 3UTR(+1490+1514) |
| 1580 | ACATAAAGGGCAGGACAGGAGACTT | SHANK3 H22 3UTR(+1493+1517) |
| 1581 | CACACATAAAGGGCAGGACAGGAGA | SHANK3 H22 3UTR(+1496+1520) |
| 1582 | CCACACACATAAAGGGCAGGACAGG | SHANK3 H22 3UTR(+1499+1523) |
| 1583 | CACCCACACACATAAAGGGCAGGAC | SHANK3 H22 3UTR(+1502+1526) |
| 1584 | TTTCACCCACACACATAAAGGGCAG | SHANK3 H22 3UTR(+1505+1529) |
| 1585 | CAGTTTCACCCACACACATAAAGGG | SHANK3 H22 3UTR(+1508+1532) |
| 1586 | ACCCAGTTTCACCCACACACATAAA | SHANK3 H22 3UTR(+1511+1535) |
| 1587 | CGCACCCAGTTTCACCCACACACAT | SHANK3 H22 3UTR(+1514+1538) |
| 1588 | AGACGCACCCAGTTTCACCCACACA | SHANK3 H22 3UTR(+1517+1541) |
| 1589 | CTCAGACGCACCCAGTTTCACCCAC | SHANK3 H22 3UTR(+1520+1544) |
| 1590 | GTGCTCAGACGCACCCAGTTTCACC | SHANK3 H22 3UTR(+1523+1547) |
| 1591 | CACGTGCTCAGACGCACCCAGTTTC | SHANK3 H22 3UTR(+1526+1550) |
| 1592 | TCCCACGTGCTCAGACGCACCCAGT | SHANK3 H22 3UTR(+1529+1553) |
| 1593 | GGCTCCCACGTGCTCAGACGCACCC | SHANK3 H22 3UTR(+1532+1556) |
| 1594 | CACGGCTCCCACGTGCTCAGACGCA | SHANK3 H22 3UTR(+1535+1559) |
| 1595 | ACACACGGCTCCCACGTGCTCAGAC | SHANK3 H22 3UTR(+1538+1562) |
| 1596 | CACACACACGGCTCCCACGTGCTCA | SHANK3 H22 3UTR(+1541+1565) |
| 1597 | AGGCACACACACGGCTCCCACGTGC | SHANK3 H22 3UTR(+1544+1568) |
| 1598 | ATCAGGCACACACACGGCTCCCACG | SHANK3 H22 3UTR(+1547+1571) |
| 1599 | GTAATCAGGCACACACACGGCTCCC | SHANK3 H22 3UTR(+1550+1574) |
| 1600 | TCAGTAATCAGGCACACACACGGCT | SHANK3 H22 3UTR(+1553+1577) |
| 1601 | CACTCAGTAATCAGGCACACACACG | SHANK3 H22 3UTR(+1556+1580) |
| 1602 | GGCCACTCAGTAATCAGGCACACAC | SHANK3 H22 3UTR(+1559+1583) |
| 1603 | GGTGGCCACTCAGTAATCAGGCACA | SHANK3 H22 3UTR(+1562+1586) |
| 1604 | CCTGGTGGCCACTCAGTAATCAGGC | SHANK3 H22 3UTR(+1565+1589) |
| 1605 | GCCCCTGGTGGCCACTCAGTAATCA | SHANK3 H22 3UTR(+1568+1592) |
| 1606 | GCGGCCCCTGGTGGCCACTCAGTAA | SHANK3 H22 3UTR(+1571+1595) |
| 1607 | AGAGCGGCCCCTGGTGGCCACTCAG | SHANK3 H22 3UTR(+1574+1598) |
| 1608 | TCCAGAGCGGCCCCTGGTGGCCACT | SHANK3 H22 3UTR(+1577+1601) |
| 1609 | TAGTCCAGAGCGGCCCCTGGTGGCC | SHANK3 H22 3UTR(+1580+1604) |
| 1610 | CGCTAGTCCAGAGCGGCCCCTGGTG | SHANK3 H22 3UTR(+1583+1607) |
| 1611 | CCGCGCTAGTCCAGAGCGGCCCCTG | SHANK3 H22 3UTR(+1586+1610) |
| 1612 | GCCCCGCGCTAGTCCAGAGCGGCCC | SHANK3 H22 3UTR(+1589+1613) |
| 1613 | ACGGCCCCGCGCTAGTCCAGAGCGG | SHANK3 H22 3UTR(+1592+1616) |
| 1614 | TCCACGGCCCCGCGCTAGTCCAGAG | SHANK3 H22 3UTR(+1595+1619) |
| 1615 | GCCTCCACGGCCCCGCGCTAGTCCA | SHANK3 H22 3UTR(+1598+1622) |
| 1616 | CACGCCTCCACGGCCCCGCGCTAGT | SHANK3 H22 3UTR(+1601+1625) |
| 1617 | GTGCACGCCTCCACGGCCCCGCGCT | SHANK3 H22 3UTR(+1604+1628) |
| 1618 | ACGGTGCACGCCTCCACGGCCCCGC | SHANK3 H22 3UTR(+1607+1631) |
| 1619 | CACACGGTGCACGCCTCCACGGCCC | SHANK3 H22 3UTR(+1610+1634) |
| 1620 | ATGCACACGGTGCACGCCTCCACGG | SHANK3 H22 3UTR(+1613+1637) |
| 1621 | CGCATGCACACGGTGCACGCCTCCA | SHANK3 H22 3UTR(+1616+1640) |
| 1622 | CCACGCATGCACACGGTGCACGCCT | SHANK3 H22 3UTR(+1619+1643) |
| 1623 | ACCCCACGCATGCACACGGTGCACG | SHANK3 H22 3UTR(+1622+1646) |
| 1624 | TACACCCCACGCATGCACACGGTGC | SHANK3 H22 3UTR(+1625+1649) |
| 1625 | AGGTACACCCCACGCATGCACACGG | SHANK3 H22 3UTR(+1628+1652) |
| 1626 | CACAGGTACACCCCACGCATGCACA | SHANK3 H22 3UTR(+1631+1655) |
| 1627 | TCTCACAGGTACACCCCACGCATGC | SHANK3 H22 3UTR(+1634+1658) |
| 1628 | TGCTCTCACAGGTACACCCCACGCA | SHANK3 H22 3UTR(+1637+1661) |
| 1629 | GGGTGCTCTCACAGGTACACCCCAC | SHANK3 H22 3UTR(+1640+1664) |
| 1630 | ACAGGGTGCTCTCACAGGTACACCC | SHANK3 H22 3UTR(+1643+1667) |
| 1631 | GAGACAGGGTGCTCTCACAGGTACA | SHANK3 H22 3UTR(+1646+1670) |
| 1632 | GAGGAGACAGGGTGCTCTCACAGGT | SHANK3 H22 3UTR(+1649+1673) |
| 1633 | GAAGAGGAGACAGGGTGCTCTCACA | SHANK3 H22 3UTR(+1652+1676) |
| 1634 | TTGGAAGAGGAGACAGGGTGCTCTC | SHANK3 H22 3UTR(+1655+1679) |
| 1635 | TCTTTGGAAGAGGAGACAGGGTGCT | SHANK3 H22 3UTR(+1658+1682) |
| 1636 | CTTTCTTTGGAAGAGGAGACAGGGT | SHANK3 H22 3UTR(+1661+1685) |
| 1637 | TGACTTTCTTTGGAAGAGGAGACAG | SHANK3 H22 3UTR(+1664+1688) |
| 1638 | CTCTGACTTTCTTTGGAAGAGGAGA | SHANK3 H22 3UTR(+1667+1691) |
| 1639 | GGCCTCTGACTTTCTTTGGAAGAGG | SHANK3 H22 3UTR(+1670+1694) |
| 1640 | GATGGCCTCTGACTTTCTTTGGAAG | SHANK3 H22 3UTR(+1673+1697) |
| 1641 | CAGGATGGCCTCTGACTTTCTTTGG | SHANK3 H22 3UTR(+1676+1700) |
| 1642 | GTGCAGGATGGCCTCTGACTTTCTT | SHANK3 H22 3UTR(+1679+1703) |
| 1643 | AGGGTGCAGGATGGCCTCTGACTTT | SHANK3 H22 3UTR(+1682+1706) |
| 1644 | CCCAGGGTGCAGGATGGCCTCTGAC | SHANK3 H22 3UTR(+1685+1709) |
| 1645 | GGACCCAGGGTGCAGGATGGCCTCT | SHANK3 H22 3UTR(+1688+1712) |
| 1646 | GCTGGACCCAGGGTGCAGGATGGCC | SHANK3 H22 3UTR(+1691+1715) |
| 1647 | ACAGCTGGACCCAGGGTGCAGGATG | SHANK3 H22 3UTR(+1694+1718) |
| 1648 | CAAACAGCTGGACCCAGGGTGCAGG | SHANK3 H22 3UTR(+1697+1721) |
| 1649 | GGGCAAACAGCTGGACCCAGGGTGC | SHANK3 H22 3UTR(+1700+1724) |
| 1650 | GCTGGGCAAACAGCTGGACCCAGGG | SHANK3 H22 3UTR(+1703+1727) |
| 1651 | CAGGCTGGGCAAACAGCTGGACCCA | SHANK3 H22 3UTR(+1706+1730) |
| 1652 | GGACAGGCTGGGCAAACAGCTGGAC | SHANK3 H22 3UTR(+1709+1733) |
| 1653 | GAAGGACAGGCTGGGCAAACAGCTG | SHANK3 H22 3UTR(+1712+1736) |
| 1654 | CTGGAAGGACAGGCTGGGCAAACAG | SHANK3 H22 3UTR(+1715+1739) |
| 1655 | GCTCTGGAAGGACAGGCTGGGCAAA | SHANK3 H22 3UTR(+1718+1742) |
| 1656 | GAGGCTCTGGAAGGACAGGCTGGGC | SHANK3 H22 3UTR(+1721+1745) |
| 1657 | GGTGAGGCTCTGGAAGGACAGGCTG | SHANK3 H22 3UTR(+1724+1748) |
| 1658 | CTGGGTGAGGCTCTGGAAGGACAGG | SHANK3 H22 3UTR(+1727+1751) |
| 1659 | AGGCTGGGTGAGGCTCTGGAAGGAC | SHANK3 H22 3UTR(+1730+1754) |
| 1660 | CTCAGGCTGGGTGAGGCTCTGGAAG | SHANK3 H22 3UTR(+1733+1757) |
| 1661 | CCGCTCAGGCTGGGTGAGGCTCTGG | SHANK3 H22 3UTR(+1736+1760) |
| 1662 | ACCCCGCTCAGGCTGGGTGAGGCTC | SHANK3 H22 3UTR(+1739+1763) |
| 1663 | GGAACCCCGCTCAGGCTGGGTGAGG | SHANK3 H22 3UTR(+1742+1766) |
| 1664 | CAGGGAACCCCGCTCAGGCTGGGTG | SHANK3 H22 3UTR(+1745+1769) |
| 1665 | CACCAGGGAACCCCGCTCAGGCTGG | SHANK3 H22 3UTR(+1748+1772) |
| 1666 | ATTCACCAGGGAACCCCGCTCAGGC | SHANK3 H22 3UTR(+1751+1775) |
| 1667 | GGGATTCACCAGGGAACCCCGCTCA | SHANK3 H22 3UTR(+1754+1778) |
| 1668 | GCAGGGATTCACCAGGGAACCCCGC | SHANK3 H22 3UTR(+1757+1781) |
| 1669 | GCAGCAGGGATTCACCAGGGAACCC | SHANK3 H22 3UTR(+1760+1784) |
| 1670 | CAAGCAGCAGGGATTCACCAGGGAA | SHANK3 H22 3UTR(+1763+1787) |
| 1671 | CCCCAAGCAGCAGGGATTCACCAGG | SHANK3 H22 3UTR(+1766+1790) |
| 1672 | CCTCCCCAAGCAGCAGGGATTCACC | SHANK3 H22 3UTR(+1769+1793) |
| 1673 | GGGCCTCCCCAAGCAGCAGGGATTC | SHANK3 H22 3UTR(+1772+1796) |
| 1674 | TTGGGGCCTCCCCAAGCAGCAGGGA | SHANK3 H22 3UTR(+1775+1799) |
| 1675 | CCCTTGGGGCCTCCCCAAGCAGCAG | SHANK3 H22 3UTR(+1778+1802) |
| 1676 | GGGCCCTTGGGGCCTCCCCAAGCAG | SHANK3 H22 3UTR(+1781+1805) |
| 1677 | AAGGGGCCCTTGGGGCCTCCCCAAG | SHANK3 H22 3UTR(+1784+1808) |
| 1678 | TCCAAGGGGCCCTTGGGGCCTCCCC | SHANK3 H22 3UTR(+1787+1811) |
| 1679 | GCCTCCAAGGGGCCCTTGGGGCCTC | SHANK3 H22 3UTR(+1790+1814) |
| 1680 | GCTGCCTCCAAGGGGCCCTTGGGGC | SHANK3 H22 3UTR(+1793+1817) |
| 1681 | GGCGCTGCCTCCAAGGGGCCCTTGG | SHANK3 H22 3UTR(+1796+1820) |
| 1682 | GGGGGCGCTGCCTCCAAGGGGCCCT | SHANK3 H22 3UTR(+1799+1823) |
| 1683 | GGTGGGGGCGCTGCCTCCAAGGGGC | SHANK3 H22 3UTR(+1802+1826) |
| 1684 | CAAGGTGGGGGCGCTGCCTCCAAGG | SHANK3 H22 3UTR(+1805+1829) |
| 1685 | GCCCAAGGTGGGGGCGCTGCCTCCA | SHANK3 H22 3UTR(+1808+1832) |
| 1686 | GAAGCCCAAGGTGGGGGCGCTGCCT | SHANK3 H22 3UTR(+1811+1835) |
| 1687 | TCAGAAGCCCAAGGTGGGGGCGCTG | SHANK3 H22 3UTR(+1814+1838) |
| 1688 | CCCTCAGAAGCCCAAGGTGGGGGCG | SHANK3 H22 3UTR(+1817+1841) |
| 1689 | ATGCCCTCAGAAGCCCAAGGTGGGG | SHANK3 H22 3UTR(+1820+1844) |
| 1690 | ATGATGCCCTCAGAAGCCCAAGGTG | SHANK3 H22 3UTR(+1823+1847) |
| 1691 | CCTATGATGCCCTCAGAAGCCCAAG | SHANK3 H22 3UTR(+1826+1850) |
| 1692 | CCCCCTATGATGCCCTCAGAAGCCC | SHANK3 H22 3UTR(+1829+1853) |
| 1693 | GGTCCCCCTATGATGCCCTCAGAAG | SHANK3 H22 3UTR(+1832+1856) |
| 1694 | AGGGGTCCCCCTATGATGCCCTCAG | SHANK3 H22 3UTR(+1835+1859) |
| 1695 | TCTAGGGGTCCCCCTATGATGCCCT | SHANK3 H22 3UTR(+1838+1862) |
| 1696 | GACTCTAGGGGTCCCCCTATGATGC | SHANK3 H22 3UTR(+1841+1865) |
| 1697 | ACTGACTCTAGGGGTCCCCCTATGA | SHANK3 H22 3UTR(+1844+1868) |
| 1698 | TGAACTGACTCTAGGGGTCCCCCTA | SHANK3 H22 3UTR(+1847+1871) |
| 1699 | TGGTGAACTGACTCTAGGGGTCCCC | SHANK3 H22 3UTR(+1850+1874) |
| 1700 | CTGTGGTGAACTGACTCTAGGGGTC | SHANK3 H22 3UTR(+1853+1877) |
| 1701 | GGCCTGTGGTGAACTGACTCTAGGG | SHANK3 H22 3UTR(+1856+1880) |
| 1702 | CAGGGCCTGTGGTGAACTGACTCTA | SHANK3 H22 3UTR(+1859+1883) |
| 1703 | CCCCAGGGCCTGTGGTGAACTGACT | SHANK3 H22 3UTR(+1862+1886) |
| 1704 | TCTCCCCAGGGCCTGTGGTGAACTG | SHANK3 H22 3UTR(+1865+1889) |
| 1705 | GACTCTCCCCAGGGCCTGTGGTGAA | SHANK3 H22 3UTR(+1868+1892) |
| 1706 | TTTGACTCTCCCCAGGGCCTGTGGT | SHANK3 H22 3UTR(+1871+1895) |
| 1707 | GTCTTTGACTCTCCCCAGGGCCTGT | SHANK3 H22 3UTR(+1874+1898) |
| 1708 | GGGGTCTTTGACTCTCCCCAGGGCC | SHANK3 H22 3UTR(+1877+1901) |
| 1709 | TCGGGGGTCTTTGACTCTCCCCAGG | SHANK3 H22 3UTR(+1880+1904) |
| 1710 | CCCTCGGGGGTCTTTGACTCTCCCC | SHANK3 H22 3UTR(+1883+1907) |
| 1711 | GCACCCTCGGGGGTCTTTGACTCTC | SHANK3 H22 3UTR(+1886+1910) |
| 1712 | TGGGCACCCTCGGGGGTCTTTGACT | SHANK3 H22 3UTR(+1889+1913) |
| 1713 | GGCTGGGCACCCTCGGGGGTCTTTG | SHANK3 H22 3UTR(+1892+1916) |
| 1714 | GGGGGCTGGGCACCCTCGGGGGTCT | SHANK3 H22 3UTR(+1895+1919) |
| 1715 | GTGGGGGGCTGGGCACCCTCGGGGG | SHANK3 H22 3UTR(+1898+1922) |
| 1716 | AGTGTGGGGGGCTGGGCACCCTCGG | SHANK3 H22 3UTR(+1901+1925) |
| 1717 | CACAGTGTGGGGGGCTGGGCACCCT | SHANK3 H22 3UTR(+1904+1928) |
| 1718 | AGTCACAGTGTGGGGGGCTGGGCAC | SHANK3 H22 3UTR(+1907+1931) |
| 1719 | AGGAGTCACAGTGTGGGGGGCTGGG | SHANK3 H22 3UTR(+1910+1934) |
| 1720 | GTGAGGAGTCACAGTGTGGGGGGCT | SHANK3 H22 3UTR(+1913+1937) |
| 1721 | AGTGTGAGGAGTCACAGTGTGGGGG | SHANK3 H22 3UTR(+1916+1940) |
| 1722 | CTGAGTGTGAGGAGTCACAGTGTGG | SHANK3 H22 3UTR(+1919+1943) |
| 1723 | TCGCTGAGTGTGAGGAGTCACAGTG | SHANK3 H22 3UTR(+1922+1946) |
| 1724 | TCATCGCTGAGTGTGAGGAGTCACA | SHANK3 H22 3UTR(+1925+1949) |
| 1725 | AGGTCATCGCTGAGTGTGAGGAGTC | SHANK3 H22 3UTR(+1928+1952) |
| 1726 | CACAGGTCATCGCTGAGTGTGAGGA | SHANK3 H22 3UTR(+1931+1955) |
| 1727 | CCCCACAGGTCATCGCTGAGTGTGA | SHANK3 H22 3UTR(+1934+1958) |
| 1728 | CCACCCCACAGGTCATCGCTGAGTG | SHANK3 H22 3UTR(+1937+1961) |
| 1729 | CCCCCACCCCACAGGTCATCGCTGA | SHANK3 H22 3UTR(+1940+1964) |
| 1730 | GGCCCCCCACCCCACAGGTCATCGC | SHANK3 H22 3UTR(+1943+1967) |
| 1731 | CAGGGCCCCCCACCCCACAGGTCAT | SHANK3 H22 3UTR(+1946+1970) |
| 1732 | TCCCAGGGCCCCCCACCCCACAGGT | SHANK3 H22 3UTR(+1949+1973) |
| 1733 | ACGTCCCAGGGCCCCCCACCCCACA | SHANK3 H22 3UTR(+1952+1976) |
| 1734 | AAAACGTCCCAGGGCCCCCCACCCC | SHANK3 H22 3UTR(+1955+1979) |
| 1735 | TTAAAAACGTCCCAGGGCCCCCCAC | SHANK3 H22 3UTR(+1958+1982) |
| 1736 | GGTTTAAAAACGTCCCAGGGCCCCC | SHANK3 H22 3UTR(+1961+1985) |
| 1737 | CTAGGTTTAAAAACGTCCCAGGGCC | SHANK3 H22 3UTR(+1964+1988) |
| 1738 | ACCCTAGGTITAAAAACGTCCCAGG | SHANK3 H22 3UTR(+1967+1991) |
| 1739 | CAAACCCTAGGTTTAAAAACGTCCC | SHANK3 H22 3UTR(+1970+1994) |
| 1740 | CTCCAAACCCTAGGTTTAAAAACGT | SHANK3 H22 3UTR(+1973+1997) |
| 1741 | AGACTCCAAACCCTAGGTTTAAAAA | SHANK3 H22 3UTR(+1976+2000) |
| 1742 | TCCAGACTCCAAACCCTAGGTTTAA | SHANK3 H22 3UTR(+1979+2003) |
| 1743 | TAGTCCAGACTCCAAACCCTAGGTT | SHANK3 H22 3UTR(+1982+2006) |
| 1744 | GCTTAGTCCAGACTCCAAACCCTAG | SHANK3 H22 3UTR(+1985+2009) |
| 1745 | GGAGCTTAGTCCAGACTCCAAACCC | SHANK3 H22 3UTR(+1988+2012) |
| 1746 | GATGGAGCTTAGTCCAGACTCCAAA | SHANK3 H22 3UTR(+1991+2015) |
| 1747 | GTGGATGGAGCTTAGTCCAGACTCC | SHANK3 H22 3UTR(+1994+2018) |
| 1748 | GACGTGGATGGAGCTTAGTCCAGAC | SHANK3 H22 3UTR(+1997+2021) |
| 1749 | AGTGACGTGGATGGAGCTTAGTCCA | SHANK3 H22 3UTR(+2000+2024) |
| 1750 | GTGAGTGACGTGGATGGAGCTTAGT | SHANK3 H22 3UTR(+2003+2027) |
| 1751 | CTTGTGAGTGACGTGGATGGAGCTT | SHANK3 H22 3UTR(+2006+2030) |
| 1752 | AAACTTGTGAGTGACGTGGATGGAG | SHANK3 H22 3UTR(+2009+2033) |
| 1753 | CAGAAACTTGTGAGTGACGTGGATG | SHANK3 H22 3UTR(+2012+2036) |
| 1754 | AAACAGAAACTTGTGAGTGACGTGG | SHANK3 H22 3UTR(+2015+2039) |
| 1755 | TATAAACAGAAACTTGTGAGTGACG | SHANK3 H22 3UTR(+2018+2042) |
| 1756 | AAATATAAACAGAAACTTGTGAGTG | SHANK3 H22 3UTR(+2021+2045) |
| 1757 | TAGAAATATAAACAGAAACTTGTGA | SHANK3 H22 3UTR(+2024+2048) |
| 1758 | AGCTAGAAATATAAACAGAAACTTG | SHANK3 H22 3UTR(+2027+2051) |
| 1759 | AAAAGCTAGAAATATAAACAGAAAC | SHANK3 H22 3UTR(+2030+2054) |
| 1760 | AAAAAAAGCTAGAAATATAAACAGA | SHANK3 H22 3UTR(+2033+2057) |
| 1761 | ATTAAAAAAAGCTAGAAATATAAAC | SHANK3 H22 3UTR(+2036+2060) |
| 1762 | TTTATTAAAAAAAGCTAGAAATATA | SHANK3 H22 3UTR(+2039+2063) |
| 1763 | TATTTTATTAAAAAAAGCTAGAAAT | SHANK3 H22 3UTR(+2042+2066) |
| 1764 | TTTTATTTTATTAAAAAAAGCTAGA | SHANK3 H22 3UTR(+2045+2069) |
| 1765 | TTTTTTTATTTTATTAAAAAAAGCT | SHANK3 H22 3UTR(+2048+2072) |
| 1766 | TTTTTTTTTTATTTTATTAAAAAAA | SHANK3 H22 3UTR(+2051+2075) |
| 1767 | TCTTTTTTTTTTTATTTTATTAAAA | SHANK3 H22 3UTR(+2054+2078) |
| 1768 | TTTTCTTTTTTTTTTTATTTTATTA | SHANK3 H22 3UTR(+2057+2081) |
| 1769 | CTGTTTTCTTTTTTTTTTTATTTTA | SHANK3 H22 3UTR(+2060+2084) |
| 1770 | CTTCTGTTTTCTTTTTTTTTTTATT | SHANK3 H22 3UTR(+2063+2087) |
| 1771 | AAACTTCTGTTTTCTTTTTTTTTTT | SHANK3 H22 3UTR(+2066+2090) |
| 1772 | TGAAAACTTCTGTTTTCTTTTTTTT | SHANK3 H22 3UTR(+2069+2093) |
| 1773 | TTGTGAAAACTTCTGTTTTCTTTTT | SHANK3 H22 3UTR(+2072+2096) |
| 1774 | GGGTTGTGAAAACTTCTGTTTTCTT | SHANK3 H22 3UTR(+2075+2099) |
| 1775 | CCTGGGTTGTGAAAACTTCTGTTTT | SHANK3 H22 3UTR(+2078+2102) |
| 1776 | GCCCCTGGGTTGTGAAAACTTCTGT | SHANK3 H22 3UTR(+2081+2105) |
| 1777 | CAGGCCCCTGGGTTGTGAAAACTTC | SHANK3 H22 3UTR(+2084+2108) |
| 1778 | TGCCAGGCCCCTGGGTTGTGAAAAC | SHANK3 H22 3UTR(+2087+2111) |
| 1779 | GCGTGCCAGGCCCCTGGGTTGTGAA | SHANK3 H22 3UTR(+2090+2114) |
| 1780 | CCGGCGTGCCAGGCCCCTGGGTTGT | SHANK3 H22 3UTR(+2093+2117) |
| 1781 | AGACCGGCGTGCCAGGCCCCTGGGT | SHANK3 H22 3UTR(+2096+2120) |
| 1782 | CACAGACCGGCGTGCCAGGCCCCTG | SHANK3 H22 3UTR(+2099+2123) |
| 1783 | AGGCACAGACCGGCGTGCCAGGCCC | SHANK3 H22 3UTR(+2102+2126) |
| 1784 | GGCAGGCACAGACCGGCGTGCCAGG | SHANK3 H22 3UTR(+2105+2129) |
| 1785 | GCGGGCAGGCACAGACCGGCGTGCC | SHANK3 H22 3UTR(+2108+2132) |
| 1786 | GGGGGGGGCAGGCACAGACCGGCGT | SHANK3 H22 3UTR(+2111+2135) |
| 1787 | GGCGGGGCGGGCAGGCACAGACCGG | SHANK3 H22 3UTR(+2114+2138) |
| 1788 | CAGGGCGGGGGGGGCAGGCACAGAC | SHANK3 H22 3UTR(+2117+2141) |
| 1789 | GGCCAGGGCGGGGGGGGCAGGCACA | SHANK3 H22 3UTR(+2120+2144) |
| 1790 | GTGGGCCAGGGCGGGGGGGGCAGGC | SHANK3 H22 3UTR(+2123+2147) |
| 1791 | CCGGTGGGCCAGGGCGGGGGGGGCA | SHANK3 H22 3UTR(+2126+2150) |
| 1792 | GGGCCGGTGGGCCAGGGCGGGGCGG | SHANK3 H22 3UTR(+2129+2153) |
| 1793 | GTGGGGCCGGTGGGCCAGGGCGGGG | SHANK3 H22 3UTR(+2132+2156) |
| 1794 | GGAGTGGGGCCGGTGGGCCAGGGCG | SHANK3 H22 3UTR(+2135+2159) |
| 1795 | CAGGGAGTGGGGCCGGTGGGCCAGG | SHANK3 H22 3UTR(+2138+2162) |
| 1796 | GCCCAGGGAGTGGGGCCGGTGGGCC | SHANK3 H22 3UTR(+2141+2165) |
| 1797 | TGTGCCCAGGGAGTGGGGCCGGTGG | SHANK3 H22 3UTR(+2144+2168) |
| 1798 | CTCTGTGCCCAGGGAGTGGGGCCGG | SHANK3 H22 3UTR(+2147+2171) |
| 1799 | TGACTCTGTGCCCAGGGAGTGGGGC | SHANK3 H22 3UTR(+2150+2174) |
| 1800 | GTGTGACTCTGTGCCCAGGGAGTGG | SHANK3 H22 3UTR(+2153+2177) |
| 1801 | TGGGTGTGACTCTGTGCCCAGGGAG | SHANK3 H22 3UTR(+2156+2180) |
| 1802 | GAGTGGGTGTGACTCTGTGCCCAGG | SHANK3 H22 3UTR(+2159+2183) |
| 1803 | GATGAGTGGGTGTGACTCTGTGCCC | SHANK3 H22 3UTR(+2162+2186) |
| 1804 | AAGGATGAGTGGGTGTGACTCTGTG | SHANK3 H22 3UTR(+2165+2189) |
| 1805 | CGGAAGGATGAGTGGGTGTGACTCT | SHANK3 H22 3UTR(+2168+2192) |
| 1806 | TGGCGGAAGGATGAGTGGGTGTGAC | SHANK3 H22 3UTR(+2171+2195) |
| 1807 | TGTTGGCGGAAGGATGAGTGGGTGT | SHANK3 H22 3UTR(+2174+2198) |
| 1808 | GACTGTTGGCGGAAGGATGAGTGGG | SHANK3 H22 3UTR(+2177+2201) |
| 1809 | CTGGACTGTTGGCGGAAGGATGAGT | SHANK3 H22 3UTR(+2180+2204) |
| 1810 | GACCTGGACTGTTGGCGGAAGGATG | SHANK3 H22 3UTR(+2183+2207) |
| 1811 | TGTGACCTGGACTGTTGGCGGAAGG | SHANK3 H22 3UTR(+2186+2210) |
| 1812 | CTGTGTGACCTGGACTGTTGGCGGA | SHANK3 H22 3UTR(+2189+2213) |
| 1813 | CTGCTGTGTGACCTGGACTGTTGGC | SHANK3 H22 3UTR(+2192+2216) |
| 1814 | CTGCTGCTGTGTGACCTGGACTGTT | SHANK3 H22 3UTR(+2195+2219) |
| 1815 | TGACTGCTGCTGTGTGACCTGGACT | SHANK3 H22 3UTR(+2198+2222) |
| 1816 | CAGTGACTGCTGCTGTGTGACCTGG | SHANK3 H22 3UTR(+2201+2225) |
| 1817 | TTACAGTGACTGCTGCTGTGTGACC | SHANK3 H22 3UTR(+2204+2228) |
| 1818 | CTGTTACAGTGACTGCTGCTGTGTG | SHANK3 H22 3UTR(+2207+2231) |
| 1819 | AGTCTGTTACAGTGACTGCTGCTGT | SHANK3 H22 3UTR(+2210+2234) |
| 1820 | GGCAGTCTGTTACAGTGACTGCTGC | SHANK3 H22 3UTR(+2213+2237) |
| 1821 | TGTGGCAGTCTGTTACAGTGACTGC | SHANK3 H22 3UTR(+2216+2240) |
| 1822 | GTATGTGGCAGTCTGTTACAGTGAC | SHANK3 H22 3UTR(+2219+2243) |
| 1823 | TGTGTATGTGGCAGTCTGTTACAGT | SHANK3 H22 3UTR(+2222+2246) |
| 1824 | GAGTGTGTATGTGGCAGTCTGTTAC | SHANK3 H22 3UTR(+2225+2249) |
| 1825 | ACCGAGTGTGTATGTGGCAGTCTGT | SHANK3 H22 3UTR(+2228+2252) |
| 1826 | GAGACCGAGTGTGTATGTGGCAGTC | SHANK3 H22 3UTR(+2231+2255) |
| 1827 | TGTGAGACCGAGTGTGTATGTGGCA | SHANK3 H22 3UTR(+2234+2258) |
| 1828 | GAGTGTGAGACCGAGTGTGTATGTG | SHANK3 H22 3UTR(+2237+2261) |
| 1829 | GGTGAGTGTGAGACCGAGTGTGTAT | SHANK3 H22 3UTR(+2240+2264) |
| 1830 | ACAGGTGAGTGTGAGACCGAGTGTG | SHANK3 H22 3UTR(+2243+2267) |
| 1831 | CCCACAGGTGAGTGTGAGACCGAGT | SHANK3 H22 3UTR(+2246+2270) |
| 1832 | AAACCCACAGGTGAGTGTGAGACCG | SHANK3 H22 3UTR(+2249+2273) |
| 1833 | CCAAAACCCACAGGTGAGTGTGAGA | SHANK3 H22 3UTR(+2252+2276) |
| 1834 | GAACCAAAACCCACAGGTGAGTGTG | SHANK3 H22 3UTR(+2255+2279) |
| 1835 | ACGGAACCAAAACCCACAGGTGAGT | SHANK3 H22 3UTR(+2258+2282) |
| 1836 | TGAACGGAACCAAAACCCACAGGTG | SHANK3 H22 3UTR(+2261+2285) |
| 1837 | AATTGAACGGAACCAAAACCCACAG | SHANK3 H22 3UTR(+2264+2288) |
| 1838 | CCAAATTGAACGGAACCAAAACCCA | SHANK3 H22 3UTR(+2267+2291) |
| 1839 | AACCCAAATTGAACGGAACCAAAAC | SHANK3 H22 3UTR(+2270+2294) |
| 1840 | AAAAACCCAAATTGAACGGAACCAA | SHANK3 H22 3UTR(+2273+2297) |
| 1841 | GTTAAAAACCCAAATTGAACGGAAC | SHANK3 H22 3UTR(+2276+2300) |
| 1842 | AAAGTTAAAAACCCAAATTGAACGG | SHANK3 H22 3UTR(+2279+2303) |
| 1843 | TGTAAAGTTAAAAACCCAAATTGAA | SHANK3 H22 3UTR(+2282+2306) |
| 1844 | CCCTGTAAAGTTAAAAACCCAAATT | SHANK3 H22 3UTR(+2285+2309) |
| 1845 | TGACCCTGTAAAGTTAAAAACCCAA | SHANK3 H22 3UTR(+2288+2312) |
| 1846 | AACTGACCCTGTAAAGTTAAAAACC | SHANK3 H22 3UTR(+2291+2315) |
| 1847 | CGGAACTGACCCTGTAAAGTTAAAA | SHANK3 H22 3UTR(+2294+2318) |
| 1848 | AAGCGGAACTGACCCTGTAAAGTTA | SHANK3 H22 3UTR(+2297+2321) |
| 1849 | GTGAAGCGGAACTGACCCTGTAAAG | SHANK3 H22 3UTR(+2300+2324) |
| 1850 | GAGGTGAAGCGGAACTGACCCTGTA | SHANK3 H22 3UTR(+2303+2327) |
| 1851 | AAGGAGGTGAAGCGGAACTGACCCT | SHANK3 H22 3UTR(+2306+2330) |
| 1852 | CAAAAGGAGGTGAAGCGGAACTGAC | SHANK3 H22 3UTR(+2309+2333) |
| 1853 | ATACAAAAGGAGGTGAAGCGGAACT | SHANK3 H22 3UTR(+2312+2336) |
| 1854 | TCCATACAAAAGGAGGTGAAGCGGA | SHANK3 H22 3UTR(+2315+2339) |
| 1855 | AACTCCATACAAAAGGAGGTGAAGC | SHANK3 H22 3UTR(+2318+2342) |
| 1856 | TGGAACTCCATACAAAAGGAGGTGA | SHANK3 H22 3UTR(+2321+2345) |
| 1857 | GGATGGAACTCCATACAAAAGGAGG | SHANK3 H22 3UTR(+2324+2348) |
| 1858 | CCCGGATGGAACTCCATACAAAAGG | SHANK3 H22 3UTR(+2327+2351) |
| 1859 | CCCCCCGGATGGAACTCCATACAAA | SHANK3 H22 3UTR(+2330+2354) |
| 1860 | AAACCCCCCGGATGGAACTCCATAC | SHANK3 H22 3UTR(+2333+2357) |
| 1861 | GTGAAACCCCCCGGATGGAACTCCA | SHANK3 H22 3UTR(+2336+2360) |
| 1862 | GGGGTGAAACCCCCCGGATGGAACT | SHANK3 H22 3UTR(+2339+2363) |
| 1863 | CAGGGGGTGAAACCCCCCGGATGGA | SHANK3 H22 3UTR(+2342+2366) |
| 1864 | GAGCAGGGGGTGAAACCCCCCGGAT | SHANK3 H22 3UTR(+2345+2369) |
| 1865 | CTGGAGCAGGGGGTGAAACCCCCCG | SHANK3 H22 3UTR(+2348+2372) |
| 1866 | GGACTGGAGCAGGGGGTGAAACCCC | SHANK3 H22 3UTR(+2351+2375) |
| 1867 | TCAGGACTGGAGCAGGGGGTGAAAC | SHANK3 H22 3UTR(+2354+2378) |
| 1868 | GCCTCAGGACTGGAGCAGGGGGTGA | SHANK3 H22 3UTR(+2357+2381) |
| 1869 | GAGGCCTCAGGACTGGAGCAGGGGG | SHANK3 H22 3UTR(+2360+2384) |
| 1870 | CAGGAGGCCTCAGGACTGGAGCAGG | SHANK3 H22 3UTR(+2363+2387) |
| 1871 | GGTCAGGAGGCCTCAGGACTGGAGC | SHANK3 H22 3UTR(+2366+2390) |
| 1872 | CAGGGTCAGGAGGCCTCAGGACTGG | SHANK3 H22 3UTR(+2369+2393) |
| 1873 | CGTCAGGGTCAGGAGGCCTCAGGAC | SHANK3 H22 3UTR(+2372+2396) |
| 1874 | CAACGTCAGGGTCAGGAGGCCTCAG | SHANK3 H22 3UTR(+2375+2399) |
| 1875 | TCACAACGTCAGGGTCAGGAGGCCT | SHANK3 H22 3UTR(+2378+2402) |
| 1876 | GTATCACAACGTCAGGGTCAGGAGG | SHANK3 H22 3UTR(+2381+2405) |
| 1877 | GGCGTATCACAACGTCAGGGTCAGG | SHANK3 H22 3UTR(+2384+2408) |
| 1878 | TGGGGCGTATCACAACGTCAGGGTC | SHANK3 H22 3UTR(+2387+2411) |
| 1879 | CTGTGGGGCGTATCACAACGTCAGG | SHANK3 H22 3UTR(+2390+2414) |
| 1880 | TCTCTGTGGGGCGTATCACAACGTC | SHANK3 H22 3UTR(+2393+2417) |
| 1881 | AGATCTCTGTGGGGCGTATCACAAC | SHANK3 H22 3UTR(+2396+2420) |
| 1882 | CATAGATCTCTGTGGGGCGTATCAC | SHANK3 H22 3UTR(+2399+2423) |
| 1883 | AAACATAGATCTCTGTGGGGCGTAT | SHANK3 H22 3UTR(+2402+2426) |
| 1884 | AAGAAACATAGATCTCTGTGGGGCG | SHANK3 H22 3UTR(+2405+2429) |
| 1885 | TATAAGAAACATAGATCTCTGTGGG | SHANK3 H22 3UTR(+2408+2432) |
| 1886 | TAATATAAGAAACATAGATCTCTGT | SHANK3 H22 3UTR(+2411+2435) |
| 1887 | TAATAATATAAGAAACATAGATCTC | SHANK3 H22 3UTR(+2414+2438) |
| 1888 | TAATAATAATATAAGAAACATAGAT | SHANK3 H22 3UTR(+2417+2441) |
| 1889 | CAATAATAATAATATAAGAAACATA | SHANK3 H22 3UTR(+2420+2444) |
| 1890 | TATCAATAATAATAATATAAGAAAC | SHANK3 H22 3UTR(+2423+2447) |
| 1891 | TATTATCAATAATAATAATATAAGA | SHANK3 H22 3UTR(+2426+2450) |
| 1892 | AATTATTATCAATAATAATAATATA | SHANK3 H22 3UTR(+2429+2453) |
| 1893 | AATAATTATTATCAATAATAATAAT | SHANK3 H22 3UTR(+2432+2456) |
| 1894 | TATAATAATTATTATCAATAATAAT | SHANK3 H22 3UTR(+2435+2459) |
| 1895 | TATTATAATAATTATTATCAATAAT | SHANK3 H22 3UTR(+2438+2462) |
| 1896 | TAATATTATAATAATTATTATCAAT | SHANK3 H22 3UTR(+2441+2465) |
| 1897 | TAATAATATTATAATAATTATTATC | SHANK3 H22 3UTR(+2444+2468) |
| 1898 | ACATAATAATATTATAATAATTATT | SHANK3 H22 3UTR(+2447+2471) |
| 1899 | ATTACATAATAATATTATAATAATT | SHANK3 H22 3UTR(+2450+2474) |
| 1900 | TTTATTACATAATAATATTATAATA | SHANK3 H22 3UTR(+2453+2477) |
| 1901 | AAATTTATTACATAATAATATTATA | SHANK3 H22 3UTR(+2456+2480) |
| 1902 | TATAAATTTATTACATAATAATATT | SHANK3 H22 3UTR(+2459+2483) |
| 1903 | TCTTATAAATTTATTACATAATAAT | SHANK3 H22 3UTR(+2462+2486) |
| 1904 | ATTTCTTATAAATTTATTACATAAT | SHANK3 H22 3UTR(+2465+2489) |
| 1905 | TTCATTTCTTATAAATTTATTACAT | SHANK3 H22 3UTR(+2468+2492) |
| 1906 | GGCTTCATTTCTTATAAATTTATTA | SHANK3 H22 3UTR(+2471+2495) |
| 1907 | CATGGCTTCATTTCTTATAAATTTA | SHANK3 H22 3UTR(+2474+2498) |
| 1908 | AGCCATGGCTTCATTTCTTATAAAT | SHANK3 H22 3UTR(+2477+2501) |
| 1909 | CTGAGCCATGGCTTCATTTCTTATA | SHANK3 H22 3UTR(+2480+2504) |
| 1910 | CAACTGAGCCATGGCTTCATTTCTT | SHANK3 H22 3UTR(+2483+2507) |
| 1911 | AGGCAACTGAGCCATGGCTTCATTT | SHANK3 H22 3UTR(+2486+2510) |
| 1912 | AGCAGGCAACTGAGCCATGGCTTCA | SHANK3 H22 3UTR(+2489+2513) |
| 1913 | TCAAGCAGGCAACTGAGCCATGGCT | SHANK3 H22 3UTR(+2492+2516) |
| 1914 | CCCTCAAGCAGGCAACTGAGCCATG | SHANK3 H22 3UTR(+2495+2519) |
| 1915 | ATCCCCTCAAGCAGGCAACTGAGCC | SHANK3 H22 3UTR(+2498+2522) |
| 1916 | AATATCCCCTCAAGCAGGCAACTGA | SHANK3 H22 3UTR(+2501+2525) |
| 1917 | ACAAATATCCCCTCAAGCAGGCAAC | SHANK3 H22 3UTR(+2504+2528) |
| 1918 | GACACAAATATCCCCTCAAGCAGGC | SHANK3 H22 3UTR(+2507+2531) |
| 1919 | ACAGACACAAATATCCCCTCAAGCA | SHANK3 H22 3UTR(+2510+2534) |
| 1920 | GGGACAGACACAAATATCCCCTCAA | SHANK3 H22 3UTR(+2513+2537) |
| 1921 | TGAGGGACAGACACAAATATCCCCT | SHANK3 H22 3UTR(+2516+2540) |
| 1922 | GTATGAGGGACAGACACAAATATCC | SHANK3 H22 3UTR(+2519+2543) |
| 1923 | CCGGTATGAGGGACAGACACAAATA | SHANK3 H22 3UTR(+2522+2546) |
| 1924 | TGGCCGGTATGAGGGACAGACACAA | SHANK3 H22 3UTR(+2525+2549) |
| 1925 | GTGTGGCCGGTATGAGGGACAGACA | SHANK3 H22 3UTR(+2528+2552) |
| 1926 | TTTGTGTGGCCGGTATGAGGGACAG | SHANK3 H22 3UTR(+2531+2555) |
| 1927 | AGGTTTGTGTGGCCGGTATGAGGGA | SHANK3 H22 3UTR(+2534+2558) |
| 1928 | AGGAGGTTTGTGTGGCCGGTATGAG | SHANK3 H22 3UTR(+2537+2561) |
| 1929 | CTGAGGAGGTTTGTGTGGCCGGTAT | SHANK3 H22 3UTR(+2540+2564) |
| 1930 | AGGCTGAGGAGGTTTGTGTGGCCGG | SHANK3 H22 3UTR(+2543+2567) |
| 1931 | TCCAGGCTGAGGAGGTTTGTGTGGC | SHANK3 H22 3UTR(+2546+2570) |
| 1932 | TGGTCCAGGCTGAGGAGGTTTGTGT | SHANK3 H22 3UTR(+2549+2573) |
| 1933 | ATGTGGTCCAGGCTGAGGAGGTTTG | SHANK3 H22 3UTR(+2552+2576) |
| 1934 | CTGACCCTGTAAAGTTAAAACCCA | SHANK3_H3′UTR(+1697+1721) |
| 1935 | GGCATAAACTCGCCGCTTGTACTGA | SHANK3 H2A(+3+27)mm |
| 1936 | GGCATAAACTCGCCGCTTGTATCGA | SHANK3 H2A(+3+27) |
| 1937 | CTCCCCGTGATCCTTTACCTTTGTG | SHANK3 H2D(+7−18) |
| 1938 | CCTCCAGGTAGGGCAGGGGCGTGTC | SHANK3 H1D(+24−1) |
| 1939 | TTACCTCCAGGTAGGGCAGGGGCGT | SHANK3 H1D(+21−4) |
| 1940 | CACTTACCTCCAGGTAGGGCAGGGG | SHANK3 H1D(+18−7) |
| 1941 | GGCCACTTACCTCCAGGTAGGGCAG | SHANK3 H1D(+15−10) |
| 1942 | GCCGGCCACTTACCTCCAGGTAGGG | SHANK3 H1D(+12−13) |
| 1943 | CGCGCCGGCCACTTACCTCCAGGTA | SHANK3 H1D(+9−16) |
| 1944 | CCCCGCGCCGGCCACTTACCTCCAG | SHANK3 H1D(+6−19) |
| 1945 | CACCCCCGCGCCGGCCACTTACCTC | SHANK3 H1D(+3−22) |
| 1946 | GCTCACCCCCGCGCCGGCCACTTAC | SHANK3 H1D(−1−25) |
| 1947 | TCAGCTCACCCCCGCGCCGGCCACT | SHANK3 H1D(−4−28) |
| 1948 | TCCTCAGCTCACCCCCGCGCCGGCC | SHANK3 H1D(−7−31) |
| 1949 | CGCTCCTCAGCTCACCCCCGCGCCG | SHANK3 H1D(−10−34) |
| 1950 | GCGCGCTCCTCAGCTCACCCCCGCG | SHANK3 H1D(−13−37) |
| 1951 | CCTGCGCGCTCCTCAGCTCACCCCC | SHANK3 H1D(−16−40) |
| 1952 | CACCCTGCGCGCTCCTCAGCTCACC | SHANK3 H1D(−19−43) |
| 1953 | ATCCACCCTGCGCGCTCCTCAGCTC | SHANK3 H1D(−22−46) |
| 1954 | GTGATCCACCCTGCGCGCTCCTCAG | SHANK3 H1D(−25−49) |
| 1955 | TTGGTGATCCACCCTGCGCGCTCCT | SHANK3 H1D(−28−52) |
| 1956 | GGCTTGGTGATCCACCCTGCGCGCT | SHANK3 H1D(−31−55) |
| 1957 | CGGGGCTTGGTGATCCACCCTGCGC | SHANK3 H1D(−34−58) |
| 1958 | CCACGGGGCTTGGTGATCCACCCTG | SHANK3 H1D(−37−61) |
| 1959 | CCGCCACGGGGCTTGGTGATCCACC | SHANK3 H1D(−40−64) |
| 1960 | GTCCCGCCACGGGGCTTGGTGATCC | SHANK3 H1D(−43−67) |
| 1961 | CTGGTCCCGCCACGGGGCTTGGTGA | SHANK3 H1D(−46−70) |
| 1962 | TCACTGGTCCCGCCACGGGGCTTGG | SHANK3 H1D(−49−73) |
| 1963 | CCTTCACTGGTCCCGCCACGGGGCT | SHANK3 H1D(−52−76) |
| 1964 | TGCCCTTCACTGGTCCCGCCACGGG | SHANK3 H1D(−55−79) |
| 1965 | CCGTGCCCTTCACTGGTCCCGCCAC | SHANK3 H1D(−58−82) |
| 1966 | CTGCCGTGCCCTTCACTGGTCCCGC | SHANK3 H1D(−61−85) |
| 1967 | CCACTGCCGTGCCCTTCACTGGTCC | SHANK3 H1D(−64−88) |
| 1968 | TCCCCACTGCCGTGCCCTTCACTGG | SHANK3 H1D(−67−91) |
| 1969 | GTTTCCCCACTGCCGTGCCCTTCAC | SHANK3 H1D(−70−94) |
| 1970 | TGTGTTTCCCCACTGCCGTGCCCTT | SHANK3 H1D(−73−97) |
| 1971 | ACTTGTGTTTCCCCACTGCCGTGCC | SHANK3 H1D(−76−100) |
| 1972 | CCCACTTGTGTTTCCCCACTGCCGT | SHANK3 H1D(−79−103) |
| 1973 | CCTCCCACTTGTGTTTCCCCACTGC | SHANK3 H1D(−82−106) |
| 1974 | ACCCCTCCCACTTGTGTTTCCCCAC | SHANK3 H1D(−85−109) |
| 1975 | CTCACCCCTCCCACTTGTGTTTCCC | SHANK3 H1D(−88−112) |
| 1976 | CCCCTCACCCCTCCCACTTGTGTTT | SHANK3 H1D(−91−115) |
| 1977 | CCACCCCTCACCCCTCCCACTTGTG | SHANK3 H1D(−94−118) |
| 1978 | CCTCCACCCCTCACCCCTCCCACTT | SHANK3 H1D(−97−121) |
| 1979 | CAGCCTCCACCCCTCACCCCTCCCA | SHANK3 H1D(−100−124) |
| 1980 | ACACAGCCTCCACCCCTCACCCCTC | SHANK3 H1D(−103−127) |
| 1981 | CACACACAGCCTCCACCCCTCACCC | SHANK3 H1D(−106−130) |
| 1982 | ACACACACACAGCCTCCACCCCTCA | SHANK3 H1D(−109−133) |
| 1983 | CACACACACACACAGCCTCCACCCC | SHANK3 H1D(−112−136) |
| 1984 | ACACACACACACACACAGCCTCCAC | SHANK3 H1D(−115−139) |
| 1985 | CACACACACACACACACACAGCCTC | SHANK3 H1D(−118−142) |
| 1986 | ACACACACACACACACACACACAGC | SHANK3 H1D(−121−145) |
| 1987 | CACACACACACACACACACACACAC | SHANK3 H1D(−124−148) |
| 1988 | ACACACACACACACACACACACACA | SHANK3 H1D(−127−151) |
| 1989 | ACCACACACACACACACACACACAC | SHANK3 H1D(−130−154) |
| 1990 | AGCACCACACACACACACACACACA | SHANK3 H1D(−133−157) |
| 1991 | CACAGCACCACACACACACACACAC | SHANK3 H1D(−136−160) |
| 1992 | GCACACAGCACCACACACACACACA | SHANK3 H1D(−139−163) |
| 1993 | TCTGCACACAGCACCACACACACAC | SHANK3 H1D(−142−166) |
| 1994 | CACTCTGCACACAGCACCACACACA | SHANK3 H1D(−145−169) |
| 1995 | CTGCACTCTGCACACAGCACCACAC | SHANK3 H1D(−148−172) |
| 1996 | TCACTGCACTCTGCACACAGCACCA | SHANK3 H1D(−151−175) |
| 1997 | CGCTCACTGCACTCTGCACACAGCA | SHANK3 H1D(−154−178) |
| 1998 | ACACGCTCACTGCACTCTGCACACA | SHANK3 H1D(−157−181) |
| 1999 | TGTACACGCTCACTGCACTCTGCAC | SHANK3 H1D(−160−184) |
| 2000 | CCCTGTACACGCTCACTGCACTCTG | SHANK3 H1D(−163−187) |
| 2001 | GCACCCTGTACACGCTCACTGCACT | SHANK3 H1D(−166−190) |
| 2002 | GCTGCACCCTGTACACGCTCACTGC | SHANK3 H1D(−169−193) |
| 2003 | CTCGCTGCACCCTGTACACGCTCAC | SHANK3 H1D(−172−196) |
| 2004 | CCGCTCGCTGCACCCTGTACACGCT | SHANK3 H1D(−175−199) |
| 2005 | TGTCCGCTCGCTGCACCCTGTACAC | SHANK3 H1D(−178−202) |
| 2006 | CTGTGTCCGCTCGCTGCACCCTGTA | SHANK3 H1D(−181−205) |
| 2007 | ACACTGTGTCCGCTCGCTGCACCCT | SHANK3 H1D(−184−208) |
| 2008 | CATACACTGTGTCCGCTCGCTGCAC | SHANK3 H1D(−187−211) |
| 2009 | TCGCATACACTGTGTCCGCTCGCTG | SHANK3 H1D(−190−214) |
| 2010 | TCATCGCATACACTGTGTCCGCTCG | SHANK3 H1D(−193−217) |
| 2011 | TACTCATCGCATACACTGTGTCCGC | SHANK3 H1D(−196−220) |
| 2012 | GCCTACTCATCGCATACACTGTGTC | SHANK3 H1D(−199−223) |
| 2013 | CGCGCCTACTCATCGCATACACTGT | SHANK3 H1D(−202−226) |
| 2014 | CACCGCGCCTACTCATCGCATACAC | SHANK3 H1D(−205−229) |
| 2015 | ACACACCGCGCCTACTCATCGCATA | SHANK3 H1D(−208−232) |
| 2016 | TGCACACACCGCGCCTACTCATCGC | SHANK3 H1D(−211−235) |
| 2017 | CACTGCACACACCGCGCCTACTCAT | SHANK3 H1D(−214−238) |
| 2018 | GCTCACTGCACACACCGCGCCTACT | SHANK3 H1D(−217−241) |
| 2019 | CCCGCTCACTGCACACACCGCGCCT | SHANK3 H1D(−220−244) |
| 2020 | CTGCCCGCTCACTGCACACACCGCG | SHANK3 H1D(−223−247) |
| 2021 | GCCCTGCCCGCTCACTGCACACACC | SHANK3 H1D(−226−250) |
| 2022 | CTCGCCCTGCCCGCTCACTGCACAC | SHANK3 H1D(−229−253) |
| 2023 | CTGCTCGCCCTGCCCGCTCACTGCA | SHANK3 H1D(−232−256) |
| 2024 | TTACTGCTCGCCCTGCCCGCTCACT | SHANK3 H1D(−235−259) |
| 2025 | TCCTTACTGCTCGCCCTGCCCGCTC | SHANK3 H1D(−238−262) |
| 2026 | ACATCCTTACTGCTCGCCCTGCCCG | SHANK3 H1D(−241−265) |
| 2027 | TGTACATCCTTACTGCTCGCCCTGC | SHANK3 H1D(−244−268) |
| 2028 | CACTGTACATCCTTACTGCTCGCCC | SHANK3 H1D(−247−271) |
| 2029 | CCACACTGTACATCCTTACTGCTCG | SHANK3 H1D(−250−274) |
| 2030 | TGCCCACACTGTACATCCTTACTGC | SHANK3 H1D(−253−277) |
| 2031 | CACTGCCCACACTGTACATCCTTAC | SHANK3 H1D(−256−280) |
| 2032 | GCACACTGCCCACACTGTACATCCT | SHANK3 H1D(−259−283) |
| 2033 | CTCGCACACTGCCCACACTGTACAT | SHANK3 H1D(−262−286) |
| 2034 | ATGCTCGCACACTGCCCACACTGTA | SHANK3 H1D(−265−289) |
| 2035 | CACATGCTCGCACACTGCCCACACT | SHANK3 H1D(−268−292) |
| 2036 | CTACACATGCTCGCACACTGCCCAC | SHANK3 H1D(−271−295) |
| 2037 | TCACTACACATGCTCGCACACTGCC | SHANK3 H1D(−274−298) |
| 2038 | TGCTCACTACACATGCTCGCACACT | SHANK3 H1D(−277−301) |
| 2039 | GCCTGCTCACTACACATGCTCGCAC | SHANK3 H1D(−280−304) |
| 2040 | ACTGCCTGCTCACTACACATGCTCG | SHANK3 H1D(−283−307) |
| 2041 | CACACTGCCTGCTCACTACACATGC | SHANK3 H1D(−286−310) |
| 2042 | GCACACACTGCCTGCTCACTACACA | SHANK3 H1D(−289−313) |
| 2043 | ACTGCACACACTGCCTGCTCACTAC | SHANK3 H1D(−292−316) |
| 2044 | CTAACTGCACACACTGCCTGCTCAC | SHANK3 H1D(−295−319) |
| 2045 | CTGCTAACTGCACACACTGCCTGCT | SHANK3 H1D(−298−322) |
| 2046 | TGCCTGCTAACTGCACACACTGCCT | SHANK3 H1D(−301−325) |
| 2047 | CTGTGCCTGCTAACTGCACACACTG | SHANK3 H1D(−304−328) |
| 2048 | ACACTGTGCCTGCTAACTGCACACA | SHANK3 H1D(−307−331) |
| 2049 | CACACACTGTGCCTGCTAACTGCAC | SHANK3 H1D(−310−334) |
| 2050 | CTGCACACACTGTGCCTGCTAACTG | SHANK3 H1D(−313−337) |
| 2051 | TCACTGCACACACTGTGCCTGCTAA | SHANK3 H1D(−316−340) |
| 2052 | CACTCACTGCACACACTGTGCCTGC | SHANK3 H1D(−319−343) |
| 2053 | GCCCACTCACTGCACACACTGTGCC | SHANK3 H1D(−322−346) |
| 2054 | ACTGCCCACTCACTGCACACACTGT | SHANK3 H1D(−325−349) |
| 2055 | CACACTGCCCACTCACTGCACACAC | SHANK3 H1D(−328−352) |
| 2056 | GTGCACACTGCCCACTCACTGCACA | SHANK3 H1D(−331−355) |
| 2057 | GCTGTGCACACTGCCCACTCACTGC | SHANK3 H1D(−334−358) |
| 2058 | CATGCTGTGCACACTGCCCACTCAC | SHANK3 H1D(−337−361) |
| 2059 | GTACATGCTGTGCACACTGCCCACT | SHANK3 H1D(−340−364) |
| 2060 | ACTGTACATGCTGTGCACACTGCCC | SHANK3 H1D(−343−367) |
| 2061 | CACACTGTACATGCTGTGCACACTG | SHANK3 H1D(−346−370) |
| 2062 | ACTCACACTGTACATGCTGTGCACA | SHANK3 H1D(−349−373) |
| 2063 | ACCACTCACACTGTACATGCTGTGC | SHANK3 H1D(−352−376) |
| 2064 | CACACCACTCACACTGTACATGCTG | SHANK3 H1D(−355−379) |
| 2065 | GCACACACCACTCACACTGTACATG | SHANK3 H1D(−358−382) |
| 2066 | ACAGCACACACCACTCACACTGTAC | SHANK3 H1D(−361−385) |
| 2067 | CACACAGCACACACCACTCACACTG | SHANK3 H1D(−364−388) |
| 2068 | CGGCACACAGCACACACCACTCACA | SHANK3 H1D(−367−391) |
| 2069 | TCACGGCACACAGCACACACCACTC | SHANK3 H1D(−370−394) |
| 2070 | TGCTCACGGCACACAGCACACACCA | SHANK3 H1D(−373−397) |
| 2071 | ACATGCTCACGGCACACAGCACACA | SHANK3 H1D(−376−400) |
| 2072 | TACACATGCTCACGGCACACAGCAC | SHANK3 H1D(−379−403) |
| 2073 | CACTACACATGCTCACGGCACACAG | SHANK3 H1D(−382−406) |
| 2074 | ACTCACTACACATGCTCACGGCACA | SHANK3 H1D(−385−409) |
| 2075 | CCCACTCACTACACATGCTCACGGC | SHANK3 H1D(−388−412) |
| 2076 | GTGCCCACTCACTACACATGCTCAC | SHANK3 H1D(−391−415) |
| 2077 | ACTGTGCCCACTCACTACACATGCT | SHANK3 H1D(−394−418) |
| 2078 | TGCACTGTGCCCACTCACTACACAT | SHANK3 H1D(−397−421) |
| 2079 | GACTGCACTGTGCCCACTCACTACA | SHANK3 H1D(−400−424) |
| 2080 | ACTGACTGCACTGTGCCCACTCACT | SHANK3 H1D(−403−427) |
| 2081 | CACACTGACTGCACTGTGCCCACTC | SHANK3 H1D(−406−430) |
| 2082 | GTGCACACTGACTGCACTGTGCCCA | SHANK3 H1D(−409−433) |
| 2083 | TTTGTGCACACTGACTGCACTGTGC | SHANK3 H1D(−412−436) |
| 2084 | TACTTTGTGCACACTGACTGCACTG | SHANK3 H1D(−415−439) |
| 2085 | GCATACTTTGTGCACACTGACTGCA | SHANK3 H1D(−418−442) |
| 2086 | ACTGCATACTTTGTGCACACTGACT | SHANK3 H1D(−421−445) |
| 2087 | CCCACTGCATACTTTGTGCACACTG | SHANK3 H1D(−424−448) |
| 2088 | GTGCCCACTGCATACTTTGTGCACA | SHANK3 H1D(−427−451) |
| 2089 | TACGTGCCCACTGCATACTTTGTGC | SHANK3 H1D(−430−454) |
| 2090 | CGGTACGTGCCCACTGCATACTTTG | SHANK3 H1D(−433−457) |
| 2091 | ACACGGTACGTGCCCACTGCATACT | SHANK3 H1D(−436−460) |
| 2092 | CACACACGGTACGTGCCCACTGCAT | SHANK3 H1D(−439−463) |
| 2093 | CTGCACACACGGTACGTGCCCACTG | SHANK3 H1D(−442−466) |
| 2094 | TCACTGCACACACGGTACGTGCCCA | SHANK3 H1D(−445−469) |
| 2095 | CACTCACTGCACACACGGTACGTGC | SHANK3 H1D(−448−472) |
| 2096 | CACCACTCACTGCACACACGGTACG | SHANK3 H1D(−451−475) |
| 2097 | GCACACCACTCACTGCACACACGGT | SHANK3 H1D(−454−478) |
| 2098 | ACTGCACACCACTCACTGCACACAC | SHANK3 H1D(−457−481) |
| 2099 | CACACTGCACACCACTCACTGCACA | SHANK3 H1D(−460−484) |
| 2100 | CTACACACTGCACACCACTCACTGC | SHANK3 H1D(−463−487) |
| 2101 | ACACTACACACTGCACACCACTCAC | SHANK3 H1D(−466−490) |
| 2102 | TGCACACTACACACTGCACACCACT | SHANK3 H1D(−469−493) |
| 2103 | CACTGCACACTACACACTGCACACC | SHANK3 H1D(−472−496) |
| 2104 | GCTCACTGCACACTACACACTGCAC | SHANK3 H1D(−475−499) |
| 2105 | ACTGCTCACTGCACACTACACACTG | SHANK3 H1D(−478−502) |
| 2106 | CACACTGCTCACTGCACACTACACA | SHANK3 H1D(−481−505) |
| 2107 | GTACACACTGCTCACTGCACACTAC | SHANK3 H1D(−484−508) |
| 2108 | GCTGTACACACTGCTCACTGCACAC | SHANK3 H1D(−487−511) |
| 2109 | AATGCTGTACACACTGCTCACTGCA | SHANK3 H1D(−490−514) |
| 2110 | TGCAATGCTGTACACACTGCTCACT | SHANK3 H1D(−493−517) |
| 2111 | CACTGCAATGCTGTACACACTGCTC | SHANK3 H1D(−496−520) |
| 2112 | CCACACTGCAATGCTGTACACACTG | SHANK3 H1D(−499−523) |
| 2113 | GCCCCACACTGCAATGCTGTACACA | SHANK3 H1D(−502−526) |
| 2114 | ACTGCCCCACACTGCAATGCTGTAC | SHANK3 H1D(−505−529) |
| 2115 | CTCACTGCCCCACACTGCAATGCTG | SHANK3 H1D(−508−532) |
| 2116 | GTACTCACTGCCCCACACTGCAATG | SHANK3 H1D(−511−535) |
| 2117 | ACTGTACTCACTGCCCCACACTGCA | SHANK3 H1D(−514−538) |
| 2118 | CACACTGTACTCACTGCCCCACACT | SHANK3 H1D(−517−541) |
| 2119 | ACTCACACTGTACTCACTGCCCCAC | SHANK3 H1D(−520−544) |
| 2120 | AACACTCACACTGTACTCACTGCCC | SHANK3 H1D(−523−547) |
| 2121 | CACAACACTCACACTGTACTCACTG | SHANK3 H1D(−526−550) |
| 2122 | AATCACAACACTCACACTGTACTCA | SHANK3 H1D(−529−553) |
| 2123 | CACAATCACAACACTCACACTGTAC | SHANK3 H1D(−532−556) |
| 2124 | TACCACAATCACAACACTCACACTG | SHANK3 H1D(−535−559) |
| 2125 | GCTTACCACAATCACAACACTCACA | SHANK3 H1D(−538−562) |
| 2126 | CCTGCTTACCACAATCACAACACTC | SHANK3 H1D(−541−565) |
| 2127 | CATCCTGCTTACCACAATCACAACA | SHANK3 H1D(−544−568) |
| 2128 | GCGCATCCTGCTTACCACAATCACA | SHANK3 H1D(−547−571) |
| 2129 | ACTGCGCATCCTGCTTACCACAATC | SHANK3 H1D(−550−574) |
| 2130 | TATACTGCGCATCCTGCTTACCACA | SHANK3 H1D(−553−577) |
| 2131 | CTGTATACTGCGCATCCTGCTTACC | SHANK3 H1D(−556−580) |
| 2132 | TCACTGTATACTGCGCATCCTGCTT | SHANK3 H1D(−559−583) |
| 2133 | TGTTCACTGTATACTGCGCATCCTG | SHANK3 H1D(−562−586) |
| 2134 | CACTGTTCACTGTATACTGCGCATC | SHANK3 H1D(−565−589) |
| 2135 | GCACACTGTTCACTGTATACTGCGC | SHANK3 H1D(−568−592) |
| 2136 | TGTGCACACTGTTCACTGTATACTG | SHANK3 H1D(−571−595) |
| 2137 | CACTGTGCACACTGTTCACTGTATA | SHANK3 H1D(−574−598) |
| 2138 | ACACACTGTGCACACTGTTCACTGT | SHANK3 H1D(−577−601) |
| 2139 | TGCACACACTGTGCACACTGTTCAC | SHANK3 H1D(−580−604) |
| 2140 | CACTGCACACACTGTGCACACTGTT | SHANK3 H1D(−583−607) |
| 2141 | CCACACTGCACACACTGTGCACACT | SHANK3 H1D(−586−610) |
| 2142 | AGCCCACACTGCACACACTGTGCAC | SHANK3 H1D(−589−613) |
| 2143 | CACAGCCCACACTGCACACACTGTG | SHANK3 H1D(−592−616) |
| 2144 | GCACACAGCCCACACTGCACACACT | SHANK3 H1D(−595−619) |
| 2145 | GTGGCACACAGCCCACACTGCACAC | SHANK3 H1D(−598−622) |
| 2146 | TCTGTGGCACACAGCCCACACTGCA | SHANK3 H1D(−601−625) |
| 2147 | GACTCTGTGGCACACAGCCCACACT | SHANK3 H1D(−604−628) |
| 2148 | ACTGACTCTGTGGCACACAGCCCAC | SHANK3 H1D(−607−631) |
| 2149 | CACACTGACTCTGTGGCACACAGCC | SHANK3 H1D(−610−634) |
| 2150 | CACCACACTGACTCTGTGGCACACA | SHANK3 H1D(−613−637) |
| 2151 | ACACACCACACTGACTCTGTGGCAC | SHANK3 H1D(−616−640) |
| 2152 | TACACACACCACACTGACTCTGTGG | SHANK3 H1D(−619−643) |
| 2153 | AACTACACACACCACACTGACTCTG | SHANK3 H1D(−622−646) |
| 2154 | TCAAACTACACACACCACACTGACT | SHANK3 H1D(−625−649) |
| 2155 | TGTTCAAACTACACACACCACACTG | SHANK3 H1D(−628−652) |
| 2156 | CACTGTTCAAACTACACACACCACA | SHANK3 H1D(−631−655) |
| 2157 | ACACACTGTTCAAACTACACACACC | SHANK3 H1D(−634−658) |
| 2158 | TGCACACACTGTTCAAACTACACAC | SHANK3 H1D(−637−661) |
| 2159 | CAATGCACACACTGTTCAAACTACA | SHANK3 H1D(−640−664) |
| 2160 | GCTCAATGCACACACTGTTCAAACT | SHANK3 H1D(−643−667) |
| 2161 | GCTGCTCAATGCACACACTGTTCAA | SHANK3 H1D(−646−670) |
| 2162 | CATGCTGCTCAATGCACACACTGTT | SHANK3 H1D(−649−673) |
| 2163 | ATCCATGCTGCTCAATGCACACACT | SHANK3 H1D(−652−676) |
| 2164 | ACCATCCATGCTGCTCAATGCACAC | SHANK3 H1D(−655−679) |
| 2165 | CACACCATCCATGCTGCTCAATGCA | SHANK3 H1D(−658−682) |
| 2166 | GTCCACACCATCCATGCTGCTCAAT | SHANK3 H1D(−661−685) |
| 2167 | AGCGTCCACACCATCCATGCTGCTC | SHANK3 H1D(−664−688) |
| 2168 | CTCAGCGTCCACACCATCCATGCTG | SHANK3 H1D(−667−691) |
| 2169 | ATGCTCAGCGTCCACACCATCCATG | SHANK3 H1D(−670−694) |
| 2170 | ACAATGCTCAGCGTCCACACCATCC | SHANK3 H1D(−673−697) |
| 2171 | AGAACAATGCTCAGCGTCCACACCA | SHANK3 H1D(−676−700) |
| 2172 | TGGAGAACAATGCTCAGCGTCCACA | SHANK3 H1D(−679−703) |
| 2173 | CCCTGGAGAACAATGCTCAGCGTCC | SHANK3 H1D(−682−706) |
| 2174 | CCTCCCTGGAGAACAATGCTCAGCG | SHANK3 H2A(−686−662) |
| 2175 | ACTCCTCCCTGGAGAACAATGCTCA | SHANK3 H2A(−683−659) |
| 2176 | CACACTCCTCCCTGGAGAACAATGC | SHANK3 H2A(−680−656) |
| 2177 | GCTCACACTCCTCCCTGGAGAACAA | SHANK3 H2A(−677−653) |
| 2178 | CGTGCTCACACTCCTCCCTGGAGAA | SHANK3 H2A(−674−650) |
| 2179 | TCTCGTGCTCACACTCCTCCCTGGA | SHANK3 H2A(−671−647) |
| 2180 | CTCTCTCGTGCTCACACTCCTCCCT | SHANK3 H2A(−668−644) |
| 2181 | GCACTCTCTCGTGCTCACACTCCTC | SHANK3 H2A(−665−641) |
| 2182 | CTGGCACTCTCTCGTGCTCACACTC | SHANK3 H2A(−662−638) |
| 2183 | CCTCTGGCACTCTCTCGTGCTCACA | SHANK3 H2A(−659−635) |
| 2184 | ACCCCTCTGGCACTCTCTCGTGCTC | SHANK3 H2A(−656−632) |
| 2185 | CACACCCCTCTGGCACTCTCTCGTG | SHANK3 H2A(−653−629) |
| 2186 | CCACACACCCCTCTGGCACTCTCTC | SHANK3 H2A(−650−626) |
| 2187 | ACACCACACACCCCTCTGGCACTCT | SHANK3 H2A(−647−623) |
| 2188 | CTCACACCACACACCCCTCTGGCAC | SHANK3 H2A(−644−620) |
| 2189 | CTGCTCACACCACACACCCCTCTGG | SHANK3 H2A(−641−617) |
| 2190 | GCCCTGCTCACACCACACACCCCTC | SHANK3 H2A(−638−614) |
| 2191 | ATAGCCCTGCTCACACCACACACCC | SHANK3 H2A(−635−611) |
| 2192 | CAGATAGCCCTGCTCACACCACACA | SHANK3 H2A(−632−608) |
| 2193 | ACACAGATAGCCCTGCTCACACCAC | SHANK3 H2A(−629−605) |
| 2194 | TGCACACAGATAGCCCTGCTCACAC | SHANK3 H2A(−626−602) |
| 2195 | ACGTGCACACAGATAGCCCTGCTCA | SHANK3 H2A(−623−599) |
| 2196 | CAAACGTGCACACAGATAGCCCTGC | SHANK3 H2A(−620−596) |
| 2197 | GAACAAACGTGCACACAGATAGCCC | SHANK3 H2A(−617−593) |
| 2198 | AAGGAACAAACGTGCACACAGATAG | SHANK3 H2A(−614−590) |
| 2199 | AGAAAGGAACAAACGTGCACACAGA | SHANK3 H2A(−611−587) |
| 2200 | TGGAGAAAGGAACAAACGTGCACAC | SHANK3 H2A(−608−584) |
| 2201 | AGCTGGAGAAAGGAACAAACGTGCA | SHANK3 H2A(−605−581) |
| 2202 | CACAGCTGGAGAAAGGAACAAACGT | SHANK3 H2A(−602−578) |
| 2203 | CTTCACAGCTGGAGAAAGGAACAAA | SHANK3 H2A(−599−575) |
| 2204 | AGACTTCACAGCTGGAGAAAGGAAC | SHANK3 H2A(−596−572) |
| 2205 | ACAAGACTTCACAGCTGGAGAAAGG | SHANK3 H2A(−593−569) |
| 2206 | TTCACAAGACTTCACAGCTGGAGAA | SHANK3 H2A(−590−566) |
| 2207 | GCCTTCACAAGACTTCACAGCTGGA | SHANK3 H2A(−587−563) |
| 2208 | TTGGCCTTCACAAGACTTCACAGCT | SHANK3 H2A(−584−560) |
| 2209 | TGGTTGGCCTTCACAAGACTTCACA | SHANK3 H2A(−581−557) |
| 2210 | ACTTGGTTGGCCTTCACAAGACTTC | SHANK3 H2A(−578−554) |
| 2211 | GGGACTTGGTTGGCCTTCACAAGAC | SHANK3 H2A(−575−551) |
| 2212 | GAGGGGACTTGGTTGGCCTTCACAA | SHANK3 H2A(−572−548) |
| 2213 | AAGGAGGGGACTTGGTTGGCCTTCA | SHANK3 H2A(−569−545) |
| 2214 | GTAAAGGAGGGGACTTGGTTGGCCT | SHANK3 H2A(−566−542) |
| 2215 | TGAGTAAAGGAGGGGACTTGGTTGG | SHANK3 H2A(−563−539) |
| 2216 | GGGTGAGTAAAGGAGGGGACTTGGT | SHANK3 H2A(−560−536) |
| 2217 | CATGGGTGAGTAAAGGAGGGGACTT | SHANK3 H2A(−557−533) |
| 2218 | ATGCATGGGTGAGTAAAGGAGGGGA | SHANK3 H2A(−554−530) |
| 2219 | ACCATGCATGGGTGAGTAAAGGAGG | SHANK3 H2A(−551−527) |
| 2220 | CACACCATGCATGGGTGAGTAAAGG | SHANK3 H2A(−548−524) |
| 2221 | CTTCACACCATGCATGGGTGAGTAA | SHANK3 H2A(−545−521) |
| 2222 | CATCTTCACACCATGCATGGGTGAG | SHANK3 H2A(−542−518) |
| 2223 | ATACATCTTCACACCATGCATGGGT | SHANK3 H2A(−539−515) |
| 2224 | TCAATACATCTTCACACCATGCATG | SHANK3 H2A(−536−512) |
| 2225 | CACTCAATACATCTTCACACCATGC | SHANK3 H2A(−533−509) |
| 2226 | AGGCACTCAATACATCTTCACACCA | SHANK3 H2A(−530−506) |
| 2227 | GCAAGGCACTCAATACATCTTCACA | SHANK3 H2A(−527−503) |
| 2228 | CTAGCAAGGCACTCAATACATCTTC | SHANK3 H2A(−524−500) |
| 2229 | TGCCTAGCAAGGCACTCAATACATC | SHANK3 H2A(−521−497) |
| 2230 | CCATGCCTAGCAAGGCACTCAATAC | SHANK3 H2A(−518−494) |
| 2231 | TCCCCATGCCTAGCAAGGCACTCAA | SHANK3 H2A(−515−491) |
| 2232 | ACGTCCCCATGCCTAGCAAGGCACT | SHANK3 H2A(−512−488) |
| 2233 | TCTACGTCCCCATGCCTAGCAAGGC | SHANK3 H2A(−509−485) |
| 2234 | CCGTCTACGTCCCCATGCCTAGCAA | SHANK3 H2A(−506−482) |
| 2235 | ACCCCGTCTACGTCCCCATGCCTAG | SHANK3 H2A(−503−479) |
| 2236 | CTGACCCCGTCTACGTCCCCATGCC | SHANK3 H2A(−500−476) |
| 2237 | GGACTGACCCCGTCTACGTCCCCAT | SHANK3 H2A(−497−473) |
| 2238 | ACAGGACTGACCCCGTCTACGTCCC | SHANK3 H2A(−494−470) |
| 2239 | TCCACAGGACTGACCCCGTCTACGT | SHANK3 H2A(−491−467) |
| 2240 | CCTTCCACAGGACTGACCCCGTCTA | SHANK3 H2A(−488−464) |
| 2241 | AGACCTTCCACAGGACTGACCCCGT | SHANK3 H2A(−485−461) |
| 2242 | CCAAGACCTTCCACAGGACTGACCC | SHANK3 H2A(−482−458) |
| 2243 | ACTCCAAGACCTTCCACAGGACTGA | SHANK3 H2A(−479−455) |
| 2244 | CAAACTCCAAGACCTTCCACAGGAC | SHANK3 H2A(−476−452) |
| 2245 | TGCCAAACTCCAAGACCTTCCACAG | SHANK3 H2A(−473−449) |
| 2246 | CCCTGCCAAACTCCAAGACCTTCCA | SHANK3 H2A(−470−446) |
| 2247 | CCACCCTGCCAAACTCCAAGACCTT | SHANK3 H2A(−467−443) |
| 2248 | CCTCCACCCTGCCAAACTCCAAGAC | SHANK3 H2A(−464−440) |
| 2249 | CCCCCTCCACCCTGCCAAACTCCAA | SHANK3 H2A(−461−437) |
| 2250 | CACCCCCCTCCACCCTGCCAAACTC | SHANK3 H2A(−458−434) |
| 2251 | GGGCACCCCCCTCCACCCTGCCAAA | SHANK3 H2A(−455−431) |
| 2252 | GTTGGGCACCCCCCTCCACCCTGCC | SHANK3 H2A(−452−428) |
| 2253 | GCAGTTGGGCACCCCCCTCCACCCT | SHANK3 H2A(−449−425) |
| 2254 | ACTGCAGTTGGGCACCCCCCTCCAC | SHANK3 H2A(−446−422) |
| 2255 | CCCACTGCAGTTGGGCACCCCCCTC | SHANK3 H2A(−443−419) |
| 2256 | ATGCCCACTGCAGTTGGGCACCCCC | SHANK3 H2A(−440−416) |
| 2257 | TCAATGCCCACTGCAGTTGGGCACC | SHANK3 H2A(−437−413) |
| 2258 | TATTCAATGCCCACTGCAGTTGGGC | SHANK3 H2A(−434−410) |
| 2259 | CTTTATTCAATGCCCACTGCAGTTG | SHANK3 H2A(−431−407) |
| 2260 | AATCTTTATTCAATGCCCACTGCAG | SHANK3 H2A(−428−404) |
| 2261 | CAAAATCTTTATTCAATGCCCACTG | SHANK3 H2A(−425−401) |
| 2262 | TCACAAAATCTTTATTCAATGCCCA | SHANK3 H2A(−422−398) |
| 2263 | AGCTCACAAAATCTTTATTCAATGC | SHANK3 H2A(−419−395) |
| 2264 | CTCAGCTCACAAAATCTTTATTCAA | SHANK3 H2A(−416−392) |
| 2265 | GAGCTCAGCTCACAAAATCTTTATT | SHANK3 H2A(−413−389) |
| 2266 | TTTGAGCTCAGCTCACAAAATCTTT | SHANK3 H2A(−410−386) |
| 2267 | AACTTTGAGCTCAGCTCACAAAATC | SHANK3 H2A(−407−383) |
| 2268 | CCCAACTTTGAGCTCAGCTCACAAA | SHANK3 H2A(−404−380) |
| 2269 | CCGCCCAACTTTGAGCTCAGCTCAC | SHANK3 H2A(−401−377) |
| 2270 | GGCCCGCCCAACTTTGAGCTCAGCT | SHANK3 H2A(−398−374) |
| 2271 | GGAGGCCCGCCCAACTTTGAGCTCA | SHANK3 H2A(−395−371) |
| 2272 | CACGGAGGCCCGCCCAACTTTGAGC | SHANK3 H2A(−392−368) |
| 2273 | CACCACGGAGGCCCGCCCAACTTTG | SHANK3 H2A(−389−365) |
| 2274 | TGCCACCACGGAGGCCCGCCCAACT | SHANK3 H2A(−386−362) |
| 2275 | CTGTGCCACCACGGAGGCCCGCCCA | SHANK3 H2A(−383−359) |
| 2276 | TTCCTGTGCCACCACGGAGGCCCGC | SHANK3 H2A(−380−356) |
| 2277 | ACTTTCCTGTGCCACCACGGAGGCC | SHANK3 H2A(−377−353) |
| 2278 | CCCACTTTCCTGTGCCACCACGGAG | SHANK3 H2A(−374−350) |
| 2279 | GACCCCACTTTCCTGTGCCACCACG | SHANK3 H2A(−371−347) |
| 2280 | ACTGACCCCACTTTCCTGTGCCACC | SHANK3 H2A(−368−344) |
| 2281 | AGAACTGACCCCACTTTCCTGTGCC | SHANK3 H2A(−365−341) |
| 2282 | GGTAGAACTGACCCCACTTTCCTGT | SHANK3 H2A(−362−338) |
| 2283 | CCAGGTAGAACTGACCCCACTTTCC | SHANK3 H2A(−359−335) |
| 2284 | TCTCCAGGTAGAACTGACCCCACTT | SHANK3 H2A(−356−332) |
| 2285 | CACTCTCCAGGTAGAACTGACCCCA | SHANK3 H2A(−353−329) |
| 2286 | CTCCACTCTCCAGGTAGAACTGACC | SHANK3 H2A(−350−326) |
| 2287 | CCCCTCCACTCTCCAGGTAGAACTG | SHANK3 H2A(−347−323) |
| 2288 | ATCCCCCTCCACTCTCCAGGTAGAA | SHANK3 H2A(−344−320) |
| 2289 | GACATCCCCCTCCACTCTCCAGGTA | SHANK3 H2A(−341−317) |
| 2290 | CTAGACATCCCCCTCCACTCTCCAG | SHANK3 H2A(−338−314) |
| 2291 | ATCCTAGACATCCCCCTCCACTCTC | SHANK3 H2A(−335−311) |
| 2292 | ACCATCCTAGACATCCCCCTCCACT | SHANK3 H2A(−332−308) |
| 2293 | CTTACCATCCTAGACATCCCCCTCC | SHANK3 H2A(−329−305) |
| 2294 | AGGCTTACCATCCTAGACATCCCCC | SHANK3 H2A(−326−302) |
| 2295 | TCCAGGCTTACCATCCTAGACATCC | SHANK3 H2A(−323−299) |
| 2296 | GATTCCAGGCTTACCATCCTAGACA | SHANK3 H2A(−320−296) |
| 2297 | CCTGATTCCAGGCTTACCATCCTAG | SHANK3 H2A(−317−293) |
| 2298 | AGGCCTGATTCCAGGCTTACCATCC | SHANK3 H2A(−314−290) |
| 2299 | TGAAGGCCTGATTCCAGGCTTACCA | SHANK3 H2A(−311−287) |
| 2300 | CTCTGAAGGCCTGATTCCAGGCTTA | SHANK3 H2A(−308−284) |
| 2301 | CCTCTCTGAAGGCCTGATTCCAGGC | SHANK3 H2A(−305−281) |
| 2302 | ACTCCTCTCTGAAGGCCTGATTCCA | SHANK3 H2A(−302−278) |
| 2303 | CCCACTCCTCTCTGAAGGCCTGATT | SHANK3 H2A(−299−275) |
| 2304 | AATCCCACTCCTCTCTGAAGGCCTG | SHANK3 H2A(−296−272) |
| 2305 | CAAAATCCCACTCCTCTCTGAAGGC | SHANK3 H2A(−293−269) |
| 2306 | CGGCAAAATCCCACTCCTCTCTGAA | SHANK3 H2A(−290−266) |
| 2307 | TCTCGGCAAAATCCCACTCCTCTCT | SHANK3 H2A(−287−263) |
| 2308 | GATTCTCGGCAAAATCCCACTCCTC | SHANK3 H2A(−284−260) |
| 2309 | CAGGATTCTCGGCAAAATCCCACTC | SHANK3 H2A(−281−257) |
| 2310 | CCCCAGGATTCTCGGCAAAATCCCA | SHANK3 H2A(−278−254) |
| 2311 | CATCCCCAGGATTCTCGGCAAAATC | SHANK3 H2A(−275−251) |
| 2312 | TCCCATCCCCAGGATTCTCGGCAAA | SHANK3 H2A(−272−248) |
| 2313 | CCTTCCCATCCCCAGGATTCTCGGC | SHANK3 H2A(−269−245) |
| 2314 | TCGCCTTCCCATCCCCAGGATTCTC | SHANK3 H2A(−266−242) |
| 2315 | CCGTCGCCTTCCCATCCCCAGGATT | SHANK3 H2A(−263−239) |
| 2316 | GTCCCGTCGCCTTCCCATCCCCAGG | SHANK3 H2A(−260−236) |
| 2317 | ACTGTCCCGTCGCCTTCCCATCCCC | SHANK3 H2A(−257−233) |
| 2318 | TGCACTGTCCCGTCGCCTTCCCATC | SHANK3 H2A(−254−230) |
| 2319 | GCCTGCACTGTCCCGTCGCCTTCCC | SHANK3 H2A(−251−227) |
| 2320 | GCAGCCTGCACTGTCCCGTCGCCTT | SHANK3 H2A(−248−224) |
| 2321 | CCCGCAGCCTGCACTGTCCCGTCGC | SHANK3 H2A(−245−221) |
| 2322 | CTGCCCGCAGCCTGCACTGTCCCGT | SHANK3 H2A(−242−218) |
| 2323 | TAGCTGCCCGCAGCCTGCACTGTCC | SHANK3 H2A(−239−215) |
| 2324 | GCCTAGCTGCCCGCAGCCTGCACTG | SHANK3 H2A(−236−212) |
| 2325 | CGTGCCTAGCTGCCCGCAGCCTGCA | SHANK3 H2A(−233−209) |
| 2326 | GCACGTGCCTAGCTGCCCGCAGCCT | SHANK3 H2A(−230−206) |
| 2327 | AAGGCACGTGCCTAGCTGCCCGCAG | SHANK3 H2A(−227−203) |
| 2328 | TGGAAGGCACGTGCCTAGCTGCCCG | SHANK3 H2A(−224−200) |
| 2329 | CGATGGAAGGCACGTGCCTAGCTGC | SHANK3 H2A(−221−197) |
| 2330 | GCCCGATGGAAGGCACGTGCCTAGC | SHANK3 H2A(−218−194) |
| 2331 | GCAGCCCGATGGAAGGCACGTGCCT | SHANK3 H2A(−215−191) |
| 2332 | GATGCAGCCCGATGGAAGGCACGTG | SHANK3 H2A(−212−188) |
| 2333 | CATGATGCAGCCCGATGGAAGGCAC | SHANK3 H2A(−209−185) |
| 2334 | AGGCATGATGCAGCCCGATGGAAGG | SHANK3 H2A(−206−182) |
| 2335 | CTCAGGCATGATGCAGCCCGATGGA | SHANK3 H2A(−203−179) |
| 2336 | ACACTCAGGCATGATGCAGCCCGAT | SHANK3 H2A(−200−176) |
| 2337 | ACCACACTCAGGCATGATGCAGCCC | SHANK3 H2A(−197−173) |
| 2338 | CCCACCACACTCAGGCATGATGCAG | SHANK3 H2A(−194−170) |
| 2339 | GCACCCACCACACTCAGGCATGATG | SHANK3 H2A(−191−167) |
| 2340 | CATGCACCCACCACACTCAGGCATG | SHANK3 H2A(−188−164) |
| 2341 | TGCCATGCACCCACCACACTCAGGC | SHANK3 H2A(−185−161) |
| 2342 | AGCTGCCATGCACCCACCACACTCA | SHANK3 H2A(−182−158) |
| 2343 | AACAGCTGCCATGCACCCACCACAC | SHANK3 H2A(−179−155) |
| 2344 | GCTAACAGCTGCCATGCACCCACCA | SHANK3 H2A(−176−152) |
| 2345 | AGAGCTAACAGCTGCCATGCACCCA | SHANK3 H2A(−173−149) |
| 2346 | GACAGAGCTAACAGCTGCCATGCAC | SHANK3 H2A(−170−146) |
| 2347 | GTGGACAGAGCTAACAGCTGCCATG | SHANK3 H2A(−167−143) |
| 2348 | ACAGTGGACAGAGCTAACAGCTGCC | SHANK3 H2A(−164−140) |
| 2349 | ACCACAGTGGACAGAGCTAACAGCT | SHANK3 H2A(−161−137) |
| 2350 | ACTACCACAGTGGACAGAGCTAACA | SHANK3 H2A(−158−134) |
| 2351 | CATACTACCACAGTGGACAGAGCTA | SHANK3 H2A(−155−131) |
| 2352 | AGTCATACTACCACAGTGGACAGAG | SHANK3 H2A(−152−128) |
| 2353 | ATCAGTCATACTACCACAGTGGACA | SHANK3 H2A(−149−125) |
| 2354 | ACCATCAGTCATACTACCACAGTGG | SHANK3 H2A(−146−122) |
| 2355 | CACACCATCAGTCATACTACCACAG | SHANK3 H2A(−143−119) |
| 2356 | GTACACACCATCAGTCATACTACCA | SHANK3 H2A(−140−116) |
| 2357 | CCTGTACACACCATCAGTCATACTA | SHANK3 H2A(−137−113) |
| 2358 | CCTCCTGTACACACCATCAGTCATA | SHANK3 H2A(−134−110) |
| 2359 | TGCCCTCCTGTACACACCATCAGTC | SHANK3 H2A(−131−107) |
| 2360 | CACTGCCCTCCTGTACACACCATCA | SHANK3 H2A(−128−104) |
| 2361 | CCTCACTGCCCTCCTGTACACACCA | SHANK3 H2A(−125−101) |
| 2362 | ACCCCTCACTGCCCTCCTGTACACA | SHANK3 H2A(−122−98) |
| 2363 | CGCACCCCTCACTGCCCTCCTGTAC | SHANK3 H2A(−119−95) |
| 2364 | CACCGCACCCCTCACTGCCCTCCTG | SHANK3 H2A(−116−92) |
| 2365 | CCACACCGCACCCCTCACTGCCCTC | SHANK3 H2A(−113−89) |
| 2366 | TGGCCACACCGCACCCCTCACTGCC | SHANK3 H2A(−110−86) |
| 2367 | TGCTGGCCACACCGCACCCCTCACT | SHANK3 H2A(−107−83) |
| 2368 | TCATGCTGGCCACACCGCACCCCTC | SHANK3 H2A(−104−80) |
| 2369 | CGCTCATGCTGGCCACACCGCACCC | SHANK3 H2A(−101−77) |
| 2370 | TCCCGCTCATGCTGGCCACACCGCA | SHANK3 H2A(−98−74) |
| 2371 | CCGTCCCGCTCATGCTGGCCACACC | SHANK3 H2A(−95−71) |
| 2372 | ACCCCGTCCCGCTCATGCTGGCCAC | SHANK3 H2A(−92−68) |
| 2373 | CAAACCCCGTCCCGCTCATGCTGGC | SHANK3 H2A(−89−65) |
| 2374 | GCACAAACCCCGTCCCGCTCATGCT | SHANK3 H2A(−86−62) |
| 2375 | CATGCACAAACCCCGTCCCGCTCAT | SHANK3 H2A(−83−59) |
| 2376 | GTCCATGCACAAACCCCGTCCCGCT | SHANK3 H2A(−80−56) |
| 2377 | TGAGTCCATGCACAAACCCCGTCCC | SHANK3 H2A(−77−53) |
| 2378 | AAGTGAGTCCATGCACAAACCCCGT | SHANK3 H2A(−74−50) |
| 2379 | AGCAAGTGAGTCCATGCACAAACCC | SHANK3 H2A(−71−47) |
| 2380 | CTGAGCAAGTGAGTCCATGCACAAA | SHANK3 H2A(−68−44) |
| 2381 | CGGCTGAGCAAGTGAGTCCATGCAC | SHANK3 H2A(−65−41) |
| 2382 | CCCCGGCTGAGCAAGTGAGTCCATG | SHANK3 H2A(−62−38) |
| 2383 | CCACCCCGGCTGAGCAAGTGAGTCC | SHANK3 H2A(−59−35) |
| 2384 | CCCCCACCCCGGCTGAGCAAGTGAG | SHANK3 H2A(−56−32) |
| 2385 | ATGCCCCCACCCCGGCTGAGCAAGT | SHANK3 H2A(−53−29) |
| 2386 | AAAATGCCCCCACCCCGGCTGAGCA | SHANK3 H2A(−50−26) |
| 2387 | GAGAAAATGCCCCCACCCCGGCTGA | SHANK3 H2A(−47−23) |
| 2388 | GTAGAGAAAATGCCCCCACCCCGGC | SHANK3 H2A(−44−20) |
| 2389 | AAGGTAGAGAAAATGCCCCCACCCC | SHANK3 H2A(−41−17) |
| 2390 | GAAAAGGTAGAGAAAATGCCCCCAC | SHANK3 H2A(−38−14) |
| 2391 | AAAGAAAAGGTAGAGAAAATGCCCC | SHANK3 H2A(−35−11) |
| 2392 | GATAAAGAAAAGGTAGAGAAAATGC | SHANK3 H2A(−32−8) |
| 2393 | TCAGATAAAGAAAAGGTAGAGAAAA | SHANK3 H2A(−29−5) |
| 2394 | TGCTCAGATAAAGAAAAGGTAGAGA | SHANK3 H2A(−26−2) |
| 2395 | ACCTCCAGGTAGGGCAG | SHANK3 H1D(+15−2) |
| 2396 | CTTACCTCCAGGTAGGG | SHANK3 H1D(+12−5) |
| 2397 | CCACTTACCTCCAGGTA | SHANK3 H1D(+9−8) |
| 2398 | CGGCCACTTACCTCCAG | SHANK3 H1D(+6−11) |
| 2399 | CGCCGGCCACTTACCTC | SHANK3 H1D(+3−14) |
| 2400 | CCGCGCCGGCCACTTAC | SHANK3 H1D(−1−17) |
| 2401 | CCCCCGCGCCGGCCACT | SHANK3 H1D(−4−20) |
| 2402 | TCACCCCCGCGCCGGCC | SHANK3 H1D(−7−23) |
| 2403 | AGCTCACCCCCGCGCCG | SHANK3 H1D(−10−26) |
| 2404 | CTCAGCTCACCCCCGCG | SHANK3 H1D(−13−29) |
| 2405 | CTCCTCAGCTCACCCCC | SHANK3 H1D(−16−32) |
| 2406 | GCGCTCCTCAGCTCACC | SHANK3 H1D(−19−35) |
| 2407 | TGCGCGCTCCTCAGCTC | SHANK3 H1D(−22−38) |
| 2408 | CCCTGCGCGCTCCTCAG | SHANK3 H1D(−25−41) |
| 2409 | CCACCCTGCGCGCTCCT | SHANK3 H1D(−28−44) |
| 2410 | GATCCACCCTGCGCGCT | SHANK3 H1D(−31−47) |
| 2411 | GGTGATCCACCCTGCGC | SHANK3 H1D(−34−50) |
| 2412 | CTTGGTGATCCACCCTG | SHANK3 H1D(−37−53) |
| 2413 | GGGCTTGGTGATCCACC | SHANK3 H1D(−40−56) |
| 2414 | ACGGGGCTTGGTGATCC | SHANK3 H1D(−43−59) |
| 2415 | GCCACGGGGCTTGGTGA | SHANK3 H1D(−46−62) |
| 2416 | CCCGCCACGGGGCTTGG | SHANK3 H1D(−49−65) |
| 2417 | GGTCCCGCCACGGGGCT | SHANK3 H1D(−52−68) |
| 2418 | ACTGGTCCCGCCACGGG | SHANK3 H1D(−55−71) |
| 2419 | TTCACTGGTCCCGCCAC | SHANK3 H1D(−58−74) |
| 2420 | CCCTTCACTGGTCCCGC | SHANK3 H1D(−61−77) |
| 2421 | GTGCCCTTCACTGGTCC | SHANK3 H1D(−64−80) |
| 2422 | GCCGTGCCCTTCACTGG | SHANK3 H1D(−67−83) |
| 2423 | ACTGCCGTGCCCTTCAC | SHANK3 H1D(−70−86) |
| 2424 | CCCACTGCCGTGCCCTT | SHANK3 H1D(−73−89) |
| 2425 | TTCCCCACTGCCGTGCC | SHANK3 H1D(−76−92) |
| 2426 | TGTTTCCCCACTGCCGT | SHANK3 H1D(−79−95) |
| 2427 | TTGTGTTTCCCCACTGC | SHANK3 H1D(−82−98) |
| 2428 | CACTTGTGTTTCCCCAC | SHANK3 H1D(−85−101) |
| 2429 | TCCCACTTGTGTTTCCC | SHANK3 H1D(−88−104) |
| 2430 | CCCTCCCACTTGTGTTT | SHANK3 H1D(−91−107) |
| 2431 | CACCCCTCCCACTTGTG | SHANK3 H1D(−94−110) |
| 2432 | CCTCACCCCTCCCACTT | SHANK3 H1D(−97−113) |
| 2433 | ACCCCTCACCCCTCCCA | SHANK3 H1D(−100−116) |
| 2434 | TCCACCCCTCACCCCTC | SHANK3 H1D(−103−119) |
| 2435 | GCCTCCACCCCTCACCC | SHANK3 H1D(−106−122) |
| 2436 | ACAGCCTCCACCCCTCA | SHANK3 H1D(−109−125) |
| 2437 | CACACAGCCTCCACCCC | SHANK3 H1D(−112−128) |
| 2438 | ACACACACAGCCTCCAC | SHANK3 H1D(−115−131) |
| 2439 | CACACACACACAGCCTC | SHANK3 H1D(−118−134) |
| 2440 | ACACACACACACACAGC | SHANK3 H1D(−121−137) |
| 2441 | CACACACACACACACAC | SHANK3 H1D(−124−140) |
| 2442 | ACACACACACACACACA | SHANK3 H1D(−127−143) |
| 2443 | CACCACACACACACACA | SHANK3 H1D(−139−155) |
| 2444 | CAGCACCACACACACAC | SHANK3 H1D(−142−158) |
| 2445 | ACACAGCACCACACACA | SHANK3 H1D(−145−161) |
| 2446 | TGCACACAGCACCACAC | SHANK3 H1D(−148−164) |
| 2447 | CTCTGCACACAGCACCA | SHANK3 H1D(−151−167) |
| 2448 | GCACTCTGCACACAGCA | SHANK3 H1D(−154−170) |
| 2449 | ACTGCACTCTGCACACA | SHANK3 H1D(−157−173) |
| 2450 | CTCACTGCACTCTGCAC | SHANK3 H1D(−160−176) |
| 2451 | ACGCTCACTGCACTCTG | SHANK3 H1D(−163−179) |
| 2452 | TACACGCTCACTGCACT | SHANK3 H1D(−166−182) |
| 2453 | CTGTACACGCTCACTGC | SHANK3 H1D(−169−185) |
| 2454 | ACCCTGTACACGCTCAC | SHANK3 H1D(−172−188) |
| 2455 | TGCACCCTGTACACGCT | SHANK3 H1D(−175−191) |
| 2456 | CGCTGCACCCTGTACAC | SHANK3 H1D(−178−194) |
| 2457 | GCTCGCTGCACCCTGTA | SHANK3 H1D(−181−197) |
| 2458 | TCCGCTCGCTGCACCCT | SHANK3 H1D(−184−200) |
| 2459 | GTGTCCGCTCGCTGCAC | SHANK3 H1D(−187−203) |
| 2460 | ACTGTGTCCGCTCGCTG | SHANK3 H1D(−190−206) |
| 2461 | TACACTGTGTCCGCTCG | SHANK3 H1D(−193−209) |
| 2462 | GCATACACTGTGTCCGC | SHANK3 H1D(−196−212) |
| 2463 | ATCGCATACACTGTGTC | SHANK3 H1D(−199−215) |
| 2464 | CTCATCGCATACACTGT | SHANK3 H1D(−202−218) |
| 2465 | CTACTCATCGCATACAC | SHANK3 H1D(−205−221) |
| 2466 | CGCCTACTCATCGCATA | SHANK3 H1D(−208−224) |
| 2467 | CCGCGCCTACTCATCGC | SHANK3 H1D(−211−227) |
| 2468 | ACACCGCGCCTACTCAT | SHANK3 H1D(−214−230) |
| 2469 | CACACACCGCGCCTACT | SHANK3 H1D(−217−233) |
| 2470 | CTGCACACACCGCGCCT | SHANK3 H1D(−220−236) |
| 2471 | TCACTGCACACACCGCG | SHANK3 H1D(−223−239) |
| 2472 | CGCTCACTGCACACACC | SHANK3 H1D(−226−242) |
| 2473 | GCCCGCTCACTGCACAC | SHANK3 H1D(−229−245) |
| 2474 | CCTGCCCGCTCACTGCA | SHANK3 H1D(−232−248) |
| 2475 | CGCCCTGCCCGCTCACT | SHANK3 H1D(−235−251) |
| 2476 | GCTCGCCCTGCCCGCTC | SHANK3 H1D(−238−254) |
| 2477 | ACTGCTCGCCCTGCCCG | SHANK3 H1D(−241−257) |
| 2478 | CTTACTGCTCGCCCTGC | SHANK3 H1D(−244−260) |
| 2479 | ATCCTTACTGCTCGCCC | SHANK3 H1D(−247−263) |
| 2480 | TACATCCTTACTGCTCG | SHANK3 H1D(−250−266) |
| 2481 | CTGTACATCCTTACTGC | SHANK3 H1D(−253−269) |
| 2482 | ACACTGTACATCCTTAC | SHANK3 H1D(−256−272) |
| 2483 | CCCACACTGTACATCCT | SHANK3 H1D(−259−275) |
| 2484 | CTGCCCACACTGTACAT | SHANK3 H1D(−262−278) |
| 2485 | ACACTGCCCACACTGTA | SHANK3 H1D(−265−281) |
| 2486 | CGCACACTGCCCACACT | SHANK3 H1D(−268−284) |
| 2487 | GCTCGCACACTGCCCAC | SHANK3 H1D(−271−287) |
| 2488 | CATGCTCGCACACTGCC | SHANK3 H1D(−274−290) |
| 2489 | ACACATGCTCGCACACT | SHANK3 H1D(−277−293) |
| 2490 | ACTACACATGCTCGCAC | SHANK3 H1D(−280−296) |
| 2491 | CTCACTACACATGCTCG | SHANK3 H1D(−283−299) |
| 2492 | CTGCTCACTACACATGC | SHANK3 H1D(−286−302) |
| 2493 | TGCCTGCTCACTACACA | SHANK3 H1D(−289−305) |
| 2494 | CACTGCCTGCTCACTAC | SHANK3 H1D(−292−308) |
| 2495 | ACACACTGCCTGCTCAC | SHANK3 H1D(−295−311) |
| 2496 | TGCACACACTGCCTGCT | SHANK3 H1D(−298−314) |
| 2497 | AACTGCACACACTGCCT | SHANK3 H1D(−301−317) |
| 2498 | GCTAACTGCACACACTG | SHANK3 H1D(−304−320) |
| 2499 | CCTGCTAACTGCACACA | SHANK3 H1D(−307−323) |
| 2500 | GTGCCTGCTAACTGCAC | SHANK3 H1D(−310−326) |
| 2501 | ACTGTGCCTGCTAACTG | SHANK3 H1D(−313−329) |
| 2502 | CACACTGTGCCTGCTAA | SHANK3 H1D(−316−332) |
| 2503 | GCACACACTGTGCCTGC | SHANK3 H1D(−319−335) |
| 2504 | ACTGCACACACTGTGCC | SHANK3 H1D(−322−338) |
| 2505 | CTCACTGCACACACTGT | SHANK3 H1D(−325−341) |
| 2506 | CCACTCACTGCACACAC | SHANK3 H1D(−328−344) |
| 2507 | TGCCCACTCACTGCACA | SHANK3 H1D(−331−347) |
| 2508 | CACTGCCCACTCACTGC | SHANK3 H1D(−334−350) |
| 2509 | GCACACTGCCCACTCAC | SHANK3 H1D(−337−353) |
| 2510 | TGTGCACACTGCCCACT | SHANK3 H1D(−340−356) |
| 2511 | TGCTGTGCACACTGCCC | SHANK3 H1D(−343−359) |
| 2512 | ACATGCTGTGCACACTG | SHANK3 H1D(−346−362) |
| 2513 | TGTACATGCTGTGCACA | SHANK3 H1D(−349−365) |
| 2514 | CACTGTACATGCTGTGC | SHANK3 H1D(−352−368) |
| 2515 | TCACACTGTACATGCTG | SHANK3 H1D(−355−371) |
| 2516 | CACTCACACTGTACATG | SHANK3 H1D(−358−374) |
| 2517 | CACCACTCACACTGTAC | SHANK3 H1D(−361−377) |
| 2518 | ACACACCACTCACACTG | SHANK3 H1D(−364−380) |
| 2519 | AGCACACACCACTCACA | SHANK3 H1D(−367−383) |
| 2520 | CACAGCACACACCACTC | SHANK3 H1D(−370−386) |
| 2521 | GCACACAGCACACACCA | SHANK3 H1D(−373−389) |
| 2522 | ACGGCACACAGCACACA | SHANK3 H1D(−376−392) |
| 2523 | CTCACGGCACACAGCAC | SHANK3 H1D(−379−395) |
| 2524 | ATGCTCACGGCACACAG | SHANK3 H1D(−382−398) |
| 2525 | CACATGCTCACGGCACA | SHANK3 H1D(−385−401) |
| 2526 | CTACACATGCTCACGGC | SHANK3 H1D(−388−404) |
| 2527 | TCACTACACATGCTCAC | SHANK3 H1D(−391−407) |
| 2528 | CACTCACTACACATGCT | SHANK3 H1D(−394−410) |
| 2529 | GCCCACTCACTACACAT | SHANK3 H1D(−397−413) |
| 2530 | TGTGCCCACTCACTACA | SHANK3 H1D(−400−416) |
| 2531 | CACTGTGCCCACTCACT | SHANK3 H1D(−403−419) |
| 2532 | CTGCACTGTGCCCACTC | SHANK3 H1D(−406−422) |
| 2533 | TGACTGCACTGTGCCCA | SHANK3 H1D(−409−425) |
| 2534 | CACTGACTGCACTGTGC | SHANK3 H1D(−412−428) |
| 2535 | GCACACTGACTGCACTG | SHANK3 H1D(−415−431) |
| 2536 | TGTGCACACTGACTGCA | SHANK3 H1D(−418−434) |
| 2537 | CTTTGTGCACACTGACT | SHANK3 H1D(−421−437) |
| 2538 | ATACTTTGTGCACACTG | SHANK3 H1D(−424−440) |
| 2539 | TGCATACTTTGTGCACA | SHANK3 H1D(−427−443) |
| 2540 | CACTGCATACTTTGTGC | SHANK3 H1D(−430−446) |
| 2541 | GCCCACTGCATACTTTG | SHANK3 H1D(−433−449) |
| 2542 | CGTGCCCACTGCATACT | SHANK3 H1D(−436−452) |
| 2543 | GTACGTGCCCACTGCAT | SHANK3 H1D(−439−455) |
| 2544 | ACGGTACGTGCCCACTG | SHANK3 H1D(−442−458) |
| 2545 | CACACGGTACGTGCCCA | SHANK3 H1D(−445−461) |
| 2546 | GCACACACGGTACGTGC | SHANK3 H1D(−448−464) |
| 2547 | ACTGCACACACGGTACG | SHANK3 H1D(−451−467) |
| 2548 | CTCACTGCACACACGGT | SHANK3 H1D(−454−470) |
| 2549 | ACACCACTCACTGCACA | SHANK3 H1D(−460−476) |
| 2550 | TGCACACCACTCACTGC | SHANK3 H1D(−463−479) |
| 2551 | CACTGCACACCACTCAC | SHANK3 H1D(−466−482) |
| 2552 | ACACACTGCACACCACT | SHANK3 H1D(−469−485) |
| 2553 | ACTACACACTGCACACC | SHANK3 H1D(−472−488) |
| 2554 | CACACTACACACTGCAC | SHANK3 H1D(−475−491) |
| 2555 | CTGCACACTACACACTG | SHANK3 H1D(−478−494) |
| 2556 | TCACTGCACACTACACA | SHANK3 H1D(−481−497) |
| 2557 | TGCTCACTGCACACTAC | SHANK3 H1D(−484−500) |
| 2558 | CACTGCTCACTGCACAC | SHANK3 H1D(−487−503) |
| 2559 | ACACACTGCTCACTGCA | SHANK3 H1D(−490−506) |
| 2560 | TGTACACACTGCTCACT | SHANK3 H1D(−493−509) |
| 2561 | TGCTGTACACACTGCTC | SHANK3 H1D(−496−512) |
| 2562 | CAATGCTGTACACACTG | SHANK3 H1D(−499−515) |
| 2563 | CTGCAATGCTGTACACA | SHANK3 H1D(−502−518) |
| 2564 | ACACTGCAATGCTGTAC | SHANK3 H1D(−505−521) |
| 2565 | CCCACACTGCAATGCTG | SHANK3 H1D(−508−524) |
| 2566 | TGCCCCACACTGCAATG | SHANK3 H1D(−511−527) |
| 2567 | CACTGCCCCACACTGCA | SHANK3 H1D(−514−530) |
| 2568 | ACTCACTGCCCCACACT | SHANK3 H1D(−517−533) |
| 2569 | TGTACTCACTGCCCCAC | SHANK3 H1D(−520−536) |
| 2570 | CACTGTACTCACTGCCC | SHANK3 H1D(−523−539) |
| 2571 | TCACACTGTACTCACTG | SHANK3 H1D(−526−542) |
| 2572 | CACTCACACTGTACTCA | SHANK3 H1D(−529−545) |
| 2573 | CAACACTCACACTGTAC | SHANK3 H1D(−532−548) |
| 2574 | TCACAACACTCACACTG | SHANK3 H1D(−535−551) |
| 2575 | CAATCACAACACTCACA | SHANK3 H1D(−538−554) |
| 2576 | CCACAATCACAACACTC | SHANK3 H1D(−541−557) |
| 2577 | TTACCACAATCACAACA | SHANK3 H1D(−544−560) |
| 2578 | TGCTTACCACAATCACA | SHANK3 H1D(−547−563) |
| 2579 | TCCTGCTTACCACAATC | SHANK3 H1D(−550−566) |
| 2580 | GCATCCTGCTTACCACA | SHANK3 H1D(−553−569) |
| 2581 | TGCGCATCCTGCTTACC | SHANK3 H1D(−556−572) |
| 2582 | TACTGCGCATCCTGCTT | SHANK3 H1D(−559−575) |
| 2583 | GTATACTGCGCATCCTG | SHANK3 H1D(−562−578) |
| 2584 | ACTGTATACTGCGCATC | SHANK3 H1D(−565−581) |
| 2585 | TTCACTGTATACTGCGC | SHANK3 H1D(−568−584) |
| 2586 | CTGTTCACTGTATACTG | SHANK3 H1D(−571−587) |
| 2587 | ACACTGTTCACTGTATA | SHANK3 H1D(−574−590) |
| 2588 | TGCACACTGTTCACTGT | SHANK3 H1D(−577−593) |
| 2589 | CTGTGCACACTGTTCAC | SHANK3 H1D(−580−596) |
| 2590 | ACACTGTGCACACTGTT | SHANK3 H1D(−583−599) |
| 2591 | CACACACTGTGCACACT | SHANK3 H1D(−586−602) |
| 2592 | CTGCACACACTGTGCAC | SHANK3 H1D(−589−605) |
| 2593 | ACACTGCACACACTGTG | SHANK3 H1D(−592−608) |
| 2594 | CCCACACTGCACACACT | SHANK3 H1D(−595−611) |
| 2595 | CAGCCCACACTGCACAC | SHANK3 H1D(−598−614) |
| 2596 | ACACAGCCCACACTGCA | SHANK3 H1D(−601−617) |
| 2597 | GGCACACAGCCCACACT | SHANK3 H1D(−604−620) |
| 2598 | TGTGGCACACAGCCCAC | SHANK3 H1D(−607−623) |
| 2599 | CTCTGTGGCACACAGCC | SHANK3 H1D(−610−626) |
| 2600 | TGACTCTGTGGCACACA | SHANK3 H1D(−613−629) |
| 2601 | CACTGACTCTGTGGCAC | SHANK3 H1D(−616−632) |
| 2602 | CCACACTGACTCTGTGG | SHANK3 H1D(−619−635) |
| 2603 | ACACCACACTGACTCTG | SHANK3 H1D(−622−638) |
| 2604 | CACACACCACACTGACT | SHANK3 H1D(−625−641) |
| 2605 | CTACACACACCACACTG | SHANK3 H1D(−628−644) |
| 2606 | AAACTACACACACCACA | SHANK3 H1D(−631−647) |
| 2607 | TTCAAACTACACACACC | SHANK3 H1D(−634−650) |
| 2608 | CTGTTCAAACTACACAC | SHANK3 H1D(−637−653) |
| 2609 | ACACTGTTCAAACTACA | SHANK3 H1D(−640−656) |
| 2610 | CACACACTGTTCAAACT | SHANK3 H1D(−643−659) |
| 2611 | ATGCACACACTGTTCAA | SHANK3 H1D(−646−662) |
| 2612 | TCAATGCACACACTGTT | SHANK3 H1D(−649−665) |
| 2613 | TGCTCAATGCACACACT | SHANK3 H1D(−652−668) |
| 2614 | TGCTGCTCAATGCACAC | SHANK3 H1D(−655−671) |
| 2615 | CCATGCTGCTCAATGCA | SHANK3 H1D(−658−674) |
| 2616 | CATCCATGCTGCTCAAT | SHANK3 H1D(−661−677) |
| 2617 | CACCATCCATGCTGCTC | SHANK3 H1D(−664−680) |
| 2618 | CCACACCATCCATGCTG | SHANK3 H1D(−667−683) |
| 2619 | CGTCCACACCATCCATG | SHANK3 H1D(−670−686) |
| 2620 | CAGCGTCCACACCATCC | SHANK3 H1D(−673−689) |
| 2621 | GCTCAGCGTCCACACCA | SHANK3 H1D(−676−692) |
| 2622 | AATGCTCAGCGTCCACA | SHANK3 H1D(−679−695) |
| 2623 | AACAATGCTCAGCGTCC | SHANK3 H1D(−682−698) |
| 2624 | GAGAACAATGCTCAGCG | SHANK3 H2A(−686−670) |
| 2625 | CTGGAGAACAATGCTCA | SHANK3 H2A(−683−667) |
| 2626 | TCCCTGGAGAACAATGC | SHANK3 H2A(−680−664) |
| 2627 | TCCTCCCTGGAGAACAA | SHANK3 H2A(−677−661) |
| 2628 | CACTCCTCCCTGGAGAA | SHANK3 H2A(−674−658) |
| 2629 | TCACACTCCTCCCTGGA | SHANK3 H2A(−671−655) |
| 2630 | TGCTCACACTCCTCCCT | SHANK3 H2A(−668−652) |
| 2631 | TCGTGCTCACACTCCTC | SHANK3 H2A(−665−649) |
| 2632 | CTCTCGTGCTCACACTC | SHANK3 H2A(−662−646) |
| 2633 | ACTCTCTCGTGCTCACA | SHANK3 H2A(−659−643) |
| 2634 | GGCACTCTCTCGTGCTC | SHANK3 H2A(−656−640) |
| 2635 | TCTGGCACTCTCTCGTG | SHANK3 H2A(−653−637) |
| 2636 | CCCTCTGGCACTCTCTC | SHANK3 H2A(−650−634) |
| 2637 | CACCCCTCTGGCACTCT | SHANK3 H2A(−647−631) |
| 2638 | ACACACCCCTCTGGCAC | SHANK3 H2A(−644−628) |
| 2639 | ACCACACACCCCTCTGG | SHANK3 H2A(−641−625) |
| 2640 | CACACCACACACCCCTC | SHANK3 H2A(−638−622) |
| 2641 | GCTCACACCACACACCC | SHANK3 H2A(−635−619) |
| 2642 | CCTGCTCACACCACACA | SHANK3 H2A(−632−616) |
| 2643 | AGCCCTGCTCACACCAC | SHANK3 H2A(−629−613) |
| 2644 | GATAGCCCTGCTCACAC | SHANK3 H2A(−626−610) |
| 2645 | ACAGATAGCCCTGCTCA | SHANK3 H2A(−623−607) |
| 2646 | CACACAGATAGCCCTGC | SHANK3 H2A(−620−604) |
| 2647 | GTGCACACAGATAGCCC | SHANK3 H2A(−617−601) |
| 2648 | AACGTGCACACAGATAG | SHANK3 H2A(−614−598) |
| 2649 | ACAAACGTGCACACAGA | SHANK3 H2A(−611−595) |
| 2650 | GGAACAAACGTGCACAC | SHANK3 H2A(−608−592) |
| 2651 | AAAGGAACAAACGTGCA | SHANK3 H2A(−605−589) |
| 2652 | GAGAAAGGAACAAACGT | SHANK3 H2A(−602−586) |
| 2653 | CTGGAGAAAGGAACAAA | SHANK3 H2A(−599−583) |
| 2654 | CAGCTGGAGAAAGGAAC | SHANK3 H2A(−596−580) |
| 2655 | TCACAGCTGGAGAAAGG | SHANK3 H2A(−593−577) |
| 2656 | ACTTCACAGCTGGAGAA | SHANK3 H2A(−590−574) |
| 2657 | AAGACTTCACAGCTGGA | SHANK3 H2A(−587−571) |
| 2658 | CACAAGACTTCACAGCT | SHANK3 H2A(−584−568) |
| 2659 | CTTCACAAGACTTCACA | SHANK3 H2A(−581−565) |
| 2660 | GGCCTTCACAAGACTTC | SHANK3 H2A(−578−562) |
| 2661 | GTTGGCCTTCACAAGAC | SHANK3 H2A(−575−559) |
| 2662 | TTGGTTGGCCTTCACAA | SHANK3 H2A(−572−556) |
| 2663 | GACTTGGTTGGCCTTCA | SHANK3 H2A(−569−553) |
| 2664 | GGGGACTTGGTTGGCCT | SHANK3 H2A(−566−550) |
| 2665 | GGAGGGGACTTGGTTGG | SHANK3 H2A(−563−547) |
| 2666 | AAAGGAGGGGACTTGGT | SHANK3 H2A(−560−544) |
| 2667 | AGTAAAGGAGGGGACTT | SHANK3 H2A(−557−541) |
| 2668 | GTGAGTAAAGGAGGGGA | SHANK3 H2A(−554−538) |
| 2669 | TGGGTGAGTAAAGGAGG | SHANK3 H2A(−551−535) |
| 2670 | GCATGGGTGAGTAAAGG | SHANK3 H2A(−548−532) |
| 2671 | CATGCATGGGTGAGTAA | SHANK3 H2A(−545−529) |
| 2672 | CACCATGCATGGGTGAG | SHANK3 H2A(−542−526) |
| 2673 | TCACACCATGCATGGGT | SHANK3 H2A(−539−523) |
| 2674 | TCTTCACACCATGCATG | SHANK3 H2A(−536−520) |
| 2675 | ACATCTTCACACCATGC | SHANK3 H2A(−533−517) |
| 2676 | AATACATCTTCACACCA | SHANK3 H2A(−530−514) |
| 2677 | CTCAATACATCTTCACA | SHANK3 H2A(−527−511) |
| 2678 | GCACTCAATACATCTTC | SHANK3 H2A(−524−508) |
| 2679 | AAGGCACTCAATACATC | SHANK3 H2A(−521−505) |
| 2680 | AGCAAGGCACTCAATAC | SHANK3 H2A(−518−502) |
| 2681 | CCTAGCAAGGCACTCAA | SHANK3 H2A(−515−499) |
| 2682 | ATGCCTAGCAAGGCACT | SHANK3 H2A(−512−496) |
| 2683 | CCCATGCCTAGCAAGGC | SHANK3 H2A(−509−493) |
| 2684 | GTCCCCATGCCTAGCAA | SHANK3 H2A(−506−490) |
| 2685 | TACGTCCCCATGCCTAG | SHANK3 H2A(−503−487) |
| 2686 | GTCTACGTCCCCATGCC | SHANK3 H2A(−500−484) |
| 2687 | CCCGTCTACGTCCCCAT | SHANK3 H2A(−497−481) |
| 2688 | GACCCCGTCTACGTCCC | SHANK3 H2A(−494−478) |
| 2689 | ACTGACCCCGTCTACGT | SHANK3 H2A(−491−475) |
| 2690 | AGGACTGACCCCGTCTA | SHANK3 H2A(−488−472) |
| 2691 | CACAGGACTGACCCCGT | SHANK3 H2A(−485−469) |
| 2692 | TTCCACAGGACTGACCC | SHANK3 H2A(−482−466) |
| 2693 | ACCTTCCACAGGACTGA | SHANK3 H2A(−479−463) |
| 2694 | AAGACCTTCCACAGGAC | SHANK3 H2A(−476−460) |
| 2695 | TCCAAGACCTTCCACAG | SHANK3 H2A(−473−457) |
| 2696 | AACTCCAAGACCTTCCA | SHANK3 H2A(−470−454) |
| 2697 | CCAAACTCCAAGACCTT | SHANK3 H2A(−467−451) |
| 2698 | CTGCCAAACTCCAAGAC | SHANK3 H2A(−464−448) |
| 2699 | ACCCTGCCAAACTCCAA | SHANK3 H2A(−461−445) |
| 2700 | TCCACCCTGCCAAACTC | SHANK3 H2A(−458−442) |
| 2701 | CCCTCCACCCTGCCAAA | SHANK3 H2A(−455−439) |
| 2702 | CCCCCCTCCACCCTGCC | SHANK3 H2A(−452−436) |
| 2703 | GCACCCCCCTCCACCCT | SHANK3 H2A(−449−433) |
| 2704 | TGGGCACCCCCCTCCAC | SHANK3 H2A(−446−430) |
| 2705 | AGTTGGGCACCCCCCTC | SHANK3 H2A(−443−427) |
| 2706 | TGCAGTTGGGCACCCCC | SHANK3 H2A(−440−424) |
| 2707 | CACTGCAGTTGGGCACC | SHANK3 H2A(−437−421) |
| 2708 | GCCCACTGCAGTTGGGC | SHANK3 H2A(−434−418) |
| 2709 | AATGCCCACTGCAGTTG | SHANK3 H2A(−431−415) |
| 2710 | TTCAATGCCCACTGCAG | SHANK3 H2A(−428−412) |
| 2711 | TTATTCAATGCCCACTG | SHANK3 H2A(−425−409) |
| 2712 | TCTTTATTCAATGCCCA | SHANK3 H2A(−422−406) |
| 2713 | AAATCTTTATTCAATGC | SHANK3 H2A(−419−403) |
| 2714 | ACAAAATCTTTATTCAA | SHANK3 H2A(−416−400) |
| 2715 | CTCACAAAATCTTTATT | SHANK3 H2A(−413−397) |
| 2716 | CAGCTCACAAAATCTTT | SHANK3 H2A(−410−394) |
| 2717 | GCTCAGCTCACAAAATC | SHANK3 H2A(−407−391) |
| 2718 | TGAGCTCAGCTCACAAA | SHANK3 H2A(−404−388) |
| 2719 | CTTTGAGCTCAGCTCAC | SHANK3 H2A(−401−385) |
| 2720 | CAACTTTGAGCTCAGCT | SHANK3 H2A(−398−382) |
| 2721 | GCCCAACTTTGAGCTCA | SHANK3 H2A(−395−379) |
| 2722 | CCCGCCCAACTTTGAGC | SHANK3 H2A(−392−376) |
| 2723 | AGGCCCGCCCAACTTTG | SHANK3 H2A(−389−373) |
| 2724 | CGGAGGCCCGCCCAACT | SHANK3 H2A(−386−370) |
| 2725 | CCACGGAGGCCCGCCCA | SHANK3 H2A(−383−367) |
| 2726 | CCACCACGGAGGCCCGC | SHANK3 H2A(−380−364) |
| 2727 | GTGCCACCACGGAGGCC | SHANK3 H2A(−377−361) |
| 2728 | CCTGTGCCACCACGGAG | SHANK3 H2A(−374−358) |
| 2729 | TTTCCTGTGCCACCACG | SHANK3 H2A(−371−355) |
| 2730 | CACTTTCCTGTGCCACC | SHANK3 H2A(−368−352) |
| 2731 | CCCCACTTTCCTGTGCC | SHANK3 H2A(−365−349) |
| 2732 | TGACCCCACTTTCCTGT | SHANK3 H2A(−362−346) |
| 2733 | AACTGACCCCACTTTCC | SHANK3 H2A(−359−343) |
| 2734 | TAGAACTGACCCCACTT | SHANK3 H2A(−356−340) |
| 2735 | AGGTAGAACTGACCCCA | SHANK3 H2A(−353−337) |
| 2736 | TCCAGGTAGAACTGACC | SHANK3 H2A(−350−334) |
| 2737 | CTCTCCAGGTAGAACTG | SHANK3 H2A(−347−331) |
| 2738 | CCACTCTCCAGGTAGAA | SHANK3 H2A(−344−328) |
| 2739 | CCTCCACTCTCCAGGTA | SHANK3 H2A(−341−325) |
| 2740 | CCCCCTCCACTCTCCAG | SHANK3 H2A(−338−322) |
| 2741 | CATCCCCCTCCACTCTC | SHANK3 H2A(−335−319) |
| 2742 | AGACATCCCCCTCCACT | SHANK3 H2A(−332−316) |
| 2743 | CCTAGACATCCCCCTCC | SHANK3 H2A(−329−313) |
| 2744 | CATCCTAGACATCCCCC | SHANK3 H2A(−326−310) |
| 2745 | TACCATCCTAGACATCC | SHANK3 H2A(−323−307) |
| 2746 | GCTTACCATCCTAGACA | SHANK3 H2A(−320−304) |
| 2747 | CAGGCTTACCATCCTAG | SHANK3 H2A(−317−301) |
| 2748 | TTCCAGGCTTACCATCC | SHANK3 H2A(−314−298) |
| 2749 | TGATTCCAGGCTTACCA | SHANK3 H2A(−311−295) |
| 2750 | GCCTGATTCCAGGCTTA | SHANK3 H2A(−308−292) |
| 2751 | AAGGCCTGATTCCAGGC | SHANK3 H2A(−305−289) |
| 2752 | CTGAAGGCCTGATTCCA | SHANK3 H2A(−302−286) |
| 2753 | TCTCTGAAGGCCTGATT | SHANK3 H2A(−299−283) |
| 2754 | TCCTCTCTGAAGGCCTG | SHANK3 H2A(−296−280) |
| 2755 | CACTCCTCTCTGAAGGC | SHANK3 H2A(−293−277) |
| 2756 | TCCCACTCCTCTCTGAA | SHANK3 H2A(−290−274) |
| 2757 | AAATCCCACTCCTCTCT | SHANK3 H2A(−287−271) |
| 2758 | GCAAAATCCCACTCCTC | SHANK3 H2A(−284−268) |
| 2759 | TCGGCAAAATCCCACTC | SHANK3 H2A(−281−265) |
| 2760 | TTCTCGGCAAAATCCCA | SHANK3 H2A(−278−262) |
| 2761 | GGATTCTCGGCAAAATC | SHANK3 H2A(−275−259) |
| 2762 | CCAGGATTCTCGGCAAA | SHANK3 H2A(−272−256) |
| 2763 | TCCCCAGGATTCTCGGC | SHANK3 H2A(−269−253) |
| 2764 | CCATCCCCAGGATTCTC | SHANK3 H2A(−266−250) |
| 2765 | TTCCCATCCCCAGGATT | SHANK3 H2A(−263−247) |
| 2766 | GCCTTCCCATCCCCAGG | SHANK3 H2A(−260−244) |
| 2767 | GTCGCCTTCCCATCCCC | SHANK3 H2A(−257−241) |
| 2768 | CCCGTCGCCTTCCCATC | SHANK3 H2A(−254−238) |
| 2769 | TGTCCCGTCGCCTTCCC | SHANK3 H2A(−251−235) |
| 2770 | CACTGTCCCGTCGCCTT | SHANK3 H2A(−248−232) |
| 2771 | CTGCACTGTCCCGTCGC | SHANK3 H2A(−245−229) |
| 2772 | AGCCTGCACTGTCCCGT | SHANK3 H2A(−242−226) |
| 2773 | CGCAGCCTGCACTGTCC | SHANK3 H2A(−239−223) |
| 2774 | GCCCGCAGCCTGCACTG | SHANK3 H2A(−236−220) |
| 2775 | GCTGCCCGCAGCCTGCA | SHANK3 H2A(−233−217) |
| 2776 | CTAGCTGCCCGCAGCCT | SHANK3 H2A(−230−214) |
| 2777 | TGCCTAGCTGCCCGCAG | SHANK3 H2A(−227−211) |
| 2778 | ACGTGCCTAGCTGCCCG | SHANK3 H2A(−224−208) |
| 2779 | GGCACGTGCCTAGCTGC | SHANK3 H2A(−221−205) |
| 2780 | GAAGGCACGTGCCTAGC | SHANK3 H2A(−218−202) |
| 2781 | ATGGAAGGCACGTGCCT | SHANK3 H2A(−215−199) |
| 2782 | CCGATGGAAGGCACGTG | SHANK3 H2A(−212−196) |
| 2783 | AGCCCGATGGAAGGCAC | SHANK3 H2A(−209−193) |
| 2784 | TGCAGCCCGATGGAAGG | SHANK3 H2A(−206−190) |
| 2785 | TGATGCAGCCCGATGGA | SHANK3 H2A(−203−187) |
| 2786 | GCATGATGCAGCCCGAT | SHANK3 H2A(−200−184) |
| 2787 | CAGGCATGATGCAGCCC | SHANK3 H2A(−197−181) |
| 2788 | ACTCAGGCATGATGCAG | SHANK3 H2A(−194−178) |
| 2789 | CACACTCAGGCATGATG | SHANK3 H2A(−191−175) |
| 2790 | CACCACACTCAGGCATG | SHANK3 H2A(−188−172) |
| 2791 | ACCCACCACACTCAGGC | SHANK3 H2A(−185−169) |
| 2792 | TGCACCCACCACACTCA | SHANK3 H2A(−182−166) |
| 2793 | CCATGCACCCACCACAC | SHANK3 H2A(−179−163) |
| 2794 | CTGCCATGCACCCACCA | SHANK3 H2A(−176−160) |
| 2795 | CAGCTGCCATGCACCCA | SHANK3 H2A(−173−157) |
| 2796 | TAACAGCTGCCATGCAC | SHANK3 H2A(−170−154) |
| 2797 | AGCTAACAGCTGCCATG | SHANK3 H2A(−167−151) |
| 2798 | CAGAGCTAACAGCTGCC | SHANK3 H2A(−164−148) |
| 2799 | GGACAGAGCTAACAGCT | SHANK3 H2A(−161−145) |
| 2800 | AGTGGACAGAGCTAACA | SHANK3 H2A(−158−142) |
| 2801 | CACAGTGGACAGAGCTA | SHANK3 H2A(−155−139) |
| 2802 | TACCACAGTGGACAGAG | SHANK3 H2A(−152−136) |
| 2803 | TACTACCACAGTGGACA | SHANK3 H2A(−149−133) |
| 2804 | TCATACTACCACAGTGG | SHANK3 H2A(−146−130) |
| 2805 | CAGTCATACTACCACAG | SHANK3 H2A(−143−127) |
| 2806 | CATCAGTCATACTACCA | SHANK3 H2A(−140−124) |
| 2807 | CACCATCAGTCATACTA | SHANK3 H2A(−137−121) |
| 2808 | ACACACCATCAGTCATA | SHANK3 H2A(−134−118) |
| 2809 | TGTACACACCATCAGTC | SHANK3 H2A(−131−115) |
| 2810 | TCCTGTACACACCATCA | SHANK3 H2A(−128−112) |
| 2811 | CCCTCCTGTACACACCA | SHANK3 H2A(−125−109) |
| 2812 | CTGCCCTCCTGTACACA | SHANK3 H2A(−122−106) |
| 2813 | TCACTGCCCTCCTGTAC | SHANK3 H2A(−119−103) |
| 2814 | CCCTCACTGCCCTCCTG | SHANK3 H2A(−116−100) |
| 2815 | CACCCCTCACTGCCCTC | SHANK3 H2A(−113−97) |
| 2816 | CCGCACCCCTCACTGCC | SHANK3 H2A(−110−94) |
| 2817 | ACACCGCACCCCTCACT | SHANK3 H2A(−107−91) |
| 2818 | GCCACACCGCACCCCTC | SHANK3 H2A(−104−88) |
| 2819 | CTGGCCACACCGCACCC | SHANK3 H2A(−101−85) |
| 2820 | ATGCTGGCCACACCGCA | SHANK3 H2A(−98−82) |
| 2821 | CTCATGCTGGCCACACC | SHANK3 H2A(−95−79) |
| 2822 | CCGCTCATGCTGGCCAC | SHANK3 H2A(−92−76) |
| 2823 | GTCCCGCTCATGCTGGC | SHANK3 H2A(−89−73) |
| 2824 | CCCGTCCCGCTCATGCT | SHANK3 H2A(−86−70) |
| 2825 | AACCCCGTCCCGCTCAT | SHANK3 H2A(−83−67) |
| 2826 | ACAAACCCCGTCCCGCT | SHANK3 H2A(−80−64) |
| 2827 | TGCACAAACCCCGTCCC | SHANK3 H2A(−77−61) |
| 2828 | CCATGCACAAACCCCGT | SHANK3 H2A(−74−58) |
| 2829 | AGTCCATGCACAAACCC | SHANK3 H2A(−71−55) |
| 2830 | GTGAGTCCATGCACAAA | SHANK3 H2A(−68−52) |
| 2831 | CAAGTGAGTCCATGCAC | SHANK3 H2A(−65−49) |
| 2832 | GAGCAAGTGAGTCCATG | SHANK3 H2A(−62−46) |
| 2833 | GCTGAGCAAGTGAGTCC | SHANK3 H2A(−59−43) |
| 2834 | CCGGCTGAGCAAGTGAG | SHANK3 H2A(−56−40) |
| 2835 | ACCCCGGCTGAGCAAGT | SHANK3 H2A(−53−37) |
| 2836 | CCCACCCCGGCTGAGCA | SHANK3 H2A(−50−34) |
| 2837 | GCCCCCACCCCGGCTGA | SHANK3 H2A(−47−31) |
| 2838 | AATGCCCCCACCCCGGC | SHANK3 H2A(−44−28) |
| 2839 | GAAAATGCCCCCACCCC | SHANK3 H2A(−41−25) |
| 2840 | AGAGAAAATGCCCCCAC | SHANK3 H2A(−38−22) |
| 2841 | GGTAGAGAAAATGCCCC | SHANK3 H2A(−35−19) |
| 2842 | AAAGGTAGAGAAAATGC | SHANK3 H2A(−32−16) |
| 2843 | AGAAAAGGTAGAGAAAA | SHANK3 H2A(−29−13) |
| 2844 | TAAAGAAAAGGTAGAGA | SHANK3 H2A(−26−10) |
| 2845 | AGATAAAGAAAAGGTAG | SHANK3 H2A(−23−7) |
| 2846 | CTCAGATAAAGAAAAGG | SHANK3 H2A(−20−4) |
| 2847 | CTGCTCAGATAAAGAAA | SHANK3 H2A(−17−1) |
| 2848 | AACTGCTCAGATAAAGAAAAGGTAG | SHANK3 H2A(−23+2) |
| 2849 | CGAAACTGCTCAGATAAAGAAAAGG | SHANK3 H2A(−20+5) |
| 2850 | TATCGAAACTGCTCAGATAAAGAAA | SHANK3 H2A(−17+8) |
| 2851 | TTGTATCGAAACTGCTCAGATAAAG | SHANK3 H2A(−14+11) |
| 2852 | CGCTTGTATCGAAACTGCTCAGATA | SHANK3 H2A(−11+14) |
| 2853 | CGCCGCTTGTATCGAAACTGCTCAG | SHANK3 H2A(−8+17) |
| 2854 | ACTCGCCGCTTGTATCGAAACTGCT | SHANK3 H2A(−5+20) |
| 2855 | TAAACTCGCCGCTTGTATCGAAACT | SHANK3 H2A(−2+23) |
| 2856 | GCATAAACTCGCCGCTTGTATCGAA | SHANK3 H2A(+2+26) |
| 2857 | TGGGCATAAACTCGCCGCTTGTATC | SHANK3 H2A(+5+29) |
| 2858 | TTCTGGGCATAAACTCGCCGCTTGT | SHANK3 H2A(+8+32) |
| 2859 | AGGTTCTGGGCATAAACTCGCCGCT | SHANK3 H2A(+11+35) |
| 2860 | ATGAGGTTCTGGGCATAAACTCGCC | SHANK3 H2A(+14+38) |
| 2861 | TCGATGAGGTTCTGGGCATAAACTC | SHANK3 H2A(+17+41) |
| 2862 | TCATCGATGAGGTTCTGGGCATAAA | SHANK3 H2A(+20+44) |
| 2863 | TTATCATCGATGAGGTTCTGGGCAT | SHANK3 H2A(+23+47) |
| 2864 | TGCTTATCATCGATGAGGTTCTGGG | SHANK3 H2A(+26+50) |
| 2865 | AACTGCTTATCATCGATGAGGTTCT | SHANK3 H2A(+29+53) |
| 2866 | GCAAACTGCTTATCATCGATGAGGT | SHANK3 H2A(+32+56) |
| 2867 | TTTGCAAACTGCTTATCATCGATGA | SHANK3 H2A(+35+59) |
| 2868 | AGCTTTGCAAACTGCTTATCATCGA | SHANK3 H2A(+38+62) |
| 2869 | TGAAGCTTTGCAAACTGCTTATCAT | SHANK3 H2A(+41+65) |
| 2870 | GTGTGAAGCTTTGCAAACTGCTTAT | SHANK3 H2A(+44+68) |
| 2871 | TTTGTGTGAAGCTTTGCAAACTGCT | SHANK3 H2A(+47+71) |
| 2872 | ACCTTTGTGTGAAGCTTTGCAAACT | SHANK3 H2D(+23−2) |
| 2873 | TTTACCTTTGTGTGAAGCTTTGCAA | SHANK3 H2D(+20−5) |
| 2874 | TCCTTTACCTTTGTGTGAAGCTTTG | SHANK3 H2D(+17−8) |
| 2875 | TGATCCTTTACCTTTGTGTGAAGCT | SHANK3 H2D(+14−11) |
| 2876 | CCGTGATCCTTTACCTTTGTGTGAA | SHANK3 H2D(+11−14) |
| 2877 | TCCCCGTGATCCTTTACCTTTGTGT | SHANK3 H2D(+8−17) |
| 2878 | CCCTCCCCGTGATCCTTTACCTTTG | SHANK3 H2D(+5−20) |
| 2879 | GCCCCCTCCCCGTGATCCTTTACCT | SHANK3 H2D(+2−23) |
| 2880 | GGAGCCCCCTCCCCGTGATCCTTTA | SHANK3 H2D(−2−26) |
| 2881 | TCAGGAGCCCCCTCCCCGTGATCCT | SHANK3 H2D(−5−29) |
| 2882 | ACCTCAGGAGCCCCCTCCCCGTGAT | SHANK3 H2D(−8−32) |
| 2883 | GGAACCTCAGGAGCCCCCTCCCCGT | SHANK3 H2D(−11−35) |
| 2884 | GAGGGAACCTCAGGAGCCCCCTCCC | SHANK3 H2D(−14−38) |
| 2885 | CAGGAGGGAACCTCAGGAGCCCCCT | SHANK3 H2D(−17−41) |
| 2886 | AGGCAGGAGGGAACCTCAGGAGCCC | SHANK3 H2D(−20−44) |
| 2887 | GGGAGGCAGGAGGGAACCTCAGGAG | SHANK3 H2D(−23−47) |
| 2888 | ACAGGGAGGCAGGAGGGAACCTCAG | SHANK3 H2D(−26−50) |
| 2889 | GCCACAGGGAGGCAGGAGGGAACCT | SHANK3 H2D(−29−53) |
| 2890 | GCAGCCACAGGGAGGCAGGAGGGAA | SHANK3 H2D(−32−56) |
| 2891 | ACAGCAGCCACAGGGAGGCAGGAGG | SHANK3 H2D(−35−59) |
| 2892 | GGGACAGCAGCCACAGGGAGGCAGG | SHANK3 H2D(−38−62) |
| 2893 | TGGGGGACAGCAGCCACAGGGAGGC | SHANK3 H2D(−41−65) |
| 2894 | GGGTGGGGGACAGCAGCCACAGGGA | SHANK3 H2D(−44−68) |
| 2895 | CTGGGGTGGGGGACAGCAGCCACAG | SHANK3 H2D(−47−71) |
| 2896 | AAGCTGGGGTGGGGGACAGCAGCCA | SHANK3 H2D(−50−74) |
| 2897 | CCCAAGCTGGGGTGGGGGACAGCAG | SHANK3 H2D(−53−77) |
| 2898 | AGCCCCAAGCTGGGGTGGGGGACAG | SHANK3 H2D(−56−80) |
| 2899 | GTCAGCCCCAAGCTGGGGGGGGGA | SHANK3 H2D(−59−83) |
| 2900 | GTGGTCAGCCCCAAGCTGGGGTGGG | SHANK3 H2D(−62−86) |
| 2901 | ACTGTGGTCAGCCCCAAGCTGGGGT | SHANK3 H2D(−65−89) |
| 2902 | GGGACTGTGGTCAGCCCCAAGCTGG | SHANK3 H2D(−68−92) |
| 2903 | TGGGGGACTGTGGTCAGCCCCAAGC | SHANK3 H2D(−71−95) |
| 2904 | AGCTGGGGGACTGTGGTCAGCCCCA | SHANK3 H2D(−74−98) |
| 2905 | TAAAGCTGGGGGACTGTGGTCAGCC | SHANK3 H2D(−77−101) |
| 2906 | AGCTAAAGCTGGGGGACTGTGGTCA | SHANK3 H2D(−80−104) |
| 2907 | CTGAGCTAAAGCTGGGGGACTGTGG | SHANK3 H2D(−83−107) |
| 2908 | GGACTGAGCTAAAGCTGGGGGACTG | SHANK3 H2D(−86−110) |
| 2909 | AATGGACTGAGCTAAAGCTGGGGGA | SHANK3 H2D(−89−113) |
| 2910 | GGAAATGGACTGAGCTAAAGCTGGG | SHANK3 H2D(−92−116) |
| 2911 | TGGGGAAATGGACTGAGCTAAAGCT | SHANK3 H2D(−95−119) |
| 2912 | TGATGGGGAAATGGACTGAGCTAAA | SHANK3 H2D(−98−122) |
| 2913 | TGATGATGGGGAAATGGACTGAGCT | SHANK3 H2D(−101−125) |
| 2914 | CCCTGATGATGGGGAAATGGACTGA | SHANK3 H2D(−104−128) |
| 2915 | GGCCCCTGATGATGGGGAAATGGAC | SHANK3 H2D(−107−131) |
| 2916 | CTGGGCCCCTGATGATGGGGAAATG | SHANK3 H2D(−110−134) |
| 2917 | GCTCTGGGCCCCTGATGATGGGGAA | SHANK3 H2D(−113−137) |
| 2918 | CAGGCTCTGGGCCCCTGATGATGGG | SHANK3 H2D(−116−140) |
| 2919 | GTACAGGCTCTGGGCCCCTGATGAT | SHANK3 H2D(−119−143) |
| 2920 | CCAGTACAGGCTCTGGGCCCCTGAT | SHANK3 H2D(−122−146) |
| 2921 | CACCCAGTACAGGCTCTGGGCCCCT | SHANK3 H2D(−125−149) |
| 2922 | CCACACCCAGTACAGGCTCTGGGCC | SHANK3 H2D(−128−152) |
| 2923 | CAGCCACACCCAGTACAGGCTCTGG | SHANK3 H2D(−131−155) |
| 2924 | CTTCAGCCACACCCAGTACAGGCTC | SHANK3 H2D(−134−158) |
| 2925 | GCCCTTCAGCCACACCCAGTACAGG | SHANK3 H2D(−137−161) |
| 2926 | CCAGCCCTTCAGCCACACCCAGTAC | SHANK3 H2D(−140−164) |
| 2927 | TGCCCAGCCCTTCAGCCACACCCAG | SHANK3 H2D(−143−167) |
| 2928 | CTGTGCCCAGCCCTTCAGCCACACC | SHANK3 H2D(−146−170) |
| 2929 | AATCTGTGCCCAGCCCTTCAGCCAC | SHANK3 H2D(−149−173) |
| 2930 | AGGAATCTGTGCCCAGCCCTTCAGC | SHANK3 H2D(−152−176) |
| 2931 | GCCAGGAATCTGTGCCCAGCCCTTC | SHANK3 H2D(−155−179) |
| 2932 | GGGGCCAGGAATCTGTGCCCAGCCC | SHANK3 H2D(−158−182) |
| 2933 | CATGGGGCCAGGAATCTGTGCCCAG | SHANK3 H2D(−161−185) |
| 2934 | AACCATGGGGCCAGGAATCTGTGCC | SHANK3 H2D(−164−188) |
| 2935 | CCCAACCATGGGGCCAGGAATCTGT | SHANK3 H2D(−167−191) |
| 2936 | GACCCCAACCATGGGGCCAGGAATC | SHANK3 H2D(−170−194) |
| 2937 | CCTGACCCCAACCATGGGGCCAGGA | SHANK3 H2D(−173−197) |
| 2938 | CCACCTGACCCCAACCATGGGGCCA | SHANK3 H2D(−176−200) |
| 2939 | GTACCACCTGACCCCAACCATGGGG | SHANK3 H2D(−179−203) |
| 2940 | ACTGTACCACCTGACCCCAACCATG | SHANK3 H2D(−182−206) |
| 2941 | ATCACTGTACCACCTGACCCCAACC | SHANK3 H2D(−185−209) |
| 2942 | CACATCACTGTACCACCTGACCCCA | SHANK3 H2D(−188−212) |
| 2943 | GAACACATCACTGTACCACCTGACC | SHANK3 H2D(−191−215) |
| 2944 | CTGGAACACATCACTGTACCACCTG | SHANK3 H2D(−194−218) |
| 2945 | TAACTGGAACACATCACTGTACCAC | SHANK3 H2D(−197−221) |
| 2946 | ATCTAACTGGAACACATCACTGTAC | SHANK3 H2D(−200−224) |
| 2947 | CTGATCTAACTGGAACACATCACTG | SHANK3 H2D(−203−227) |
| 2948 | AGTCTGATCTAACTGGAACACATCA | SHANK3 H2D(−206−230) |
| 2949 | AAGAGTCTGATCTAACTGGAACACA | SHANK3 H2D(−209−233) |
| 2950 | GGCAAGAGTCTGATCTAACTGGAAC | SHANK3 H2D(−212−236) |
| 2951 | GTCGGCAAGAGTCTGATCTAACTGG | SHANK3 H2D(−215−239) |
| 2952 | GGGGTCGGCAAGAGTCTGATCTAAC | SHANK3 H2D(−218−242) |
| 2953 | AGGGGGGTCGGCAAGAGTCTGATCT | SHANK3 H2D(−221−245) |
| 2954 | GCCAGGGGGGTCGGCAAGAGTCTGA | SHANK3 H2D(−224−248) |
| 2955 | TAAGCCAGGGGGGTCGGCAAGAGTC | SHANK3 H2D(−227−251) |
| 2956 | CCCTAAGCCAGGGGGGTCGGCAAGA | SHANK3 H2D(−230−254) |
| 2957 | AGCCCCTAAGCCAGGGGGGTCGGCA | SHANK3 H2D(−233−257) |
| 2958 | TCCAGCCCCTAAGCCAGGGGGGTCG | SHANK3 H2D(−236−260) |
| 2959 | CACTCCAGCCCCTAAGCCAGGGGGG | SHANK3 H2D(−239−263) |
| 2960 | GGACACTCCAGCCCCTAAGCCAGGG | SHANK3 H2D(−242−266) |
| 2961 | ACAGGACACTCCAGCCCCTAAGCCA | SHANK3 H2D(−245−269) |
| 2962 | CTCACAGGACACTCCAGCCCCTAAG | SHANK3 H2D(−248−272) |
| 2963 | CTTCTCACAGGACACTCCAGCCCCT | SHANK3 H2D(−251−275) |
| 2964 | CAGCTTCTCACAGGACACTCCAGCC | SHANK3 H2D(−254−278) |
| 2965 | ACCCAGCTTCTCACAGGACACTCCA | SHANK3 H2D(−257−281) |
| 2966 | TCCACCCAGCTTCTCACAGGACACT | SHANK3 H2D(−260−284) |
| 2967 | CTCTCCACCCAGCTTCTCACAGGAC | SHANK3 H2D(−263−287) |
| 2968 | TCCCTCTCCACCCAGCTTCTCACAG | SHANK3 H2D(−266−290) |
| 2969 | CACTCCCTCTCCACCCAGCTTCTCA | SHANK3 H2D(−269−293) |
| 2970 | GTCCACTCCCTCTCCACCCAGCTTC | SHANK3 H2D(−272−296) |
| 2971 | CTTGTCCACTCCCTCTCCACCCAGC | SHANK3 H2D(−275−299) |
| 2972 | ATGCTTGTCCACTCCCTCTCCACCC | SHANK3 H2D(−278−302) |
| 2973 | CAGATGCTTGTCCACTCCCTCTCCA | SHANK3 H2D(−281−305) |
| 2974 | CATCAGATGCTTGTCCACTCCCTCT | SHANK3 H2D(−284−308) |
| 2975 | TCACATCAGATGCTTGTCCACTCCC | SHANK3 H2D(−287−311) |
| 2976 | CCATCACATCAGATGCTTGTCCACT | SHANK3 H2D(−290−314) |
| 2977 | CAGCCATCACATCAGATGCTTGTCC | SHANK3 H2D(−293−317) |
| 2978 | CGACAGCCATCACATCAGATGCTTG | SHANK3 H2D(−296−320) |
| 2979 | TCCCGACAGCCATCACATCAGATGC | SHANK3 H2D(−299−323) |
| 2980 | TTGTCCCGACAGCCATCACATCAGA | SHANK3 H2D(−302−326) |
| 2981 | GCCTTGTCCCGACAGCCATCACATC | SHANK3 H2D(−305−329) |
| 2982 | GGTGCCTTGTCCCGACAGCCATCAC | SHANK3 H2D(−308−332) |
| 2983 | CTGGGTGCCTTGTCCCGACAGCCAT | SHANK3 H2D(−311−335) |
| 2984 | ATGCTGGGTGCCTTGTCCCGACAGC | SHANK3 H2D(−314−338) |
| 2985 | TCGATGCTGGGTGCCTTGTCCCGAC | SHANK3 H2D(−317−341) |
| 2986 | CACTCGATGCTGGGTGCCTTGTCCC | SHANK3 H2D(−320−344) |
| 2987 | GACCACTCGATGCTGGGTGCCTTGT | SHANK3 H2D(−323−347) |
| 2988 | GTCGACCACTCGATGCTGGGTGCCT | SHANK3 H2D(−326−350) |
| 2989 | CTGGTCGACCACTCGATGCTGGGTG | SHANK3 H2D(−329−353) |
| 2990 | GCACTGGTCGACCACTCGATGCTGG | SHANK3 H2D(−332−356) |
| 2991 | CTAGCACTGGTCGACCACTCGATGC | SHANK3 H2D(−335−359) |
| 2992 | TGCCTAGCACTGGTCGACCACTCGA | SHANK3 H2D(−338−362) |
| 2993 | AAATGCCTAGCACTGGTCGACCACT | SHANK3 H2D(−341−365) |
| 2994 | CACAAATGCCTAGCACTGGTCGACC | SHANK3 H2D(−344−368) |
| 2995 | AATCACAAATGCCTAGCACTGGTCG | SHANK3 H2D(−347−371) |
| 2996 | TCTAATCACAAATGCCTAGCACTGG | SHANK3 H2D(−350−374) |
| 2997 | AGGTCTAATCACAAATGCCTAGCAC | SHANK3 H2D(−353−377) |
| 2998 | GAGAGGTCTAATCACAAATGCCTAG | SHANK3 H2D(−356−380) |
| 2999 | AATGAGAGGTCTAATCACAAATGCC | SHANK3 H2D(−359−383) |
| 3000 | ATTAATGAGAGGTCTAATCACAAAT | SHANK3 H2D(−362−386) |
| 3001 | AGGATTAATGAGAGGTCTAATCACA | SHANK3 H2D(−365−389) |
| 3002 | AAGAGGATTAATGAGAGGTCTAATC | SHANK3 H2D(−368−392) |
| 3003 | TGGAAGAGGATTAATGAGAGGTCTA | SHANK3 H2D(−371−395) |
| 3004 | CTCTGGAAGAGGATTAATGAGAGGT | SHANK3 H2D(−374−398) |
| 3005 | TCCCTCTGGAAGAGGATTAATGAGA | SHANK3 H2D(−377−401) |
| 3006 | TAGTCCCTCTGGAAGAGGATTAATG | SHANK3 H2D(−380−404) |
| 3007 | TAATAGTCCCTCTGGAAGAGGATTA | SHANK3 H2D(−383−407) |
| 3008 | TTATAATAGTCCCTCTGGAAGAGGA | SHANK3 H2D(−386−410) |
| 3009 | GGGTTATAATAGTCCCTCTGGAAGA | SHANK3 H2D(−389−413) |
| 3010 | ACAGGGTTATAATAGTCCCTCTGGA | SHANK3 H2D(−392−416) |
| 3011 | GAAACAGGGTTATAATAGTCCCTCT | SHANK3 H2D(−395−419) |
| 3012 | TGTGAAACAGGGTTATAATAGTCCC | SHANK3 H2D(−398−422) |
| 3013 | ATCTGTGAAACAGGGTTATAATAGT | SHANK3 H2D(−401−425) |
| 3014 | CCCATCTGTGAAACAGGGTTATAAT | SHANK3 H2D(−404−428) |
| 3015 | GTCCCCATCTGTGAAACAGGGTTAT | SHANK3 H2D(−407−431) |
| 3016 | TGTGTCCCCATCTGTGAAACAGGGT | SHANK3 H2D(−410−434) |
| 3017 | CTCTGTGTCCCCATCTGTGAAACAG | SHANK3 H2D(−413−437) |
| 3018 | GTACTCTGTGTCCCCATCTGTGAAA | SHANK3 H2D(−416−440) |
| 3019 | CGGGTACTCTGTGTCCCCATCTGTG | SHANK3 H2D(−419−443) |
| 3020 | AGTCGGGTACTCTGTGTCCCCATCT | SHANK3 H2D(−422−446) |
| 3021 | CACAGTCGGGTACTCTGTGTCCCCA | SHANK3 H2D(−425−449) |
| 3022 | CTACACAGTCGGGTACTCTGTGTCC | SHANK3 H2D(−428−452) |
| 3023 | TGTCTACACAGTCGGGTACTCTGTG | SHANK3 H2D(−431−455) |
| 3024 | CACTGTCTACACAGTCGGGTACTCT | SHANK3 H2D(−434−458) |
| 3025 | CTTCACTGTCTACACAGTCGGGTAC | SHANK3 H2D(−437−461) |
| 3026 | CAGCTTCACTGTCTACACAGTCGGG | SHANK3 H2D(−440−464) |
| 3027 | CCCCAGCTTCACTGTCTACACAGTC | SHANK3 H2D(−443−467) |
| 3028 | CAGCCCCAGCTTCACTGTCTACACA | SHANK3 H2D(−446−470) |
| 3029 | GTTCAGCCCCAGCTTCACTGTCTAC | SHANK3 H2D(−449−473) |
| 3030 | TGGGTTCAGCCCCAGCTTCACTGTC | SHANK3 H2D(−452−476) |
| 3031 | ATCTGGGTTCAGCCCCAGCTTCACT | SHANK3 H2D(−455−479) |
| 3032 | CAGATCTGGGTTCAGCCCCAGCTTC | SHANK3 H2D(−458−482) |
| 3033 | AGACAGATCTGGGTTCAGCCCCAGC | SHANK3 H2D(−461−485) |
| 3034 | ATCAGACAGATCTGGGTTCAGCCCC | SHANK3 H2D(−464−488) |
| 3035 | GGAATCAGACAGATCTGGGTTCAGC | SHANK3 H2D(−467−491) |
| 3036 | CCTGGAATCAGACAGATCTGGGTTC | SHANK3 H2D(−470−494) |
| 3037 | GGGCCTGGAATCAGACAGATCTGGG | SHANK3 H2D(−473−497) |
| 3038 | ACAGGGCCTGGAATCAGACAGATCT | SHANK3 H2D(−476−500) |
| 3039 | GGCACAGGGCCTGGAATCAGACAGA | SHANK3 H2D(−479−503) |
| 3040 | CTGGGCACAGGGCCTGGAATCAGAC | SHANK3 H2D(−482−506) |
| 3041 | TGCCTGGGCACAGGGCCTGGAATCA | SHANK3 H2D(−485−509) |
| 3042 | GCATGCCTGGGCACAGGGCCTGGAA | SHANK3 H2D(−488−512) |
| 3043 | AAGGCATGCCTGGGCACAGGGCCTG | SHANK3 H2D(−491−515) |
| 3044 | TCAAAGGCATGCCTGGGCACAGGGC | SHANK3 H2D(−494−518) |
| 3045 | ACCTCAAAGGCATGCCTGGGCACAG | SHANK3 H2D(−497−521) |
| 3046 | GACACCTCAAAGGCATGCCTGGGCA | SHANK3 H2D(−500−524) |
| 3047 | GTAGACACCTCAAAGGCATGCCTGG | SHANK3 H2D(−503−527) |
| 3048 | GCAGTAGACACCTCAAAGGCATGCC | SHANK3 H2D(−506−530) |
| 3049 | CCAGCAGTAGACACCTCAAAGGCAT | SHANK3 H2D(−509−533) |
| 3050 | CACCCAGCAGTAGACACCTCAAAGG | SHANK3 H2D(−512−536) |
| 3051 | TGGCACCCAGCAGTAGACACCTCAA | SHANK3 H2D(−515−539) |
| 3052 | GGGTGGCACCCAGCAGTAGACACCT | SHANK3 H2D(−518−542) |
| 3053 | TGGGGGTGGCACCCAGCAGTAGACA | SHANK3 H2D(−521−545) |
| 3054 | AGTTGGGGGTGGCACCCAGCAGTAG | SHANK3 H2D(−524−548) |
| 3055 | CCCAGTTGGGGGTGGCACCCAGCAG | SHANK3 H2D(−527−551) |
| 3056 | AGGCCCAGTTGGGGGTGGCACCCAG | SHANK3 H2D(−530−554) |
| 3057 | GTCAGGCCCAGTTGGGGGTGGCACC | SHANK3 H2D(−533−557) |
| 3058 | GGGGTCAGGCCCAGTTGGGGGTGGC | SHANK3 H2D(−536−560) |
| 3059 | TTTGGGGTCAGGCCCAGTTGGGGGT | SHANK3 H2D(−539−563) |
| 3060 | GCATTTGGGGTCAGGCCCAGTTGGG | SHANK3 H2D(−542−566) |
| 3061 | AGAGCATTTGGGGTCAGGCCCAGTT | SHANK3 H2D(−545−569) |
| 3062 | TCAAGAGCATTTGGGGTCAGGCCCA | SHANK3 H2D(−548−572) |
| 3063 | CCCTCAAGAGCATTTGGGGTCAGGC | SHANK3 H2D(−551−575) |
| 3064 | CCCCCCTCAAGAGCATTTGGGGTCA | SHANK3 H2D(−554−578) |
| 3065 | GTGCCCCCCTCAAGAGCATTTGGGG | SHANK3 H2D(−557−581) |
| 3066 | AAGGTGCCCCCCTCAAGAGCATTTG | SHANK3 H2D(−560−584) |
| 3067 | AGTAAGGTGCCCCCCTCAAGAGCAT | SHANK3 H2D(−563−587) |
| 3068 | AATAGTAAGGTGCCCCCCTCAAGAG | SHANK3 H2D(−566−590) |
| 3069 | GGAAATAGTAAGGTGCCCCCCTCAA | SHANK3 H2D(−569−593) |
| 3070 | GCGGGAAATAGTAAGGTGCCCCCCT | SHANK3 H2D(−572−596) |
| 3071 | TCAGCGGGAAATAGTAAGGTGCCCC | SHANK3 H2D(−575−599) |
| 3072 | CATTCAGCGGGAAATAGTAAGGTGC | SHANK3 H2D(−578−602) |
| 3073 | TGACATTCAGCGGGAAATAGTAAGG | SHANK3 H2D(−581−605) |
| 3074 | CTCTGACATTCAGCGGGAAATAGTA | SHANK3 H2D(−584−608) |
| 3075 | CCCCTCTGACATTCAGCGGGAAATA | SHANK3 H2D(−587−611) |
| 3076 | CTGCCCCTCTGACATTCAGCGGGAA | SHANK3 H2D(−590−614) |
| 3077 | ACCCTGCCCCTCTGACATTCAGCGG | SHANK3 H2D(−593−617) |
| 3078 | CCCACCCTGCCCCTCTGACATTCAG | SHANK3 H2D(−596−620) |
| 3079 | GCACCCACCCTGCCCCTCTGACATT | SHANK3 H2D(−599−623) |
| 3080 | GTGGCACCCACCCTGCCCCTCTGAC | SHANK3 H2D(−602−626) |
| 3081 | GCTGTGGCACCCACCCTGCCCCTCT | SHANK3 H2D(−605−629) |
| 3082 | GGGGCTGTGGCACCCACCCTGCCCC | SHANK3 H2D(−608−632) |
| 3083 | GGAGGGGCTGTGGCACCCACCCTGC | SHANK3 H2D(−611−635) |
| 3084 | TGGGGAGGGGCTGTGGCACCCACCC | SHANK3 H2D(−614−638) |
| 3085 | CCCTGGGGAGGGGCTGTGGCACCCA | SHANK3 H2D(−617−641) |
| 3086 | AGCCCCTGGGGAGGGGCTGTGGCAC | SHANK3 H2D(−620−644) |
| 3087 | GGAAGCCCCTGGGGAGGGGCTGTGG | SHANK3 H2D(−623−647) |
| 3088 | GGTGGAAGCCCCTGGGGAGGGGCTG | SHANK3 H2D(−626−650) |
| 3089 | GCGGGTGGAAGCCCCTGGGGAGGGG | SHANK3 H2D(−629−653) |
| 3090 | GCTGCGGGTGGAAGCCCCTGGGGAG | SHANK3 H2D(−632−656) |
| 3091 | TGAGCTGCGGGTGGAAGCCCCTGGG | SHANK3 H2D(−635−659) |
| 3092 | CTGTGAGCTGCGGGTGGAAGCCCCT | SHANK3 H2D(−638−662) |
| 3093 | GGACTGTGAGCTGCGGGTGGAAGCC | SHANK3 H2D(−641−665) |
| 3094 | GCTGGACTGTGAGCTGCGGGTGGAA | SHANK3 H2D(−644−668) |
| 3095 | CCTGCTGGACTGTGAGCTGCGGGTG | SHANK3 H2D(−647−671) |
| 3096 | GTGCCTGCTGGACTGTGAGCTGCGG | SHANK3 H2D(−650−674) |
| 3097 | CAAGTGCCTGCTGGACTGTGAGCTG | SHANK3 H2D(−653−677) |
| 3098 | AAACAAGTGCCTGCTGGACTGTGAG | SHANK3 H2D(−656−680) |
| 3099 | AGCAAACAAGTGCCTGCTGGACTGT | SHANK3 H2D(−659−683) |
| 3100 | TCCAGCAAACAAGTGCCTGCTGGAC | SHANK3 H2D(−662−686) |
| 3101 | GTATCCAGCAAACAAGTGCCTGCTG | SHANK3 H2D(−665−689) |
| 3102 | AAAGTATCCAGCAAACAAGTGCCTG | SHANK3 H2D(−668−692) |
| 3103 | CATAAAGTATCCAGCAAACAAGTGC | SHANK3 H2D(−671−695) |
| 3104 | GCCCATAAAGTATCCAGCAAACAAG | SHANK3 H2D(−674−698) |
| 3105 | ACCGCCCATAAAGTATCCAGCAAAC | SHANK3 H2D(−677−701) |
| 3106 | GAGACCGCCCATAAAGTATCCAGCA | SHANK3 H2D(−680−704) |
| 3107 | CTCGAGACCGCCCATAAAGTATCCA | SHANK3 H2D(−683−707) |
| 3108 | GAGCTCGAGACCGCCCATAAAGTAT | SHANK3 H2D(−686−710) |
| 3109 | CCTGAGCTCGAGACCGCCCATAAAG | SHANK3 H2D(−689−713) |
| 3110 | TCTCCTGAGCTCGAGACCGCCCATA | SHANK3 H2D(−692−716) |
| 3111 | CCCTCTCCTGAGCTCGAGACCGCCC | SHANK3 H2D(−695−719) |
| 3112 | GACCCCTCTCCTGAGCTCGAGACCG | SHANK3 H2D(−698−722) |
| 3113 | TCTGACCCCTCTCCTGAGCTCGAGA | SHANK3 H2D(−701−725) |
| 3114 | CATTCTGACCCCTCTCCTGAGCTCG | SHANK3 H2D(−704−728) |
| 3115 | CTCCATTCTGACCCCTCTCCTGAGC | SHANK3 H2D(−707−731) |
| 3116 | ACCCTCCATTCTGACCCCTCTCCTG | SHANK3 H2D(−710−734) |
| 3117 | GAGACCCTCCATTCTGACCCCTCTC | SHANK3 H2D(−713−737) |
| 3118 | CCAGAGACCCTCCATTCTGACCCCT | SHANK3 H2D(−716−740) |
| 3119 | GCTCCAGAGACCCTCCATTCTGACC | SHANK3 H2D(−719−743) |
| 3120 | TGGGCTCCAGAGACCCTCCATTCTG | SHANK3 H2D(−722−746) |
| 3121 | TCCTGGGCTCCAGAGACCCTCCATT | SHANK3 H2D(−725−749) |
| 3122 | CTCTCCTGGGCTCCAGAGACCCTCC | SHANK3 H2D(−728−752) |
| 3123 | TTCCTCTCCTGGGCTCCAGAGACCC | SHANK3 H2D(−731−755) |
| 3124 | TGATTCCTCTCCTGGGCTCCAGAGA | SHANK3 H2D(−734−758) |
| 3125 | TTCTGATTCCTCTCCTGGGCTCCAG | SHANK3 H2D(−737−761) |
| 3126 | CAGTTCTGATTCCTCTCCTGGGCTC | SHANK3 H2D(−740−764) |
| 3127 | TGACAGTTCTGATTCCTCTCCTGGG | SHANK3 H2D(−743−767) |
| 3128 | GGCTGACAGTTCTGATTCCTCTCCT | SHANK3 H2D(−746−770) |
| 3129 | CAGGGCTGACAGTTCTGATTCCTCT | SHANK3 H2D(−749−773) |
| 3130 | ACACAGGGCTGACAGTTCTGATTCC | SHANK3 H2D(−752−776) |
| 3131 | AGGACACAGGGCTGACAGTTCTGAT | SHANK3 H2D(−755−779) |
| 3132 | ATTAGGACACAGGGCTGACAGTTCT | SHANK3 H2D(−758−782) |
| 3133 | ACCATTAGGACACAGGGCTGACAGT | SHANK3 H2D(−761−785) |
| 3134 | GGGACCATTAGGACACAGGGCTGAC | SHANK3 H2D(−764−788) |
| 3135 | GATGGGACCATTAGGACACAGGGCT | SHANK3 H2D(−767−791) |
| 3136 | ACTGATGGGACCATTAGGACACAGG | SHANK3 H2D(−770−794) |
| 3137 | GTCACTGATGGGACCATTAGGACAC | SHANK3 H2D(−773−797) |
| 3138 | AAAGTCACTGATGGGACCATTAGGA | SHANK3 H2D(−776−800) |
| 3139 | TCCAAAGTCACTGATGGGACCATTA | SHANK3 H2D(−779−803) |
| 3140 | GGCTCCAAAGTCACTGATGGGACCA | SHANK3 H2D(−782−806) |
| 3141 | TGAGGCTCCAAAGTCACTGATGGGA | SHANK3 H2D(−785−809) |
| 3142 | GCCTGAGGCTCCAAAGTCACTGATG | SHANK3 H2D(−788−812) |
| 3143 | GAAGCCTGAGGCTCCAAAGTCACTG | SHANK3 H2D(−791−815) |
| 3144 | ACAGAAGCCTGAGGCTCCAAAGTCA | SHANK3 H2D(−794−818) |
| 3145 | GGCACAGAAGCCTGAGGCTCCAAAG | SHANK3 H2D(−797−821) |
| 3146 | CCTGGCACAGAAGCCTGAGGCTCCA | SHANK3 H2D(−800−824) |
| 3147 | AAGCCTGGCACAGAAGCCTGAGGCT | SHANK3 H2D(−803−827) |
| 3148 | GAAAAGCCTGGCACAGAAGCCTGAG | SHANK3 H2D(−806−830) |
| 3149 | GCAGAAAAGCCTGGCACAGAAGCCT | SHANK3 H2D(−809−833) |
| 3150 | GCAGCAGAAAAGCCTGGCACAGAAG | SHANK3 H2D(−812−836) |
| 3151 | GGGGCAGCAGAAAAGCCTGGCACAG | SHANK3 H2D(−815−839) |
| 3152 | CCTGGGGCAGCAGAAAAGCCTGGCA | SHANK3 H2D(−818−842) |
| 3153 | TGCCCTGGGGCAGCAGAAAAGCCTG | SHANK3 H2D(−821−845) |
| 3154 | GCCTGCCCTGGGGCAGCAGAAAAGC | SHANK3 H2D(−824−848) |
| 3155 | ACCGCCTGCCCTGGGGCAGCAGAAA | SHANK3 H2D(−827−851) |
| 3156 | ACCACCGCCTGCCCTGGGGCAGCAG | SHANK3 H2D(−830−854) |
| 3157 | TTGACCACCGCCTGCCCTGGGGCAG | SHANK3 H2D(−833−857) |
| 3158 | CCGTTGACCACCGCCTGCCCTGGGG | SHANK3 H2D(−836−860) |
| 3159 | AGGCCGTTGACCACCGCCTGCCCTG | SHANK3 H2D(−839−863) |
| 3160 | CCAAGGCCGTTGACCACCGCCTGCC | SHANK3 H2D(−842−866) |
| 3161 | GTGCCAAGGCCGTTGACCACCGCCT | SHANK3 H2D(−845−869) |
| 3162 | ATAGTGCCAAGGCCGTTGACCACCG | SHANK3 H2D(−848−872) |
| 3163 | GCCATAGTGCCAAGGCCGTTGACCA | SHANK3 H2D(−851−875) |
| 3164 | CCAGCCATAGTGCCAAGGCCGTTGA | SHANK3 H2D(−854−878) |
| 3165 | TGCCCAGCCATAGTGCCAAGGCCGT | SHANK3 H2D(−857−881) |
| 3166 | CTCTGCCCAGCCATAGTGCCAAGGC | SHANK3 H2D(−860−884) |
| 3167 | GGACTCTGCCCAGCCATAGTGCCAA | SHANK3 H2D(−863−887) |
| 3168 | AGTGGACTCTGCCCAGCCATAGTGC | SHANK3 H2D(−866−890) |
| 3169 | CACAGTGGACTCTGCCCAGCCATAG | SHANK3 H2D(−869−893) |
| 3170 | TTCCACAGTGGACTCTGCCCAGCCA | SHANK3 H2D(−872−896) |
| 3171 | CCTTTCCACAGTGGACTCTGCCCAG | SHANK3 H2D(−875−899) |
| 3172 | GGACCTTTCCACAGTGGACTCTGCC | SHANK3 H2D(−878−902) |
| 3173 | GGGGGACCTTTCCACAGTGGACTCT | SHANK3 H2D(−881−905) |
| 3174 | AGAGGGGGACCTTTCCACAGTGGAC | SHANK3 H2D(−884−908) |
| 3175 | AGGAGAGGGGGACCTTTCCACAGTG | SHANK3 H2D(−887−911) |
| 3176 | GGCAGGAGAGGGGGACCTTTCCACA | SHANK3 H2D(−890−914) |
| 3177 | AGTGGCAGGAGAGGGGGACCTTTCC | SHANK3 H2D(−893−917) |
| 3178 | GCCAGTGGCAGGAGAGGGGGACCTT | SHANK3 H2D(−896−920) |
| 3179 | AGGGCCAGTGGCAGGAGAGGGGGAC | SHANK3 H2D(−899−923) |
| 3180 | GTGAGGGCCAGTGGCAGGAGAGGGG | SHANK3 H2D(−902−926) |
| 3181 | CAAGTGAGGGCCAGTGGCAGGAGAG | SHANK3 H2D(−905−929) |
| 3182 | GGTCAAGTGAGGGCCAGTGGCAGGA | SHANK3 H2D(−908−932) |
| 3183 | CAAGGTCAAGTGAGGGCCAGTGGCA | SHANK3 H2D(−911−935) |
| 3184 | TGACAAGGTCAAGTGAGGGCCAGTG | SHANK3 H2D(−914−938) |
| 3185 | GGCTGACAAGGTCAAGTGAGGGCCA | SHANK3 H2D(−917−941) |
| 3186 | CCAGGCTGACAAGGTCAAGTGAGGG | SHANK3 H2D(−920−944) |
| 3187 | AACCCAGGCTGACAAGGTCAAGTGA | SHANK3 H2D(−923−947) |
| 3188 | AGGAACCCAGGCTGACAAGGTCAAG | SHANK3 H2D(−926−950) |
| 3189 | GGTAGGAACCCAGGCTGACAAGGTC | SHANK3 H2D(−929−953) |
| 3190 | TGGGGTAGGAACCCAGGCTGACAAG | SHANK3 H2D(−932−956) |
| 3191 | CCATGGGGTAGGAACCCAGGCTGAC | SHANK3 H2D(−935−959) |
| 3192 | TGGCCATGGGGTAGGAACCCAGGCT | SHANK3 H2D(−938−962) |
| 3193 | AGGTGGCCATGGGGTAGGAACCCAG | SHANK3 H2D(−941−965) |
| 3194 | GAGAGGTGGCCATGGGGTAGGAACC | SHANK3 H2D(−944−968) |
| 3195 | AGGGAGAGGTGGCCATGGGGTAGGA | SHANK3 H3A(−945−921) |
| 3196 | CGGAGGGAGAGGTGGCCATGGGGTA | SHANK3 H3A(−942−918) |
| 3197 | ATCCGGAGGGAGAGGTGGCCATGGG | SHANK3 H3A(−939−915) |
| 3198 | GAGATCCGGAGGGAGAGGTGGCCAT | SHANK3 H3A(−936−912) |
| 3199 | AGAGAGATCCGGAGGGAGAGGTGGC | SHANK3 H3A(−933−909) |
| 3200 | TGAAGAGAGATCCGGAGGGAGAGGT | SHANK3 H3A(−930−906) |
| 3201 | CACTGAAGAGAGATCCGGAGGGAGA | SHANK3 H3A(−927−903) |
| 3202 | GGTCACTGAAGAGAGATCCGGAGGG | SHANK3 H3A(−924−900) |
| 3203 | GTTGGTCACTGAAGAGAGATCCGGA | SHANK3 H3A(−921−897) |
| 3204 | CTTGTTGGTCACTGAAGAGAGATCC | SHANK3 H3A(−918−894) |
| 3205 | TGTCTTGTTGGTCACTGAAGAGAGA | SHANK3 H3A(−915−891) |
| 3206 | TCATGTCTTGTTGGTCACTGAAGAG | SHANK3 H3A(−912−888) |
| 3207 | CACTCATGTCTTGTTGGTCACTGAA | SHANK3 H3A(−909−885) |
| 3208 | AGTCACTCATGTCTTGTTGGTCACT | SHANK3 H3A(−906−882) |
| 3209 | GTGAGTCACTCATGTCTTGTTGGTC | SHANK3 H3A(−903−879) |
| 3210 | AGAGTGAGTCACTCATGTCTTGTTG | SHANK3 H3A(−900−876) |
| 3211 | TTCAGAGTGAGTCACTCATGTCTTG | SHANK3 H3A(−897−873) |
| 3212 | TACTTCAGAGTGAGTCACTCATGTC | SHANK3 H3A(−894−870) |
| 3213 | ACCTACTTCAGAGTGAGTCACTCAT | SHANK3 H3A(−891−867) |
| 3214 | GAGACCTACTTCAGAGTGAGTCACT | SHANK3 H3A(−888−864) |
| 3215 | AACGAGACCTACTTCAGAGTGAGTC | SHANK3 H3A(−885−861) |
| 3216 | ACAAACGAGACCTACTTCAGAGTGA | SHANK3 H3A(−882−858) |
| 3217 | AAAACAAACGAGACCTACTTCAGAG | SHANK3 H3A(−879−855) |
| 3218 | CTAAAAACAAACGAGACCTACTTCA | SHANK3 H3A(−876−852) |
| 3219 | ATCCTAAAAACAAACGAGACCTACT | SHANK3 H3A(−873−849) |
| 3220 | CTCATCCTAAAAACAAACGAGACCT | SHANK3 H3A(−870−846) |
| 3221 | AGGCTCATCCTAAAAACAAACGAGA | SHANK3 H3A(−867−843) |
| 3222 | TTCAGGCTCATCCTAAAAACAAACG | SHANK3 H3A(−864−840) |
| 3223 | AAGTTCAGGCTCATCCTAAAAACAA | SHANK3 H3A(−861−837) |
| 3224 | AAGAAGTTCAGGCTCATCCTAAAAA | SHANK3 H3A(−858−834) |
| 3225 | TGTAAGAAGTTCAGGCTCATCCTAA | SHANK3 H3A(−855−831) |
| 3226 | CTATGTAAGAAGTTCAGGCTCATCC | SHANK3 H3A(−852−828) |
| 3227 | GGGCTATGTAAGAAGTTCAGGCTCA | SHANK3 H3A(−849−825) |
| 3228 | CACGGGCTATGTAAGAAGTTCAGGC | SHANK3 H3A(−846−822) |
| 3229 | AAACACGGGCTATGTAAGAAGTTCA | SHANK3 H3A(−843−819) |
| 3230 | CAGAAACACGGGCTATGTAAGAAGT | SHANK3 H3A(−840−816) |
| 3231 | AAGCAGAAACACGGGCTATGTAAGA | SHANK3 H3A(−837−813) |
| 3232 | GGCAAGCAGAAACACGGGCTATGTA | SHANK3 H3A(−834−810) |
| 3233 | CCAGGCAAGCAGAAACACGGGCTAT | SHANK3 H3A(−831−807) |
| 3234 | AGCCCAGGCAAGCAGAAACACGGGC | SHANK3 H3A(−828−804) |
| 3235 | TGGAGCCCAGGCAAGCAGAAACACG | SHANK3 H3A(−825−801) |
| 3236 | AGCTGGAGCCCAGGCAAGCAGAAAC | SHANK3 H3A(−822−798) |
| 3237 | GCCAGCTGGAGCCCAGGCAAGCAGA | SHANK3 H3A(−819−795) |
| 3238 | AAGGCCAGCTGGAGCCCAGGCAAGC | SHANK3 H3A(−816−792) |
| 3239 | GGAAAGGCCAGCTGGAGCCCAGGCA | SHANK3 H3A(−813−789) |
| 3240 | GAGGGAAAGGCCAGCTGGAGCCCAG | SHANK3 H3A(−810−786) |
| 3241 | GGAGAGGGAAAGGCCAGCTGGAGCC | SHANK3 H3A(−807−783) |
| 3242 | AGGGGAGAGGGAAAGGCCAGCTGGA | SHANK3 H3A(−804−780) |
| 3243 | ACCAGGGGAGAGGGAAAGGCCAGCT | SHANK3 H3A(−801−777) |
| 3244 | ACTACCAGGGGAGAGGGAAAGGCCA | SHANK3 H3A(−798−774) |
| 3245 | CTCACTACCAGGGGAGAGGGAAAGG | SHANK3 H3A(−795−771) |
| 3246 | GTGCTCACTACCAGGGGAGAGGGAA | SHANK3 H3A(−792−768) |
| 3247 | GAAGTGCTCACTACCAGGGGAGAGG | SHANK3 H3A(−789−765) |
| 3248 | GAGGAAGTGCTCACTACCAGGGGAG | SHANK3 H3A(−786−762) |
| 3249 | GAGGAGGAAGTGCTCACTACCAGGG | SHANK3 H3A(−783−759) |
| 3250 | AAAGAGGAGGAAGTGCTCACTACCA | SHANK3 H3A(−780−756) |
| 3251 | GTGAAAGAGGAGGAAGTGCTCACTA | SHANK3 H3A(−777−753) |
| 3252 | GCAGTGAAAGAGGAGGAAGTGCTCA | SHANK3 H3A(−774−750) |
| 3253 | CAGGCAGTGAAAGAGGAGGAAGTGC | SHANK3 H3A(−771−747) |
| 3254 | GGCCAGGCAGTGAAAGAGGAGGAAG | SHANK3 H3A(−768−744) |
| 3255 | GCGGGCCAGGCAGTGAAAGAGGAGG | SHANK3 H3A(−765−741) |
| 3256 | CAGGCGGGCCAGGCAGTGAAAGAGG | SHANK3 H3A(−762−738) |
| 3257 | CCTCAGGCGGGCCAGGCAGTGAAAG | SHANK3 H3A(−759−735) |
| 3258 | CCTCCTCAGGCGGGCCAGGCAGTGA | SHANK3 H3A(−756−732) |
| 3259 | CAACCTCCTCAGGCGGGCCAGGCAG | SHANK3 H3A(−753−729) |
| 3260 | AGGCAACCTCCTCAGGCGGGCCAGG | SHANK3 H3A(−750−726) |
| 3261 | GGGAGGCAACCTCCTCAGGCGGGCC | SHANK3 H3A(−747−723) |
| 3262 | CTTGGGAGGCAACCTCCTCAGGCGG | SHANK3 H3A(−744−720) |
| 3263 | TGCCTTGGGAGGCAACCTCCTCAGG | SHANK3 H3A(−741−717) |
| 3264 | CCCTGCCTTGGGAGGCAACCTCCTC | SHANK3 H3A(−738−714) |
| 3265 | GACCCCTGCCTTGGGAGGCAACCTC | SHANK3 H3A(−735−711) |
| 3266 | AGGGACCCCTGCCTTGGGAGGCAAC | SHANK3 H3A(−732−708) |
| 3267 | CATAGGGACCCCTGCCTTGGGAGGC | SHANK3 H3A(−729−705) |
| 3268 | TGCCATAGGGACCCCTGCCTTGGGA | SHANK3 H3A(−726−702) |
| 3269 | GGGTGCCATAGGGACCCCTGCCTTG | SHANK3 H3A(−723−699) |
| 3270 | GGAGGGTGCCATAGGGACCCCTGCC | SHANK3 H3A(−720−696) |
| 3271 | AGGGGAGGGTGCCATAGGGACCCCT | SHANK3 H3A(−717−693) |
| 3272 | AATAGGGGAGGGTGCCATAGGGACC | SHANK3 H3A(−714−690) |
| 3273 | AGAAATAGGGGAGGGTGCCATAGGG | SHANK3 H3A(−711−687) |
| 3274 | CTCAGAAATAGGGGAGGGTGCCATA | SHANK3 H3A(−708−684) |
| 3275 | TACCTCAGAAATAGGGGAGGGTGCC | SHANK3 H3A(−705−681) |
| 3276 | GTGTACCTCAGAAATAGGGGAGGGT | SHANK3 H3A(−702−678) |
| 3277 | CCAGTGTACCTCAGAAATAGGGGAG | SHANK3 H3A(−699−675) |
| 3278 | CACCCAGTGTACCTCAGAAATAGGG | SHANK3 H3A(−696−672) |
| 3279 | AAACACCCAGTGTACCTCAGAAATA | SHANK3 H3A(−693−669) |
| 3280 | CATAAACACCCAGTGTACCTCAGAA | SHANK3 H3A(−690−666) |
| 3281 | TTCCATAAACACCCAGTGTACCTCA | SHANK3 H3A(−687−663) |
| 3282 | CCCTTCCATAAACACCCAGTGTACC | SHANK3 H3A(−684−660) |
| 3283 | GGGCCCTTCCATAAACACCCAGTGT | SHANK3 H3A(−681−657) |
| 3284 | CCGGGGCCCTTCCATAAACACCCAG | SHANK3 H3A(−678−654) |
| 3285 | AGGCCGGGGCCCTTCCATAAACACC | SHANK3 H3A(−675−651) |
| 3286 | CAAAGGCCGGGGCCCTTCCATAAAC | SHANK3 H3A(−672−648) |
| 3287 | GGCCAAAGGCCGGGGCCCTTCCATA | SHANK3 H3A(−669−645) |
| 3288 | CCTGGCCAAAGGCCGGGGCCCTTCC | SHANK3 H3A(−666−642) |
| 3289 | TGCCCTGGCCAAAGGCCGGGGCCCT | SHANK3 H3A(−663−639) |
| 3290 | AGGTGCCCTGGCCAAAGGCCGGGGC | SHANK3 H3A(−660−636) |
| 3291 | GCAAGGTGCCCTGGCCAAAGGCCGG | SHANK3 H3A(−657−633) |
| 3292 | AAGGCAAGGTGCCCTGGCCAAAGGC | SHANK3 H3A(−654−630) |
| 3293 | CACAAGGCAAGGTGCCCTGGCCAAA | SHANK3 H3A(−651−627) |
| 3294 | ACACACAAGGCAAGGTGCCCTGGCC | SHANK3 H3A(−648−624) |
| 3295 | CACACACACAAGGCAAGGTGCCCTG | SHANK3 H3A(−645−621) |
| 3296 | CACCACACACACAAGGCAAGGTGCC | SHANK3 H3A(−642−618) |
| 3297 | GGACACCACACACACAAGGCAAGGT | SHANK3 H3A(−639−615) |
| 3298 | TACGGACACCACACACACAAGGCAA | SHANK3 H3A(−636−612) |
| 3299 | CACTACGGACACCACACACACAAGG | SHANK3 H3A(−633−609) |
| 3300 | CGGCACTACGGACACCACACACACA | SHANK3 H3A(−630−606) |
| 3301 | AGCCGGCACTACGGACACCACACAC | SHANK3 H3A(−627−603) |
| 3302 | CCCAGCCGGCACTACGGACACCACA | SHANK3 H3A(−624−600) |
| 3303 | TTCCCCAGCCGGCACTACGGACACC | SHANK3 H3A(−621−597) |
| 3304 | CACTTCCCCAGCCGGCACTACGGAC | SHANK3 H3A(−618−594) |
| 3305 | CCTCACTTCCCCAGCCGGCACTACG | SHANK3 H3A(−615−591) |
| 3306 | AGGCCTCACTTCCCCAGCCGGCACT | SHANK3 H3A(−612−588) |
| 3307 | ACAAGGCCTCACTTCCCCAGCCGGC | SHANK3 H3A(−609−585) |
| 3308 | CCCACAAGGCCTCACTTCCCCAGCC | SHANK3 H3A(−606−582) |
| 3309 | CACCCCACAAGGCCTCACTTCCCCA | SHANK3 H3A(−603−579) |
| 3310 | ACTCACCCCACAAGGCCTCACTTCC | SHANK3 H3A(−600−576) |
| 3311 | TTCACTCACCCCACAAGGCCTCACT | SHANK3 H3A(−597−573) |
| 3312 | TTATTCACTCACCCCACAAGGCCTC | SHANK3 H3A(−594−570) |
| 3313 | AGTTTATTCACTCACCCCACAAGGC | SHANK3 H3A(−591−567) |
| 3314 | CCCAGTTTATTCACTCACCCCACAA | SHANK3 H3A(−588−564) |
| 3315 | TCACCCAGTTTATTCACTCACCCCA | SHANK3 H3A(−585−561) |
| 3316 | CATTCACCCAGTTTATTCACTCACC | SHANK3 H3A(−582−558) |
| 3317 | ACTCATTCACCCAGTTTATTCACTC | SHANK3 H3A(−579−555) |
| 3318 | TGTACTCATTCACCCAGTTTATTCA | SHANK3 H3A(−576−552) |
| 3319 | CCTTGTACTCATTCACCCAGTTTAT | SHANK3 H3A(−573−549) |
| 3320 | TGGCCTTGTACTCATTCACCCAGTT | SHANK3 H3A(−570−546) |
| 3321 | TGATGGCCTTGTACTCATTCACCCA | SHANK3 H3A(−567−543) |
| 3322 | CTTTGATGGCCTTGTACTCATTCAC | SHANK3 H3A(−564−540) |
| 3323 | TGACTTTGATGGCCTTGTACTCATT | SHANK3 H3A(−561−537) |
| 3324 | ACATGACTTTGATGGCCTTGTACTC | SHANK3 H3A(−558−534) |
| 3325 | GTGACATGACTTTGATGGCCTTGTA | SHANK3 H3A(−555−531) |
| 3326 | CCTGTGACATGACTTTGATGGCCTT | SHANK3 H3A(−552−528) |
| 3327 | CATCCTGTGACATGACTTTGATGGC | SHANK3 H3A(−549−525) |
| 3328 | ACCCATCCTGTGACATGACTTTGAT | SHANK3 H3A(−546−522) |
| 3329 | GGGACCCATCCTGTGACATGACTTT | SHANK3 H3A(−543−519) |
| 3330 | CCAGGGACCCATCCTGTGACATGAC | SHANK3 H3A(−540−516) |
| 3331 | ACCCCAGGGACCCATCCTGTGACAT | SHANK3 H3A(−537−513) |
| 3332 | CCCACCCCAGGGACCCATCCTGTGA | SHANK3 H3A(−534−510) |
| 3333 | ACCCCCACCCCAGGGACCCATCCTG | SHANK3 H3A(−531−507) |
| 3334 | CAGACCCCCACCCCAGGGACCCATC | SHANK3 H3A(−528−504) |
| 3335 | CCGCAGACCCCCACCCCAGGGACCC | SHANK3 H3A(−525−501) |
| 3336 | CCACCGCAGACCCCCACCCCAGGGA | SHANK3 H3A(−522−498) |
| 3337 | ACCCCACCGCAGACCCCCACCCCAG | SHANK3 H3A(−519−495) |
| 3338 | CTCACCCCACCGCAGACCCCCACCC | SHANK3 H3A(−516−492) |
| 3339 | CAGCTCACCCCACCGCAGACCCCCA | SHANK3 H3A(−513−489) |
| 3340 | CTCCAGCTCACCCCACCGCAGACCC | SHANK3 H3A(−510−486) |
| 3341 | CCTCTCCAGCTCACCCCACCGCAGA | SHANK3 H3A(−507−483) |
| 3342 | CTTCCTCTCCAGCTCACCCCACCGC | SHANK3 H3A(−504−480) |
| 3343 | CATCTTCCTCTCCAGCTCACCCCAC | SHANK3 H3A(−501−477) |
| 3344 | GCCCATCTTCCTCTCCAGCTCACCC | SHANK3 H3A(−498−474) |
| 3345 | TCGGCCCATCTTCCTCTCCAGCTCA | SHANK3 H3A(−495−471) |
| 3346 | TCTTCGGCCCATCTTCCTCTCCAGC | SHANK3 H3A(−492−468) |
| 3347 | CCTTCTTCGGCCCATCTTCCTCTCC | SHANK3 H3A(−489−465) |
| 3348 | GCTCCTTCTTCGGCCCATCTTCCTC | SHANK3 H3A(−486−462) |
| 3349 | CCTGCTCCTTCTTCGGCCCATCTTC | SHANK3 H3A(−483−459) |
| 3350 | CAACCTGCTCCTTCTTCGGCCCATC | SHANK3 H3A(−480−456) |
| 3351 | AGACAACCTGCTCCTTCTTCGGCCC | SHANK3 H3A(−477−453) |
| 3352 | CTGAGACAACCTGCTCCTTCTTCGG | SHANK3 H3A(−474−450) |
| 3353 | ACCCTGAGACAACCTGCTCCTTCTT | SHANK3 H3A(−471−447) |
| 3354 | AGCACCCTGAGACAACCTGCTCCTT | SHANK3 H3A(−468−444) |
| 3355 | CTCAGCACCCTGAGACAACCTGCTC | SHANK3 H3A(−465−441) |
| 3356 | ATCCTCAGCACCCTGAGACAACCTG | SHANK3 H3A(−462−438) |
| 3357 | TGAATCCTCAGCACCCTGAGACAAC | SHANK3 H3A(−459−435) |
| 3358 | CCCTGAATCCTCAGCACCCTGAGAC | SHANK3 H3A(−456−432) |
| 3359 | CAGCCCTGAATCCTCAGCACCCTGA | SHANK3 H3A(−453−429) |
| 3360 | CAGCAGCCCTGAATCCTCAGCACCC | SHANK3 H3A(−450−426) |
| 3361 | CCACAGCAGCCCTGAATCCTCAGCA | SHANK3 H3A(−447−423) |
| 3362 | GCCCCACAGCAGCCCTGAATCCTCA | SHANK3 H3A(−444−420) |
| 3363 | CCAGCCCCACAGCAGCCCTGAATCC | SHANK3 H3A(−441−417) |
| 3364 | GAACCAGCCCCACAGCAGCCCTGAA | SHANK3 H3A(−438−414) |
| 3365 | CAGGAACCAGCCCCACAGCAGCCCT | SHANK3 H3A(−435−411) |
| 3366 | AGGCAGGAACCAGCCCCACAGCAGC | SHANK3 H3A(−432−408) |
| 3367 | CCCAGGCAGGAACCAGCCCCACAGC | SHANK3 H3A(−429−405) |
| 3368 | CCACCCAGGCAGGAACCAGCCCCAC | SHANK3 H3A(−426−402) |
| 3369 | GGTCCACCCAGGCAGGAACCAGCCC | SHANK3 H3A(−423−399) |
| 3370 | CGAGGTCCACCCAGGCAGGAACCAG | SHANK3 H3A(−420−396) |
| 3371 | GCCCGAGGTCCACCCAGGCAGGAAC | SHANK3 H3A(−417−393) |
| 3372 | AGAGCCCGAGGTCCACCCAGGCAGG | SHANK3 H3A(−414−390) |
| 3373 | AGAAGAGCCCGAGGTCCACCCAGGC | SHANK3 H3A(−411−387) |
| 3374 | CCTAGAAGAGCCCGAGGTCCACCCA | SHANK3 H3A(−408−384) |
| 3375 | TGGCCTAGAAGAGCCCGAGGTCCAC | SHANK3 H3A(−405−381) |
| 3376 | GCCTGGCCTAGAAGAGCCCGAGGTC | SHANK3 H3A(−402−378) |
| 3377 | CCTGCCTGGCCTAGAAGAGCCCGAG | SHANK3 H3A(−399−375) |
| 3378 | CGACCTGCCTGGCCTAGAAGAGCCC | SHANK3 H3A(−396−372) |
| 3379 | CCCCGACCTGCCTGGCCTAGAAGAG | SHANK3 H3A(−393−369) |
| 3380 | CTGCCCCGACCTGCCTGGCCTAGAA | SHANK3 H3A(−390−366) |
| 3381 | CTGCTGCCCCGACCTGCCTGGCCTA | SHANK3 H3A(−387−363) |
| 3382 | GCCCTGCTGCCCCGACCTGCCTGGC | SHANK3 H3A(−384−360) |
| 3383 | CCGGCCCTGCTGCCCCGACCTGCCT | SHANK3 H3A(−381−357) |
| 3384 | TCTCCGGCCCTGCTGCCCCGACCTG | SHANK3 H3A(−378−354) |
| 3385 | GTCTCTCCGGCCCTGCTGCCCCGAC | SHANK3 H3A(−375−351) |
| 3386 | TCCGTCTCTCCGGCCCTGCTGCCCC | SHANK3 H3A(−372−348) |
| 3387 | GGCTCCGTCTCTCCGGCCCTGCTGC | SHANK3 H3A(−369−345) |
| 3388 | CCTGGCTCCGTCTCTCCGGCCCTGC | SHANK3 H3A(−366−342) |
| 3389 | TGCCCTGGCTCCGTCTCTCCGGCCC | SHANK3 H3A(−363−339) |
| 3390 | CACTGCCCTGGCTCCGTCTCTCCGG | SHANK3 H3A(−360−336) |
| 3391 | AGTCACTGCCCTGGCTCCGTCTCTC | SHANK3 H3A(−357−333) |
| 3392 | CCCAGTCACTGCCCTGGCTCCGTCT | SHANK3 H3A(−354−330) |
| 3393 | AGGCCCAGTCACTGCCCTGGCTCCG | SHANK3 H3A(−351−327) |
| 3394 | TCCAGGCCCAGTCACTGCCCTGGCT | SHANK3 H3A(−348−324) |
| 3395 | CACTCCAGGCCCAGTCACTGCCCTG | SHANK3 H3A(−345−321) |
| 3396 | CCCCACTCCAGGCCCAGTCACTGCC | SHANK3 H3A(−342−318) |
| 3397 | GTCCCCCACTCCAGGCCCAGTCACT | SHANK3 H3A(−339−315) |
| 3398 | CAAGTCCCCCACTCCAGGCCCAGTC | SHANK3 H3A(−336−312) |
| 3399 | AAGCAAGTCCCCCACTCCAGGCCCA | SHANK3 H3A(−333−309) |
| 3400 | CCCAAGCAAGTCCCCCACTCCAGGC | SHANK3 H3A(−330−306) |
| 3401 | GGCCCCAAGCAAGTCCCCCACTCCA | SHANK3 H3A(−327−303) |
| 3402 | TGGGGCCCCAAGCAAGTCCCCCACT | SHANK3 H3A(−324−300) |
| 3403 | TGGTGGGGCCCCAAGCAAGTCCCCC | SHANK3 H3A(−321−297) |
| 3404 | CCCTGGTGGGGCCCCAAGCAAGTCC | SHANK3 H3A(−318−294) |
| 3405 | TCACCCTGGTGGGGCCCCAAGCAAG | SHANK3 H3A(−315−291) |
| 3406 | AGGTCACCCTGGTGGGGCCCCAAGC | SHANK3 H3A(−312−288) |
| 3407 | GCCAGGTCACCCTGGTGGGGCCCCA | SHANK3 H3A(−309−285) |
| 3408 | AAGGCCAGGTCACCCTGGTGGGGCC | SHANK3 H3A(−306−282) |
| 3409 | ACCAAGGCCAGGTCACCCTGGTGGG | SHANK3 H3A(−303−279) |
| 3410 | CTCACCAAGGCCAGGTCACCCTGGT | SHANK3 H3A(−300−276) |
| 3411 | CCCCTCACCAAGGCCAGGTCACCCT | SHANK3 H3A(−297−273) |
| 3412 | TAGCCCCTCACCAAGGCCAGGTCAC | SHANK3 H3A(−294−270) |
| 3413 | AAGTAGCCCCTCACCAAGGCCAGGT | SHANK3 H3A(−291−267) |
| 3414 | CCCAAGTAGCCCCTCACCAAGGCCA | SHANK3 H3A(−288−264) |
| 3415 | GTACCCAAGTAGCCCCTCACCAAGG | SHANK3 H3A(−285−261) |
| 3416 | CTTGTACCCAAGTAGCCCCTCACCA | SHANK3 H3A(−282−258) |
| 3417 | CAGCTTGTACCCAAGTAGCCCCTCA | SHANK3 H3A(−279−255) |
| 3418 | TGTCAGCTTGTACCCAAGTAGCCCC | SHANK3 H3A(−276−252) |
| 3419 | GACTGTCAGCTTGTACCCAAGTAGC | SHANK3 H3A(−273−249) |
| 3420 | CACGACTGTCAGCTTGTACCCAAGT | SHANK3 H3A(−270−246) |
| 3421 | AGTCACGACTGTCAGCTTGTACCCA | SHANK3 H3A(−267−243) |
| 3422 | ACCAGTCACGACTGTCAGCTTGTAC | SHANK3 H3A(−264−240) |
| 3423 | CAAACCAGTCACGACTGTCAGCTTG | SHANK3 H3A(−261−237) |
| 3424 | AGCCAAACCAGTCACGACTGTCAGC | SHANK3 H3A(−258−234) |
| 3425 | GATAGCCAAACCAGTCACGACTGTC | SHANK3 H3A(−255−231) |
| 3426 | CCTGATAGCCAAACCAGTCACGACT | SHANK3 H3A(−252−228) |
| 3427 | CGCCCTGATAGCCAAACCAGTCACG | SHANK3 H3A(−249−225) |
| 3428 | TTGCGCCCTGATAGCCAAACCAGTC | SHANK3 H3A(−246−222) |
| 3429 | TCCTTGCGCCCTGATAGCCAAACCA | SHANK3 H3A(−243−219) |
| 3430 | CCTTCCTTGCGCCCTGATAGCCAAA | SHANK3 H3A(−240−216) |
| 3431 | AGCCCTTCCTTGCGCCCTGATAGCC | SHANK3 H3A(−237−213) |
| 3432 | CAAAGCCCTTCCTTGCGCCCTGATA | SHANK3 H3A(−234−210) |
| 3433 | GCTCAAAGCCCTTCCTTGCGCCCTG | SHANK3 H3A(−231−207) |
| 3434 | ACGGCTCAAAGCCCTTCCTTGCGCC | SHANK3 H3A(−228−204) |
| 3435 | TGCACGGCTCAAAGCCCTTCCTTGC | SHANK3 H3A(−225−201) |
| 3436 | CCATGCACGGCTCAAAGCCCTTCCT | SHANK3 H3A(−222−198) |
| 3437 | GGCCCATGCACGGCTCAAAGCCCTT | SHANK3 H3A(−219−195) |
| 3438 | GTGGGCCCATGCACGGCTCAAAGCC | SHANK3 H3A(−216−192) |
| 3439 | CGGGTGGGCCCATGCACGGCTCAAA | SHANK3 H3A(−213−189) |
| 3440 | ACTCGGGTGGGCCCATGCACGGCTC | SHANK3 H3A(−210−186) |
| 3441 | CCCACTCGGGTGGGCCCATGCACGG | SHANK3 H3A(−207−183) |
| 3442 | ATTCCCACTCGGGTGGGCCCATGCA | SHANK3 H3A(−204−180) |
| 3443 | CCAATTCCCACTCGGGTGGGCCCAT | SHANK3 H3A(−201−177) |
| 3444 | GCCCCAATTCCCACTCGGGTGGGCC | SHANK3 H3A(−198−174) |
| 3445 | ACGGCCCCAATTCCCACTCGGGTGG | SHANK3 H3A(−195−171) |
| 3446 | ACCACGGCCCCAATTCCCACTCGGG | SHANK3 H3A(−192−168) |
| 3447 | CCCACCACGGCCCCAATTCCCACTC | SHANK3 H3A(−189−165) |
| 3448 | ACTCCCACCACGGCCCCAATTCCCA | SHANK3 H3A(−186−162) |
| 3449 | TGCACTCCCACCACGGCCCCAATTC | SHANK3 H3A(−183−159) |
| 3450 | TCCTGCACTCCCACCACGGCCCCAA | SHANK3 H3A(−180−156) |
| 3451 | CGGTCCTGCACTCCCACCACGGCCC | SHANK3 H3A(−177−153) |
| 3452 | CCACGGTCCTGCACTCCCACCACGG | SHANK3 H3A(−174−150) |
| 3453 | CAACCACGGTCCTGCACTCCCACCA | SHANK3 H3A(−171−147) |
| 3454 | TGTCAACCACGGTCCTGCACTCCCA | SHANK3 H3A(−168−144) |
| 3455 | AATTGTCAACCACGGTCCTGCACTC | SHANK3 H3A(−165−141) |
| 3456 | CACAATTGTCAACCACGGTCCTGCA | SHANK3 H3A(−162−138) |
| 3457 | CATCACAATTGTCAACCACGGTCCT | SHANK3 H3A(−159−135) |
| 3458 | TGACATCACAATTGTCAACCACGGT | SHANK3 H3A(−156−132) |
| 3459 | ACCTGACATCACAATTGTCAACCAC | SHANK3 H3A(−153−129) |
| 3460 | GTCACCTGACATCACAATTGTCAAC | SHANK3 H3A(−150−126) |
| 3461 | CCTGTCACCTGACATCACAATTGTC | SHANK3 H3A(−147−123) |
| 3462 | TGACCTGTCACCTGACATCACAATT | SHANK3 H3A(−144−120) |
| 3463 | GTCTGACCTGTCACCTGACATCACA | SHANK3 H3A(−141−117) |
| 3464 | CCAGTCTGACCTGTCACCTGACATC | SHANK3 H3A(−138−114) |
| 3465 | CTACCAGTCTGACCTGTCACCTGAC | SHANK3 H3A(−135−111) |
| 3466 | TCCCTACCAGTCTGACCTGTCACCT | SHANK3 H3A(−132−108) |
| 3467 | ACATCCCTACCAGTCTGACCTGTCA | SHANK3 H3A(−129−105) |
| 3468 | GCCACATCCCTACCAGTCTGACCTG | SHANK3 H3A(−126−102) |
| 3469 | CCCGCCACATCCCTACCAGTCTGAC | SHANK3 H3A(−123−99) |
| 3470 | ACCCCCGCCACATCCCTACCAGTCT | SHANK3 H3A(−120−96) |
| 3471 | GCAACCCCCGCCACATCCCTACCAG | SHANK3 H3A(−117−93) |
| 3472 | CAGGCAACCCCCGCCACATCCCTAC | SHANK3 H3A(−114−90) |
| 3473 | CTTCAGGCAACCCCCGCCACATCCC | SHANK3 H3A(−111−87) |
| 3474 | CACCTTCAGGCAACCCCCGCCACAT | SHANK3 H3A(−108−84) |
| 3475 | GGCCACCTTCAGGCAACCCCCGCCA | SHANK3 H3A(−105−81) |
| 3476 | TCAGGCCACCTTCAGGCAACCCCCG | SHANK3 H3A(−102−78) |
| 3477 | GCCTCAGGCCACCTTCAGGCAACCC | SHANK3 H3A(−99−75) |
| 3478 | CAAGCCTCAGGCCACCTTCAGGCAA | SHANK3 H3A(−96−72) |
| 3479 | CGGCAAGCCTCAGGCCACCTTCAGG | SHANK3 H3A(−93−69) |
| 3480 | CTCCGGCAAGCCTCAGGCCACCTTC | SHANK3 H3A(−90−66) |
| 3481 | TTCCTCCGGCAAGCCTCAGGCCACC | SHANK3 H3A(−87−63) |
| 3482 | GCCTTCCTCCGGCAAGCCTCAGGCC | SHANK3 H3A(−84−60) |
| 3483 | CCCGCCTTCCTCCGGCAAGCCTCAG | SHANK3 H3A(−81−57) |
| 3484 | TCACCCGCCTTCCTCCGGCAAGCCT | SHANK3 H3A(−78−54) |
| 3485 | ACATCACCCGCCTTCCTCCGGCAAG | SHANK3 H3A(−75−51) |
| 3486 | TGAACATCACCCGCCTTCCTCCGGC | SHANK3 H3A(−72−48) |
| 3487 | ATCTGAACATCACCCGCCTTCCTCC | SHANK3 H3A(−69−45) |
| 3488 | ATCATCTGAACATCACCCGCCTTCC | SHANK3 H3A(−66−42) |
| 3489 | TCCATCATCTGAACATCACCCGCCT | SHANK3 H3A(−63−39) |
| 3490 | GCCTCCATCATCTGAACATCACCCG | SHANK3 H3A(−60−36) |
| 3491 | AAGGCCTCCATCATCTGAACATCAC | SHANK3 H3A(−57−33) |
| 3492 | ACCAAGGCCTCCATCATCTGAACAT | SHANK3 H3A(−54−30) |
| 3493 | GGCACCAAGGCCTCCATCATCTGAA | SHANK3 H3A(−51−27) |
| 3494 | CCTGGCACCAAGGCCTCCATCATCT | SHANK3 H3A(−48−24) |
| 3495 | CAGCCTGGCACCAAGGCCTCCATCA | SHANK3 H3A(−45−21) |
| 3496 | AGTCAGCCTGGCACCAAGGCCTCCA | SHANK3 H3A(−42−18) |
| 3497 | GTCAGTCAGCCTGGCACCAAGGCCT | SHANK3 H3A(−39−15) |
| 3498 | GCCGTCAGTCAGCCTGGCACCAAGG | SHANK3 H3A(−36−12) |
| 3499 | CCGGCCGTCAGTCAGCCTGGCACCA | SHANK3 H3A(−33−9) |
| 3500 | ACACCGGCCGTCAGTCAGCCTGGCA | SHANK3 H3A(−30−6) |
| 3501 | GGAACACCGGCCGTCAGTCAGCCTG | SHANK3 H3A(−27−3) |
| 3502 | AAACTGCTCAGATAAAG | SHANK3 H2A(−14+3) |
| 3503 | TCGAAACTGCTCAGATA | SHANK3 H2A(−11+6) |
| 3504 | GTATCGAAACTGCTCAG | SHANK3 H2A(−8+9) |
| 3505 | CTTGTATCGAAACTGCT | SHANK3 H2A(−5+12) |
| 3506 | CCGCTTGTATCGAAACT | SHANK3 H2A(−2+15) |
| 3507 | TCGCCGCTTGTATCGAA | SHANK3 H2A(+2+18) |
| 3508 | AACTCGCCGCTTGTATC | SHANK3 H2A(+5+21) |
| 3509 | ATAAACTCGCCGCTTGT | SHANK3 H2A(+8+24) |
| 3510 | GGCATAAACTCGCCGCT | SHANK3 H2A(+11+27) |
| 3511 | CTGGGCATAAACTCGCC | SHANK3 H2A(+14+30) |
| 3512 | GTTCTGGGCATAAACTC | SHANK3 H2A(+17+33) |
| 3513 | GAGGTTCTGGGCATAAA | SHANK3 H2A(+20+36) |
| 3514 | GATGAGGTTCTGGGCAT | SHANK3 H2A(+23+39) |
| 3515 | ATCGATGAGGTTCTGGG | SHANK3 H2A(+26+42) |
| 3516 | ATCATCGATGAGGTTCT | SHANK3 H2A(+29+45) |
| 3517 | CTTATCATCGATGAGGT | SHANK3 H2A(+32+48) |
| 3518 | CTGCTTATCATCGATGA | SHANK3 H2A(+35+51) |
| 3519 | AAACTGCTTATCATCGA | SHANK3 H2A(+38+54) |
| 3520 | TGCAAACTGCTTATCAT | SHANK3 H2A(+41+57) |
| 3521 | CTTTGCAAACTGCTTAT | SHANK3 H2A(+44+60) |
| 3522 | AAGCTTTGCAAACTGCT | SHANK3 H2A(+47+63) |
| 3523 | GTGAAGCTTTGCAAACT | SHANK3 H2A(+50+66) |
| 3524 | TGTGTGAAGCTTTGCAA | SHANK3 H2A(+53+69) |
| 3525 | CTTTGTGTGAAGCTTTG | SHANK3 H2A(+56+72) |
| 3526 | TACCTTTGTGTGAAGCT | SHANK3 H2D(+14−3) |
| 3527 | CTTTACCTTTGTGTGAA | SHANK3 H2D(+11−6) |
| 3528 | ATCCTTTACCTTTGTGT | SHANK3 H2D(+8−9) |
| 3529 | GTGATCCTTTACCTTTG | SHANK3 H2D(+5−12) |
| 3530 | CCCGTGATCCTTTACCT | SHANK3 H2D(+2−15) |
| 3531 | CTCCCCGTGATCCTTTA | SHANK3 H2D(−2−18) |
| 3532 | CCCCTCCCCGTGATCCT | SHANK3 H2D(−5−21) |
| 3533 | AGCCCCCTCCCCGTGAT | SHANK3 H2D(−8−24) |
| 3534 | AGGAGCCCCCTCCCCGT | SHANK3 H2D(−11−27) |
| 3535 | CTCAGGAGCCCCCTCCC | SHANK3 H2D(−14−30) |
| 3536 | AACCTCAGGAGCCCCCT | SHANK3 H2D(−17−33) |
| 3537 | GGGAACCTCAGGAGCCC | SHANK3 H2D(−20−36) |
| 3538 | GGAGGGAACCTCAGGAG | SHANK3 H2D(−23−39) |
| 3539 | GCAGGAGGGAACCTCAG | SHANK3 H2D(−26−42) |
| 3540 | GAGGCAGGAGGGAACCT | SHANK3 H2D(−29−45) |
| 3541 | AGGGAGGCAGGAGGGAA | SHANK3 H2D(−32−48) |
| 3542 | CACAGGGAGGCAGGAGG | SHANK3 H2D(−35−51) |
| 3543 | AGCCACAGGGAGGCAGG | SHANK3 H2D(−38−54) |
| 3544 | AGCAGCCACAGGGAGGC | SHANK3 H2D(−41−57) |
| 3545 | GACAGCAGCCACAGGGA | SHANK3 H2D(−44−60) |
| 3546 | GGGGACAGCAGCCACAG | SHANK3 H2D(−47−63) |
| 3547 | GTGGGGGACAGCAGCCA | SHANK3 H2D(−50−66) |
| 3548 | GGGGTGGGGGACAGCAG | SHANK3 H2D(−53−69) |
| 3549 | GCTGGGGTGGGGGACAG | SHANK3 H2D(−56−72) |
| 3550 | CAAGCTGGGGTGGGGGA | SHANK3 H2D(−59−75) |
| 3551 | CCCCAAGCTGGGGTGGG | SHANK3 H2D(−62−78) |
| 3552 | CAGCCCCAAGCTGGGGT | SHANK3 H2D(−65−81) |
| 3553 | GGTCAGCCCCAAGCTGG | SHANK3 H2D(−68−84) |
| 3554 | TGTGGTCAGCCCCAAGC | SHANK3 H2D(−71−87) |
| 3555 | GACTGTGGTCAGCCCCA | SHANK3 H2D(−74−90) |
| 3556 | GGGGACTGTGGTCAGCC | SHANK3 H2D(−77−93) |
| 3557 | CTGGGGGACTGTGGTCA | SHANK3 H2D(−80−96) |
| 3558 | AAGCTGGGGGACTGTGG | SHANK3 H2D(−83−99) |
| 3559 | CTAAAGCTGGGGGACTG | SHANK3 H2D(−86−102) |
| 3560 | GAGCTAAAGCTGGGGGA | SHANK3 H2D(−89−105) |
| 3561 | ACTGAGCTAAAGCTGGG | SHANK3 H2D(−92−108) |
| 3562 | TGGACTGAGCTAAAGCT | SHANK3 H2D(−95−111) |
| 3563 | AAATGGACTGAGCTAAA | SHANK3 H2D(−98−114) |
| 3564 | GGGAAATGGACTGAGCT | SHANK3 H2D(−101−117) |
| 3565 | ATGGGGAAATGGACTGA | SHANK3 H2D(−104−120) |
| 3566 | ATGATGGGGAAATGGAC | SHANK3 H2D(−107−123) |
| 3567 | CTGATGATGGGGAAATG | SHANK3 H2D(−110−126) |
| 3568 | CCCCTGATGATGGGGAA | SHANK3 H2D(−113−129) |
| 3569 | GGGCCCCTGATGATGGG | SHANK3 H2D(−116−132) |
| 3570 | TCTGGGCCCCTGATGAT | SHANK3 H2D(−119−135) |
| 3571 | GGCTCTGGGCCCCTGAT | SHANK3 H2D(−122−138) |
| 3572 | ACAGGCTCTGGGCCCCT | SHANK3 H2D(−125−141) |
| 3573 | AGTACAGGCTCTGGGCC | SHANK3 H2D(−128−144) |
| 3574 | CCCAGTACAGGCTCTGG | SHANK3 H2D(−131−147) |
| 3575 | ACACCCAGTACAGGCTC | SHANK3 H2D(−134−150) |
| 3576 | GCCACACCCAGTACAGG | SHANK3 H2D(−137−153) |
| 3577 | TCAGCCACACCCAGTAC | SHANK3 H2D(−140−156) |
| 3578 | CCTTCAGCCACACCCAG | SHANK3 H2D(−143−159) |
| 3579 | AGCCCTTCAGCCACACC | SHANK3 H2D(−146−162) |
| 3580 | CCCAGCCCTTCAGCCAC | SHANK3 H2D(−149−165) |
| 3581 | GTGCCCAGCCCTTCAGC | SHANK3 H2D(−152−168) |
| 3582 | TCTGTGCCCAGCCCTTC | SHANK3 H2D(−155−171) |
| 3583 | GAATCTGTGCCCAGCCC | SHANK3 H2D(−158−174) |
| 3584 | CAGGAATCTGTGCCCAG | SHANK3 H2D(−161−177) |
| 3585 | GGCCAGGAATCTGTGCC | SHANK3 H2D(−164−180) |
| 3586 | TGGGGCCAGGAATCTGT | SHANK3 H2D(−167−183) |
| 3587 | CCATGGGGCCAGGAATC | SHANK3 H2D(−170−186) |
| 3588 | CAACCATGGGGCCAGGA | SHANK3 H2D(−173−189) |
| 3589 | CCCCAACCATGGGGCCA | SHANK3 H2D(−176−192) |
| 3590 | TGACCCCAACCATGGGG | SHANK3 H2D(−179−195) |
| 3591 | ACCTGACCCCAACCATG | SHANK3 H2D(−182−198) |
| 3592 | ACCACCTGACCCCAACC | SHANK3 H2D(−185−201) |
| 3593 | TGTACCACCTGACCCCA | SHANK3 H2D(−188−204) |
| 3594 | CACTGTACCACCTGACC | SHANK3 H2D(−191−207) |
| 3595 | CATCACTGTACCACCTG | SHANK3 H2D(−194−210) |
| 3596 | ACACATCACTGTACCAC | SHANK3 H2D(−197−213) |
| 3597 | GGAACACATCACTGTAC | SHANK3 H2D(−200−216) |
| 3598 | ACTGGAACACATCACTG | SHANK3 H2D(−203−219) |
| 3599 | CTAACTGGAACACATCA | SHANK3 H2D(−206−222) |
| 3600 | GATCTAACTGGAACACA | SHANK3 H2D(−209−225) |
| 3601 | TCTGATCTAACTGGAAC | SHANK3 H2D(−212−228) |
| 3602 | GAGTCTGATCTAACTGG | SHANK3 H2D(−215−231) |
| 3603 | CAAGAGTCTGATCTAAC | SHANK3 H2D(−218−234) |
| 3604 | CGGCAAGAGTCTGATCT | SHANK3 H2D(−221−237) |
| 3605 | GGTCGGCAAGAGTCTGA | SHANK3 H2D(−224−240) |
| 3606 | GGGGGTCGGCAAGAGTC | SHANK3 H2D(−227−243) |
| 3607 | CAGGGGGGTCGGCAAGA | SHANK3 H2D(−230−246) |
| 3608 | AGCCAGGGGGGTCGGCA | SHANK3 H2D(−233−249) |
| 3609 | CTAAGCCAGGGGGGTCG | SHANK3 H2D(−236−252) |
| 3610 | CCCCTAAGCCAGGGGGG | SHANK3 H2D(−239−255) |
| 3611 | CAGCCCCTAAGCCAGGG | SHANK3 H2D(−242−258) |
| 3612 | CTCCAGCCCCTAAGCCA | SHANK3 H2D(−245−261) |
| 3613 | ACACTCCAGCCCCTAAG | SHANK3 H2D(−248−264) |
| 3614 | AGGACACTCCAGCCCCT | SHANK3 H2D(−251−267) |
| 3615 | CACAGGACACTCCAGCC | SHANK3 H2D(−254−270) |
| 3616 | TCTCACAGGACACTCCA | SHANK3 H2D(−257−273) |
| 3617 | GCTTCTCACAGGACACT | SHANK3 H2D(−260−276) |
| 3618 | CCAGCTTCTCACAGGAC | SHANK3 H2D(−263−279) |
| 3619 | CACCCAGCTTCTCACAG | SHANK3 H2D(−266−282) |
| 3620 | CTCCACCCAGCTTCTCA | SHANK3 H2D(−269−285) |
| 3621 | CCTCTCCACCCAGCTTC | SHANK3 H2D(−272−288) |
| 3622 | CTCCCTCTCCACCCAGC | SHANK3 H2D(−275−291) |
| 3623 | CCACTCCCTCTCCACCC | SHANK3 H2D(−278−294) |
| 3624 | TGTCCACTCCCTCTCCA | SHANK3 H2D(−281−297) |
| 3625 | GCTTGTCCACTCCCTCT | SHANK3 H2D(−284−300) |
| 3626 | GATGCTTGTCCACTCCC | SHANK3 H2D(−287−303) |
| 3627 | TCAGATGCTTGTCCACT | SHANK3 H2D(−290−306) |
| 3628 | ACATCAGATGCTTGTCC | SHANK3 H2D(−293−309) |
| 3629 | ATCACATCAGATGCTTG | SHANK3 H2D(−296−312) |
| 3630 | GCCATCACATCAGATGC | SHANK3 H2D(−299−315) |
| 3631 | ACAGCCATCACATCAGA | SHANK3 H2D(−302−318) |
| 3632 | CCGACAGCCATCACATC | SHANK3 H2D(−305−321) |
| 3633 | GTCCCGACAGCCATCAC | SHANK3 H2D(−308−324) |
| 3634 | CTTGTCCCGACAGCCAT | SHANK3 H2D(−311−327) |
| 3635 | TGCCTTGTCCCGACAGC | SHANK3 H2D(−314−330) |
| 3636 | GGGTGCCTTGTCCCGAC | SHANK3 H2D(−317−333) |
| 3637 | GCTGGGTGCCTTGTCCC | SHANK3 H2D(−320−336) |
| 3638 | GATGCTGGGTGCCTTGT | SHANK3 H2D(−323−339) |
| 3639 | CTCGATGCTGGGTGCCT | SHANK3 H2D(−326−342) |
| 3640 | CCACTCGATGCTGGGTG | SHANK3 H2D(−329−345) |
| 3641 | CGACCACTCGATGCTGG | SHANK3 H2D(−332−348) |
| 3642 | GGTCGACCACTCGATGC | SHANK3 H2D(−335−351) |
| 3643 | ACTGGTCGACCACTCGA | SHANK3 H2D(−338−354) |
| 3644 | AGCACTGGTCGACCACT | SHANK3 H2D(−341−357) |
| 3645 | CCTAGCACTGGTCGACC | SHANK3 H2D(−344−360) |
| 3646 | ATGCCTAGCACTGGTCG | SHANK3 H2D(−347−363) |
| 3647 | CAAATGCCTAGCACTGG | SHANK3 H2D(−350−366) |
| 3648 | TCACAAATGCCTAGCAC | SHANK3 H2D(−353−369) |
| 3649 | TAATCACAAATGCCTAG | SHANK3 H2D(−356−372) |
| 3650 | GTCTAATCACAAATGCC | SHANK3 H2D(−359−375) |
| 3651 | GAGGTCTAATCACAAAT | SHANK3 H2D(−362−378) |
| 3652 | TGAGAGGTCTAATCACA | SHANK3 H2D(−365−381) |
| 3653 | TAATGAGAGGTCTAATC | SHANK3 H2D(−368−384) |
| 3654 | GATTAATGAGAGGTCTA | SHANK3 H2D(−371−387) |
| 3655 | GAGGATTAATGAGAGGT | SHANK3 H2D(−374−390) |
| 3656 | GAAGAGGATTAATGAGA | SHANK3 H2D(−377−393) |
| 3657 | CTGGAAGAGGATTAATG | SHANK3 H2D(−380−396) |
| 3658 | CCTCTGGAAGAGGATTA | SHANK3 H2D(−383−399) |
| 3659 | GTCCCTCTGGAAGAGGA | SHANK3 H2D(−386−402) |
| 3660 | ATAGTCCCTCTGGAAGA | SHANK3 H2D(−389−405) |
| 3661 | ATAATAGTCCCTCTGGA | SHANK3 H2D(−392−408) |
| 3662 | GTTATAATAGTCCCTCT | SHANK3 H2D(−395−411) |
| 3663 | AGGGTTATAATAGTCCC | SHANK3 H2D(−398−414) |
| 3664 | AACAGGGTTATAATAGT | SHANK3 H2D(−401−417) |
| 3665 | TGAAACAGGGTTATAAT | SHANK3 H2D(−404−420) |
| 3666 | CTGTGAAACAGGGTTAT | SHANK3 H2D(−407−423) |
| 3667 | CATCTGTGAAACAGGGT | SHANK3 H2D(−410−426) |
| 3668 | CCCCATCTGTGAAACAG | SHANK3 H2D(−413−429) |
| 3669 | TGTCCCCATCTGTGAAA | SHANK3 H2D(−416−432) |
| 3670 | CTGTGTCCCCATCTGTG | SHANK3 H2D(−419−435) |
| 3671 | ACTCTGTGTCCCCATCT | SHANK3 H2D(−422−438) |
| 3672 | GGTACTCTGTGTCCCCA | SHANK3 H2D(−425−441) |
| 3673 | TCGGGTACTCTGTGTCC | SHANK3 H2D(−428−444) |
| 3674 | CAGTCGGGTACTCTGTG | SHANK3 H2D(−431−447) |
| 3675 | ACACAGTCGGGTACTCT | SHANK3 H2D(−434−450) |
| 3676 | TCTACACAGTCGGGTAC | SHANK3 H2D(−437−453) |
| 3677 | CTGTCTACACAGTCGGG | SHANK3 H2D(−440−456) |
| 3678 | TCACTGTCTACACAGTC | SHANK3 H2D(−443−459) |
| 3679 | GCTTCACTGTCTACACA | SHANK3 H2D(−446−462) |
| 3680 | CCAGCTTCACTGTCTAC | SHANK3 H2D(−449−465) |
| 3681 | GCCCCAGCTTCACTGTC | SHANK3 H2D(−452−468) |
| 3682 | TCAGCCCCAGCTTCACT | SHANK3 H2D(−455−471) |
| 3683 | GGTTCAGCCCCAGCTTC | SHANK3 H2D(−458−474) |
| 3684 | CTGGGTTCAGCCCCAGC | SHANK3 H2D(−461−477) |
| 3685 | GATCTGGGTTCAGCCCC | SHANK3 H2D(−464−480) |
| 3686 | ACAGATCTGGGTTCAGC | SHANK3 H2D(−467−483) |
| 3687 | CAGACAGATCTGGGTTC | SHANK3 H2D(−470−486) |
| 3688 | AATCAGACAGATCTGGG | SHANK3 H2D(−473−489) |
| 3689 | TGGAATCAGACAGATCT | SHANK3 H2D(−476−492) |
| 3690 | GCCTGGAATCAGACAGA | SHANK3 H2D(−479−495) |
| 3691 | AGGGCCTGGAATCAGAC | SHANK3 H2D(−482−498) |
| 3692 | CACAGGGCCTGGAATCA | SHANK3 H2D(−485−501) |
| 3693 | GGGCACAGGGCCTGGAA | SHANK3 H2D(−488−504) |
| 3694 | CCTGGGCACAGGGCCTG | SHANK3 H2D(−491−507) |
| 3695 | ATGCCTGGGCACAGGGC | SHANK3 H2D(−494−510) |
| 3696 | GGCATGCCTGGGCACAG | SHANK3 H2D(−497−513) |
| 3697 | AAAGGCATGCCTGGGCA | SHANK3 H2D(−500−516) |
| 3698 | CTCAAAGGCATGCCTGG | SHANK3 H2D(−503−519) |
| 3699 | CACCTCAAAGGCATGCC | SHANK3 H2D(−506−522) |
| 3700 | AGACACCTCAAAGGCAT | SHANK3 H2D(−509−525) |
| 3701 | AGTAGACACCTCAAAGG | SHANK3 H2D(−512−528) |
| 3702 | AGCAGTAGACACCTCAA | SHANK3 H2D(−515−531) |
| 3703 | CCCAGCAGTAGACACCT | SHANK3 H2D(−518−534) |
| 3704 | GCACCCAGCAGTAGACA | SHANK3 H2D(−521−537) |
| 3705 | GTGGCACCCAGCAGTAG | SHANK3 H2D(−524−540) |
| 3706 | GGGGTGGCACCCAGCAG | SHANK3 H2D(−527−543) |
| 3707 | TTGGGGGTGGCACCCAG | SHANK3 H2D(−530−546) |
| 3708 | CAGTTGGGGGTGGCACC | SHANK3 H2D(−533−549) |
| 3709 | GCCCAGTTGGGGGTGGC | SHANK3 H2D(−536−552) |
| 3710 | CAGGCCCAGTTGGGGGT | SHANK3 H2D(−539−555) |
| 3711 | GGTCAGGCCCAGTTGGG | SHANK3 H2D(−542−558) |
| 3712 | TGGGGTCAGGCCCAGTT | SHANK3 H2D(−545−561) |
| 3713 | ATTTGGGGTCAGGCCCA | SHANK3 H2D(−548−564) |
| 3714 | AGCATTTGGGGTCAGGC | SHANK3 H2D(−551−567) |
| 3715 | AAGAGCATTTGGGGTCA | SHANK3 H2D(−554−570) |
| 3716 | CTCAAGAGCATTTGGGG | SHANK3 H2D(−557−573) |
| 3717 | CCCCTCAAGAGCATTTG | SHANK3 H2D(−560−576) |
| 3718 | GCCCCCCTCAAGAGCAT | SHANK3 H2D(−563−579) |
| 3719 | GGTGCCCCCCTCAAGAG | SHANK3 H2D(−566−582) |
| 3720 | TAAGGTGCCCCCCTCAA | SHANK3 H2D(−569−585) |
| 3721 | TAGTAAGGTGCCCCCCT | SHANK3 H2D(−572−588) |
| 3722 | AAATAGTAAGGTGCCCC | SHANK3 H2D(−575−591) |
| 3723 | GGGAAATAGTAAGGTGC | SHANK3 H2D(−578−594) |
| 3724 | AGCGGGAAATAGTAAGG | SHANK3 H2D(−581−597) |
| 3725 | TTCAGCGGGAAATAGTA | SHANK3 H2D(−584−600) |
| 3726 | ACATTCAGCGGGAAATA | SHANK3 H2D(−587−603) |
| 3727 | CTGACATTCAGCGGGAA | SHANK3 H2D(−590−606) |
| 3728 | CCTCTGACATTCAGCGG | SHANK3 H2D(−593−609) |
| 3729 | GCCCCTCTGACATTCAG | SHANK3 H2D(−596−612) |
| 3730 | CCTGCCCCTCTGACATT | SHANK3 H2D(−599−615) |
| 3731 | CACCCTGCCCCTCTGAC | SHANK3 H2D(−602−618) |
| 3732 | ACCCACCCTGCCCCTCT | SHANK3 H2D(−605−621) |
| 3733 | GGCACCCACCCTGCCCC | SHANK3 H2D(−608−624) |
| 3734 | TGTGGCACCCACCCTGC | SHANK3 H2D(−611−627) |
| 3735 | GGCTGTGGCACCCACCC | SHANK3 H2D(−614−630) |
| 3736 | AGGGGCTGTGGCACCCA | SHANK3 H2D(−617−633) |
| 3737 | GGGAGGGGCTGTGGCAC | SHANK3 H2D(−620−636) |
| 3738 | CTGGGGAGGGGCTGTGG | SHANK3 H2D(−623−639) |
| 3739 | CCCCTGGGGAGGGGCTG | SHANK3 H2D(−626−642) |
| 3740 | AAGCCCCTGGGGAGGGG | SHANK3 H2D(−629−645) |
| 3741 | TGGAAGCCCCTGGGGAG | SHANK3 H2D(−632−648) |
| 3742 | GGGTGGAAGCCCCTGGG | SHANK3 H2D(−635−651) |
| 3743 | TGCGGGTGGAAGCCCCT | SHANK3 H2D(−638−654) |
| 3744 | AGCTGCGGGTGGAAGCC | SHANK3 H2D(−641−657) |
| 3745 | GTGAGCTGCGGGTGGAA | SHANK3 H2D(−644−660) |
| 3746 | ACTGTGAGCTGCGGGTG | SHANK3 H2D(−647−663) |
| 3747 | TGGACTGTGAGCTGCGG | SHANK3 H2D(−650−666) |
| 3748 | TGCTGGACTGTGAGCTG | SHANK3 H2D(−653−669) |
| 3749 | GCCTGCTGGACTGTGAG | SHANK3 H2D(−656−672) |
| 3750 | AGTGCCTGCTGGACTGT | SHANK3 H2D(−659−675) |
| 3751 | ACAAGTGCCTGCTGGAC | SHANK3 H2D(−662−678) |
| 3752 | CAAACAAGTGCCTGCTG | SHANK3 H2D(−665−681) |
| 3753 | CAGCAAACAAGTGCCTG | SHANK3 H2D(−668−684) |
| 3754 | ATCCAGCAAACAAGTGC | SHANK3 H2D(−671−687) |
| 3755 | AGTATCCAGCAAACAAG | SHANK3 H2D(−674−690) |
| 3756 | TAAAGTATCCAGCAAAC | SHANK3 H2D(−677−693) |
| 3757 | CCATAAAGTATCCAGCA | SHANK3 H2D(−680−696) |
| 3758 | CGCCCATAAAGTATCCA | SHANK3 H2D(−683−699) |
| 3759 | GACCGCCCATAAAGTAT | SHANK3 H2D(−686−702) |
| 3760 | CGAGACCGCCCATAAAG | SHANK3 H2D(−689−705) |
| 3761 | GCTCGAGACCGCCCATA | SHANK3 H2D(−692−708) |
| 3762 | TGAGCTCGAGACCGCCC | SHANK3 H2D(−695−711) |
| 3763 | TCCTGAGCTCGAGACCG | SHANK3 H2D(−698−714) |
| 3764 | CTCTCCTGAGCTCGAGA | SHANK3 H2D(−701−717) |
| 3765 | CCCCTCTCCTGAGCTCG | SHANK3 H2D(−704−720) |
| 3766 | TGACCCCTCTCCTGAGC | SHANK3 H2D(−707−723) |
| 3767 | TTCTGACCCCTCTCCTG | SHANK3 H2D(−710−726) |
| 3768 | CCATTCTGACCCCTCTC | SHANK3 H2D(−713−729) |
| 3769 | CCTCCATTCTGACCCCT | SHANK3 H2D(−716−732) |
| 3770 | GACCCTCCATTCTGACC | SHANK3 H2D(−719−735) |
| 3771 | AGAGACCCTCCATTCTG | SHANK3 H2D(−722−738) |
| 3772 | TCCAGAGACCCTCCATT | SHANK3 H2D(−725−741) |
| 3773 | GGCTCCAGAGACCCTCC | SHANK3 H2D(−728−744) |
| 3774 | CTGGGCTCCAGAGACCC | SHANK3 H2D(−731−747) |
| 3775 | CTCCTGGGCTCCAGAGA | SHANK3 H2D(−734−750) |
| 3776 | CCTCTCCTGGGCTCCAG | SHANK3 H2D(−737−753) |
| 3777 | ATTCCTCTCCTGGGCTC | SHANK3 H2D(−740−756) |
| 3778 | CTGATTCCTCTCCTGGG | SHANK3 H2D(−743−759) |
| 3779 | GTTCTGATTCCTCTCCT | SHANK3 H2D(−746−762) |
| 3780 | ACAGTTCTGATTCCTCT | SHANK3 H2D(−749−765) |
| 3781 | CTGACAGTTCTGATTCC | SHANK3 H2D(−752−768) |
| 3782 | GGGCTGACAGTTCTGAT | SHANK3 H2D(−755−771) |
| 3783 | ACAGGGCTGACAGTTCT | SHANK3 H2D(−758−774) |
| 3784 | GACACAGGGCTGACAGT | SHANK3 H2D(−761−777) |
| 3785 | TAGGACACAGGGCTGAC | SHANK3 H2D(−764−780) |
| 3786 | CATTAGGACACAGGGCT | SHANK3 H2D(−767−783) |
| 3787 | GACCATTAGGACACAGG | SHANK3 H2D(−770−786) |
| 3788 | TGGGACCATTAGGACAC | SHANK3 H2D(−773−789) |
| 3789 | TGATGGGACCATTAGGA | SHANK3 H2D(−776−792) |
| 3790 | CACTGATGGGACCATTA | SHANK3 H2D(−779−795) |
| 3791 | AGTCACTGATGGGACCA | SHANK3 H2D(−782−798) |
| 3792 | CAAAGTCACTGATGGGA | SHANK3 H2D(−785−801) |
| 3793 | CTCCAAAGTCACTGATG | SHANK3 H2D(−788−804) |
| 3794 | AGGCTCCAAAGTCACTG | SHANK3 H2D(−791−807) |
| 3795 | CTGAGGCTCCAAAGTCA | SHANK3 H2D(−794−810) |
| 3796 | AGCCTGAGGCTCCAAAG | SHANK3 H2D(−797−813) |
| 3797 | AGAAGCCTGAGGCTCCA | SHANK3 H2D(−800−816) |
| 3798 | CACAGAAGCCTGAGGCT | SHANK3 H2D(−803−819) |
| 3799 | TGGCACAGAAGCCTGAG | SHANK3 H2D(−806−822) |
| 3800 | GCCTGGCACAGAAGCCT | SHANK3 H2D(−809−825) |
| 3801 | AAAGCCTGGCACAGAAG | SHANK3 H2D(−812−828) |
| 3802 | AGAAAAGCCTGGCACAG | SHANK3 H2D(−815−831) |
| 3803 | AGCAGAAAAGCCTGGCA | SHANK3 H2D(−818−834) |
| 3804 | GGCAGCAGAAAAGCCTG | SHANK3 H2D(−821−837) |
| 3805 | TGGGGCAGCAGAAAAGC | SHANK3 H2D(−824−840) |
| 3806 | CCCTGGGGCAGCAGAAA | SHANK3 H2D(−827−843) |
| 3807 | CTGCCCTGGGGCAGCAG | SHANK3 H2D(−830−846) |
| 3808 | CGCCTGCCCTGGGGCAG | SHANK3 H2D(−833−849) |
| 3809 | CACCGCCTGCCCTGGGG | SHANK3 H2D(−836−852) |
| 3810 | GACCACCGCCTGCCCTG | SHANK3 H2D(−839−855) |
| 3811 | GTTGACCACCGCCTGCC | SHANK3 H2D(−842−858) |
| 3812 | GCCGTTGACCACCGCCT | SHANK3 H2D(−845−861) |
| 3813 | AAGGCCGTTGACCACCG | SHANK3 H2D(−848−864) |
| 3814 | GCCAAGGCCGTTGACCA | SHANK3 H2D(−851−867) |
| 3815 | AGTGCCAAGGCCGTTGA | SHANK3 H2D(−854−870) |
| 3816 | CATAGTGCCAAGGCCGT | SHANK3 H2D(−857−873) |
| 3817 | AGCCATAGTGCCAAGGC | SHANK3 H2D(−860−876) |
| 3818 | CCCAGCCATAGTGCCAA | SHANK3 H2D(−863−879) |
| 3819 | CTGCCCAGCCATAGTGC | SHANK3 H2D(−866−882) |
| 3820 | ACTCTGCCCAGCCATAG | SHANK3 H2D(−869−885) |
| 3821 | TGGACTCTGCCCAGCCA | SHANK3 H2D(−872−888) |
| 3822 | CAGTGGACTCTGCCCAG | SHANK3 H2D(−875−891) |
| 3823 | CCACAGTGGACTCTGCC | SHANK3 H2D(−878−894) |
| 3824 | TTTCCACAGTGGACTCT | SHANK3 H2D(−881−897) |
| 3825 | ACCTTTCCACAGTGGAC | SHANK3 H2D(−884−900) |
| 3826 | GGGACCTTTCCACAGTG | SHANK3 H2D(−887−903) |
| 3827 | AGGGGGACCTTTCCACA | SHANK3 H2D(−890−906) |
| 3828 | GAGAGGGGGACCTTTCC | SHANK3 H2D(−893−909) |
| 3829 | CAGGAGAGGGGGACCTT | SHANK3 H2D(−896−912) |
| 3830 | TGGCAGGAGAGGGGGAC | SHANK3 H2D(−899−915) |
| 3831 | CAGTGGCAGGAGAGGGG | SHANK3 H2D(−902−918) |
| 3832 | GGCCAGTGGCAGGAGAG | SHANK3 H2D(−905−921) |
| 3833 | GAGGGCCAGTGGCAGGA | SHANK3 H2D(−908−924) |
| 3834 | AGTGAGGGCCAGTGGCA | SHANK3 H2D(−911−927) |
| 3835 | TCAAGTGAGGGCCAGTG | SHANK3 H2D(−914−930) |
| 3836 | AGGTCAAGTGAGGGCCA | SHANK3 H2D(−917−933) |
| 3837 | ACAAGGTCAAGTGAGGG | SHANK3 H2D(−920−936) |
| 3838 | CTGACAAGGTCAAGTGA | SHANK3 H2D(−923−939) |
| 3839 | AGGCTGACAAGGTCAAG | SHANK3 H2D(−926−942) |
| 3840 | CCCAGGCTGACAAGGTC | SHANK3 H2D(−929−945) |
| 3841 | GAACCCAGGCTGACAAG | SHANK3 H2D(−932−948) |
| 3842 | TAGGAACCCAGGCTGAC | SHANK3 H2D(−935−951) |
| 3843 | GGGTAGGAACCCAGGCT | SHANK3 H2D(−938−954) |
| 3844 | ATGGGGTAGGAACCCAG | SHANK3 H2D(−941−957) |
| 3845 | GCCATGGGGTAGGAACC | SHANK3 H2D(−944−960) |
| 3846 | GTGGCCATGGGGTAGGA | SHANK3 H3A(−945−929) |
| 3847 | GAGGTGGCCATGGGGTA | SHANK3 H3A(−942−926) |
| 3848 | GGAGAGGTGGCCATGGG | SHANK3 H3A(−939−923) |
| 3849 | GAGGGAGAGGTGGCCAT | SHANK3 H3A(−936−920) |
| 3850 | CCGGAGGGAGAGGTGGC | SHANK3 H3A(−933−917) |
| 3851 | GATCCGGAGGGAGAGGT | SHANK3 H3A(−930−914) |
| 3852 | AGAGATCCGGAGGGAGA | SHANK3 H3A(−927−911) |
| 3853 | AAGAGAGATCCGGAGGG | SHANK3 H3A(−924−908) |
| 3854 | CTGAAGAGAGATCCGGA | SHANK3 H3A(−921−905) |
| 3855 | TCACTGAAGAGAGATCC | SHANK3 H3A(−918−902) |
| 3856 | TGGTCACTGAAGAGAGA | SHANK3 H3A(−915−899) |
| 3857 | TGTTGGTCACTGAAGAG | SHANK3 H3A(−912−896) |
| 3858 | TCTTGTTGGTCACTGAA | SHANK3 H3A(−909−893) |
| 3859 | ATGTCTTGTTGGTCACT | SHANK3 H3A(−906−890) |
| 3860 | CTCATGTCTTGTTGGTC | SHANK3 H3A(−903−887) |
| 3861 | TCACTCATGTCTTGTTG | SHANK3 H3A(−900−884) |
| 3862 | GAGTCACTCATGTCTTG | SHANK3 H3A(−897−881) |
| 3863 | AGTGAGTCACTCATGTC | SHANK3 H3A(−894−878) |
| 3864 | CAGAGTGAGTCACTCAT | SHANK3 H3A(−891−875) |
| 3865 | CTTCAGAGTGAGTCACT | SHANK3 H3A(−888−872) |
| 3866 | CTACTTCAGAGTGAGTC | SHANK3 H3A(−885−869) |
| 3867 | GACCTACTTCAGAGTGA | SHANK3 H3A(−882−866) |
| 3868 | CGAGACCTACTTCAGAG | SHANK3 H3A(−879−863) |
| 3869 | AAACGAGACCTACTTCA | SHANK3 H3A(−876−860) |
| 3870 | AACAAACGAGACCTACT | SHANK3 H3A(−873−857) |
| 3871 | AAAAACAAACGAGACCT | SHANK3 H3A(−870−854) |
| 3872 | CCTAAAAACAAACGAGA | SHANK3 H3A(−867−851) |
| 3873 | CATCCTAAAAACAAACG | SHANK3 H3A(−864−848) |
| 3874 | GCTCATCCTAAAAACAA | SHANK3 H3A(−861−845) |
| 3875 | CAGGCTCATCCTAAAAA | SHANK3 H3A(−858−842) |
| 3876 | GTTCAGGCTCATCCTAA | SHANK3 H3A(−855−839) |
| 3877 | GAAGTTCAGGCTCATCC | SHANK3 H3A(−852−836) |
| 3878 | TAAGAAGTTCAGGCTCA | SHANK3 H3A(−849−833) |
| 3879 | ATGTAAGAAGTTCAGGC | SHANK3 H3A(−846−830) |
| 3880 | GCTATGTAAGAAGTTCA | SHANK3 H3A(−843−827) |
| 3881 | CGGGCTATGTAAGAAGT | SHANK3 H3A(−840−824) |
| 3882 | ACACGGGCTATGTAAGA | SHANK3 H3A(−837−821) |
| 3883 | GAAACACGGGCTATGTA | SHANK3 H3A(−834−818) |
| 3884 | GCAGAAACACGGGCTAT | SHANK3 H3A(−831−815) |
| 3885 | CAAGCAGAAACACGGGC | SHANK3 H3A(−828−812) |
| 3886 | AGGCAAGCAGAAACACG | SHANK3 H3A(−825−809) |
| 3887 | CCCAGGCAAGCAGAAAC | SHANK3 H3A(−822−806) |
| 3888 | GAGCCCAGGCAAGCAGA | SHANK3 H3A(−819−803) |
| 3889 | CTGGAGCCCAGGCAAGC | SHANK3 H3A(−816−800) |
| 3890 | CAGCTGGAGCCCAGGCA | SHANK3 H3A(−813−797) |
| 3891 | GGCCAGCTGGAGCCCAG | SHANK3 H3A(−810−794) |
| 3892 | AAAGGCCAGCTGGAGCC | SHANK3 H3A(−807−791) |
| 3893 | GGGAAAGGCCAGCTGGA | SHANK3 H3A(−804−788) |
| 3894 | AGAGGGAAAGGCCAGCT | SHANK3 H3A(−801−785) |
| 3895 | GGGAGAGGGAAAGGCCA | SHANK3 H3A(−798−782) |
| 3896 | CAGGGGAGAGGGAAAGG | SHANK3 H3A(−795−779) |
| 3897 | TACCAGGGGAGAGGGAA | SHANK3 H3A(−792−776) |
| 3898 | CACTACCAGGGGAGAGG | SHANK3 H3A(−789−773) |
| 3899 | GCTCACTACCAGGGGAG | SHANK3 H3A(−786−770) |
| 3900 | AGTGCTCACTACCAGGG | SHANK3 H3A(−783−767) |
| 3901 | GGAAGTGCTCACTACCA | SHANK3 H3A(−780−764) |
| 3902 | GGAGGAAGTGCTCACTA | SHANK3 H3A(−777−761) |
| 3903 | AGAGGAGGAAGTGCTCA | SHANK3 H3A(−774−758) |
| 3904 | GAAAGAGGAGGAAGTGC | SHANK3 H3A(−771−755) |
| 3905 | AGTGAAAGAGGAGGAAG | SHANK3 H3A(−768−752) |
| 3906 | GGCAGTGAAAGAGGAGG | SHANK3 H3A(−765−749) |
| 3907 | CCAGGCAGTGAAAGAGG | SHANK3 H3A(−762−746) |
| 3908 | GGGCCAGGCAGTGAAAG | SHANK3 H3A(−759−743) |
| 3909 | GGCGGGCCAGGCAGTGA | SHANK3 H3A(−756−740) |
| 3910 | TCAGGCGGGCCAGGCAG | SHANK3 H3A(−753−737) |
| 3911 | TCCTCAGGCGGGCCAGG | SHANK3 H3A(−750−734) |
| 3912 | ACCTCCTCAGGCGGGCC | SHANK3 H3A(−747−731) |
| 3913 | GCAACCTCCTCAGGCGG | SHANK3 H3A(−744−728) |
| 3914 | GAGGCAACCTCCTCAGG | SHANK3 H3A(−741−725) |
| 3915 | TGGGAGGCAACCTCCTC | SHANK3 H3A(−738−722) |
| 3916 | CCTTGGGAGGCAACCTC | SHANK3 H3A(−735−719) |
| 3917 | CTGCCTTGGGAGGCAAC | SHANK3 H3A(−732−716) |
| 3918 | CCCCTGCCTTGGGAGGC | SHANK3 H3A(−729−713) |
| 3919 | GGACCCCTGCCTTGGGA | SHANK3 H3A(−726−710) |
| 3920 | TAGGGACCCCTGCCTTG | SHANK3 H3A(−723−707) |
| 3921 | CCATAGGGACCCCTGCC | SHANK3 H3A(−720−704) |
| 3922 | GTGCCATAGGGACCCCT | SHANK3 H3A(−717−701) |
| 3923 | AGGGTGCCATAGGGACC | SHANK3 H3A(−714−698) |
| 3924 | GGGAGGGTGCCATAGGG | SHANK3 H3A(−711−695) |
| 3925 | TAGGGGAGGGTGCCATA | SHANK3 H3A(−708−692) |
| 3926 | AAATAGGGGAGGGTGCC | SHANK3 H3A(−705−689) |
| 3927 | CAGAAATAGGGGAGGGT | SHANK3 H3A(−702−686) |
| 3928 | CCTCAGAAATAGGGGAG | SHANK3 H3A(−699−683) |
| 3929 | GTACCTCAGAAATAGGG | SHANK3 H3A(−696−680) |
| 3930 | AGTGTACCTCAGAAATA | SHANK3 H3A(−693−677) |
| 3931 | CCCAGTGTACCTCAGAA | SHANK3 H3A(−690−674) |
| 3932 | ACACCCAGTGTACCTCA | SHANK3 H3A(−687−671) |
| 3933 | TAAACACCCAGTGTACC | SHANK3 H3A(−684−668) |
| 3934 | CCATAAACACCCAGTGT | SHANK3 H3A(−681−665) |
| 3935 | CTTCCATAAACACCCAG | SHANK3 H3A(−678−662) |
| 3936 | GCCCTTCCATAAACACC | SHANK3 H3A(−675−659) |
| 3937 | GGGGCCCTTCCATAAAC | SHANK3 H3A(−672−656) |
| 3938 | GCCGGGGCCCTTCCATA | SHANK3 H3A(−669−653) |
| 3939 | AAGGCCGGGGCCCTTCC | SHANK3 H3A(−666−650) |
| 3940 | CCAAAGGCCGGGGCCCT | SHANK3 H3A(−663−647) |
| 3941 | TGGCCAAAGGCCGGGGC | SHANK3 H3A(−660−644) |
| 3942 | CCCTGGCCAAAGGCCGG | SHANK3 H3A(−657−641) |
| 3943 | GTGCCCTGGCCAAAGGC | SHANK3 H3A(−654−638) |
| 3944 | AAGGTGCCCTGGCCAAA | SHANK3 H3A(−651−635) |
| 3945 | GGCAAGGTGCCCTGGCC | SHANK3 H3A(−648−632) |
| 3946 | CAAGGCAAGGTGCCCTG | SHANK3 H3A(−645−629) |
| 3947 | ACACAAGGCAAGGTGCC | SHANK3 H3A(−642−626) |
| 3948 | CACACACAAGGCAAGGT | SHANK3 H3A(−639−623) |
| 3949 | CCACACACACAAGGCAA | SHANK3 H3A(−636−620) |
| 3950 | ACACCACACACACAAGG | SHANK3 H3A(−633−617) |
| 3951 | CGGACACCACACACACA | SHANK3 H3A(−630−614) |
| 3952 | CTACGGACACCACACAC | SHANK3 H3A(−627−611) |
| 3953 | GCACTACGGACACCACA | SHANK3 H3A(−624−608) |
| 3954 | CCGGCACTACGGACACC | SHANK3 H3A(−621−605) |
| 3955 | CAGCCGGCACTACGGAC | SHANK3 H3A(−618−602) |
| 3956 | CCCCAGCCGGCACTACG | SHANK3 H3A(−615−599) |
| 3957 | CTTCCCCAGCCGGCACT | SHANK3 H3A(−612−596) |
| 3958 | TCACTTCCCCAGCCGGC | SHANK3 H3A(−609−593) |
| 3959 | GCCTCACTTCCCCAGCC | SHANK3 H3A(−606−590) |
| 3960 | AAGGCCTCACTTCCCCA | SHANK3 H3A(−603−587) |
| 3961 | CACAAGGCCTCACTTCC | SHANK3 H3A(−600−584) |
| 3962 | CCCCACAAGGCCTCACT | SHANK3 H3A(−597−581) |
| 3963 | TCACCCCACAAGGCCTC | SHANK3 H3A(−594−578) |
| 3964 | CACTCACCCCACAAGGC | SHANK3 H3A(−591−575) |
| 3965 | ATTCACTCACCCCACAA | SHANK3 H3A(−588−572) |
| 3966 | TTTATTCACTCACCCCA | SHANK3 H3A(−585−569) |
| 3967 | CAGTTTATTCACTCACC | SHANK3 H3A(−582−566) |
| 3968 | ACCCAGTTTATTCACTC | SHANK3 H3A(−579−563) |
| 3969 | TTCACCCAGTTTATTCA | SHANK3 H3A(−576−560) |
| 3970 | TCATTCACCCAGTTTAT | SHANK3 H3A(−573−557) |
| 3971 | TACTCATTCACCCAGTT | SHANK3 H3A(−570−554) |
| 3972 | TTGTACTCATTCACCCA | SHANK3 H3A(−567−551) |
| 3973 | GCCTTGTACTCATTCAC | SHANK3 H3A(−564−548) |
| 3974 | ATGGCCTTGTACTCATT | SHANK3 H3A(−561−545) |
| 3975 | TTGATGGCCTTGTACTC | SHANK3 H3A(−558−542) |
| 3976 | ACTTTGATGGCCTTGTA | SHANK3 H3A(−555−539) |
| 3977 | ATGACTTTGATGGCCTT | SHANK3 H3A(−552−536) |
| 3978 | GACATGACTTTGATGGC | SHANK3 H3A(−549−533) |
| 3979 | TGTGACATGACTTTGAT | SHANK3 H3A(−546−530) |
| 3980 | TCCTGTGACATGACTTT | SHANK3 H3A(−543−527) |
| 3981 | CCATCCTGTGACATGAC | SHANK3 H3A(−540−524) |
| 3982 | GACCCATCCTGTGACAT | SHANK3 H3A(−537−521) |
| 3983 | AGGGACCCATCCTGTGA | SHANK3 H3A(−534−518) |
| 3984 | CCCAGGGACCCATCCTG | SHANK3 H3A(−531−515) |
| 3985 | CACCCCAGGGACCCATC | SHANK3 H3A(−528−512) |
| 3986 | CCCCACCCCAGGGACCC | SHANK3 H3A(−525−509) |
| 3987 | GACCCCCACCCCAGGGA | SHANK3 H3A(−522−506) |
| 3988 | GCAGACCCCCACCCCAG | SHANK3 H3A(−519−503) |
| 3989 | ACCGCAGACCCCCACCC | SHANK3 H3A(−516−500) |
| 3990 | CCCACCGCAGACCCCCA | SHANK3 H3A(−513−497) |
| 3991 | CACCCCACCGCAGACCC | SHANK3 H3A(−510−494) |
| 3992 | GCTCACCCCACCGCAGA | SHANK3 H3A(−507−491) |
| 3993 | CCAGCTCACCCCACCGC | SHANK3 H3A(−504−488) |
| 3994 | TCTCCAGCTCACCCCAC | SHANK3 H3A(−501−485) |
| 3995 | TCCTCTCCAGCTCACCC | SHANK3 H3A(−498−482) |
| 3996 | TCTTCCTCTCCAGCTCA | SHANK3 H3A(−495−479) |
| 3997 | CCATCTTCCTCTCCAGC | SHANK3 H3A(−492−476) |
| 3998 | GGCCCATCTTCCTCTCC | SHANK3 H3A(−489−473) |
| 3999 | TTCGGCCCATCTTCCTC | SHANK3 H3A(−486−470) |
| 4000 | TTCTTCGGCCCATCTTC | SHANK3 H3A(−483−467) |
| 4001 | TCCTTCTTCGGCCCATC | SHANK3 H3A(−480−464) |
| 4002 | TGCTCCTTCTTCGGCCC | SHANK3 H3A(−477−461) |
| 4003 | ACCTGCTCCTTCTTCGG | SHANK3 H3A(−474−458) |
| 4004 | ACAACCTGCTCCTTCTT | SHANK3 H3A(−471−455) |
| 4005 | GAGACAACCTGCTCCTT | SHANK3 H3A(−468−452) |
| 4006 | CCTGAGACAACCTGCTC | SHANK3 H3A(−465−449) |
| 4007 | CACCCTGAGACAACCTG | SHANK3 H3A(−462−446) |
| 4008 | CAGCACCCTGAGACAAC | SHANK3 H3A(−459−443) |
| 4009 | CCTCAGCACCCTGAGAC | SHANK3 H3A(−456−440) |
| 4010 | AATCCTCAGCACCCTGA | SHANK3 H3A(−453−437) |
| 4011 | CTGAATCCTCAGCACCC | SHANK3 H3A(−450−434) |
| 4012 | GCCCTGAATCCTCAGCA | SHANK3 H3A(−447−431) |
| 4013 | GCAGCCCTGAATCCTCA | SHANK3 H3A(−444−428) |
| 4014 | ACAGCAGCCCTGAATCC | SHANK3 H3A(−441−425) |
| 4015 | CCCACAGCAGCCCTGAA | SHANK3 H3A(−438−422) |
| 4016 | AGCCCCACAGCAGCCCT | SHANK3 H3A(−435−419) |
| 4017 | ACCAGCCCCACAGCAGC | SHANK3 H3A(−432−416) |
| 4018 | GGAACCAGCCCCACAGC | SHANK3 H3A(−429−413) |
| 4019 | GCAGGAACCAGCCCCAC | SHANK3 H3A(−426−410) |
| 4020 | CAGGCAGGAACCAGCCC | SHANK3 H3A(−423−407) |
| 4021 | ACCCAGGCAGGAACCAG | SHANK3 H3A(−420−404) |
| 4022 | TCCACCCAGGCAGGAAC | SHANK3 H3A(−417−401) |
| 4023 | AGGTCCACCCAGGCAGG | SHANK3 H3A(−414−398) |
| 4024 | CCGAGGTCCACCCAGGC | SHANK3 H3A(−411−395) |
| 4025 | AGCCCGAGGTCCACCCA | SHANK3 H3A(−408−392) |
| 4026 | AAGAGCCCGAGGTCCAC | SHANK3 H3A(−405−389) |
| 4027 | TAGAAGAGCCCGAGGTC | SHANK3 H3A(−402−386) |
| 4028 | GCCTAGAAGAGCCCGAG | SHANK3 H3A(−399−383) |
| 4029 | CTGGCCTAGAAGAGCCC | SHANK3 H3A(−396−380) |
| 4030 | TGCCTGGCCTAGAAGAG | SHANK3 H3A(−393−377) |
| 4031 | ACCTGCCTGGCCTAGAA | SHANK3 H3A(−390−374) |
| 4032 | CCGACCTGCCTGGCCTA | SHANK3 H3A(−387−371) |
| 4033 | GCCCCGACCTGCCTGGC | SHANK3 H3A(−384−368) |
| 4034 | GCTGCCCCGACCTGCCT | SHANK3 H3A(−381−365) |
| 4035 | CCTGCTGCCCCGACCTG | SHANK3 H3A(−378−362) |
| 4036 | GGCCCTGCTGCCCCGAC | SHANK3 H3A(−375−359) |
| 4037 | TCCGGCCCTGCTGCCCC | SHANK3 H3A(−372−356) |
| 4038 | CTCTCCGGCCCTGCTGC | SHANK3 H3A(−369−353) |
| 4039 | CGTCTCTCCGGCCCTGC | SHANK3 H3A(−366−350) |
| 4040 | CTCCGTCTCTCCGGCCC | SHANK3 H3A(−363−347) |
| 4041 | TGGCTCCGTCTCTCCGG | SHANK3 H3A(−360−344) |
| 4042 | CCCTGGCTCCGTCTCTC | SHANK3 H3A(−357−341) |
| 4043 | CTGCCCTGGCTCCGTCT | SHANK3 H3A(−354−338) |
| 4044 | TCACTGCCCTGGCTCCG | SHANK3 H3A(−351−335) |
| 4045 | CAGTCACTGCCCTGGCT | SHANK3 H3A(−348−332) |
| 4046 | GCCCAGTCACTGCCCTG | SHANK3 H3A(−345−329) |
| 4047 | CAGGCCCAGTCACTGCC | SHANK3 H3A(−342−326) |
| 4048 | CTCCAGGCCCAGTCACT | SHANK3 H3A(−339−323) |
| 4049 | CCACTCCAGGCCCAGTC | SHANK3 H3A(−336−320) |
| 4050 | CCCCCACTCCAGGCCCA | SHANK3 H3A(−333−317) |
| 4051 | AGTCCCCCACTCCAGGC | SHANK3 H3A(−330−314) |
| 4052 | GCAAGTCCCCCACTCCA | SHANK3 H3A(−327−311) |
| 4053 | CAAGCAAGTCCCCCACT | SHANK3 H3A(−324−308) |
| 4054 | CCCCAAGCAAGTCCCCC | SHANK3 H3A(−321−305) |
| 4055 | GGGCCCCAAGCAAGTCC | SHANK3 H3A(−318−302) |
| 4056 | GTGGGGCCCCAAGCAAG | SHANK3 H3A(−315−299) |
| 4057 | CTGGTGGGGCCCCAAGC | SHANK3 H3A(−312−296) |
| 4058 | ACCCTGGTGGGGCCCCA | SHANK3 H3A(−309−293) |
| 4059 | GTCACCCTGGTGGGGCC | SHANK3 H3A(−306−290) |
| 4060 | CAGGTCACCCTGGTGGG | SHANK3 H3A(−303−287) |
| 4061 | GGCCAGGTCACCCTGGT | SHANK3 H3A(−300−284) |
| 4062 | CAAGGCCAGGTCACCCT | SHANK3 H3A(−297−281) |
| 4063 | CACCAAGGCCAGGTCAC | SHANK3 H3A(−294−278) |
| 4064 | CCTCACCAAGGCCAGGT | SHANK3 H3A(−291−275) |
| 4065 | GCCCCTCACCAAGGCCA | SHANK3 H3A(−288−272) |
| 4066 | GTAGCCCCTCACCAAGG | SHANK3 H3A(−285−269) |
| 4067 | CAAGTAGCCCCTCACCA | SHANK3 H3A(−282−266) |
| 4068 | ACCCAAGTAGCCCCTCA | SHANK3 H3A(−279−263) |
| 4069 | TGTACCCAAGTAGCCCC | SHANK3 H3A(−276−260) |
| 4070 | GCTTGTACCCAAGTAGC | SHANK3 H3A(−273−257) |
| 4071 | TCAGCTTGTACCCAAGT | SHANK3 H3A(−270−254) |
| 4072 | CTGTCAGCTTGTACCCA | SHANK3 H3A(−267−251) |
| 4073 | CGACTGTCAGCTTGTAC | SHANK3 H3A(−264−248) |
| 4074 | TCACGACTGTCAGCTTG | SHANK3 H3A(−261−245) |
| 4075 | CAGTCACGACTGTCAGC | SHANK3 H3A(−258−242) |
| 4076 | AACCAGTCACGACTGTC | SHANK3 H3A(−255−239) |
| 4077 | CCAAACCAGTCACGACT | SHANK3 H3A(−252−236) |
| 4078 | TAGCCAAACCAGTCACG | SHANK3 H3A(−249−233) |
| 4079 | TGATAGCCAAACCAGTC | SHANK3 H3A(−246−230) |
| 4080 | CCCTGATAGCCAAACCA | SHANK3 H3A(−243−227) |
| 4081 | GCGCCCTGATAGCCAAA | SHANK3 H3A(−240−224) |
| 4082 | CTTGCGCCCTGATAGCC | SHANK3 H3A(−237−221) |
| 4083 | TTCCTTGCGCCCTGATA | SHANK3 H3A(−234−218) |
| 4084 | CCCTTCCTTGCGCCCTG | SHANK3 H3A(−231−215) |
| 4085 | AAGCCCTTCCTTGCGCC | SHANK3 H3A(−228−212) |
| 4086 | TCAAAGCCCTTCCTTGC | SHANK3 H3A(−225−209) |
| 4087 | GGCTCAAAGCCCTTCCT | SHANK3 H3A(−222−206) |
| 4088 | CACGGCTCAAAGCCCTT | SHANK3 H3A(−219−203) |
| 4089 | ATGCACGGCTCAAAGCC | SHANK3 H3A(−216−200) |
| 4090 | CCCATGCACGGCTCAAA | SHANK3 H3A(−213−197) |
| 4091 | GGGCCCATGCACGGCTC | SHANK3 H3A(−210−194) |
| 4092 | GGTGGGCCCATGCACGG | SHANK3 H3A(−207−191) |
| 4093 | TCGGGTGGGCCCATGCA | SHANK3 H3A(−204−188) |
| 4094 | CACTCGGGTGGGCCCAT | SHANK3 H3A(−201−185) |
| 4095 | TCCCACTCGGGTGGGCC | SHANK3 H3A(−198−182) |
| 4096 | AATTCCCACTCGGGTGG | SHANK3 H3A(−195−179) |
| 4097 | CCCAATTCCCACTCGGG | SHANK3 H3A(−192−176) |
| 4098 | GGCCCCAATTCCCACTC | SHANK3 H3A(−189−173) |
| 4099 | CACGGCCCCAATTCCCA | SHANK3 H3A(−186−170) |
| 4100 | CACCACGGCCCCAATTC | SHANK3 H3A(−183−167) |
| 4101 | TCCCACCACGGCCCCAA | SHANK3 H3A(−180−164) |
| 4102 | CACTCCCACCACGGCCC | SHANK3 H3A(−177−161) |
| 4103 | CTGCACTCCCACCACGG | SHANK3 H3A(−174−158) |
| 4104 | GTCCTGCACTCCCACCA | SHANK3 H3A(−171−155) |
| 4105 | ACGGTCCTGCACTCCCA | SHANK3 H3A(−168−152) |
| 4106 | ACCACGGTCCTGCACTC | SHANK3 H3A(−165−149) |
| 4107 | TCAACCACGGTCCTGCA | SHANK3 H3A(−162−146) |
| 4108 | TTGTCAACCACGGTCCT | SHANK3 H3A(−159−143) |
| 4109 | CAATTGTCAACCACGGT | SHANK3 H3A(−156−140) |
| 4110 | TCACAATTGTCAACCAC | SHANK3 H3A(−153−137) |
| 4111 | ACATCACAATTGTCAAC | SHANK3 H3A(−150−134) |
| 4112 | CTGACATCACAATTGTC | SHANK3 H3A(−147−131) |
| 4113 | CACCTGACATCACAATT | SHANK3 H3A(−144−128) |
| 4114 | TGTCACCTGACATCACA | SHANK3 H3A(−141−125) |
| 4115 | ACCTGTCACCTGACATC | SHANK3 H3A(−138−122) |
| 4116 | CTGACCTGTCACCTGAC | SHANK3 H3A(−135−119) |
| 4117 | AGTCTGACCTGTCACCT | SHANK3 H3A(−132−116) |
| 4118 | ACCAGTCTGACCTGTCA | SHANK3 H3A(−129−113) |
| 4119 | CCTACCAGTCTGACCTG | SHANK3 H3A(−126−110) |
| 4120 | ATCCCTACCAGTCTGAC | SHANK3 H3A(−123−107) |
| 4121 | CACATCCCTACCAGTCT | SHANK3 H3A(−120−104) |
| 4122 | CGCCACATCCCTACCAG | SHANK3 H3A(−117−101) |
| 4123 | CCCCGCCACATCCCTAC | SHANK3 H3A(−114−98) |
| 4124 | AACCCCCGCCACATCCC | SHANK3 H3A(−111−95) |
| 4125 | GGCAACCCCCGCCACAT | SHANK3 H3A(−108−92) |
| 4126 | TCAGGCAACCCCCGCCA | SHANK3 H3A(−105−89) |
| 4127 | CCTTCAGGCAACCCCCG | SHANK3 H3A(−102−86) |
| 4128 | CCACCTTCAGGCAACCC | SHANK3 H3A(−99−83) |
| 4129 | AGGCCACCTTCAGGCAA | SHANK3 H3A(−96−80) |
| 4130 | CTCAGGCCACCTTCAGG | SHANK3 H3A(−93−77) |
| 4131 | AGCCTCAGGCCACCTTC | SHANK3 H3A(−90−74) |
| 4132 | GCAAGCCTCAGGCCACC | SHANK3 H3A(−87−71) |
| 4133 | CCGGCAAGCCTCAGGCC | SHANK3 H3A(−84−68) |
| 4134 | CCTCCGGCAAGCCTCAG | SHANK3 H3A(−81−65) |
| 4135 | CTTCCTCCGGCAAGCCT | SHANK3 H3A(−78−62) |
| 4136 | CGCCTTCCTCCGGCAAG | SHANK3 H3A(−75−59) |
| 4137 | ACCCGCCTTCCTCCGGC | SHANK3 H3A(−72−56) |
| 4138 | ATCACCCGCCTTCCTCC | SHANK3 H3A(−69−53) |
| 4139 | AACATCACCCGCCTTCC | SHANK3 H3A(−66−50) |
| 4140 | CTGAACATCACCCGCCT | SHANK3 H3A(−63−47) |
| 4141 | CATCTGAACATCACCCG | SHANK3 H3A(−60−44) |
| 4142 | CATCATCTGAACATCAC | SHANK3 H3A(−57−41) |
| 4143 | CTCCATCATCTGAACAT | SHANK3 H3A(−54−38) |
| 4144 | GGCCTCCATCATCTGAA | SHANK3 H3A(−51−35) |
| 4145 | CAAGGCCTCCATCATCT | SHANK3 H3A(−48−32) |
| 4146 | CACCAAGGCCTCCATCA | SHANK3 H3A(−45−29) |
| 4147 | TGGCACCAAGGCCTCCA | SHANK3 H3A(−42−26) |
| 4148 | GCCTGGCACCAAGGCCT | SHANK3 H3A(−39−23) |
| 4149 | TCAGCCTGGCACCAAGG | SHANK3 H3A(−36−20) |
| 4150 | CAGTCAGCCTGGCACCA | SHANK3 H3A(−33−17) |
| 4151 | CGTCAGTCAGCCTGGCA | SHANK3 H3A(−30−14) |
| 4152 | GGCCGTCAGTCAGCCTG | SHANK3 H3A(−27−11) |
| 4153 | ACCGGCCGTCAGTCAGC | SHANK3 H3A(−24−8) |
| 4154 | AACACCGGCCGTCAGTC | SHANK3 H3A(−21−5) |
| 4155 | TGGAACACCGGCCGTCA | SHANK3 H3A(−18−2) |
| 4156 | CTGGAACACCGGCCGTCAGTCAGCC | SHANK3 H3A(−25−1) |
| 4157 | CGCCTGGAACACCGGCCGTCAGTCA | SHANK3 H3A(−22+3) |
| 4158 | GTTCGCCTGGAACACCGGCCGTCAG | SHANK3 H3A(−19+6) |
| 4159 | CAGGTTCGCCTGGAACACCGGCCGT | SHANK3 H3A(−16+9) |
| 4160 | CTTCAGGTTCGCCTGGAACACCGGC | SHANK3 H3A(−13+12) |
| 4161 | CTTCTTCAGGTTCGCCTGGAACACC | SHANK3 H3A(−10+15) |
| 4162 | GAACTTCTTCAGGTTCGCCTGGAAC | SHANK3 H3A(−7+18) |
| 4163 | CCTGGAACACCGGCCGT | SHANK3 H3A(−16+1) |
| 4164 | TCGCCTGGAACACCGGC | SHANK3 H3A(−13+4) |
| 4165 | GGTTCGCCTGGAACACC | SHANK3 H3A(−10+7) |
| 4166 | TCAGGTTCGCCTGGAAC | SHANK3 H3A(−7+10) |
| 4167 | TCTTCAGGTTCGCCTGG | SHANK3 H3A(−4+13) |
| 4168 | ACTTCTTCAGGTTCGCC | SHANK3 H3A(−1+16) |
| 4169 | ATGGAAGACTGGGCAGCTGCAGTGG | SHANK3_H3′UTR(+506+531) |
| 4170 | GTGGCAGTTGCAGTGAGTGGTCGGA | SHANK3_H3′UTR(+533+557)mm1 |
| 4171 | GTGAGGTGCCGGAGATGGAAGACTG | SHANK3 H3`UTR(+521+545)mm1 |
| 4172 | GCAGTGAGGGGCCGGAGATGGAAGA | SHANK3_H3`UTR (+524+548) |
| 4173 | GCAGTGAGGGTCCGGAGATGGAAGA | SHANK3_H3`UTR (+524+548)mm1 |
| 4174 | GCAGTGAGGTGCCGTAGATGGAAGA | SHANK3_H3′UTR (+524+548)mm2 |
| 4175 | TGGAAAAGCCCATAGTTGAGCGCGT | SHANK3 H1A(+239+263) |
| 4176 | GTCCAGGCGCAGGCACTTCTGCAGC | SHANK3 H1A(+150+174) |
| 4177 | GCTCCTCATCCAGGAACTTGCC | SHANK3 H1A(+286+307) |
| 4178 | CCCATAGTTGAGCGCGTCCTGGAGG | SHANK3 H1A(+231+255) |
| 4179 | CCCATAGTTGAGCGCGTCCTTAATG | SHANK3 H1A(+231+255)mm |
| 4180 | CTCATCCAGGAACTTGCCTTAG | SHANK3 H1A(+282+303)mm |
| 4181 | CTCATCCAGGAACTTGCCGGCG | SHANK3 H1A(+282+303) |
| 4182 | CTTTGTGTGAAGCTTTGCAAACTGC | SHANK3 H2A(+48+72) |
| 4183 | ATCATCGATGAGGTTCTGGGCATAA | SHANK3 H2A(+21+45) |
| 4184 | GAGCGGAGCCTCCAGTGCGGTGTGT | SHANK3 5′UTR(−2069−2045) |
| 4185 | GGCGAGAAGTGGAAAGAAAAACA | SHANK3_5′UTR (−858−837)mm |
| 4186 | GGGCACGAAGACCAAGGTAGGG | SHANK3_5′UTR (−879−857) |
| 12646 | GGCATAAACTCGCCTGT | SHANK3 H2A(+3+19)mm |
| 12647 | GGCATAAACTCGCCGCAGG | SHANK3 H2A(+3+21)mm |
| 12648 | GGCATAAACTCGCCGCTTG | SHANK3 H2A(+3+21) |
| 12649 | GGTTCTGGGCATAAACT | SHANK3 H2A(+22+40) |
| 12650 | GGTTCTGGGCATAAACTCG | SHANK3 H2A(+20+40) |
| 12651 | TTCTGGGCATAAACTCGCC | SHANK3 H2A(+8+26) |
| 12652 | GCATAAACTCGCCGCTTGT | SHANK3 H2A(+12+32) |
| 12653 | GCATAAACTCGCCGAAT | SHANK3 H2A(+14+32)mm |
| 12654 | GCATAAACTCGCCGCTGAT | SHANK3 H2A(+12+32)mm |
| 12655 | AGCGGAACTGACCCTGT | SHANK3_H22P(+1720+1736) |
| 12656 | GAAGCGGAACTGACCCTGT | SHANK3_H22P(+1718+1736) |
| 12657 | AGCTGAACTGACTCTGT | SHANK3_H22P(+1720+1736)mm |
| 12658 | GAAGCTGAACTGACTCTGT | SHANK3_H22P(+1718+1736)mm |
| 12659 | AGGGCACTAAGCAATGTA | SHANK3_H22A(+746+763) |
| 12660 | TCCTGACTGTGCGCTGGG | SHANK3_H22A(+783+800) |
| 12661 | GGAGGTGAAGCGGAACTGACCCTGT | SHANK3_H3′UTR(+1712+1736) |
| 12662 | TGAAGCGGAACTGACCCTGTAAAGT | SHANK3_H3′UTR(+1707+1731) |
| 12663 | CCTGTAAAGTTAAAAACCCAAATTG | SHANK3_H3′UTR(+1692+1716) |
| 12664 | CTTGTATCGAAACTGCTCAGATAAA | SHANK3 H2A(−13+12) |
| 12665 | AAACTCGCCGCTTGTATCGAAACTG | SHANK3 H2A(−3+22) |
| 12666 | TTCTGGGCATAAACTCGCCGCTGTT | SHANK3 H2A(+8+32)mm |
| 12667 | ATCATCGATGAGGTTCTGGGCACGA | SHANK3 H2A(+21+45)mm |
| 12668 | CTTTGCAAACTGCTTATCATCGATG | SHANK3 H2A(+36+60) |
| 12669 | CGTGATCCTTTACCTTTGTGTGAAG | SHANK3 H2D(+12−13) |
| 12670 | GCCACCTCCTCGTGATCCTTTACCT | SHANK3 H2D(+2−23)mm |
| 12671 | CAGGAGCCACCTCCTCGTGATCCTT | SHANK3 H2D(−4−28)mm |
| 12688 | GGAGGTGAAGCTGAACTGACTCTGT | SHANK3_H3′UTR(+1712+1736)m |
| 12689 | GTGTCAGGCAAAGCCAAGGCTCTGG | SHANK3 H17D(−22−46)mm |
| 12690 | AAGCCAAGGCCCTGTGGACACAGGC | SHANK3 H17D(−12−36)mm |
| 12691 | AAGGCCCTGTGGACACAGGCAGATG | SHANK3 H17D(−7−31)mm |
| 12692 | GGGTCAGGCAAAGCCAAGGCCCTGG | SHANK3 H17D(−22−46) |
| 12693 | AGGCAAAGCCAAGGCCCTGTGGACA | SHANK3 H17D(−17−41)mm |

TABLE 5
Exemplary PMO and MOE Oligonucleotide Sequences Targeting
SHANK3 mRNA 5′ UTR or 3′ UTR or SHANK3 5′ PNCR

| SEQ | | | PMO/ |
|---|---|---|---|
| ID NO | ASO Sequence | ASO Name | MOE |
| 293 | TTGTATCGAAACTCCAG | SHANK3 H1: 2 5UTR(+355+371) | MOE27 |
| 299 | TGGGCATAAACTCGCCG | SHANK3 H2 5UTR(+373+389) | MOE33 |
| 301 | AGGTTCTGGGCATAAAC | SHANK3 H2 5UTR(+379+395) | MOE35 |
| 302 | ATGAGGTTCTGGGCATA | SHANK3 H2 5UTR(+382+398) | MOE36 |
| 304 | TCATCGATGAGGTTCTG | SHANK3 H2 5UTR(+388+404) | MOE38 |
| 305 | TTATCATCGATGAGGTT | SHANK3 H2 5UTR(+391+407) | MOE39 |
| 306 | TGCTTATCATCGATGAG | SHANK3 H2 5UTR(+394+410) | MOE40 |
| 307 | AACTGCTTATCATCGAT | SHANK3 H2 5UTR(+397+413) | MOE41 |
| 308 | GCAAACTGCTTATCATC | SHANK3 H2 5UTR(+400+416) | MOE42 |
| 309 | TTTGCAAACTGCTTATC | SHANK3 H2 5UTR(+403+419) | MOE43 |
| 311 | TGAAGCTTTGCAAACTG | SHANK3 H2 5UTR(+409+425) | MOE45 |
| 313 | TTTGTGTGAAGCTTTGC | SHANK3 H2 5UTR(+415+431) | MOE47 |
| 315 | TTCGCCTTTGTGTGAAG | SHANK3 H2: 3 5UTR(+421+437) | MOE49 |
| 318 | TTCTTCAGGTTCGCCTT | SHANK3 H2: 3 5UTR(+430+446) | MOE52 |
| 606 | CGATGAGGTTCTGGGCATAAACTCG | SHANK3 H2 5UTR(+376+400) | PMO33 |
| 797 | GGCCGGAGATGGAAGAC | SHANK3 H22 3UTR(+1115+1131) | MOE60 |
| 1193 | GCGGAACTGACCCTGTA | SHANK3 H22 3UTR(+2303+2319) | MOE55 |
| 1195 | GGTGAAGCGGAACTGAC | SHANK3 H22 3UTR(+2309+2325) | MOE57 |
| 1847 | CGGAACTGACCCTGTAAAGTTAAAA | SHANK3 H22 3UTR(+2294+2318) | PMO40 |
| 1934 | CTGACCCTGTAAAGTTAAAACCCA | SHANK3_H3′UTR(+1697+1721) | PMO6 |
| 1935 | GGCATAAACTCGCCGCTTGTACTGA | SHANK3 H2A(+3+27) | PMO34 |
| 1936 | GGCATAAACTCGCCGCTTGTATCGA | SHANK3 H2A(+3+27)mm | PMO35 |
| 1937 | CTCCCCGTGATCCTTTACCTTTGTG | SHANK3 H2D(+7−18) | PMO37 |
| 2858 | TTCTGGGCATAAACTCGCCGCTTGT | SHANK3 H2A(+8+32) | PMO65 |
| 2874 | TCCTTTACCTTTGTGTGAAGCTTTG | SHANK3 H2D(+17−8) | PMO70 |
| 3510 | GGCATAAACTCGCCGCT | SHANK3 H2A(+3+19) | MOE5 |
| 12644 | GCACAGCAAGGAGGCTCCATCTCAG | SHANK3 H20A(−27−3) | PMO17 |
| 12666 | TTCTGGGCATAAACTCGCCGCTGTT | SHANK3 H2A(+8+32)mm | PMO66 |
| 12669 | CGTGATCCTTTACCTTTGTGTGAAG | SHANK3 H2D(+12−13) | PMO71 |
| 12671 | CAGGAGCCACCTCCTCGTGATCCTT | SHANK3 H2D(−4−28)mm | PMO73 |
| 12688 | GGAGGTGAAGCTGAACTGACTCTGT | SHANK3_H3′UTR(+1712+1736)m | PMO43 |
| 12690 | AAGCCAAGGCCCTGTGGACACAGGC | SHANK3 H17D(−12−36)mm | PMO53 |

CPP Amino Acid Sequence (Artificial/Synthetic)
SEQ ID NO: 12694
RRSRTARAGRPGRNSSRPSAPR

The sequence listing for this application is provided in five separate accompanying .xml format files, the entire contents of each of which are incorporated herein by reference. The sequence listing .xml file names, (WIPO Sequence program auto-generated) sequence numbering ranges, corresponding Applicant-assigned SEQ ID NO ranges of each of these, and the first sequence in each file is summarised in Table 6 below.

TABLE 6
Sequence listing file (auto-generated) numbering
ranges and corresponding SEQ ID NOS

| | Auto- | | |
|---|---|---|---|
| | generated | | |
| | sequence | | |
| | numbering | Corre- | |
| | range in | spond- | |
| | Correspond- | ing | |
| File Name | ing | SEQ ID NO | First |
| (.xml file) | file | range | Sequence in file |
| 536829PCT | 1-12694 | 1-12694 | SEQ ID NO: 1 |
| 1 of 5 | | | (auto-numbered as Seq. 1) |
| | | | CACCACTCAGGCCAGGCCAG . . . |
| | | | (first 20 bases only; |
| | | | sequence continues) |
| 536829PCT | 1-7307 | 12695-20001 | SEQ ID NO: 12695 |
| 2 of 5 | | | (auto-numbered as Seq. 1) |
| | | | CAGGGCGGCGGGGCTCA |
| 536829PCT | 1-8000 | 20002-28001 | SEQ ID NO: 20002 |
| 3 of 5 | | | (auto-numbered as Seq. 1) |
| | | | CAGTTTTGTTAATAAGA |
| 536829PCT | 1-8000 | 28002-36001 | SEQ ID NO: 28002 |
| 4 of 5 | | | (auto-numbered as Seq. 1) |
| | | | CGTGAGTCACGCACACACCAGGCTT |
| 536829PCT | 1-3536 | 36002-39537 | SEQ ID NO: 36002 |
| 5 of 5 | | | (auto-numbered as Seq. 1) |
| | | | AGAAGTCTGTTGTCAGTTGGACCAC |

If any discrepancies are identified between (i) the accompanying sequence listing files sequences, the numbering corresponding to the SEQ ID NOs, or the corresponding headings/names provided in the accompanying sequence listing files; and (ii) the sequences and SEQ ID NOs disclosed in the present specification and Appendix therein, the sequences and SEQ ID NOs (and SEQ ID NO ranges) provided in the present specification and Appendix are to be accepted as correct.

1. An antisense oligonucleotide that binds within a targeted portion of the:
(i) 3′ UTR of a SHANK3 mRNA;
(ii) 5′ untranslated region (UTR) of a SHANK3 mRNA; or
(iii) 5′-proximal non-coding region (PNCR) of a SHANK3 pre-mRNA;
whereby binding of the antisense oligonucleotide within the targeted portion in a mammalian cell results in an increased level of SHANK3 protein in the mammalian cell.

2. A vector for expression, in a mammalian neuron, of an antisense RNA (AR) that binds within a targeted portion of the:
(i) 3′ UTR of a SHANK3 mRNA;
(ii) 5′ untranslated region (UTR) of a SHANK3 mRNA; or
(iii) 5′-proximal non-coding region (PNCR) of a SHANK3 pre-mRNA;
whereby binding of the AR within the targeted portion in a mammalian cell results in an increased level of SHANK3 protein in the mammalian cell.

3. (canceled)

4. (canceled)

5. (canceled)

6. (canceled)

7. (canceled)

8. (canceled)

9. (canceled)

10. (canceled)

11. The antisense oligonucleotide according to claim 1, wherein the antisense oligonucleotide comprises a backbone modification.

12. The antisense oligonucleotide according to claim 11 wherein the antisense oligonucleotide comprises a backbone modification comprising a phosphorothioate linkage or a phosphorodiamidate linkage or wherein the antisense oligonucleotide comprises a phosphorodiamidate morpholino, a locked nucleic acid, a peptide nucleic acid, a 2′-O-methyl, a 2′-fluoro, or a 2′-O-methoxyethyl moiety.

13. (canceled)

14. The antisense oligonucleotide according to claim 11, wherein the antisense oligonucleotide comprises at least one modified sugar moiety or wherein the antisense oligonucleotide comprises a 2′-O-methoxyethyl moiety.

15. (canceled)

16. (canceled)

17. (canceled)

18. (canceled)

19. The antisense oligonucleotide according to claim 1, wherein the nucleotide sequence of the antisense oligonucleotide consists of 17 to 30 nucleotides.

20. (canceled)

21. The antisense oligonucleotide according to claim 1, wherein the antisense oligonucleotide is linked to a cell-penetrating peptide (CPP).

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. The antisense oligonucleotide according to claim 1, wherein the nucleotide sequence of the antisense oligonucleotide corresponds to any one of SEQ ID NOs: 1847, 1193, 293, 299, 301, 302, 304-309, 311, 313, 315, 318, 606, 797, 1195, 1934-1937, 2858, 2874, 3510, 12644, 12666, 12669, 12671, 12688, or 12690.

35. (canceled)

36. (canceled)

37. The antisense oligonucleotide according to claim 1, wherein the binding is within a targeted portion of the 3′ UTR corresponding to SEQ ID NO: 2.

38. (canceled)

39. (canceled)

40. (canceled)

41. (canceled)

42. The antisense oligonucleotide, vector, or composition according to claim 37, wherein the nucleotide sequence of the antisense oligonucleotide corresponds to any one of SEQ ID NOs: 623-1934, 4169-4174, 12645, 12655-12663, or 12688.

43. The antisense oligonucleotide, vector, or composition according to claim 37, wherein the nucleotide sequence of the antisense oligonucleotide corresponds to any one of SEQ ID NOs: 1847, 1193, 1852, 1934, 12661-12663, or 12688.

44. The antisense oligonucleotide according to claim 1, further comprising a delivery nanocarrier, wherein the nanocarrier is complexed with the antisense oligonucleotide.

45. (canceled)

46. (canceled)

47. (canceled)

48. A method for preventing or treating a condition associated with SHANK3 haploinsufficiency, the method comprising administering to a subject in need thereof a therapeutically effective amount of the antisense oligonucleotide of claim 1.

49. The method according to claim 48, wherein the condition is Phelan-McDermid syndrome, an autism spectrum disorder, schizophrenia, or an intellectual disability.

50. (canceled)

51. (canceled)

52. (canceled)

53. (canceled)

54. (canceled)

55. (canceled)

56. (canceled)

57. (canceled)

58. (canceled)

59. (canceled)

60. (canceled)
